(19) United States
(12) Patent Application Publication
(10) Pub. No.: US 2024/0034731 A1
(43) Pub. Date: Feb. 1, 2024

(54) AZA-QUINAZOLINE COMPOUNDS AND METHODS OF USE

(71) Applicant: Iambic Therapeutics, Inc., LOS ANGELES, CA (US)

(72) Inventors: William VERNIER, Vista, CA (US); Quynh Nhu NGUYEN, Redwood City, CA (US); Nomaan REZAYEE, San Diego, CA (US); Laurent GOMEZ, San Diego, CA (US); Chao ZHANG, San Diego, CA (US); Thomas Francis MILLER, III, La Jolla, CA (US); Frederick Roy MANBY, Bristol (GB)

(21) Appl. No.: 18/357,320

(22) Filed: Jul. 24, 2023

Related U.S. Application Data
(63) Continuation of application No. PCT/US23/70457, filed on Jul. 18, 2023.
(60) Provisional application No. 63/512,046, filed on Jul. 5, 2023.
(60) Provisional application No. 63/390,251, filed on Jul. 18, 2022.

(51) Int. Cl. C07D 471/04; C07D 519/00; A61P 35/00

(52) U.S. Cl. CPC C07D 471/04; C07D 519/00; A61P 35/00

(57) ABSTRACT

Substituted aza-quinazoline compounds, conjugates, and pharmaceutical compositions for use in the treatment of cancer are disclosed herein. The disclosed compounds are useful, among other things, in the inhibition of CDK. In certain aspects, the disclosure generally relates to substituted quinolinone amide compounds or salts of Formula (I), (IA), (IAA), (IAAA), (IB), (IBB), (IC), (ICC), (ID), (IDD), (IE), and (IEE) and pharmaceutical compositions thereof.

### CROSS-REFERENCE TO RELATED APPLICATIONS

This application is continuation of International Application Number PCT/US23/70457, filed Jul. 18, 2023, which claims the benefit of U.S. Provisional Patent Application No. 63/390,251 filed Jul. 18, 2022, and U.S. Provisional Patent Application No. 63/512,046 filed Jul. 5, 2023. The entire contents of the aforementioned patent applications are incorporated herein by reference.

### BACKGROUND OF THE INVENTION

Division and proliferation of mammalian cells mediated by the cell cycle is an important and fundamental biological process, which controls production and generation of cells with critical biological functions. Cell cycle is a highly regulated process and responds to a complex set of cell signals within the cell and externally. The complex network of cell signaling, including components promoting and suppressing cancer, plays a key role controlling the cell cycle. Gain-of-function of tumor-promoting components or loss-of-function of tumor-suppressing products can lead to unregulated cell cycle and subsequently tumorigenesis.

Cyclins and cyclin-dependent kinases (CDKs) are crucial for driving and controlling cell cycle transitions and cell division (34176404). Cyclin is a family of proteins whose expression levels vary at different stages in the cell cycle. Cyclins bind and activate CDKs during different stages of cell cycle, of which the progression is tightly synchronized involving sequential activation of several cyclin-CDK complexes. Of more than 20 CDKs discovered so far, CDK1, 2, 4, 6 have been reported to play a direct role in cell cycle progression. CDK4-cyclin D and CDK6-cyclin D complexes are essential for entry in G1 phase of cell cycle. CDK2-cyclin E complex regulates progression from G1 into S phase, while CDK2-cyclin A is required during S phase. CDK1-cyclin A complex promotes entry into M phase, and mitosis is further regulated by CDK1-cyclin B complex. Progressive phosphorylation of retinoblastoma (Rb) by CDK4-cyclin D, CDK6-cyclin D and CDK2-cyclin E releases the G1 transcription factor, E2F, and promotes S-phase entry. Activation of CDK2-cyclin A during early S-phase promotes phosphorylation of endogenous substrates that permit DNA replication and inactivation of E2F, for S-phase completion.

Dysregulation of cell-cycle machinery is a hallmark of cancer, leading to overactivation of CDKs and uncontrolled cell division and proliferation. Genetic alterations of the genes encoding cyclin D, CDK4/6, and CDK4/6-inhibiting proteins (such as p21, p27) all contribute to tumorigenesis. Cyclin E, the regulatory cyclin for CDK2, is frequently overexpressed in cancer. Since tumor development is closely related to gene mutation and deregulation of CDK and its regulators, CDK inhibitors are useful for anticancer therapy. CDK inhibitors have been developed as cancer therapy since the early 90s, with multiple FDA-approved drugs (Palbociclib, ribociclib and abemaciclib). However, these early generation CDK inhibitors on the market have poor selectivity and high toxicity (such as myelosuppression), leading to adverse effects limiting clinical dosing level for further patient benefit. There remains an unmet medical need to develop novel CDK inhibitors with better selectivity and less side effects for normal cells.

### SUMMARY OF THE INVENTION

The present disclosure generally relates to substituted quinolinone amide compounds or salts of Formula (I), (IA), (IAA), (IAAA), (IB), (IBB), (IC), (ICC), (ID), (IDD), (IE), and (IEE) and pharmaceutical compositions thereof. The substituted quinolinone amide compounds or salts of Formula (I), (IA), (IAA), (IAAA), (IB), (IBB), (IC), (ICC), (ID), (IDD), (IE), and (IEE) disclosed herein may be used for the treatment of abnormal cell growth, such as cancer, in a subject in need thereof.

In some aspects, methods of treating cancer may comprise administering a compound or pharmaceutically acceptable salt of any one of Formula (I), (IA), (IAA), (IAAA), (IB), (IBB), (IC), (ICC), (ID), (IDD), (IE), and (IEE) in an individual in need thereof.

In certain aspects, the disclosure provides a compound re resented by Formula (I0):

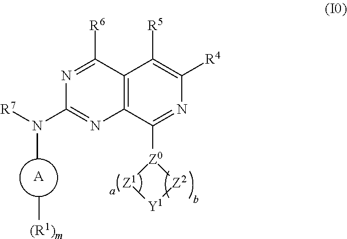

wherein,
A is a ring selected from optionally substituted carbocycle, optionally substituted 4- to 8-membered heterocycle, and optionally substituted isoindoline;
Z⁰ is —C(H)— or nitrogen;
each of Z¹, Z², and Y¹ are independently selected from —C(R²)₂—, —C(O)—, —NR³—, —N(C(O)R²)—, —NS(O₂)R², —O—, —S—, —S(O)—, and —S(O)₂—;
each of a and b are independently selected from 1, 2, 3, and 4;
each R is independently selected from halogen, —CN, —NO₂, optionally substituted alkyl, optionally substituted alkenyl, optionally substituted alkynyl, optionally substituted carbocycle, and optionally substituted heterocycle;
m is selected from 0 to 5;
each R² is independently selected from hydrogen, halogen, —CN, —OH, optionally substituted alkoxy, optionally substituted alkyl, optionally substituted cycloalkyl, optionally substituted —O-cycloalkyl, and optionally substituted heterocycloalkyl, or two R² substituents come together to form an optionally substituted heterocycle or an optionally substituted carbocycle, or R² and R³ substituents come together to form an optionally substituted heterocycle;
each R³ is independently selected from hydrogen, optionally substituted alkyl, optionally substituted C₃-4 carbocycle, and optionally substituted 3- to 4-membered heterocycloalkyl;
R⁴ is selected from hydrogen, halogen, —CN, optionally substituted C₁-4 alkyl, optionally substituted C₃-4 carbocycle, and optionally substituted 3- to 4-membered heterocycloalkyl;
each of R⁵, R⁶, is independently selected from hydrogen, halogen, —CN, optionally substituted C₁-4 alkyl, optionally substituted C₃-4 carbocycle, and optionally substituted 3- to 4-membered heterocyclcoalkyl; and
R⁷ is selected from hydrogen and optionally substituted C₁-4 alkyl, and
wherein if A is optionally substituted phenyl, m is from 1 to 5 and at least one R¹ is a heterocycloalkyl,
wherein if A is optionally substituted pyridine, optionally substituted pyridazine, or optionally substituted pyrimidine, R⁴ is selected from hydrogen, halogen, and —CN,
wherein if A is an optionally substituted piperidine sulfonamide, then either (i) Y¹ is —C(R²)₂— and the two R² substituents come together to form a ring selected from an optionally substituted heterocycle and an optionally substituted carbocycle, or (ii) R⁴ is selected from hydrogen, halogen, methyl, and —CN, and
wherein if A-R¹ is

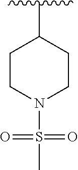 [SMILES: *C1CCN(S(C)(=O)=O)CC1]

and Z⁰ is CH, then R⁴ is methyl or cyclopropyl.

In certain aspects, the disclosure provides a pharmaceutical composition comprising a compound described herein and a pharmaceutically acceptable excipient.

In certain aspects, the disclosure provides a method of treating cancer comprising administering to a subject in need thereof a compound or pharmaceutical composition described herein. In certain aspects, the disclosure provides a method of inhibiting a cyclin dependent kinase (CDK) in a cell with a compound or pharmaceutically acceptable salt or the pharmaceutical composition described herein.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Basic functions for cell regulation, cell division, and cell proliferation are controlled by cyclin-dependent kinases (CDKs) activated by regulatory subunits such as cyclins. CDK inhibitors are useful in the treatment of cancer due to CDKs role in cell regulation. It has been shown that increased activity or transient abnormal activation of CDKs leads to the development of tumors; development of tumors are often associated with changes in the CDKs or regulators of CDKs.

CDKs bind to cyclin, which a regulatory protein., and without cyclin, it has little kinase activity. The cyclin-CDK complex is an active kinase typically modulated by phosphorylation and other binding proteins. There are currently 21 CDKs and 5 CDK-like genes that are known in the human genome. While many of the CDKs have been linked to transcription, CDK2, CDK4, and CDK6 are associated with the cell cycle. CDK2 is associated with DNA replication in higher eukaryotes whereas CDK4 and CDK6 are associated with various growth-regulatory signals.

CDK2 overexpression is associated with abnormal regulation of the cell cycle. Cyclin E, the cyclin partner of CDK2, binds to CDK2 to form an active kinase complex. The CDK2-cyclin E complex is important in the regulation of the G1/S transition, centrosome replication, and histone biosynthesis. Progressive phosphorylation can release the G1 transcription factor E2F and promote entry into the S phase. Another cyclin partner of CDK2, cyclin A, can bind and activate CDK2 during the initial phase of the S phase, and promote endogenous substrate phosphorylation, which allows DNA replication and E2F inactivation to complete the S phase.

CDK4 and CDK6 are also associated with the cell cycle. CDK4 and CDK6 inhibitors can arrest the cell cycle form the G1 to S phase by blocking phosphorylation of Rb protein and inhibiting proliferation of Rb-positive tumor cells. Besides cell cycle activity, CDK4 and CDK6 inhibitors can also suppress tumor growth through other mechanisms including, but not limited to inducing senescence, promoting anti-tumor immune responses, regulation of cell metabolism, and enhancing cytostasis caused by signaling pathway inhibitors.

### Definitions

Unless defined otherwise, all technical and scientific terms used herein have the same meaning as is commonly understood by one of skill in the art to which this invention belongs.

As used in the specification and claims, the singular form “a”, “an” and “the” includes plural references unless the context clearly dictates otherwise.

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Heteroalkyl” refers to an alkyl group, as defined above, having from one or more carbon atoms replaced with a heteroatom, such as wherein the heteroatom is individually selected from N, O and S at each replacement location Additional heteroatoms can also be useful, including, but not limited to B, Al, Si and P. The heteroatoms can also be oxidized, such as, but not limited to, —S(O)— and —S(O)₂—. For example, heteroalkyl can include ethers, thioethers and alkyl-amines. Heteroalkyl consisting of the stated number of carbon atoms and may include one or more heteroatoms selected from the group consisting of O, N, Si and S, wherein the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Two heteroatoms may be consecutive, such as, for example, —CH₂NHOCH₃ and —CH₂OSi(CH₃)₃. Heteroalkyl can include any stated number of carbon atoms as defined herein and in the definition of alkyl.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises five to eight carbon atoms (e.g., C₅-C₈ alkenylene). In other embodiments, an alkenylene comprises two to five carbon atoms (e.g., C₂-C₅ alkenylene). In other embodiments, an alkenylene comprises three to five carbon atoms (e.g., C₃-C₅ alkenylene).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene).

“Heteroalkylene” refers to a straight or branched divalent heteroalkyl chain linking the rest of the molecule to a radical group, consisting of heteroatoms such as N, O and S. Additional heteroatoms can also be useful, including, but not limited to, B, Al, Si and P. The heteroalkylene chain is attached to the rest of the molecule through a single bond and to the radical group through a single bond. In certain embodiments, a heteroalkylene comprises one heteroatom. In certain embodiments, a heteroalkylene comprises two heteroatoms. In certain embodiments, a heteroalkylene comprises three heteroatoms. In certain embodiments, a heteroalkylene comprises four heteroatoms. In certain embodiments, a heteroalkylene comprises fie heteroatoms. In certain embodiments, the heteroatoms can be N, O, S, Si, or P, or a combination thereof. In certain embodiments, the heteroatoms can be N O, or S, or a combination thereof. In certain embodiments, the heteroatoms can be N, O, or a combination thereof.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively.

The term “carbocycle” as used herein refers to a saturated, unsaturated or aromatic ring in which each atom of the ring is carbon. Carbocycle includes 3- to 10-membered monocyclic rings, 5 to 12-membered bicyclic rings, 5- to 12-membered spiro bicycles, and 5- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle further includes spiro bicyclic rings such as spiropentane. A bicyclic carbocycle includes any combination of ring sizes such as 3-3 spiro ring systems, 4-4 spiro ring systems, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, naphthyl, and bicyclo[1.1.1]pentanyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, 5- to 12-membered spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, spiropentane, norbornyl (i.e., bicyclo[2.2.1]heptanyl), decalinyl, 7,7 dimethyl bicyclo[2.2.1]heptanyl, bicyclo[1.1.1]pentanyl, and the like.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 5- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, 5- to 12-membered spiro bicycles, and 5- to 12-membered bridged rings. A monocylic heterocycle includes any saturated, unsaturated, and aromatic rings as valence permits. A monocyclic heterocycle includes but is not limited to, oxetane, azetidine, furan, tetrahydrofuran, pyrrole, pyrrolidine, pyran, piperidine, piperazine, imidazole, thiazole, morpholine, pyridine, and pyrimidine. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Examples of fused ring systems include, but are not limited to, isoindoline, isoquinoline, tetrahydroisoquinoline, 3-azabicyclo[3.1.0]hexane and 6-oxa-3-azabicyclo[3.1.1]heptane. A bicyclic heterocycle further includes spiro bicyclic rings, e.g., 5 to 12-membered spiro bicycles, such as but not limited to 2-azaspiro[3.3]heptane, 5-azaspiro[2.4]heptane, 2-oxa-6-azaspiro[3.3]heptane, 2,6-diazaspiro[3.3]heptane, 1-thia-6-azaspiro[3.3]heptane, 6-azaspiro[3.4]octane, 2,6-diazaspiro[3.4]octane, 2-thia-6-azaspiro[3.4]octane, 4-oxa-7-azaspiro[2.5]octane, 2-azaspiro[4.4]nonane, 2,7-diazaspiro[4.4]nonane, 2-oxa-6-azaspiro[3.5]nonane, 7-oxa-2-azaspiro[3.5]nonane, 2-azaspiro[4.5]decane, 2,8-diazaspiro[4.5]decane, 8-oxa-2-azaspiro[4.5]decane, and 2-oxa-7-azaspiro[4.5]decane.

The term “heteroaryl” refers to a radical derived from a 5 to 18 membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is aromatic, i.e., it contains a cyclic, delocalized (4n+2) π-electron system in accordance with the Huckel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, benzimidazolyl, 1,3-benzodioxolyl, benzofuranyl, benzoxazolyl, benzo[d]thiazolyl, benzodioxinyl, benzopyranyl, benzopyranonyl, benzofuranyl, benzofuranonyl, benzothienyl (benzothiophenyl), benzotriazolyl, furanyl, isothiazolyl, imidazolyl, indazolyl, indolyl, indazolyl, isoindolyl, indolinyl, isoindolinyl, pyrrolyl, pyrazolyl, pyridinyl, pyridopyrimidinyl, pyrazinyl, pyrimidinyl, pyridazinyl, pyrrolyl, quinazolinyl, quinolinyl, isoquinolinyl, tetrahydroquinolinyl, and thiophenyl (i.e. thienyl).

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, 5- to 12-membered spiro bicycles, and 5- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, azetidinyl, dioxolanyl, decahydroisoquinolyl, imidazolinyl, imidazolidinyl, isothiazolidinyl, isoxazolidinyl, morpholinyl, octahydroindolyl, octahydroisoindolyl, oxazolidinyl, oxetanyl, piperidinyl, piperazinyl, pyrrolidinyl, pyrazolidinyl, quinuclidinyl, thiazolidinyl, tetrahydrofuryl, trithianyl, tetrahydropyranyl, thiomorpholinyl, 3-azabicyclo[3.1.0]hexane, 2-azaspiro[3.3]heptane, 5-azaspiro[2.4]heptane, 2-oxa-6-azaspiro[3.3]heptane, 2,6-diazaspiro[3.3]heptane, 6-oxa-3-azabicyclo[3.1.1]heptane, 1-thia-6-azaspiro[3.3]heptane, 6-azaspiro[3.4]octane, 2,6-diazaspiro[3.4]octane, 2-thia-6-azaspiro[3.4]octane, 4-oxa-7-azaspiro[2.5]octane, 2-azaspiro[4.4]nonane, 2,7-diazaspiro[4.4]nonane, 2-oxa-6-azaspiro[3.5]nonane, 7-oxa-2-azaspiro[3.5]nonane, 2-azaspiro[4.5]decane, 2,8-diazaspiro[4.5]decane, 8-oxa-2-azaspiro[4.5]decane, 2-oxa-7-azaspiro[4.5]decane, and 1,1-dioxo-thiomorpholinyl.

The term “heterocycloalkenyl” refers to an unsaturated ring with carbon atoms and at least one heteroatom and there is at least one double bond between two ring carbons. Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms. The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, spirocyclic and non-spirocyclic, aromatic and non-aromatic substituents of organic compounds. The permissible substituents can be one or more and the same or different for appropriate organic compounds.

In some embodiments, each substituent may individually include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

Double bonds to oxygen atoms, such as oxo groups, are represented herein as both “═O” and “(O)”. Double bonds to nitrogen atoms are represented as both “═NR” and “(NR)”. Double bonds to sulfur atoms are represented as both “═S” and “(S)”.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treatment via administration of a compound described herein does not require the involvement of a medical professional.

Table of Abbreviations

| | |
|---|---|
| ACN, MeCN | Acetonitrile |
| ° C. | Degree Celsius |
| BOC | tert-butoxycarbonyl |
| Cmpd | Compound |
| DCM | Dichloromethane |
| DIEA or DIPEA | Diisopropylethylamine |
| DMF | Dimethylformamide |
| DMSO | Dimethylsulfoxide |
| EA, ETOAc, ETAC | Ethyl acetate |
| EDC, EDAC or | 1-Ethyl-3-(3- |
| EDCI | dimethylaminopropyl)carbodiimide |
| ESI | Electrospray ionization |
| HATU | Hexafluorophosphate Azabenzotriazole |
| | Tetramethyl Uronium |
| HOBt | Hydroxybenzotriazole |
| HPLC | High Performance Liquid Chromatography |
| IC₅₀ | Half minimal (50%) inhibitory |
| | concentration |
| LCMS | Liquid Chromatography Mass |
| | Spectrometry |
| [M + H+]+ or | Mass peak plus hydrogen |
| (MH)+ | |
| [M − H−]−or (MH)− | Mass peak minus hydrogen |
| Me | Methyl |
| MeOH | Methanol |
| MS | Mass spectrometry |
| N | Normal |
| PE | Petroleum ether |
| PMB | (4-methoxyphenyl)methanamine or para- |
| | methoxy benzyl |
| Prep-TLC | Preparative thin layer chromatography |
| PyBroP | bromotri(pyrrolidino)phosphonium |
| | hexafluorophosphate |
| RP | Reverse phase |
| RP-HPLC | Reverse phase high pressure liquid |
| | chromatography |
| RT or rt | Room temperature |
| TLC | Thin-layer chromatography |
| THF | Tetrahydrofuran |
| TFA | Trifluoroacetic acid |

### Compounds

The following is a discussion of compounds and salts thereof that may be used in the methods of the disclosure. In certain embodiments, the compounds and salts are described in Formulas (I), (IA), (IAA), (IAAA), (IB), (IBB), (IC), (ICC), (ID), (IDD), (IE), and (IEE).

In one aspect, disclosed herein is a compound represented by Formula (I):

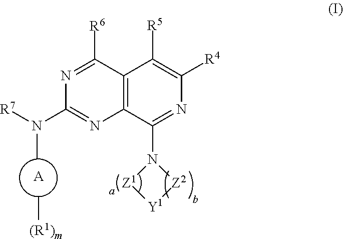

wherein,
A is a ring selected from optionally substituted carbocycle, optionally substituted 4- to 6 membered heterocycle, and optionally substituted isoindoline;
   each of Z¹, Z², and Y¹ are independently selected from —C(R²)₂—, —C(O)—, —NR³—, —N(C(O)R²)—, —NS(O₂)R², —O—, —S—, —S(O)—, and —S(O)₂—;
   each of a and b are independently selected from 1, 2, 3, and 4;
each R¹ is independently selected from halogen, —CN, —NO₂, optionally substituted alkyl, optionally substituted alkenyl, optionally substituted alkynyl, optionally substituted carbocycle, and optionally substituted heterocycle;
   m is selected from 0 to 5;
each R² is independently selected from hydrogen, halogen, —CN, —OH, —O—C₁₋₄ alkyl, optionally substituted alkyl, optionally substituted cycloalkyl, and optionally substituted heterocycloalkyl, or two R² substituents come together to form an optionally substituted heterocycle or an optionally substituted carbocycle, or R² and R³ substituents come together to form an optionally substituted heterocycle;
each R³ is independently selected from hydrogen, optionally substituted alkyl, optionally substituted C₃₋₄ carbocycle, and optionally substituted 3- to 4-membered heterocyclcoalkyl;
R⁴ is selected from hydrogen, halogen, —CN, optionally substituted C₁₋₄ alkyl, optionally substituted C₃₋₄ carbocycle, and optionally substituted 3- to 4-membered heterocyclcoalkyl;
each of R⁵, R⁶, is independently selected from hydrogen, halogen, —CN, optionally substituted C₁₋₄ alkyl, optionally substituted C₃₋₄ carbocycle, and optionally substituted 3- to 4-membered heterocyclcoalkyl; and
R⁷ is selected from hydrogen and optionally substituted C₁₋₄ alkyl, and
   wherein if A is optionally substituted phenyl, m is from 1 to 5 and at least one R¹ is a heterocycloalkyl,
   wherein if A is optionally substituted pyridine or optionally substituted pyrimidine, R⁴ is selected from hydrogen, halogen, and —CN, and
   wherein if A is an optionally substituted piperidine sulfonamide, then either (i) Y¹ is —C(R²)₂— and the two R² substituents come together to form a ring selected from an optionally substituted heterocycle and an optionally substituted carbocycle, or (ii) R⁴ is selected from hydrogen, halogen, and —CN.

Ring A can be any suitable ring known by one of skill in the art. In some embodiments, A is a ring selected from optionally substituted carbocycle, optionally substituted 4- to 10-membered heterocycle, and optionally substituted isoindoline. In some embodiments, A is a ring selected from optionally substituted carbocycle, optionally substituted 4- to 8-membered heterocycle, and optionally substituted isoindoline. In some embodiments, A is a ring selected from optionally substituted carbocycle, optionally substituted 4- to 6-membered heterocycle, and optionally substituted isoindoline. In some embodiments, A is selected from optionally substituted azetidine, optionally substituted piperidine, optionally substituted azabicyclo[3.1.0]hexane, optionally substituted phenyl, optionally substituted pyridine, optionally substituted pyrazole, optionally substituted isoindoline, and optionally substituted tetrahydroisoquinoline. In some embodiments, A is selected from optionally substituted pyridine, optionally substituted azabicyclo[3.1.0]hexane, optionally substituted azetidine, and optionally substituted tetrahydroisoquinoline. In some embodiments, A is not optimally substituted pyridine. In some embodiments, A is not optimally substituted pyrimidine.

In some embodiments, A is substituted with methyl, —SO₂Me, methylpiperidine, methylpiperazine, methylazaspiro[3.3]heptane, methyldiazaspiro[3.3]heptane, or a combination thereof. In some embodiments, A is substituted with methyl, —SO₂Me, —SO₂Me, methylpiperidine, methylpiperazine, or a combination thereof. In some embodiments, A is substituted with methyl, —SO₂Me, or a combination thereof. In some embodiments, A is substituted with —SO₂Me.

Z¹ and Z² can be any suitable functional group known by one of skill in the art. In some embodiments, Z¹ and Z² are independently selected from —C(R²)₂—, —C(O)—, —NR³—, —N(C(O)R²)—, —NS(O₂)R², —O—, —S—, —S(O)—, and —S(O)₂—. In some embodiments, Z¹ and Z² are independently selected from —C(R²)₂—, —C(O)—, —NR³—, —NS(O₂)R²—, —O—, and —S—. In some embodiments, Z¹ and Z² are independently selected from —C(R²)₂—, —NR³—, —O—, and —S—. In some embodiments, Z¹ and Z² are independently —C(R²)₂—.

Variables a and b can be any suitable number known by one of skill in the art. In some embodiments, each of a and b are independently selected from 1, 2, 3, and 4. In some embodiments, each of a and b are independently 1, 2, and 3. In some embodiments, each of a and b are independently selected from 1 and 2.

Y¹ can be any suitable functional group known by one of skill in the art. In some embodiments, Y¹ is selected from —C(R²)₂—, —C(O)—, —NR³—, —N(C(O)R²)—, —NS(O₂)R², —O—, —S—, —S(O)—, and —S(O)₂—. In some embodiments, Y¹ is selected from —C(R²)₂—, —C(O)—, —NR³—, —NS(O₂)R²—, —O—, and —S—. In some embodiments, Y¹ is selected from —C(R²)₂—, —NR³—, —O—, and —S—. In some embodiments, Y¹ is selected from —C(R²)₂— and —NR³. In some embodiments, Y¹ is —C(R²)₂— and two R² substituents come together to form a ring selected from an optionally substituted heterocycle and an optionally substituted carbocycle. In some embodiments, Y¹ is —C(R²)₂— and the two R² substituents come together to form a ring selected from an optionally substituted heterocycle.

In some embodiments, the N containing heterocyclic ring depicted as

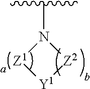

in Formula (I) is selected from optionally substituted azetidine, optionally substituted pyrrolidine, optionally substituted piperidine, optionally substituted piperazine, optionally substituted morpholine, optionally substituted tetrahydrothienopyrroledioxide, and optionally substituted dihydroindole. In some embodiments, the N containing heterocyclic ring depicted as

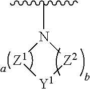

in Formula (I) is selected from

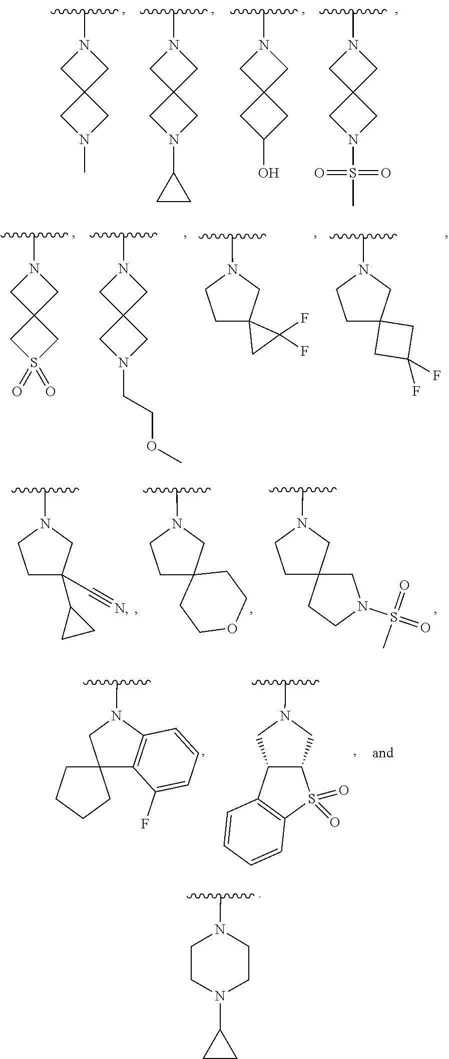

Variable m can be any suitable number own by one of skill in the art. In some embodiments m is selected from 0 to 5. In some embodiments, m is selected from 0 to 3. In some embodiments, m is selected from 0 to 2. In some embodiments, m is selected from 0 to 1. In some embodiments m is selected from 1 to 5. In some embodiments, m is selected from 1 to 3. In some embodiments, m is selected from 1 to 2. In some embodiments, m is 0. In some embodiments, m is 1. In some embodiments, m is 2. In some embodiments, m is 3.

R¹ can be any suitable functional group known by one of skill in the art. In some embodiments, each R¹ is independently selected from halogen, —CN, —NO₂, optionally substituted alkyl, optionally substituted alkenyl, optionally substituted alkynyl, optionally substituted carbocycle, and optionally substituted heterocycle. In some embodiments, each R¹ is independently selected from optionally substituted alkyl, optionally substituted carbocycle, and optionally substituted heterocycle. In some embodiments, each R¹ is independently selected from optionally substituted alkyl and optionally substituted heterocycle. In some embodiments, each R¹ is independently selected from methyl, optionally substituted piperidine, optionally substituted piperazine, optionally substituted azaspiro[3.3]heptane, and optionally substituted diazaspiro[3.3]heptane. In some embodiments, each R¹ is independently selected from methyl, ethyl, and optionally substituted diazaspiro[3.3]heptane.

R² can be any suitable functional group known by one of skill in the art. In some embodiments, each R² is independently selected from hydrogen, halogen, —CN, —OH, —O—C₁₋₄ alkyl, optionally substituted alkyl, optionally substituted cycloalkyl, and optionally substituted heterocycloalkyl, or two R² substituents come together to form an optionally substituted heterocycle or an optionally substituted carbocycle, or R² and R³ substituents come together to form an optionally substituted heterocycle. In some embodiments, each R² is independently selected from hydrogen, halogen, —CN, optionally substituted cycloalkyl, and optionally substituted heterocycloalkyl. In some embodiments, wherein each R² is independently selected from hydrogen, halogen, —CN, cyclopropyl, cyclobutyl, optionally substituted cycloalkyl, and optionally substituted heterocycloalkyl. In some embodiments, each R² is independently selected from hydrogen, fluoro, chloro, bromo, —CN, cyclopropyl, cyclobutyl, oxetane, and azetidine. In some embodiments, each R² is independently selected from hydrogen, fluoro, —CN, cyclopropyl, cyclobutyl, optionally substituted oxetane, and optionally substituted azetidine. In some embodiments, each R² is selected from hydrogen, fluoro, —CN and cyclopropyl. In some embodiments, each R² is selected from hydrogen, —CN and cyclopropyl.

In some embodiments, or two R² substituents come together to form an optionally substituted heterocycle or an optionally substituted carbocycle. In some embodiments, two R² substituents come together to form an optionally substituted heterocycle. In some embodiments, two R² substituents come together to form an optionally substituted carbocycle. In some embodiments, two R² substituents come together such that this

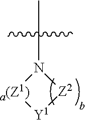

structure is

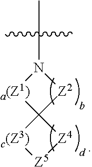

Each of Z¹, Z², Z³, Z⁴ and Z⁵ can be any suitable functional group known by one of skill in the art. In some embodiments, each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond. Each of Z¹ and Z² can be any functional group as described previously.

In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3, and 4. In some embodiments, each of a, b, c, and d are independently 1, 2, and 3. In some embodiments, each of a, b, c, and d are independently selected from 1 and 2.

In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

R³ can be any suitable functional group known by one of skill in the art. In some embodiments, each R³ is independently selected from hydrogen, optionally substituted alkyl, optionally substituted C₃₋₄ carbocycle, and optionally substituted 3- to 4-membered heterocyclcoalkyl, or R² and R³ substituents come together to form an optionally substituted heterocycle. In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁴ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁴ is selected from hydrogen, halogen, —CN, optionally substituted C₁₋₂ alkyl, and optionally substituted C₃₋₄ carbocycle. In some embodiments, R⁴ is selected from hydrogen and optionally substituted C₁ alkyl. In some embodiments, R⁴ is selected from hydrogen, methyl, and —CHF₂. In some embodiments, R⁴ is selected from hydrogen, halogen, and —CN. In some embodiments, R⁴ is selected from hydrogen. In some embodiments, R⁴ is not optionally substituted phenyl. In some embodiments, R⁴ is not optionally substituted alkyl.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁵ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁵ is selected from hydrogen, halogen, and optionally substituted C₁₋₂ alkyl. In some embodiments, R⁵ is selected from hydrogen, fluoro, chloro, methyl, ethyl, and propyl. In some embodiments, R⁵ is selected from hydrogen and fluoro.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁶ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁶ is selected from hydrogen, halogen, and optionally substituted C₁₋₂ alkyl. In some embodiments, R⁶ is selected from hydrogen, fluoro, chloro, methyl, ethyl, and propyl. In some embodiments, R⁶ is selected from hydrogen and fluoro. In some embodiments, R⁶ is hydrogen.

R⁷ can be any suitable functional group known by one of skill in the art. In some embodiments, R⁷ is selected from hydrogen and optionally substituted C₁₋₄ alkyl. In some embodiments, R⁷ is selected from hydrogen, methyl, ethyl, and propyl. In some embodiments, R⁷ is hydrogen.

In some embodiments, the compound, or a pharmaceutically acceptable salt or solvate thereof has the structure of Formula (IA):

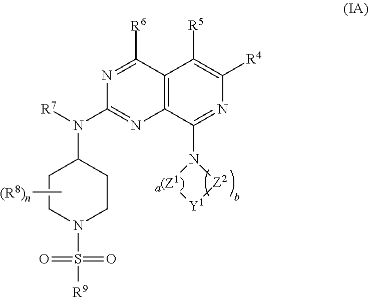

wherein,
R⁸ is selected from halogen, —CN, and optionally substituted C₁₋₄ alkyl;
R⁹ is selected from optionally substituted C₁₋₄ alkyl, optionally substituted C₃₋₆ carbocycle, and 3- to 6-membered heterocycloalkyl; and
n is selected from 0 to 9;
   wherein either (i) Y¹ is —C(R²)₂— and the two R² substituents come together to form a ring selected from an optionally substituted heterocycle and an optionally substituted carbocycle, or (ii) R⁴ is selected from hydrogen, halogen, and —CN.

In some embodiments, or two R² substituents come together to form an optionally substituted heterocycle or an optionally substituted carbocycle. In some embodiments, two R² substituents come together to form an optionally substituted heterocycle. In some embodiments, two R² substituents come together to form an optionally substituted carbocycle. In some embodiments, two R² substituents come together such that this

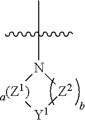

structure is

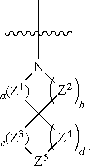

Each of Z¹, Z², Z³, Z⁴ and Z⁵ can be any suitable functional group known by one d skill in the art. In some embodiments, each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond. Each of Z¹ and Z² can be any functional group as described previously.

In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3, and 4. In some embodiments, each of a, b, c, and d are independently 1, 2, and 3. In some embodiments, each of a, b, c, and d are independently selected from 1 and 2.

In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁴ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁴ is selected from hydrogen, halogen, —CN, optionally substituted C₁₋₂ alkyl, and optionally substituted C₃₋₄ carbocycle. In some embodiments, R⁴ is selected from hydrogen and optionally substituted C₁ alkyl. In some embodiments, R⁴ is selected from hydrogen, methyl, and —CHF₂. In some embodiments, R⁴ is selected from hydrogen, halogen, and —CN. In some embodiments, R⁴ is selected from hydrogen. In some embodiments, R⁴ is not optionally substituted phenyl. In some embodiments, R⁴ is not optionally substituted alkyl.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁵ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁵ is selected from hydrogen, halogen, and optionally substituted C₁₋₂ alkyl. In some embodiments, R⁵ is selected from hydrogen, fluoro, chloro, methyl, ethyl, and propyl. In some embodiments, R⁵ is selected from hydrogen and fluoro.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁶ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁶ is selected from hydrogen, halogen, and optionally substituted C₁₋₂ alkyl. In some embodiments, R⁶ is selected from hydrogen, fluoro, chloro, methyl, ethyl, and propyl. In some embodiments, R⁶ is selected from hydrogen and fluoro. In some embodiments, R⁶ is hydrogen.

R⁷ can be any suitable functional group known by one of skill in the art. In some embodiments, R⁷ is selected from hydrogen and optionally substituted C₁₋₄ alkyl. In some embodiments, R⁷ is selected from hydrogen, methyl, ethyl, and propyl. In some embodiments, R⁷ is hydrogen.

R⁸ can be any suitable functional group known by one of skill in the art. In some embodiments, R⁸ is selected from halogen, —CN, and optionally substituted C₁₋₄ alkyl. In some embodiments, R⁸ is selected from halogen and optionally substituted C₁₋₄ alkyl. In some embodiments, R⁸ is selected from optionally substituted C₁₋₄ alkyl. In some embodiments, R⁸ is selected from methyl, ethyl, and propyl.

R⁹ can be any suitable functional group known by one of skill in the art. In some embodiments, R⁹ is selected from optionally substituted C₁₋₄ alkyl, optionally substituted C₃₋₆ carbocycle, and 3- to 6-membered heterocycloalkyl. In some embodiments, R⁹ is selected from optionally substituted C₁₋₄ alkyl and optionally substituted C₃₋₆ carbocycle. In some embodiments, R⁹ is selected from optionally substituted C₁₋₄ alkyl. In some embodiments, R⁹ is selected from methyl, ethyl, and propyl.

Variable n can be any suitable number known by one of skill in the art. In some embodiments, n is selected from 0 to 9. In some embodiments, n is selected from 0 to 5. In some embodiments, n is selected from 0 to 3. In some embodiments n is selected from 0 to 2. In some embodiments n is 0 or 1. In some embodiments, n is 0.

In some embodiments, the compound, or a pharmaceutically acceptable salt or solvate thereof has the structure of Formula (IAA):

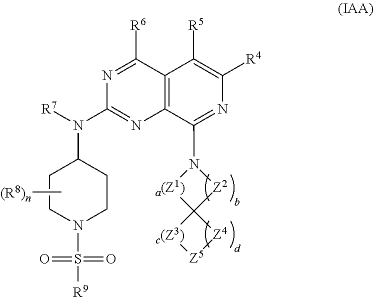

wherein,
R⁸ is selected from halogen, —CN, and optionally substituted C₁₋₄ alkyl;
R⁹ is selected from optionally substituted C₁₋₄ alkyl, optionally substituted C₃₋₆ carbocycle, and 3- to 6-membered heterocycloalkyl;
n is selected from 0 to 9;
each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond; and
each of a, b, c, and d are independently selected from 1, 2, 3, and 4.

Each of Z¹, Z², Z³, Z⁴ and Z⁵ can be any suitable functional group known by one of skill in the art. In some embodiments, each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond. Each of Z¹ and Z² can be any functional group as described previously.

In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3, and 4. In some embodiments, each of a, b, c, and d are independently 1, 2, and 3. In some embodiments, each of a, b, c, and d are independently selected from 1 and 2.

In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁴ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁴ is selected from hydrogen, halogen, —CN, optionally substituted C₁₋₂ alkyl, and optionally substituted C₃₋₄ carbocycle. In some embodiments, R⁴ is selected from hydrogen and optionally substituted C₁ alkyl. In some embodiments, R⁴ is selected from hydrogen, methyl, and —CHF₂. In some embodiments, R⁴ is selected from hydrogen, halogen, and —CN. In some embodiments, R⁴ is selected from hydrogen. In some embodiments, R⁴ is not optionally substituted phenyl. In some embodiments, R⁴ is not optionally substituted alkyl.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁵ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁵ is selected from hydrogen, halogen, and optionally substituted C₁₋₂ alkyl. In some embodiments, R⁵ is selected from hydrogen, fluoro, chloro, methyl, ethyl, and propyl. In some embodiments, R⁵ is selected from hydrogen and fluoro.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁶ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁶ is selected from hydrogen, halogen, and optionally substituted C₁₋₂ alkyl. In some embodiments, R⁶ is selected from hydrogen, fluoro, chloro, methyl, ethyl, and propyl. In some embodiments, R⁶ is selected from hydrogen and fluoro. In some embodiments, R⁶ is hydrogen.

R⁷ can be any suitable functional group known by one of skill in the art. In some embodiments, R⁷ is selected from hydrogen and optionally substituted C₁₋₄ alkyl. In some embodiments, R⁷ is selected from hydrogen, methyl, ethyl, and propyl. In some embodiments, R⁷ is hydrogen.

R⁸ can be any suitable functional group known by one of skill in the art. In some embodiments, R⁸ is selected from halogen, —CN, and optionally substituted C₁₋₄ alkyl. In some embodiments, R⁸ is selected from halogen and optionally substituted C₁₋₄ alkyl. In some embodiments, R⁸ is selected from optionally substituted C₁₋₄ alkyl. In some embodiments, R⁸ is selected from methyl, ethyl, and propyl.

R⁹ can be any suitable functional group known by one of skill in the art. In some embodiments, R⁹ is selected from optionally substituted C₁₋₄ alkyl, optionally substituted C₃₋₆ carbocycle, 3- to 6-membered heterocycloalkyl, and optionally substituted C₅₋₆ heteroaryl. In some embodiments, R⁹ is selected from optionally substituted C₁₋₄ alkyl, optionally substituted C₃₋₆ carbocycle, and optionally substituted C₅₋₆ heteroaryl. In some embodiments, R⁹ is optionally substituted C₅₋₆ heteroaryl. In some embodiments, R⁹ is optionally substituted C₅ heteroaryl. In some embodiments, R⁹ is optionally substituted C₆ heteroaryl. In some embodiments, R⁹ is optionally substituted pyrazole. In some embodiments, R⁹ is selected from optionally substituted C₁₋₄ alkyl and optionally substituted C₃₋₆ carbocycle. In some embodiments, R⁹ is selected from optionally substituted C₁₋₄ alkyl. In some embodiments, R⁹ is selected from methyl, ethyl, and propyl. In some embodiments, R⁹ is cyclopropyl, cyclobutyl, cyclopentyl, or cyclohexyl. In some embodiments, R⁹ is cyclopropyl, cyclobutyl.

Variable n can be any suitable number known by one of skill in the art. In some embodiments, n is selected from 0 to 9. In some embodiments, n is selected from 0 to 5. In some embodiments, n is selected from 0 to 3. In some embodiments n is selected from 0 to 2. In some embodiments n is 0 or 1. In some embodiments, n is 0.

In some embodiments, the compound, or a pharmaceutically acceptable salt or solvate thereof has the structure of Formula (IAAA):

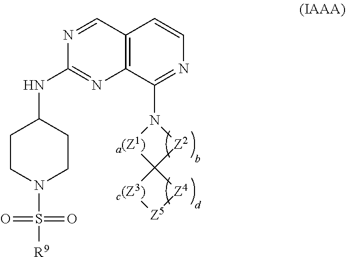

wherein,
R⁹ is selected from optionally substituted C₁₋₄ alkyl, optionally substituted C₃₋₆ carbocycle, and 3- to 6-membered heterocycloalkyl.

Each of Z¹, Z², Z³, Z⁴ and Z⁵ can be any suitable functional group known by one of skill in the art. In some embodiments, each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond. Each of Z¹ and Z² can be any functional group as described previously.

In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3, and 4. In some embodiments, each of a, b, c, and d are independently 1, 2, and 3. In some embodiments, each of a, b, c, and d are independently selected from 1 and 2. In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

R⁹ can be any suitable functional group known by one of skill in the art. In some embodiments, R⁹ is selected from optionally substituted optionally substituted C₃₋₆ carbocycle and 3 to 6-membered heterocycloalkyl. In some embodiments, R⁹ is selected from optionally substituted optionally substituted C₃₋₄ carbocycle and 5- to 6-membered heterocycloalkyl. In some embodiments, R⁹ is selected from optionally substituted cyclopropyl and optionally substituted pyrazole.

In some embodiments, the N containing heterocyclic ring depicted as

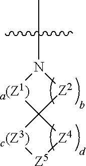

is selected from

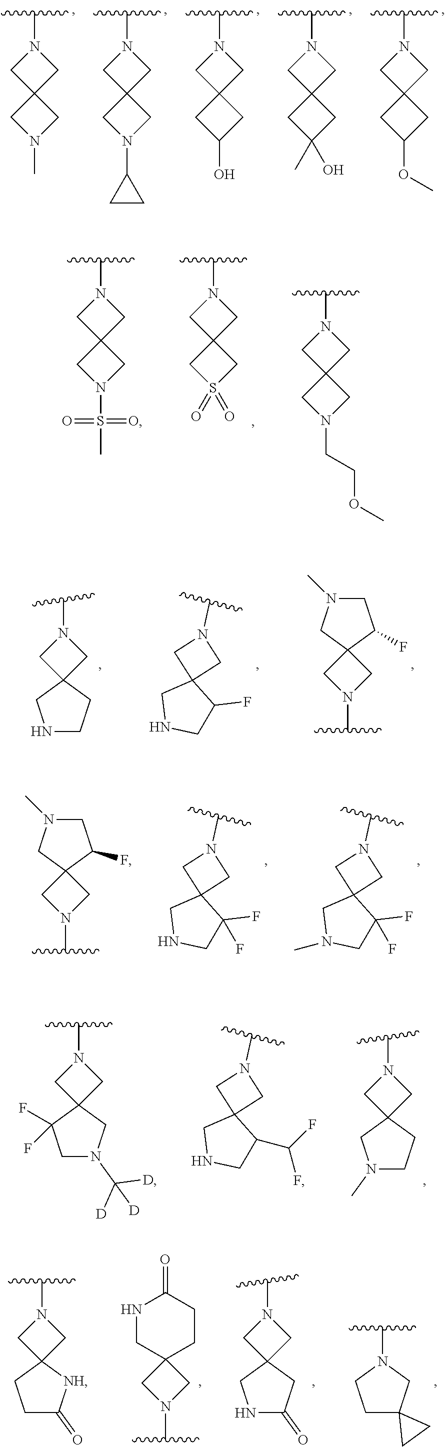

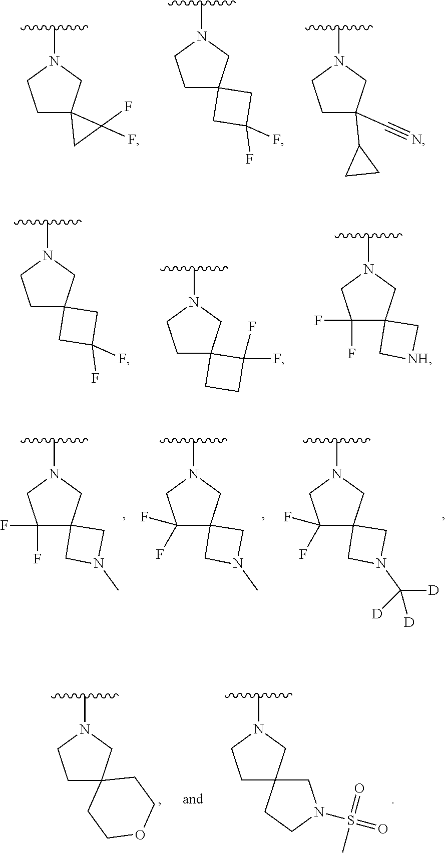

In some embodiments, the compound, or a pharmaceutically acceptable salt or solvate thereof has the structure of Formula (IB).

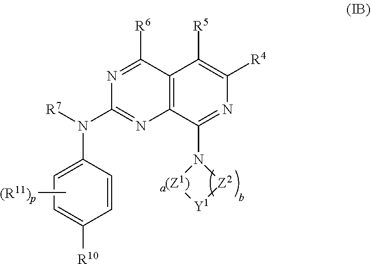

wherein,
R¹⁰ is optionally substituted heterocycloalkyl;
R¹¹ is selected from halogen, —CN, and optionally substituted C₁₋₄ alkyl; and
p is selected from 0 to 4.

In some embodiments, or two R² substituents come together to form an optionally substituted heterocycle or an optionally substituted carbocycle. In some embodiments, two R² substituents come together to form an optionally substituted heterocycle. In some embodiments, two R² substituents come together to form an optionally substituted carbocycle. In some embodiments, two R² substituents come together such that this

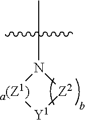

structure is

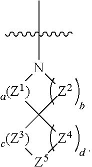

Each of Z¹, Z², Z³, Z⁴ and Z⁵ can be any suitable functional group known by one of skill in the art. In some embodiments, each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond. Each of Z¹ and Z² can be any functional group as described previously.

In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3, and 4. In some embodiments, each of a, b, c, and d are independently 1, 2, and 3. In some embodiments, each of a, b, c, and d are independently selected from 1 and 2. In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁴ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁴ is selected from hydrogen, halogen, —CN, optionally substituted C₁₋₂ alkyl, and optionally substituted C₃₋₄ carbocycle. In some embodiments, R⁴ is selected from hydrogen and optionally substituted C₁ alkyl. In some embodiments, R⁴ is selected from hydrogen, methyl, and —CHF₂. In some embodiments, R⁴ is selected from hydrogen, halogen, and —CN. In some embodiments, R⁴ is selected from hydrogen. In some embodiments, R⁴ is not optionally substituted phenyl. In some embodiments, R⁴ is not optionally substituted alkyl.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁵ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁵ is selected from hydrogen, halogen, and optionally substituted C₁₋₂ alkyl. In some embodiments, R⁵ is selected from hydrogen, fluoro, chloro, methyl, ethyl, and propyl. In some embodiments, R⁵ is selected from hydrogen and fluoro.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁶ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁶ is selected from hydrogen, halogen, and optionally substituted C₁₋₂ alkyl. In some embodiments, R⁶ is selected from hydrogen, fluoro, chloro, methyl, ethyl, and propyl. In some embodiments, R⁶ is selected from hydrogen and fluoro. In some embodiments, R⁶ is hydrogen.

R⁷ can be any suitable functional group known by one of skill in the art. In some embodiments, R⁷ is selected from hydrogen and optionally substituted C₁₋₄ alkyl. In some embodiments, R⁷ is selected from hydrogen, methyl, ethyl, and propyl. In some embodiments, R⁷ is hydrogen.

Variable n can be any suitable number known by one of skill in the art. In some embodiments, n is selected from 0 to 9. In some embodiments, n is selected from 0 to 5. In some embodiments, n is selected from 0 to 3. In some embodiments n is selected from 0 to 2. In some embodiments n is 0 or 1. In some embodiments, n is 0.

R¹⁰ can be any suitable functional group known by one of skill in the art. In some embodiments, R¹⁰ is optionally substituted heterocycloalkyl. In some embodiments, R¹⁰ is selected from optionally substituted piperazine, optionally substituted piperidine, and optionally substituted 2,6-diazaspiro[3.3]heptane. In some embodiments, R¹⁰ is selected from optionally substituted piperazine and optionally substituted 2,6-diazaspiro[3.3]heptane. In some embodiments, R¹⁰ is selected from methylpiperazine and methyl-2,6-diazaspiro[3.3]heptane.

R¹¹ can be any suitable functional group known by one of skill in the art. In some embodiments, R¹¹ is selected from halogen, —CN, and optionally substituted C₁₋₄ alkyl. In some embodiments, R¹¹ is selected from halogen and optionally substituted C₁₋₄ alkyl. In some embodiments, R¹¹ is selected from optionally substituted C₁₋₄ alkyl. In some embodiments, R¹¹ is selected from methyl, ethyl, and propyl.

Variable p can be any suitable number known by one of skill in the art. In some embodiments, p is selected from 0 to 4. In some embodiments, p is selected from 0 to 3. In some embodiments, p is selected from 0 to 2. In some embodiments, p is 0 or 1. In some embodiments, p is 0.

In some embodiments, the compound, or a pharmaceutically acceptable salt or solvate thereof has the structure of Formula (IBB):

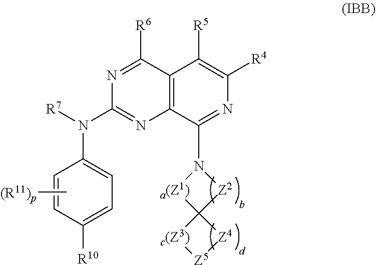

wherein,
R¹⁰ is optionally substituted heterocycloalkyl;
R¹¹ is selected from halogen, —CN, and optionally substituted C₁₋₄ alkyl;
p is selected from 0 to 4;
each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond; and
each of a, b, c, and d are independently selected from 1, 2, 3, and 4.

Each of Z¹, Z², Z³, Z⁴ and Z⁵ can be any suitable functional group known by one of skill in the art. In some embodiments, each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond. Each of Z¹ and Z² can be any functional group as described previously.

In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3, and 4. In some embodiments, each of a, b, c, and d are independently 1, 2, and 3. In some embodiments, each of a, b, c, and d are independently selected from 1 and 2. In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁴ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁴ is selected from hydrogen, halogen, —CN, optionally substituted C₁₋₂ alkyl, and optionally substituted C₃₋₄ carbocycle. In some embodiments, R⁴ is selected from hydrogen and optionally substituted C₁ alkyl. In some embodiments, R⁴ is selected from hydrogen, methyl, and —CHF₂. In some embodiments, R⁴ is selected from hydrogen, halogen, and —CN. In some embodiments, R⁴ is selected from hydrogen. In some embodiments, R⁴ is not optionally substituted phenyl. In some embodiments, R⁴ is not optionally substituted alkyl.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁵ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁵ is selected from hydrogen, halogen, and optionally substituted C₁₋₂ alkyl. In some embodiments, R⁵ is selected from hydrogen, fluoro, chloro, methyl, ethyl, and propyl. In some embodiments, R⁵ is selected from hydrogen and fluoro.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁶ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁶ is selected from hydrogen, halogen, and optionally substituted C₁₋₂ alkyl. In some embodiments, R⁶ is selected from hydrogen, fluoro, chloro, methyl, ethyl, and propyl. In some embodiments, R⁶ is selected from hydrogen and fluoro. In some embodiments, R⁶ is hydrogen.

R⁷ can be any suitable functional group known by one of skill in the art. In some embodiments, R⁷ is selected from hydrogen and optionally substituted C₁₋₄ alkyl. In some embodiments, R⁷ is selected from hydrogen, methyl, ethyl, and propyl. In some embodiments, R⁷ is hydrogen.

Variable n can be any suitable number known by one of skill in the art. In some embodiments, n is selected from 0 to 9. In some embodiments, n is selected from 0 to 5. In some embodiments, n is selected from 0 to 3. In some embodiments n is selected from 0 to 2. In some embodiments n is 0 or 1. In some embodiments, n is 0.

R¹⁰ can be any suitable functional group known by one of skill in the art. In some embodiments, R¹⁰ is optionally substituted heterocycloalkyl. In some embodiments, R¹⁰ is selected from optionally substituted piperazine, optionally substituted piperidine, and optionally substituted 2,6-diazaspiro[3.3]heptane. In some embodiments, R¹⁰ is selected from optionally substituted piperazine and optionally substituted 2,6-diazaspiro[3.3]heptane. In some embodiments, R¹⁰ is selected from methylpiperazine and methyl-2,6-diazaspiro[3.3]heptane.

R¹¹ can be any suitable functional group known by one of skill in the art. In some embodiments, R¹¹ is selected from halogen, —CN, and optionally substituted C₁₋₄ alkyl. In some embodiments, R¹¹ is selected from halogen and optionally substituted C₁₋₄ alkyl. In some embodiments, R¹¹ is selected from optionally substituted C₁₋₄ alkyl. In some embodiments, R¹¹ is selected from methyl, ethyl, and propyl.

Variable p can be any suitable number known by one of skill in the art. In some embodiments, p is selected from 0 to 4. In some embodiments, p is selected from 0 to 3. In some embodiments, p is selected from 0 to 2. In some embodiments, p is 0 or 1. In some embodiments, p is 0.

In some embodiments, the compound, or a pharmaceutically acceptable salt or solvate thereof has the structure of Formula (IC):

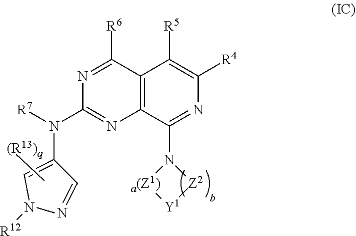

wherein,
R¹² is selected from an optionally substituted heterocycloalkyl and optionally substituted cycloalkyl; or R¹² and R¹³ come together to form an optionally substituted heterocycle;
R¹³ is selected from halogen, —CN, and optionally substituted C₁₋₄ alkyl; and
q is selected from 0 to 2.

In some embodiments, or two R² substituents come together to form an optionally substituted heterocycle or an optionally substituted carbocycle. In some embodiments, two R² substituents come together to form an optionally substituted heterocycle. In some embodiments, two R² substituents come together to form an optionally substituted carbocycle. In some embodiments, two R² substituents come together such that this

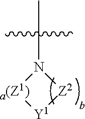

structure is

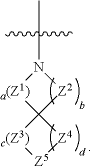

Each of Z¹, Z², Z³, Z⁴ and Z⁵ can be any suitable functional group known by one of skill in the art. In some embodiments, each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond. Each of Z¹ and Z² can be any functional group as described previously.

In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3, and 4. In some embodiments, each of a, b, c, and d are independently 1, 2, and 3. In some embodiments, each of a, b, c, and d are independently selected from 1 and 2.

In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁴ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁴ is selected from hydrogen, halogen, —CN, optionally substituted C₁₋₂ alkyl, and optionally substituted C₃₋₄ carbocycle. In some embodiments, R⁴ is selected from hydrogen and optionally substituted C₁ alkyl. In some embodiments, R⁴ is selected from hydrogen, methyl, and —CHF₂. In some embodiments, R⁴ is selected from hydrogen, halogen, and —CN. In some embodiments, R⁴ is selected from hydrogen. In some embodiments, R⁴ is not optionally substituted phenyl. In some embodiments, R⁴ is not optionally substituted alkyl.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁵ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁵ is selected from hydrogen, halogen, and optionally substituted C₁₋₂ alkyl. In some embodiments, R⁵ is selected from hydrogen, fluoro, chloro, methyl, ethyl, and propyl. In some embodiments, R⁵ is selected from hydrogen and fluoro.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁶ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁶ is selected from hydrogen, halogen, and optionally substituted C₁₋₂ alkyl. In some embodiments, R⁶ is selected from hydrogen, fluoro, chloro, methyl, ethyl, and propyl. In some embodiments, R⁶ is selected from hydrogen and fluoro. In some embodiments, R⁶ is hydrogen.

R⁷ can be any suitable functional group known by one of skill in the art. In some embodiments, R⁷ is selected from hydrogen and optionally substituted C₁₋₄ alkyl. In some embodiments, R⁷ is selected from hydrogen, methyl, ethyl, and propyl. In some embodiments, R⁷ is hydrogen.

R¹² can be any suitable functional group known by one of skill in the art. In some embodiments, R¹² is optionally substituted heterocycloalkyl; or R¹² and R¹³ come together to form an optionally substituted heterocycle. In some embodiments, R¹² is optionally substituted 5- to 6-membered heterocycle, or R¹² and R¹³ come together to form an optionally substituted heterocycle or an optionally substituted carbocycle. In some embodiments, R¹² is optionally substituted 5- to 6-membered heterocycle. In some embodiments, R¹² is selected from optionally substituted piperidine, optionally substituted piperazine, and optionally substituted 2,6-diazaspiro[3.3]heptane. In some embodiments, R¹² is selected from optionally substituted piperidine and optionally substituted 2,6-diazaspiro[3.3]heptane. In some embodiments, R¹² is selected from methylpiperazine and methyl-2,6-diazaspiro[3.3]heptane.

R¹³ can be any suitable functional group known by one of skill in the art. In some embodiments, R¹³ is selected from halogen, —CN, and optionally substituted C₁₋₄ alkyl; or R¹² and R¹³ come together to form an optionally substituted heterocycle. In some embodiments, R¹³ is selected from halogen and optionally substituted C₁₋₄ alkyl. In some embodiments, R¹³ is selected from methyl, ethyl, and propyl.

In some embodiments, R¹² and R¹³ come together to form an optionally substituted heterocycle or an optionally substituted carbocycle. In some embodiments, R¹² and R¹³ come together to form an optionally substituted heterocycle. In some embodiments, R¹² and R¹³ come together such that

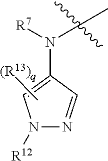

structure is

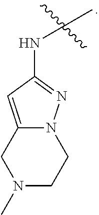 [SMILES: *Nc1cc2n(n1)CCN(C)C2]

Variable q can be any number known by one of skill in the art. In some embodiments, q is selected from 0 to 2. In some embodiments, q is 0 or 1. In some embodiments, q is 0. In some embodiments, q is 1.

In some embodiments, the compound, or a pharmaceutically acceptable salt or solvate thereof has the structure of Formula (ICC):

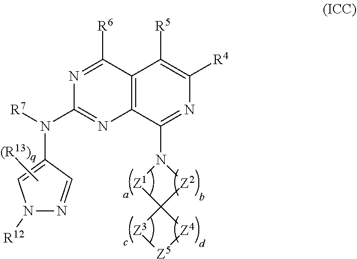

wherein,
R¹² is optionally substituted heterocycloalkyl; or R¹² and R¹³ come together to form an optionally substituted heterocycle;
R¹³ is selected from halogen, —CN, and optionally substituted C₁₋₄ alkyl; q is selected from 0 to 2; and
each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond; and
each of a, b, c, and d are independently selected from 1, 2, 3, and 4.

Each of Z¹, Z², Z³, Z⁴ and Z⁵ can be any suitable functional group known by one of skill in the art. In some embodiments, each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond. Each of Z¹ and Z² can be any functional group as described previously.

In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3, and 4. In some embodiments, each of a, b, c, and d are independently 1, 2, and 3. In some embodiments, each of a, b, c, and d are independently selected from 1 and 2.

In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁴ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁴ is selected from hydrogen, halogen, —CN, optionally substituted C₁₋₂ alkyl, and optionally substituted C₃₋₄ carbocycle. In some embodiments, R⁴ is selected from hydrogen and optionally substituted C₁ alkyl. In some embodiments, R⁴ is selected from hydrogen, methyl, and —CHF₂. In some embodiments, R⁴ is selected from hydrogen, halogen, and —CN. In some embodiments, R⁴ is selected from hydrogen. In some embodiments, R⁴ is not optionally substituted phenyl. In some embodiments, R⁴ is not optionally substituted alkyl.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁵ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁵ is selected from hydrogen, halogen, and optionally substituted C₁₋₂ alkyl. In some embodiments, R⁵ is selected from hydrogen, fluoro, chloro, methyl, ethyl, and propyl. In some embodiments, R⁵ is selected from hydrogen and fluoro.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁶ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁶ is selected from hydrogen, halogen, and optionally substituted C₁₋₂ alkyl. In some embodiments, R⁶ is selected from hydrogen, fluoro, chloro, methyl, ethyl, and propyl. In some embodiments, R⁶ is selected from hydrogen and fluoro. In some embodiments, R⁶ is hydrogen.

R⁷ can be any suitable functional group known by one of skill in the art. In some embodiments, R⁷ is selected from hydrogen and optionally substituted C₁₋₄ alkyl. In some embodiments, R⁷ is selected from hydrogen, methyl, ethyl, and propyl. In some embodiments, R⁷ is hydrogen.

R¹² can be any suitable functional group known by one of skill in the art. In some embodiments, R¹² is optionally substituted heterocycloalkyl; or R¹² and R¹³ come together to form an optionally substituted heterocycle. In some embodiments, R¹² is optionally substituted 5- to 6-membered heterocycle, or R¹² and R¹³ come together to form an optionally substituted heterocycle or an optionally substituted carbocycle. In some embodiments, R¹² is optionally substituted 5- to 6-membered heterocycle. In some embodiments, R¹² is selected from optionally substituted piperidine, optionally substituted piperazine, and optionally substituted 2,6-diazaspiro[3.3]heptane. In some embodiments, R¹² is selected from optionally substituted piperidine and optionally substituted 2,6-diazaspiro[3.3]heptane. In some embodiments, R¹² is selected from methylpiperazine and methyl-2,6-diazaspiro[3.3]heptane.

R¹³ can be any suitable functional group known by one of skill in the art. In some embodiments, R¹³ is selected from halogen, —CN, and optionally substituted C₁₋₄ alkyl; or R¹² and R¹³ come together to form an optionally substituted heterocycle. In some embodiments, R¹³ is selected from halogen and optionally substituted C₁₋₄ alkyl. In some embodiments, R¹³ is selected from methyl, ethyl, and propyl.

In some embodiments, R¹² and R¹³ come together to form an optionally substituted heterocycle or an optionally substituted carbocycle. In some embodiments, R¹² and R¹³ come together to form an optionally substituted heterocycle. In some embodiments, R¹² and R¹³ come together such that

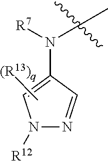

structure is

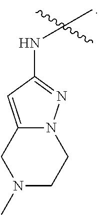 [SMILES: *Nc1cc2n(n1)CCN(C)C2]

Variable q can be any number known by one of skill in the art. In some embodiments, q is selected from 0 to 2. In some embodiments, q is 0 or 1. In some embodiments, q is 0. In some embodiments, q is 1.

In some embodiments, the compound, or a pharmaceutically acceptable salt or solvate thereof has the structure of Formula (ID):

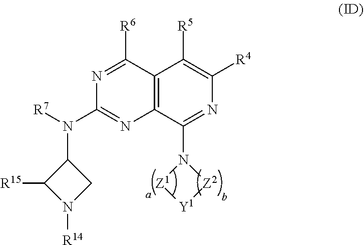

wherein,
R¹⁴ is selected from —SOR¹⁶—, and optionally substituted heterocycloalkyl;
R¹⁵ is selected from hydrogen, halogen, —CN, and optionally substituted C₁₋₄ alkyl; and
R¹⁶ is selected from optionally substituted C₁₋₄ alkyl, optionally substituted C₃₋₆ carbocycle, and optionally substituted 3- to 6-membered heterocycloalkyl

In some embodiments, or two R² substituents come together to form an optionally substituted heterocycle or an optionally substituted carbocycle. In some embodiments, two R² substituents come together to form an optionally substituted heterocycle. In some embodiments, two R² substituents come together to form an optionally substituted carbocycle. In some embodiments, two R² substituents come together such that this

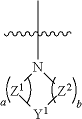

structure is

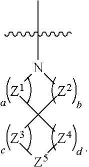

Each of Z¹, Z², Z³, Z⁴ and Z⁵ can be any suitable functional group known y one of skill in the art. In some embodiments, each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond. Each of Z¹ and Z² can be any functional group as described previously.

In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3, and 4. In some embodiments, each of a, b, c, and d are independently 1, 2, and 3. In some embodiments, each of a, b, c, and d are independently selected from 1 and 2.

In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁴ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁴ is selected from hydrogen, halogen, —CN, optionally substituted C₁₋₂ alkyl, and optionally substituted C₃₋₄ carbocycle. In some embodiments, R⁴ is selected from hydrogen and optionally substituted C₁ alkyl. In some embodiments, R⁴ is selected from hydrogen, methyl, and —CHF₂. In some embodiments, R⁴ is selected from hydrogen, halogen, and —CN. In some embodiments, R⁴ is selected from hydrogen. In some embodiments, R⁴ is not optionally substituted phenyl. In some embodiments, R⁴ is not optionally substituted alkyl.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁵ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁵ is selected from hydrogen, halogen, and optionally substituted C₁₋₂ alkyl. In some embodiments, R⁵ is selected from hydrogen, fluoro, chloro, methyl, ethyl, and propyl. In some embodiments, R⁵ is selected from hydrogen and fluoro.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁶ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁶ is selected from hydrogen, halogen, and optionally substituted C₁₋₂ alkyl. In some embodiments, R⁶ is selected from hydrogen, fluoro, chloro, methyl, ethyl, and propyl. In some embodiments, R⁶ is selected from hydrogen and fluoro. In some embodiments, R⁶ is hydrogen.

R⁷ can be any suitable functional group known by one of skill in the art. In some embodiments, R⁷ is selected from hydrogen and optionally substituted C₁₋₄ alkyl. In some embodiments, R⁷ is selected from hydrogen, methyl, ethyl, and propyl. In some embodiments, R⁷ is hydrogen.

R¹⁴ can be any suitable functional group known by one of skill in the art. In some embodiments, R¹⁴ is selected from —SOR¹⁶—, and optionally substituted heterocycloalkyl. In some embodiments, R¹⁴ is —SOR¹⁶—. In some embodiments, R¹⁴ is selected from optionally substituted heterocycloalkyl.

R¹⁵ can be any suitable functional group known by one of skill in the art. In some embodiments, R¹⁵ is selected from hydrogen, halogen, —CN, and optionally substituted C₁₋₄ alkyl. In some embodiments, R¹⁵ is selected from hydrogen, halogen, and optionally substituted C₁₋₄ alkyl. In some embodiments, R¹⁵ is selected from hydrogen and optionally substituted C₁₋₄ alkyl. In some embodiments, R¹⁵ is hydrogen. In some embodiments, R¹⁵ is optionally substituted C₁₋₄ alkyl. In some embodiments, R¹⁵ is methyl, ethyl, and propyl.

R¹⁶ can be any suitable functional group known by one of skill in the art. In some embodiments, R¹⁶ is selected from optionally substituted C₁₋₄ alkyl, optionally substituted C₃₋₆ carbocycle, and optionally substituted 3- to 6-membered heterocycloalkyl. In some embodiments, R¹⁶ is optionally substituted C₃₋₆ carbocycle and optionally substituted 3- to 6-membered heterocycloalkyl. In some embodiments, R¹⁶ is selected from optionally substituted C₁₋₄ alkyl. In some embodiments, R¹⁶ is selected from methyl, ethyl, and propyl.

In some embodiments, the compound, or a pharmaceutically acceptable salt or solvate thereof has the structure of Formula (IDD):

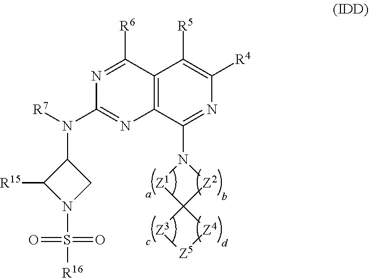

wherein,
   R¹⁶ selected from optionally substituted C₁₋₄ alkyl, optionally substituted C₃₋₆ carbocycle, and optionally substituted 3- to 6-membered heterocycloalkyl.

Each of Z¹, Z², Z³, Z⁴ and Z⁵ can be any suitable functional group known by one of skill in the art. In some embodiments, each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond. Each of Z¹ and Z² can be any functional group as described previously.

In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3, and 4. In some embodiments, each of a, b, c, and d are independently 1, 2, and 3. In some embodiments, each of a, b, c, and d are independently selected from 1 and 2.

In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁴ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁴ is selected from hydrogen, halogen, —CN, optionally substituted C₁₋₂ alkyl, and optionally substituted C₃₋₄ carbocycle. In some embodiments, R⁴ is selected from hydrogen and optionally substituted C₁ alkyl. In some embodiments, R⁴ is selected from hydrogen, methyl, and —CHF₂. In some embodiments, R⁴ is selected from hydrogen, halogen, and —CN. In some embodiments, R⁴ is selected from hydrogen. In some embodiments, R⁴ is not optionally substituted phenyl. In some embodiments, R⁴ is not optionally substituted alkyl.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁵ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁵ is selected from hydrogen, halogen, and optionally substituted C₁₋₂ alkyl. In some embodiments, R⁵ is selected from hydrogen, fluoro, chloro, methyl, ethyl, and propyl. In some embodiments, R⁵ is selected from hydrogen and fluoro.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁶ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁶ is selected from hydrogen, halogen, and optionally substituted C₁₋₂ alkyl. In some embodiments, R⁶ is selected from hydrogen, fluoro, chloro, methyl, ethyl, and propyl. In some embodiments, R⁶ is selected from hydrogen and fluoro. In some embodiments, R⁶ is hydrogen.

R⁷ can be any suitable functional group known by one of skill in the art. In some embodiments, R⁷ is selected from hydrogen and optionally substituted C₁₋₄ alkyl. In some embodiments, R⁷ is selected from hydrogen, methyl, ethyl, and propyl. In some embodiments, R⁷ is hydrogen. R¹⁵ can be any suitable functional group known by one of skill in the art. In some embodiments, R¹⁵ is selected from hydrogen, halogen, —CN, and optionally substituted C₁₋₄ alkyl. In some embodiments, R¹⁵ is selected from hydrogen, halogen, and optionally substituted C₁₋₄ alkyl. In some embodiments, R¹⁵ is selected from hydrogen and optionally substituted C₁₋₄ alkyl. In some embodiments, R¹⁵ is hydrogen. In some embodiments, R¹⁵ is optionally substituted C₁₋₄ alkyl. In some embodiments, R¹⁵ is methyl, ethyl, and propyl.

R¹⁶ can be any suitable functional group known by one of skill in the art. In some embodiments, R¹⁶ is selected from optionally substituted C₁₋₄ alkyl, optionally substituted C₃₋₆ carbocycle, and optionally substituted 3- to 6-membered heterocycloalkyl. In some embodiments, R¹⁶ is optionally substituted C₃₋₆ carbocycle and optionally substituted 3- to 6-membered heterocycloalkyl. In some embodiments, R¹⁶ is selected from optionally substituted C₁₋₄ alkyl. In some embodiments, R¹⁶ is selected from methyl, ethyl, and propyl.

In some embodiments, the compound, or a pharmaceutically acceptable salt or solvate thereof has the structure of Formula (IE):

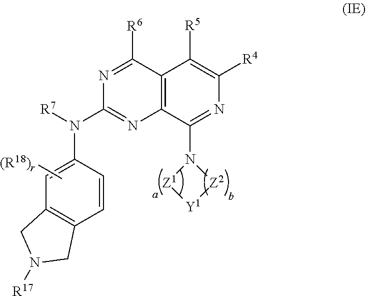

wherein,
R¹⁷ is selected from —SOR¹⁹—, optionally substituted alkyl, optionally substituted carbocycle, and optionally substituted heterocycloalkyl;
R¹⁸ is selected from halogen, —CN, and optionally substituted C₁₋₄ alkyl;
R¹⁹ is selected from optionally substituted C₁₋₄ alkyl, optionally substituted C₃₋₆ carbocycle, and optionally substituted 3- to 6-membered heterocycloalkyl; and
r is selected from 0 to 5.

In some embodiments, or two R² substituents come together to form an optionally substituted heterocycle or an optionally substituted carbocycle. In some embodiments, two R² substituents come together to form an optionally substituted heterocycle. In some embodiments, two R² substituents come together to form an optionally substituted carbocycle. In some embodiments, two R² substituents come together such that this

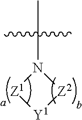

structure is

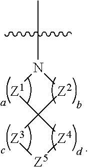

Each of Z¹, Z², Z³, Z⁴ and Z⁵ can be any suitable functional group known by one of skill in the art. In some embodiments, each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond. Each of Z¹ and Z² can be any functional group as described previously.

In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3, and 4. In some embodiments, each of a, b, c, and d are independently 1, 2, and 3. In some embodiments, each of a, b, c, and d are independently selected from 1 and 2.

In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁴ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁴ is selected from hydrogen, halogen, —CN, optionally substituted C₁₋₂ alkyl, and optionally substituted C₃₋₄ carbocycle. In some embodiments, R⁴ is selected from hydrogen and optionally substituted C₁ alkyl. In some embodiments, R⁴ is selected from hydrogen, methyl, and —CHF₂. In some embodiments, R⁴ is selected from hydrogen, halogen, and —CN. In some embodiments, R⁴ is selected from hydrogen. In some embodiments, R⁴ is not optionally substituted phenyl. In some embodiments, R⁴ is not optionally substituted alkyl.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁵ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁵ is selected from hydrogen, halogen, and optionally substituted C₁₋₂ alkyl. In some embodiments, R⁵ is selected from hydrogen, fluoro, chloro, methyl, ethyl, and propyl. In some embodiments, R⁵ is selected from hydrogen and fluoro.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁶ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁶ is selected from hydrogen, halogen, and optionally substituted C₁₋₂ alkyl. In some embodiments, R⁶ is selected from hydrogen, fluoro, chloro, methyl, ethyl, and propyl. In some embodiments, R⁶ is selected from hydrogen and fluoro. In some embodiments, R⁶ is hydrogen.

R⁷ can be any suitable functional group known by one of skill in the art. In some embodiments, R⁷ is selected from hydrogen and optionally substituted C₁₋₄ alkyl. In some embodiments, R⁷ is selected from hydrogen, methyl, ethyl, and propyl. In some embodiments, R⁷ is hydrogen. R¹⁴ can be any suitable functional group known by one of skill in the art. In some embodiments, R¹⁴ is selected from —SOR¹⁶—, and optionally substituted heterocycloalkyl. In some embodiments, R¹⁴ is —SOR¹⁶—. In some embodiments, R¹⁴ is selected from optionally substituted heterocycloalkyl.

R¹⁷ can be any suitable functional group known by one of skill in the art. In some embodiments, R¹⁷ is selected from —SOR¹⁹—, optionally substituted alkyl, optionally substituted carbocycle, and optionally substituted heterocycloalkyl. In some embodiments, R¹⁷ is selected from —SOR¹⁹—, optionally substituted alkyl, and optionally substituted 3- to 5 membered heterocycloalkyl. In some embodiments, R¹⁷ is selected from —SOR¹⁹—, methyl, and optionally substituted 4-membered heterocycloalkyl. In some embodiments, R¹⁷ is selected from —SOR¹⁹—, methyl, and 1-(methylsulfonyl)azetidine. In some embodiments, R¹⁷ is —SOR¹⁹—. In some embodiments, R¹⁷ is methyl. In some embodiments, R¹⁷ is 1-(methylsulfonyl)azetidine.

R¹⁸ can be any suitable functional group known by one of skill in the art. In some embodiments, R¹⁸ is selected from halogen, —CN, and optionally substituted C₁₋₄ alkyl. In some embodiments, R¹⁸ is selected from halogen and optionally substituted C₁₋₄ alkyl. In some embodiments, R¹⁸ is selected from optionally substituted C₁₋₄ alkyl. In some embodiments, R¹⁸ is selected from methyl, ethyl, and propyl.

R¹⁹ can be any suitable functional group known by one of skill in the art. In some embodiments, R¹⁹ is selected from optionally substituted C₁₋₄ alkyl, optionally substituted C₃₋₆ carbocycle, and optionally substituted 3- to 6-membered heterocycloalkyl. In some embodiments, R¹⁹ is selected from optionally substituted C₃₋₆ carbocycle and optionally substituted 3- to 6-membered heterocycloalkyl. In some embodiments, R¹⁹ is optionally substituted alkyl. In some embodiments, R¹⁹ is selected from methyl, ethyl, and propyl. In some embodiments, R¹⁹ is methyl.

Variable r can be any suitable number known by one of skill in the art. In some embodiments, r is selected from 0 to 5. In some embodiments, r is selected from 0 to 3. In some embodiments, r is selected from 0 to 2. In some embodiments, r is 0 or 1. In some embodiments, r is 0.

In some embodiments, the compound, or a pharmaceutically acceptable salt or solvate thereof has the structure of Formula (IEE):

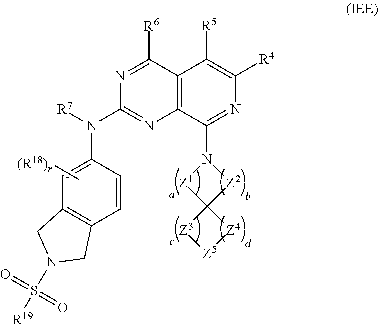

wherein,
R¹⁹ is selected from optionally substituted C₁₋₄ alkyl, optionally substituted C₃₋₆ carbocycle, and optionally substituted 3- to 6-membered heterocycloalkyl.

Each of Z¹, Z², Z³, Z⁴ and Z⁵ can be any suitable functional group known by one of skill in the art. In some embodiments, each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond. Each of Z¹ and Z² can be any functional group as described previously.

In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3, and 4. In some embodiments, each of a, b, c, and d are independently 1, 2, and 3. In some embodiments, each of a, b, c, and d are independently selected from 1 and 2.

In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

In some embodiments, each R³ is independently selected from hydrogen, optionally substituted alkyl, optionally substituted C₃₋₄ carbocycle, and optionally substituted 3- to 4-membered heterocyclcoalkyl. In some embodiments, each R³ is independently selected from optionally substituted alkyl, optionally substituted C₃₋₄ carbocycle, and optionally substituted 3- to 4-membered heterocyclcoalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁴ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁴ is selected from hydrogen, halogen, —CN, optionally substituted C₁₋₂ alkyl, and optionally substituted C₃₋₄ carbocycle. In some embodiments, R⁴ is selected from hydrogen and optionally substituted C₁ alkyl. In some embodiments, R⁴ is selected from hydrogen, methyl, and —CHF₂. In some embodiments, R⁴ is selected from hydrogen, halogen, and —CN. In some embodiments, R⁴ is selected from hydrogen. In some embodiments, R⁴ is not optionally substituted phenyl. In some embodiments, R⁴ is not optionally substituted alkyl.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁵ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁵ is selected from hydrogen, halogen, and optionally substituted C₁₋₂ alkyl. In some embodiments, R⁵ is selected from hydrogen, fluoro, chloro, methyl, ethyl, and propyl. In some embodiments, R⁵ is selected from hydrogen and fluoro.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocyclcoalkyl. In some embodiments, R⁶ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁶ is selected from hydrogen, halogen, and optionally substituted C₁₋₂ alkyl. In some embodiments, R⁶ is selected from hydrogen, fluoro, chloro, methyl, ethyl, and propyl. In some embodiments, R⁶ is selected from hydrogen and fluoro. In some embodiments, R⁶ is hydrogen.

R⁷ can be any suitable functional group known by one of skill in the art. In some embodiments, R⁷ is selected from hydrogen and optionally substituted C₁₋₄ alkyl. In some embodiments, R⁷ is selected from hydrogen, methyl, ethyl, and propyl. In some embodiments, R⁷ is hydrogen. R¹⁴ can be any suitable functional group known by one of skill in the art. In some embodiments, R¹⁴ is selected from —SOR¹⁶—, and optionally substituted heterocycloalkyl. In some embodiments, R¹⁴ is —SOR¹⁶—. In some embodiments, R¹⁴ is selected from optionally substituted heterocycloalkyl.

R¹⁸ can be any suitable functional group known by one of skill in the art. In some embodiments, R¹⁸ is selected from halogen, —CN, and optionally substituted C₁₋₄ alkyl. In some embodiments, R¹⁸ is selected from halogen and optionally substituted C₁₋₄ alkyl. In some embodiments, R¹⁸ is selected from optionally substituted C₁₋₄ alkyl. In some embodiments, R¹⁸ is selected from methyl, ethyl, and propyl.

R¹⁹ can be any suitable functional group known by one of skill in the art. In some embodiments, R¹⁹ is selected from optionally substituted C₁₋₄ alkyl, optionally substituted C₃₋₆ carbocycle, and optionally substituted 3- to 6-membered heterocycloalkyl. In some embodiments, R¹⁹ is selected from optionally substituted C₃₋₆ carbocycle and optionally substituted 3- to 6-membered heterocycloalkyl. In some embodiments, R¹⁹ is optionally substituted alkyl. In some embodiments, R¹⁹ is selected from methyl, ethyl, and propyl. In some embodiments, R¹⁹ is methyl.

Variable r can be any suitable number known by one of skill in the art. In some embodiments, r is selected from 0 to 5. In some embodiments, r is selected from 0 to 3. In some embodiments, r is selected from 0 to 2. In some embodiments, r is 0 or 1. In some embodiments, r is 0.

In some embodiments, the compound is selected from:

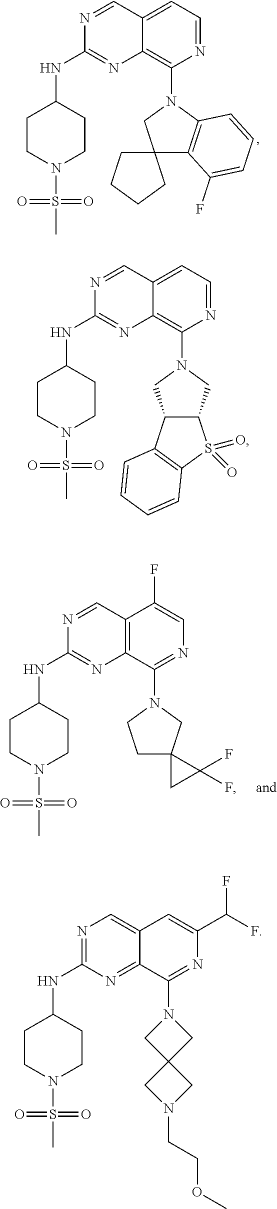 [SMILES: CS(=O)(=O)N1CCC(Nc2ncc3ccnc(N4CC5(CCCC5)c5c(F)cccc54)c3n2)CC1 ; CS(=O)(=O)N1CCC(Nc2ncc3ccnc(N4C[C@H]5c6ccccc6S(=O)(=O)[C@H]5C4)c3n2)CC1 ; CS(=O)(=O)N1CCC(Nc2ncc3c(F)cnc(N4CCC5(C4)CC5(F)F)c3n2)CC1 ; "and" ; COCCN1CC2(C1)CN(c1nc(C(F)F)cc3cnc(NC4CCN(S(C)(=O)=O)CC4)nc13)C2]

In some embodiments, the compound is selected from:

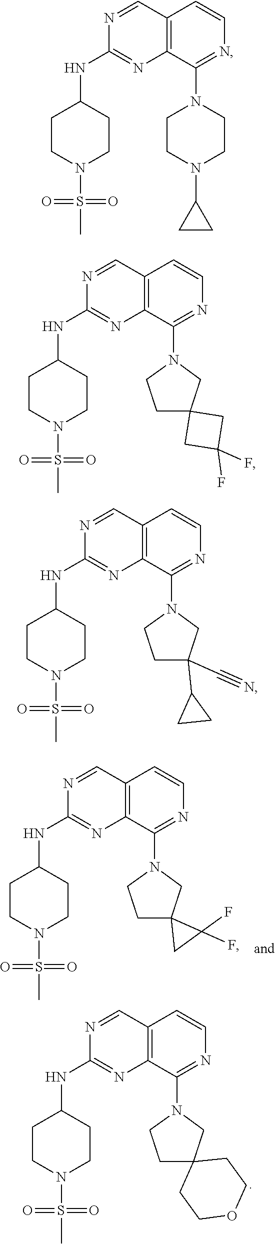

In some embodiments, the compound is selected from:

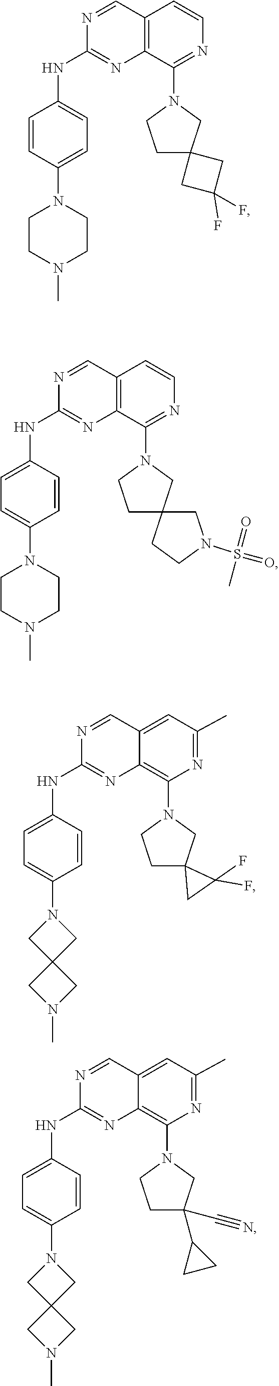

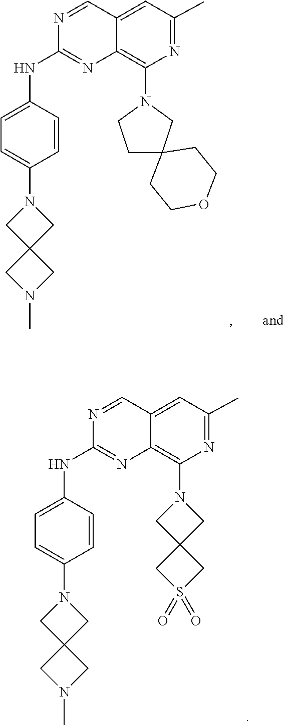 [SMILES: Cc1cc2cnc(Nc3ccc(N4CC5(CN(C)C5)C4)cc3)nc2c(N2CCC3(CCOCC3)C2)n1 ; Cc1cc2cnc(Nc3ccc(N4CC5(CN(C)C5)C4)cc3)nc2c(N2CC3(C2)CS(=O)(=O)C3)n1]

In some embodiments, the compound is selected from:

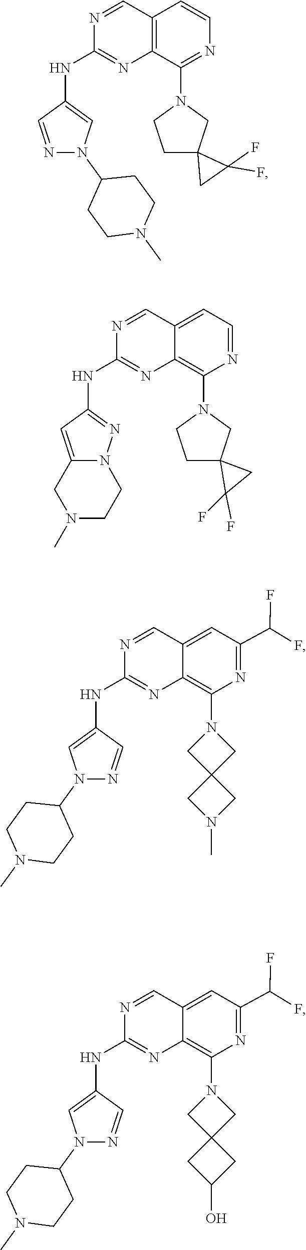 [SMILES: CN1CCC(n2cc(Nc3ncc4ccnc(N5CCC6(C5)CC6(F)F)c4n3)cn2)CC1 ; CN1CCn2nc(Nc3ncc4ccnc(N5CCC6(C5)CC6(F)F)c4n3)cc2C1 ; CN1CCC(n2cc(Nc3ncc4cc(C(F)F)nc(N5CC6(CN(C)C6)C5)c4n3)cn2)CC1 ; CN1CCC(n2cc(Nc3ncc4cc(C(F)F)nc(N5CC6(CC(O)C6)C5)c4n3)cn2)CC1]

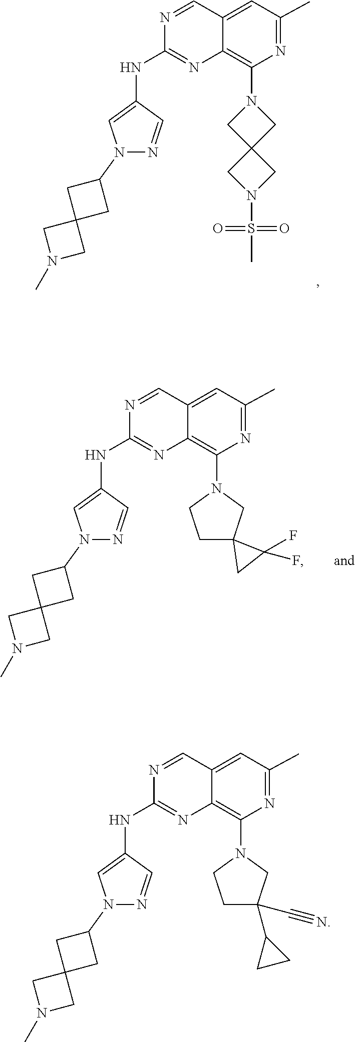

In some embodiments, the compound is selected from:

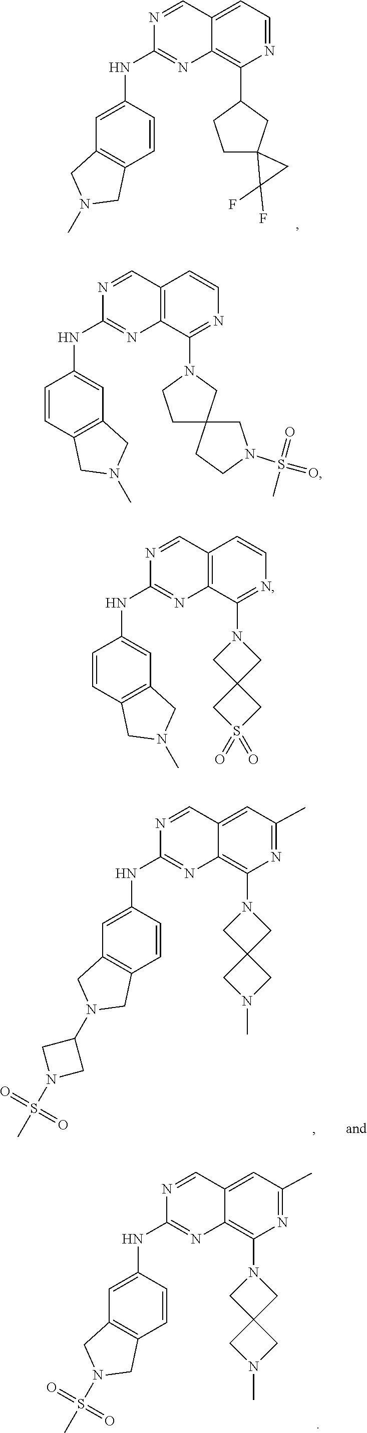

In some embodiments, the compound is selected from:

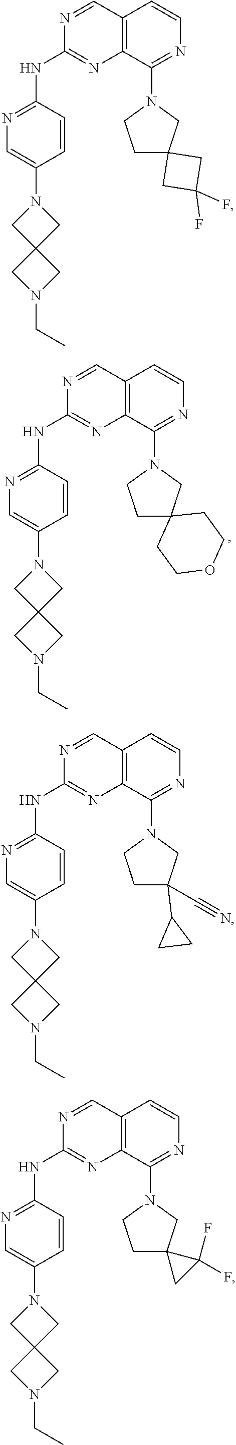

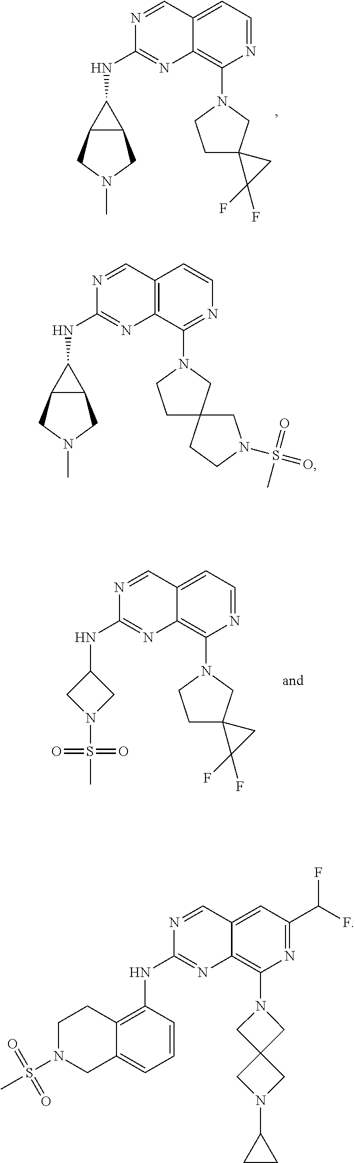 [SMILES: CN1C[C@@H]2[C@H](C1)[C@H]2Nc1ncc2ccnc(N3CCC4(C3)CC4(F)F)c2n1 ; CN1C[C@@H]2[C@H](C1)[C@H]2Nc1ncc2ccnc(N3CCC4(CCN(S(C)(=O)=O)C4)C3)c2n1 ; CS(=O)(=O)N1CC(Nc2ncc3ccnc(N4CCC5(C4)CC5(F)F)c3n2)C1 ; CS(=O)(=O)N1CCc2c(cccc2Nc2ncc3cc(C(F)F)nc(N4CC5(C4)CN(C4CC4)C5)c3n2)C1]

Additional embodiments of the compounds of this disclosure include the following:

Embodiment 1 relates to a compound, or a pharmaceutically acceptable salt or solvate thereof, having the structure of Formula (I):

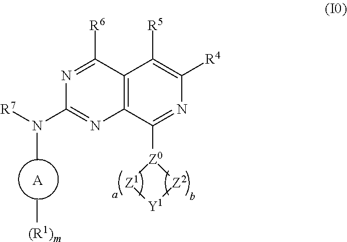

wherein,
A is a ring selected from optionally substituted carbocycle, optionally substituted 4- to 8-membered heterocycle, optionally substituted tetrahydro-triazolopyrazine, and optionally substituted isoindoline;
Z⁰ is —C(H)— or nitrogen;
each of Z¹, Z², and Y¹ are independently selected from —C(R²)₂—, —C(O)—, —NR³—, —N(C(O)R²)—, —NS(O₂)R², —O—, —S—, —S(O)—, and —S(O)₂—;
each of a and b are independently selected from 1, 2, 3, and 4;
each R¹ is independently selected from halogen, —CN, —NO₂, optionally substituted alkyl, optionally substituted alkenyl, optionally substituted alkynyl, optionally substituted carbocycle, and optionally substituted heterocycle;
m is selected from 0 to 5;
each R² is independently selected from hydrogen, halogen, —CN, —OH, optionally substituted alkoxy, optionally substituted alkyl, optionally substituted cycloalkyl, optionally substituted —O-cycloalkyl, and optionally substituted heterocycloalkyl, or two R² substituents come together to form an optionally substituted heterocycle or an optionally substituted carbocycle, or R² and R³ substituents come together to form an optionally substituted heterocycle;
each R³ is independently selected from hydrogen, optionally substituted alkyl, optionally substituted C₃₋₄ carbocycle, and optionally substituted 3- to 4-membered heterocycloalkyl;
R⁴ is selected from hydrogen, halogen, —CN, optionally substituted C₁₋₄ alkyl, optionally substituted C₃₋₄ carbocycle, and optionally substituted 3- to 4-membered heterocycloalkyl;
each of R⁵, R⁶, is independently selected from hydrogen, halogen, —CN, optionally substituted C₁₋₄ alkyl, optionally substituted C₃₋₄ carbocycle, and optionally substituted 3- to 4-membered heterocyclcoalkyl; and
R⁷ is selected from hydrogen and optionally substituted C₁₋₄ alkyl, and
wherein if A is optionally substituted phenyl, m is from 1 to 5 and at least one R is a heterocycloalkyl,
wherein if A is optionally substituted pyridine, optionally substituted pyridazine, or optionally substituted pyrimidine, R⁴ is selected from hydrogen, halogen, and —CN,
wherein if A is an optionally substituted piperidine sulfonamide, then either (i) Y¹ is —C(R²)₂— and the two R² substituents come together to form a ring selected from an optionally substituted heterocycle and an optionally substituted carbocycle, or (ii) R⁴ is selected from hydrogen, halogen, methyl, and —CN, and
wherein if A-R¹ is

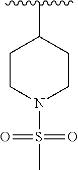 [SMILES: *C1CCN(S(C)(=O)=O)CC1]

and Z⁰ is CH, then R⁴ is methyl or cyclopropyl.

Embodiment 2 relates to the compound, or a pharmaceutically acceptable salt or solvate thereof, of Embodiment 1 having the structure of Formula (I):

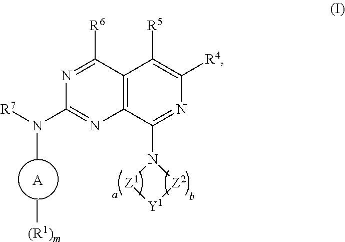

wherein, A is a ring selected from optionally substituted carbocycle, optionally substituted 4- to 6-membered heterocycle, and optionally substituted isoindoline.

Embodiment 3 relates to the compound, or a pharmaceutically acceptable salt or solvate thereof, of Embodiments 1 or 2 having the structure of any one of Formulae (IA), (IB), (IC), (ID), or (IE):

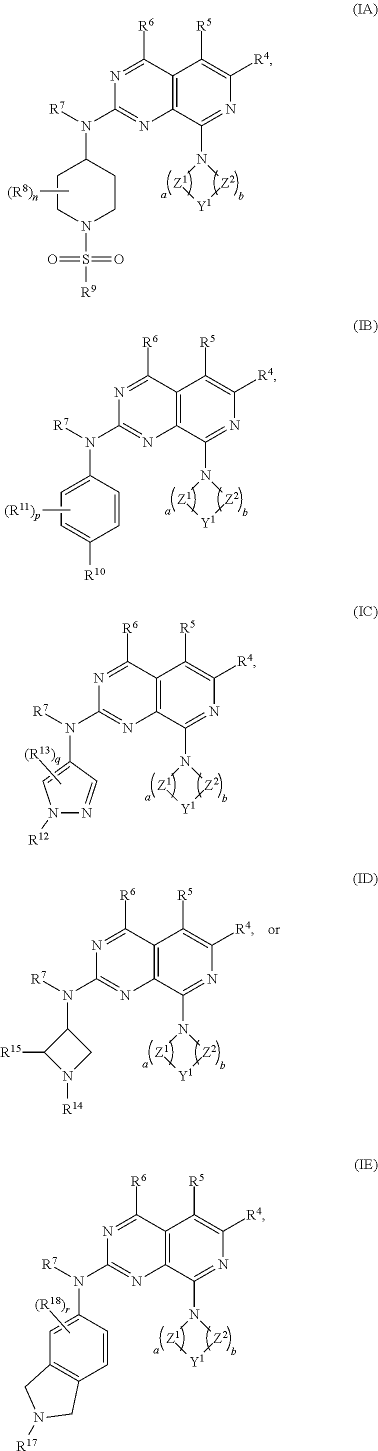

wherein,
R⁸ is selected from halogen, —CN, and optionally substituted C₁₋₄ alkyl;
R⁹ is selected from optionally substituted C₃₋₆ carbocycle, optionally substituted C₅₋₆ heteroaryl, and 3- to 6-membered heterocycloalkyl;
R¹⁰ is optionally substituted alkyl or optionally substituted heterocycloalkyl;
R¹¹ is selected from halogen, —CN, and optionally substituted C₁₋₄ alkyl;
R¹² is selected from an optionally substituted heterocycloalkyl, optionally substituted heterocycloalkylalkyl, optionally substituted cycloalkyl, and optionally substituted cycloalkylalkyl; or R¹² and R¹³ come together to form an optionally substituted heterocycle;
R¹³ is selected from halogen, —CN, and optionally substituted C₁₋₄ alkyl;
n is selected from 0 to 9;
R¹⁴ is selected from —SOR¹⁶—, and optionally substituted heterocycloalkyl;
R¹⁵ is selected from hydrogen, halogen, —CN, and optionally substituted C₁₋₄ alkyl;
R¹⁶ is selected from optionally substituted C₁₋₄ alkyl, optionally substituted C₃₋₆ carbocycle, and optionally substituted 3- to 6-membered heterocycloalkyl;
R¹⁷ is selected from —SOR¹⁹—, optionally substituted alkyl, optionally substituted carbocycle, and optionally substituted heterocycloalkyl;
R¹⁸ is selected from halogen, —CN, and optionally substituted C₁₋₄ alkyl;
R¹⁹ is selected from optionally substituted C₁₋₄ alkyl, optionally substituted C₃₋₆ carbocycle, and optionally substituted 3- to 6-membered heterocycloalkyl;
r is selected from 0 to 5.
p is selected from 0 to 4;
q is selected from 0 to 2; and
wherein when the compound is of formula (IA), either (i) Y¹ is —C(R²)₂— and the two R² substituents come together to form a ring selected from an optionally substituted heterocycle and an optionally substituted carbocycle, or (ii) R⁴ is selected from hydrogen, halogen, and —CN.

Embodiment 4 relates to the compound, or a pharmaceutically acceptable salt or solvate thereof, of Embodiment 3 having the structure of Formula (IA).

Embodiment 5 relates to the compound, or a pharmaceutically acceptable salt or solvate thereof, of Embodiment 3 having the structure of Formula (IB).

Embodiment 6 relates to the compound, or a pharmaceutically acceptable salt or solvate thereof, of Embodiment 3 having the structure of Formula (IC).

Embodiment 7 relates to the compound, or a pharmaceutically acceptable salt or solvate thereof, of Embodiment 3 having the structure of Formula (IE).

Embodiment 8 relates to the compound, or a pharmaceutically acceptable salt or solvate thereof, of any one of Embodiments 1-7, wherein Z¹ and Z² are independently selected from —C(R²)₂—, —NR³—, —O—, and —S—.

Embodiment 9 relates to the compound, or a pharmaceutically acceptable salt or solvate thereof, of Embodiment 8, wherein Z¹ and Z² are independently —C(R²)₂—.

Embodiment 10 relates to the compound, or a pharmaceutically acceptable salt or solvate thereof, of Embodiment 9, wherein each of a and b are independently selected from 1 and 2.

Embodiment 11 relates to the compound, or a pharmaceutically acceptable salt or solvate thereof, of any one of Embodiments 1-10, wherein Y¹ is selected from —C(R²)₂—, —NR³—, —NS(O₂)R², —O—, —S(O)₂—, and —S—.

Embodiment 12 relates to the compound, or a pharmaceutically acceptable salt or solvate thereof, of Embodiment 11, wherein Y¹ is selected from —C(R²)₂— and —NR³.

Embodiment 13 relates to the compound, or a pharmaceutically acceptable salt or solvate thereof, of any one of Embodiments 1-12, wherein each R² is independently selected from hydrogen, OH, halogen, —CN, optionally substituted alkyl, optionally substituted —O-alkyl, optionally substituted cycloalkyl, and optionally substituted heterocycloalkyl.

Embodiment 14 relates to the compound, or a pharmaceutically acceptable salt or solvate thereof, of Embodiment 13, wherein each R² is independently selected from hydrogen, —CN, cyclopropyl, cyclobutyl, optionally substituted cycloalkyl, and optionally substituted heterocycloalkyl.

Embodiment 15 relates to the compound, or a pharmaceutically acceptable salt or solvate thereof, of Embodiment 14, wherein each R² is independently selected from hydrogen, —CN and cyclopropyl.

Embodiment 16 relates to the compound, or a pharmaceutically acceptable salt or solvate thereof, of any one of Embodiments 1-15, wherein two R² substituents come together to form an optionally substituted heterocycle or an optionally substituted carbocycle.

Embodiment 17 relates to the compound, or a pharmaceutically acceptable salt or solvate thereof, of any one of Embodiments 1-16, wherein each R³ is selected from fluoro, optionally substituted alkyl, cyclopropyl, cyclobutyl, optionally substituted oxetane, and optionally substituted azetidine.

Embodiment 18 relates to the compound, or a pharmaceutically acceptable salt or solvate thereof, of Embodiment 17, wherein each R³ is selected from methyl, methoxyethylene, CD3, cyclopropyl and cyclobutyl.

Embodiment 19 relates to the compound, or a pharmaceutically acceptable salt or solvate thereof, of Embodiment 18, wherein R³ is cyclopropyl.

Embodiment 20 relates to the compound, or a pharmaceutically acceptable salt or solvate thereof, of Embodiment 1-3 having the structure of any one of Formulae (IAA), (IBB), (ICC), (IDD), or (IEE):

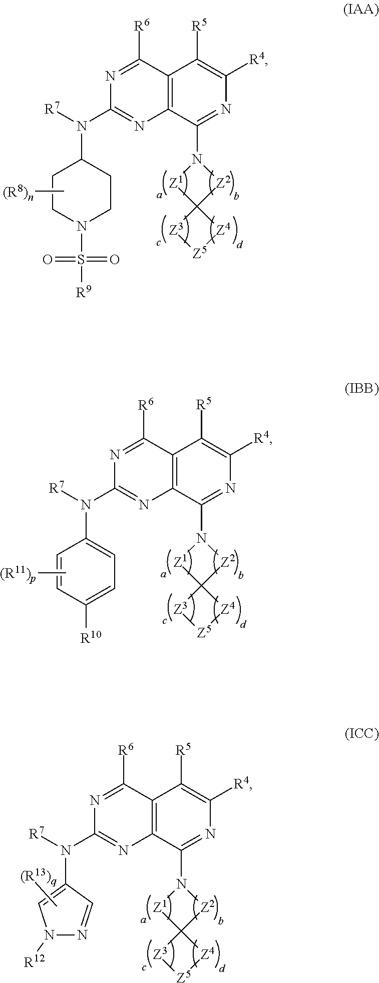

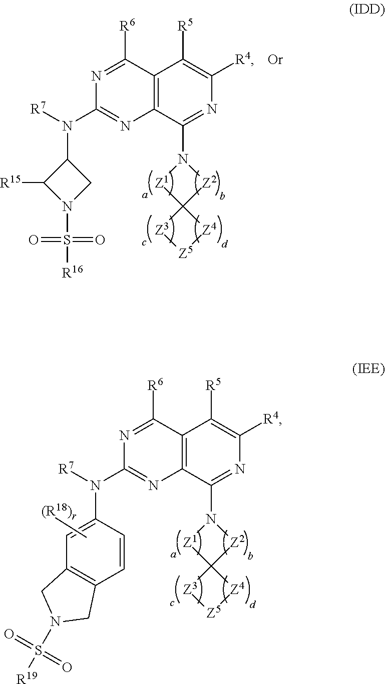

wherein,
R⁸ is selected from halogen, —CN, and optionally substituted C₁₋₄ alkyl;
R⁹ is selected from optionally substituted C₃₋₆ carbocycle, optionally substituted C₅₋₆ heteroaryl, and 3- to 6-membered heterocycloalkyl;
R¹⁰ is optionally substituted heterocycloalkyl;
R¹¹ is selected from halogen, —CN, and optionally substituted C₁₋₄ alkyl;
R¹² is optionally substituted heterocycloalkyl; or R¹¹ and R¹³ come together to form an optionally substituted heterocycle;
R¹³ is selected from halogen, —CN, and optionally substituted C₁₋₄ alkyl;
R¹⁵ is selected from hydrogen, halogen, —CN, and optionally substituted C₁₋₄ alkyl;
R¹⁶ is selected from optionally substituted C₁₋₄ alkyl, optionally substituted C₃₋₆ carbocycle, and optionally substituted 3- to 6-membered heterocycloalkyl;
R¹⁹ is selected from optionally substituted C₁₋₄ alkyl, optionally substituted C₃₋₆ carbocycle, and optionally substituted 3- to 6-membered heterocycloalkyl;
n is selected from 0 to 9;
p is selected from 0 to 4;
q is selected from 0 to 2;
each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond; and
each of a, b, c, and d are independently selected from 1, 2, 3, and 4.

Embodiment 21 relates to the compound, or a pharmaceutically acceptable salt or solvate thereof, of Embodiment 20 having the structure of Formula (IAA).

Embodiment 22 relates to the compound, or a pharmaceutically acceptable salt or solvate thereof, of Embodiment 20 having the structure of Formula (IBB).

Embodiment 23 relates to the compound, or a pharmaceutically acceptable salt or solvate thereof, of Embodiment 20 having the structure of Formula (ICC).

Embodiment 24 relates to the compound, or a pharmaceutically acceptable salt or solvate thereof, of Embodiment 20 having the structure of Formula (IEE).

Embodiment 25 relates to the compound, or a pharmaceutically acceptable salt or solvate thereof, of any one of Embodiments 16-24, wherein each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond.

Embodiment 26 relates to the compound, or a pharmaceutically acceptable salt or solvate thereof, of Embodiment 25, wherein each of Z¹, Z², Z³, Z⁴ and Z⁵ are independently selected from —C(R²)₂—, —NR³—, —O—, and —S(O)₂—, wherein Z⁵ is additionally selected from a bond.

Embodiment 27 relates to the compound, or a pharmaceutically acceptable salt or solvate thereof, of any one of Embodiments 16-26, wherein each R² is independently selected from hydrogen, halogen, —CN, OH, optionally substituted alkyl, optionally substituted cycloalkyl, and optionally substituted heterocycloalkyl, or two R² substituents come together to form an optionally substituted heterocycle or an optionally substituted carbocycle.

Embodiment 28 relates to the compound, or a pharmaceutically acceptable salt or solvate thereof, of Embodiment 27, wherein each R² is selected from hydrogen, fluoro, CN, cyclopropyl, cyclobutyl, —C₁₋₄ alkyl, —C₁₋₄ haloalkyl, —O—C₁-4 alkyl, —C₁₋₄ alkylene-O—C₁₋₃ alkyl, —C₁₋₄ alkylene-OH, optionally substituted oxetane, and optionally substituted azetidine.

Embodiment 29 relates to the compound, or a pharmaceutically acceptable salt or solvate thereof, of any one of Embodiments 16-28, wherein R³ is selected from hydrogen, —(CH₂)₂OMe, —S(O)₂CH₃, —C₁₋₄ alkylene-O—C₁₋₃ alkyl, C₁₋₄ alkyl, cyclopropyl, cyclobutyl, optionally substituted oxetane, and optionally substituted azetidine.

Embodiment 30 relates to the compound, or a pharmaceutically acceptable salt or solvate thereof, of Embodiment 29, wherein R³ is selected from hydrogen, —(CH₂)₂OMe, C₁₋₄ alkyl, cyclopropyl, cyclobutyl, optionally substituted oxetane, and optionally substituted azetidine.

Embodiment 31 relates to the compound, or a pharmaceutically acceptable salt or solvate thereof, of Embodiment 30, wherein R³ is selected from hydrogen, methyl, ethyl, propyl, CD3, and cyclopropyl.

Embodiment 32 relates to the compound, or a pharmaceutically acceptable salt or solvate thereof, of any one of Embodiments 16-31, wherein each a, b, c, and d are each independently selected from 1 and 2.

Embodiment 33 relates to the compound, or a pharmaceutically acceptable salt or solvate thereof, of Embodiment 1 or 2, wherein A is selected from optionally substituted cyclohexane, optionally substituted pyridine, optionally substituted piperidine, optionally substituted tetrahydropyran, optionally substituted azabicyclo[3.1.0]hexane, optionally substituted azetidine, and optionally substituted tetrahydroisoquinoline.

Embodiment 34 relates to the compound, or a pharmaceutically acceptable salt or solvate thereof, of Embodiment 33, wherein A is optionally substituted piperidine.

Embodiment 35 relates to the compound, or a pharmaceutically acceptable salt or solvate thereof, of Embodiment 34, wherein A is substituted with —SO₂R⁹, wherein R⁹ is selected from optionally substituted C₃₋₆ carbocycle, optionally substituted C₅₋₆ heteroaryl, and 3- to 6-membered heterocycloalkyl

Embodiment 36 relates to the compound, or a pharmaceutically acceptable salt or solvate thereof, of Embodiment 34 or 35, wherein m is selected from 0 to 1.

Embodiment 37 relates to the compound, or a pharmaceutically acceptable salt or solvate thereof, of any one of Embodiments 34-36, wherein each R¹ is independently selected from optionally substituted alkyl, optionally substituted carbocycle, and optionally substituted heterocycle.

Embodiment 38 relates to the compound, or a pharmaceutically acceptable salt or solvate thereof, of Embodiment 37, wherein each R¹ is independently selected from optionally substituted alkyl and optionally substituted heterocycle.

Embodiment 39 relates to the compound, or a pharmaceutically acceptable salt or solvate thereof, of Embodiment 37, wherein each R¹ is independently selected from methyl, ethyl, and optionally substituted diazaspiro[3.3]heptane.

Embodiment 40 relates to the compound, or a pharmaceutically acceptable salt or solvate thereof, of any one of Embodiments 1-13, and 34-39 wherein Z¹ and Z² are independently selected from —C(R²)₂—, —NR³—, —O—, and —S—.

Embodiment 41 relates to the compound, or a pharmaceutically acceptable salt or solvate thereof, of Embodiment 40, wherein Z¹ and Z² are each —C(R²)₂—.

Embodiment 42 relates to the compound, or a pharmaceutically acceptable salt or solvate thereof, of Embodiment 40 or 41, wherein each of a and b are independently selected from 1 and 2.

Embodiment 43 relates to the compound, or a pharmaceutically acceptable salt or solvate thereof, of any one of Embodiments 1-13, and 34-42, wherein Y¹ is selected from —C(R²)₂—, —NR³—, —O—, and —S—.

Embodiment 44 relates to the compound, or a pharmaceutically acceptable salt or solvate thereof, of Embodiment 43, wherein Y¹ is selected from —C(R²)₂— and —NR³.

Embodiment 45 relates to the compound, or a pharmaceutically acceptable salt or solvate thereof, of Embodiment 44, wherein Y¹ is —C(R²)₂— and two R² substituents come together to form a ring selected from an optionally substituted heterocycle and an optionally substituted carbocycle.

Embodiment 46 relates to the compound, or a pharmaceutically acceptable salt or solvate thereof, of Embodiment 46, wherein Y¹ is —C(R²)₂— and the two R² substituents come together to form a ring selected from an optionally substituted heterocycle.

Embodiment 47 relates to the compound, or a pharmaceutically acceptable salt or solvate thereof, of any one of Embodiments 1-19, and 34-46, wherein the N containing heterocyclic ring depicted as

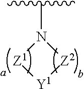

is selected from optionally substituted azetidine, optionally substituted pyrrolidine, optionally substituted piperidine, optionally substituted piperazine, optionally substituted morpholine, optionally substituted tetrahydrothienopyrroledioxide, and optionally substituted dihydroindole.

Embodiment 48 relates to the compound, or a pharmaceutically acceptable salt or solvate thereof, of any one of Embodiments 1-19, and 34-46, wherein the N containing heterocyclic ring depicted as

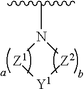

in Formula (I), (IB), (IC), (ID), and (IE) is selected from

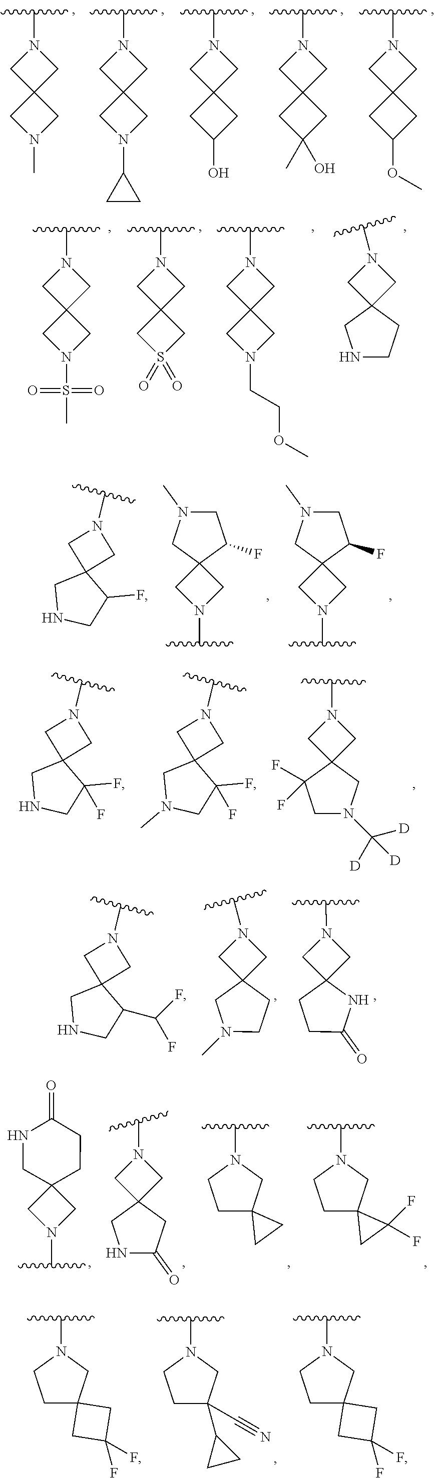

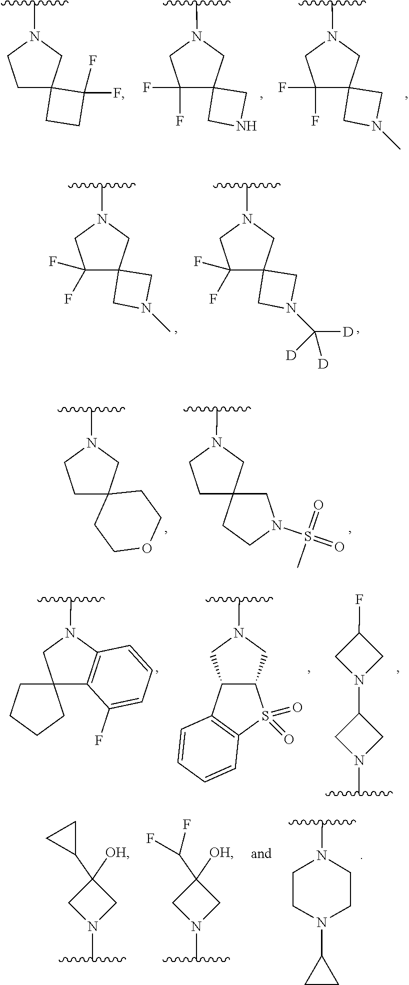

Embodiment 48(a) relates to the compound, or a pharmaceutically acceptable salt or solvate thereof, of Embodiment 48, wherein the N containing heterocyclic ring depicted as

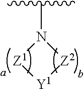

### in Formula (I), (IB), (IC), (ID), and (IE) is

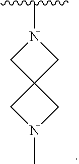 [SMILES: *N1CC2(C1)CN(C)C2]

Embodiment 48(b) relates to the compound, or a pharmaceutically acceptable salt or solvate thereof, of Embodiment 48, wherein the N containing heterocyclic ring depicted as

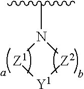

### in Formula (I), (IB), (IC), (ID), and (IE) is

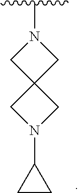 [SMILES: *N1CC2(C1)CN(C1CC1)C2]

Embodiment 48(c) relates to the compound, or a pharmaceutically acceptable salt or solvate thereof, of Embodiment 48, wherein the N containing heterocyclic ring depicted as

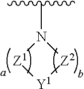

### in Formula (I), (IB), (IC), (ID), and (IE) is

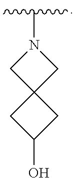 [SMILES: *N1CC2(CC(O)C2)C1]

Embodiment 48(d) relates to the compound, or a pharmaceutically acceptable salt or solvate thereof, of Embodiment 48, wherein the N containing heterocyclic ring depicted as

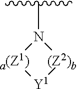

### in Formula (I), (IB), (IC), (ID), and (IE) is

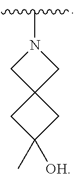 [SMILES: *N1CC2(C1)CC(C)(O)C2]

Embodiment 48(e) relates to the compound, or a pharmaceutically acceptable salt or solvate thereof, of Embodiment 48, wherein the N containing heterocyclic ring depicted as

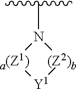

### in Formula (I), (IB), (IC), (ID), and (IE) is P,

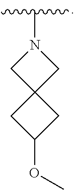 [SMILES: *N1CC2(CC(OC)C2)C1]

Embodiment 48(f) relates to the compound, or a pharmaceutically acceptable salt or solvate thereof, of Embodiment 48, wherein the N containing heterocyclic ring depicted as

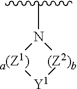

### in Formula (I), (IB), (IC), (ID), and (IE) is

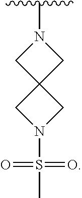 [SMILES: *N1CC2(C1)CN(S(C)(=O)=O)C2]

Embodiment 48(g) relates to the compound, or a pharmaceutically acceptable salt or solvate thereof, of Embodiment 48, wherein the N containing heterocyclic ring depicted as

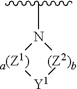

### in Formula (I), (IB), (IC), (ID), and (IE) is

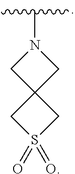 [SMILES: *N1CC2(C1)CS(=O)(=O)C2]

Embodiment 48(h) relates to the compound, or a pharmaceutically acceptable salt or solvate thereof, of Embodiment 48, wherein the N containing heterocyclic ring depicted as

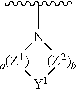

### in Formula (I), (IB), (IC), (ID), and (IE) is

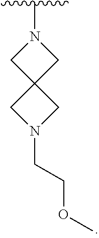 [SMILES: *N1CC2(C1)CN(CCOC)C2]

Embodiment 48(i) relates to the compound, or a pharmaceutically acceptable salt or solvate thereof, of Embodiment 48, wherein the N containing heterocyclic ring depicted as

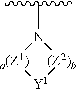

### Formula (I), (IB), (IC), (ID), and (IE) is

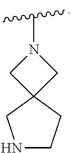 [SMILES: *N1CC2(CCNC2)C1]

Embodiment 48(j) relates to the compound, or a pharmaceutically acceptable salt or solvate thereof, of Embodiment 48, wherein the N containing heterocyclic ring depicted as

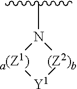

### in Formula (I), (IB), (IC), (ID), and (IE) is

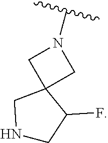 [SMILES: *N1CC2(CNCC2F)C1]

Embodiment 48(k) relates to the compound, or a pharmaceutically acceptable salt or solvate thereof, of Embodiment 48, wherein the N containing heterocyclic ring depicted as

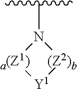

### in Formula (I), (IB), (IC), (ID), and (IE) is

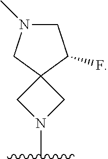 [SMILES: *N1CC2(C1)CN(C)C[C@@H]2F]

Embodiment 48(1) relates to the compound, or a pharmaceutically acceptable salt or solvate thereof, of Embodiment 48, wherein the N containing heterocyclic ring depicted as

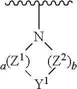

### in Formula (I), (IB), (IC), (ID), and (IE) is

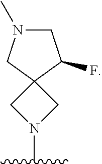 [SMILES: *N1CC2(C1)CN(C)C[C@H]2F]

Embodiment 48(m) relates to the compound, or a pharmaceutically acceptable salt or solvate thereof, of Embodiment 48, wherein the N containing heterocyclic ring depicted as

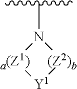

### in Formula (I), (IB), (IC), (ID), and (IE) is

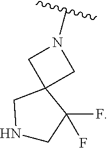 [SMILES: *N1CC2(CNCC2(F)F)C1]

Embodiment 48(n) relates to the compound, or a pharmaceutically acceptable salt or solvate thereof, of Embodiment 48, wherein the N containing heterocyclic ring depicted as

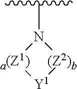

### in Formula (I), (IB), (IC), (ID), and (IE) is

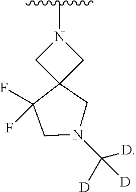

Embodiment 48(o) relates to the compound, or a pharmaceutically acceptable salt or solvate thereof, of Embodiment 48, wherein the N containing heterocyclic ring depicted as

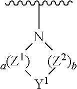

### in Formula (I), (IB), (IC), (ID), and (IE) is

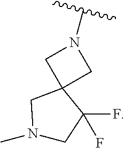 [SMILES: *N1CC2(C1)CN(C)CC2(F)F]

Embodiment 48(p) relates to the compound, or a pharmaceutically acceptable salt or solvate thereof, of Embodiment 48, wherein the N containing heterocyclic ring depicted as

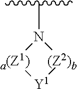

### Formula (I), (IB), (IC), (ID), and (IE) is

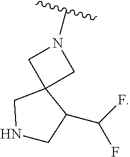 [SMILES: *N1CC2(CNCC2C(F)F)C1]

Embodiment 48(q) relates to the compound, or a pharmaceutically acceptable salt or solvate thereof, of Embodiment 48, wherein the N containing heterocyclic ring depicted as

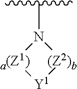

### in Formula (I), (IB), (IC), (ID), and (IE) is

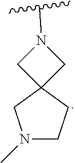 [SMILES: *N1CC2(CCN(C)C2)C1]

Embodiment 48(r) relates to the compound, or a pharmaceutically acceptable salt or solvate thereof, of Embodiment 48, wherein the N containing heterocyclic ring depicted as

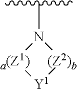

### in Formula (I), (IB), (IC), (ID), and (IE) is

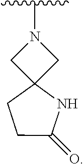 [SMILES: *N1CC2(CCC(=O)N2)C1]

Embodiment 48(s) relates to the compound, or a pharmaceutically acceptable salt or solvate thereof, of Embodiment 48, wherein the N containing heterocyclic ring depicted as

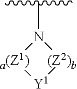

### in Formula (I), (IB), (IC), (ID), and (IE) is

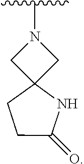 [SMILES: *N1CC2(CCC(=O)N2)C1]

Embodiment 48(t) relates to the compound, or a pharmaceutically acceptable salt or solvate thereof, of Embodiment 48, wherein the N containing heterocyclic ring depicted as

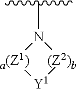

### in Formula (I), (IB), (IC), (ID), and (IE) is

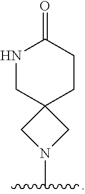 [SMILES: *N1CC2(CCC(=O)NC2)C1]

Embodiment 48(u) relates to the compound, or a pharmaceutically acceptable salt or solvate thereof, of Embodiment 48, wherein the N containing heterocyclic ring depicted as

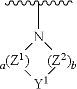

### in Formula (I), (IB), (IC), (ID), and (IE) is

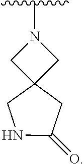 [SMILES: *N1CC2(CNC(=O)C2)C1]

Embodiment 48(v) relates to the compound, or a pharmaceutically acceptable salt or solvate thereof, of Embodiment 48, wherein the N containing heterocyclic ring depicted as

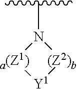

### in Formula (I), (IB), (IC), (ID), and (IE) is

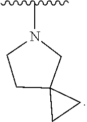 [SMILES: *N1CCC2(CC2)C1]

Embodiment 48(w) relates to the compound, or a pharmaceutically acceptable salt or solvate thereof, of Embodiment 48, wherein the N containing heterocyclic ring depicted as

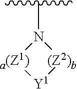

### in Formula (I), (IB), (IC), (ID), and (IE) is

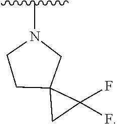 [SMILES: *N1CCC2(C1)CC2(F)F]

Embodiment 48(x) relates to the compound, or a pharmaceutically acceptable salt or solvate thereof, of Embodiment 48, wherein the N containing heterocyclic ring depicted as

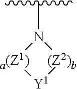

### in Formula (I), (IB), (IC), (ID), and (IE) is

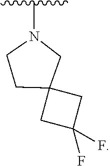 [SMILES: *N1CCC2(C1)CC(F)(F)C2]

Embodiment 48(y) relates to the compound, or a pharmaceutically acceptable salt or solvate thereof, of Embodiment 48, wherein the N containing heterocyclic ring depicted as

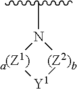

### in Formula (IA), (IB), (IC), (ID), and (IE) is

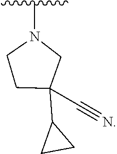 [SMILES: *N1CCC(C#N)(C2CC2)C1]

Embodiment 48(z) relates to the compound, or a pharmaceutically acceptable salt or solvate thereof, of Embodiment 48, wherein the N containing heterocyclic ring depicted as

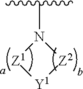

### in Formula (IA), (IB), (IC), (ID), and (IE) is

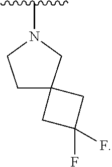 [SMILES: *N1CCC2(C1)CC(F)(F)C2]

Embodiment 48(aa) relates to the compound, or a pharmaceutically acceptable salt or solvate thereof, of Embodiment 48, wherein the N containing heterocyclic ring depicted as

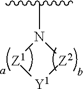

### in Formula (IA), (IB), (IC), (ID), and (IE) is

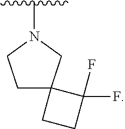 [SMILES: *N1CCC2(CCC2(F)F)C1]

Embodiment 48(ab) relates to the compound, or a pharmaceutically acceptable salt or solvate thereof, of Embodiment 48, wherein the N containing heterocyclic ring depicted as

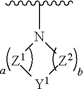

### in Formula (IA), (IB), (IC), (ID), and (IE) is

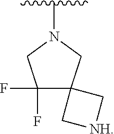 [SMILES: *N1CC(F)(F)C2(CNC2)C1]

Embodiment 48(ac) relates to the compound, or a pharmaceutically acceptable salt or solvate thereof, of Embodiment 48, wherein the N containing heterocyclic ring depicted as

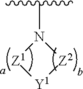

### in Formula (IA), (IB), (IC), (ID), and (IE) is

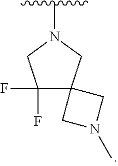 [SMILES: *N1CC(F)(F)C2(C1)CN(C)C2]

Embodiment 48(ad) relates to the compound, or a pharmaceutically acceptable salt or solvate thereof, of Embodiment 48, wherein the N containing heterocyclic ring depicted as

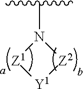

### in Formula (IA), (IB), (IC), (ID), and (IE) is

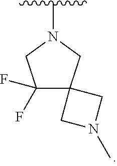 [SMILES: *N1CC(F)(F)C2(C1)CN(C)C2]

Embodiment 48(ae) relates to the compound, or a pharmaceutically acceptable salt or solvate thereof, of Embodiment 48, wherein the N containing heterocyclic ring depicted as

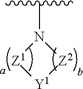

### in Formula (IA), (IB), (IC), (ID), and (IE) is

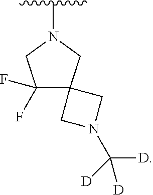

Embodiment 48(af) relates to the compound, or a pharmaceutically acceptable salt or solvate thereof, of Embodiment 48, wherein the N containing heterocyclic ring depicted as

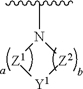

### in Formula (IA), (IB), (IC), (ID), and (IE) is

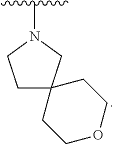 [SMILES: *N1CCC2(CCOCC2)C1]

Embodiment 48(ag) relates to the compound, pharmaceutically acceptable salt or solvate thereof, of Embodiment 48, wherein the N containing heterocyclic ring depicted as

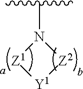

### in Formula (IA), (IB), (IC), (ID), and (IE) is

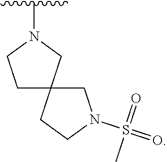 [SMILES: *N1CCC2(CCN(S(C)(=O)=O)C2)C1]

Embodiment 48(ah) relates to the compound, or a pharmaceutically acceptable salt or solvate thereof, of Embodiment 48, wherein the N containing heterocyclic ring depicted as

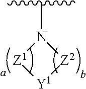

### in Formula (IA), (IB), (IC), (ID), and (IE) is

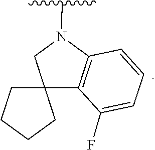 [SMILES: *N1CC2(CCCC2)c2c(F)cccc21]

Embodiment 48(ai) relates to the compound, or a pharmaceutically acceptable salt or solvate thereof, of Embodiment 48, wherein the N containing heterocyclic ring depicted as

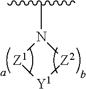

### in Formula (IA), (IB), (IC), (ID), and (IE) is

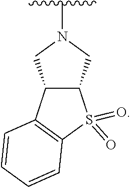 [SMILES: *N1C[C@H]2c3ccccc3S(=O)(=O)[C@H]2C1]

Embodiment 48(aj) relates to the compound, or a pharmaceutically acceptable salt or solvate thereof, of Embodiment 48, wherein the N containing heterocyclic ring depicted as

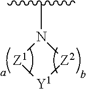

### in Formula (IA), (IB), (IC), (ID), and (IE) is

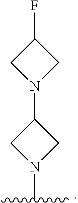 [SMILES: *N1CC(N2CC(F)C2)C1]

Embodiment 48(ak) relates to the compound, or a pharmaceutically acceptable salt or solvate thereof, of Embodiment 48, wherein the N containing heterocyclic ring depicted as

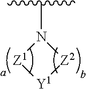

### in Formula (IA), (IB), (IC), (ID), and (IE) is

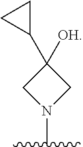 [SMILES: *N1CC(O)(C2CC2)C1]

Embodiment 48(al) relates to the compound, or a pharmaceutically acceptable salt or solvate thereof, of Embodiment 48, wherein the N containing heterocyclic ring depicted as

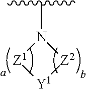

### in Formula (IA), (IB), (IC), (ID), and (IE) is

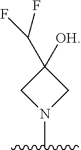 [SMILES: *N1CC(O)(C(F)F)C1]

Embodiment 48(am) relates to the compound, or a pharmaceutically acceptable salt or solvate thereof, of Embodiment 48, wherein the N containing heterocyclic ring depicted as

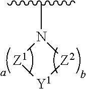

### in Formula (IA), (IB), (IC), (ID), and (IE) is

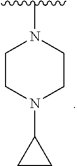 [SMILES: *N1CCN(C2CC2)CC1]

Embodiment 49 relates to the compound, or a pharmaceutically acceptable salt or solvate thereof, of any one of Embodiments 1-32, 34-48, or 48(a)-48(aj), wherein R⁴ is selected from hydrogen, halogen, optionally substituted C₁₋₂ alkyl, optionally substituted C₃₋₄ carbocycle, and optionally substituted 3- to 4 membered heterocycloalkyl.

Embodiment 50 relates to the compound, or a pharmaceutically acceptable salt or solvate thereof, of Embodiment 49, wherein R⁴ is selected from hydrogen and optionally substituted C₁ alkyl.

Embodiment 51 relates to the compound, or a pharmaceutically acceptable salt or solvate thereof, of Embodiment 50, wherein R⁴ is selected from hydrogen, methyl, and —CHF₂.

Embodiment 52 relates to the compound, or a pharmaceutically acceptable salt or solvate thereof, of any one of Embodiments 1-32, 34-48, or 48(a)-48(aj), wherein R⁴ is selected from hydrogen, halogen, and —CN.

Embodiment 53 relates to the compound, or a pharmaceutically acceptable salt or solvate thereof, of any one of Embodiments 1-32, 34-48, or 48(a)-48(aj), wherein R⁴ is selected from hydrogen.

Embodiment 54 relates to the compound, or a pharmaceutically acceptable salt or solvate thereof, of any one of Embodiments 1-32, 34-48, or 48(a)-48(aj), wherein R⁴ is not optionally substituted phenyl.

Embodiment 55 relates to the compound, or a pharmaceutically acceptable salt or solvate thereof, of any one of Embodiments 1-32, 34-48, or 48(a)-48(aj), wherein R⁴ is not optionally substituted alkyl.

Embodiment 56 relates to the compound, or a pharmaceutically acceptable salt or solvate thereof, of any one of Embodiments 1-32, 34-39, or 50-55, wherein R⁵ is selected from hydrogen, halogen, optionally substituted C₁₋₂ alkyl, optionally substituted C₃₋₄ carbocycle, and optionally substituted 3- to 4 membered heterocycloalkyl.

Embodiment 57 relates to the compound, or a pharmaceutically acceptable salt or solvate thereof, of Embodiment 56, wherein R⁵ is selected from hydrogen, halogen, and optionally substituted C₁₋₂ alkyl.

Embodiment 58 relates to the compound, or a pharmaceutically acceptable salt or solvate thereof, of Embodiment 57, wherein R⁵ is selected from hydrogen and fluoro.

Embodiment 59 relates to the compound, or a pharmaceutically acceptable salt or solvate thereof, of any one of Embodiments 1-32, 34-39, or 50-58, wherein R⁶ is selected from hydrogen, halogen, optionally substituted C₁₋₂ alkyl, optionally substituted C₃₋₄ carbocycle, and optionally substituted 3- to 4 membered heterocycloalkyl.

Embodiment 60 relates to the compound, or a pharmaceutically acceptable salt or solvate thereof, of Embodiment 59, wherein R⁶ is selected from hydrogen, halogen, and optionally substituted C₁₋₂ alkyl.

Embodiment 61 relates to the compound, or a pharmaceutically acceptable salt or solvate thereof, of Embodiment 60, wherein R⁶ is hydrogen.

Embodiment 62 relates to the compound, or a pharmaceutically acceptable salt or solvate thereof, of any one of Embodiments 1-32, 34-39, or 50-61, wherein R⁷ is hydrogen.

Embodiment 63 relates to the compound, or a pharmaceutically acceptable salt or solvate thereof, of any one of Embodiments 3-4, 8-21, or 25-32, wherein R⁹ is selected from cyclopentyl, methylcyclopentyl, cyclobutylmethylene, cyclopentylmethylene, and n-methylpyrazolyl.

Embodiment 63(a) relates to the compound, or a pharmaceutically acceptable salt or solvate thereof, of Embodiment 63 wherein R⁹ is cyclopentyl.

Embodiment 63(b) relates to the compound, or a pharmaceutically acceptable salt or solvate thereof, of Embodiment 63 wherein R⁹ is methylcyclopentyl.

Embodiment 63(c) relates to the compound, or a pharmaceutically acceptable salt or solvate thereof, of Embodiment 63 wherein R⁹ is cyclobutylmethylene.

Embodiment 63(d) relates to the compound, or a pharmaceutically acceptable salt or solvate thereof, of Embodiment 63 wherein R⁹ is cyclopentylmethylene.

Embodiment 63(e) relates to the compound, or a pharmaceutically acceptable salt or solvate thereof, of Embodiment 63 wherein R⁹ is n-methylpyrazolyl.

Embodiment 64 relates to the compound, or a pharmaceutically acceptable salt or solvate thereof, of Embodiment 1, wherein the compound is selected from Table 1.

Embodiment 65 relates to the compound, or a pharmaceutically acceptable salt or solvate thereof, of Embodiment 2, wherein the compound is selected from Table 1.

Embodiment 66 relates to the compound, or a pharmaceutically acceptable salt or solvate thereof, of Embodiment 3, wherein the compound is selected from Table 1.

Embodiment 66(a) of this disclosure relates to Formula (IA), or a pharmaceutically acceptable salt thereof, of Embodiment 66 selected from the compounds in Table 1.

Embodiment 66(b) of this disclosure relates to Formula (IB), or a pharmaceutically acceptable salt thereof, of Embodiment 66 selected from the compounds in Table 1.

Embodiment 66(c) of this disclosure relates to Formula (IC), or a pharmaceutically acceptable salt thereof, of Embodiment 66 selected from the compounds in Table 1.

Embodiment 66(d) of this disclosure relates to Formula (ID), or a pharmaceutically acceptable salt thereof, of Embodiment 66 selected from the compounds in Table 1.

Embodiment 66(e) of this disclosure relates to Formula (IE), or a pharmaceutically acceptable salt thereof, of Embodiment 66 selected from the compounds in Table 1.

Embodiment 67 relates to the compound, or a pharmaceutically acceptable salt or solvate thereof, of Embodiment 20, wherein the compound is selected from Table 1.

Embodiment 67(a) of this disclosure relates to Formula (IAA), or a pharmaceutically acceptable salt thereof, of Embodiment 66 selected from the compounds in Table 1.

Embodiment 67(b) of this disclosure relates to Formula (IBB), or a pharmaceutically acceptable salt thereof, of Embodiment 66 selected from the compounds in Table 1.

Embodiment 67(c) of this disclosure relates to Formula (ICC), or a pharmaceutically acceptable salt thereof, of Embodiment 66 selected from the compounds in Table 1.

Embodiment 67(d) of this disclosure relates to Formula (IDD), or a pharmaceutically acceptable salt thereof, of Embodiment 66 selected from the compounds in Table 1.

Embodiment 67(e) of this disclosure relates to Formula (IEE), or a pharmaceutically acceptable salt thereof, of Embodiment 66 selected from the compounds in Table 1.

The compounds disclosed herein, in some embodiments, are used in different enriched isotopic forms, e.g., enriched in the content of ²H, ³H, 11C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (3H), iodine-125 (¹²⁵I) or carbon-14 (¹⁴C). Isotopic substitution with ²H, 11C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quatemary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J Pharmaceutics, 37, 87 (1987); J. Larsen et al., Int. J Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Therapeutic Applications

Methods of administration of a compound or pharmaceutically acceptable salt of Formula (I), (IA), (IAA), (IAAA), (IB), (IBB), (IC), (ICC), (ID), (IDD), (IE), or (IEE) discussed herein may be used for the treatment of cancer. In some embodiments, disclosed herein are methods to treat solid tumors. Examples of cancer include but are not limited to ovarian cancer, breast cancer, colon cancer, and brain cancer.

In some embodiments disclosed herein are methods to treat cancer by the administration of a compound or pharmaceutically acceptable salt of Formula (I), (IA), (IAA), (IAAA), (IB), (IBB), (IC), (ICC), (ID), (IDD), (IE), or (IEE). In some embodiments, disclosed herein is a method of treating cancer, comprising administering to a subject in need thereof the pharmaceutical composition described herein. In some embodiments, the cancer is a solid tumor. In some embodiments, the cancer is selected from ovarian cancer, breast cancer, colon cancer, and brain cancer. In some embodiments the cancer is ovarian cancer or breast cancer.

In some embodiments, disclosed herein is a method of inhibiting a cyclin dependent kinase (CDK) in a cell by the administration of a compound or pharmaceutically acceptable salt of Formula (I), (IA), (IAA), (IAAA), (IB), (IBB), (IC), (ICC), (ID), (IDD), (IE), or (IEE). In some embodiments, disclosed herein is a method of inhibiting a cyclin dependent kinase (CDK) in a cell with a compound or pharmaceutically acceptable salt of any one of the compounds described herein or the pharmaceutical composition described herein.

The CDK can be any suitable CDK known by one of skill in the art. In some embodiments, CDK is selected from CDK 2, CDK 4, CD6, or any combination thereof. In some embodiments, the CDK is selected from CDK2, CDK4, CDK6, CDK 2/4, CDK 2/6, CDK 4/6, and CDK 2/4/6. In some embodiments, the CDK is selected from CDK 2/4, CDK 2/6, CDK 4/6, and CDK 2/4/6.

Additional embodiments of the therapeutic applications of this disclosure include the following:

Embodiment 69 relates to a method of treating cancer, comprising administering to a subject in need thereof a compound, or a pharmaceutically acceptable salt thereof, according to any one of Embodiments 1-67, or any sub-embodiments thereof, or the pharmaceutical composition of Embodiment 68.

Embodiment 70 relates to the method of Embodiment 69, wherein the cancer is a solid tumor.

Embodiment 71 relates to the method of Embodiment 69 or 70, wherein the cancer is selected from ovarian cancer, breast cancer, colon cancer, and brain cancer.

Embodiment 72 relates to the method of Embodiment 71, wherein the cancer is ovarian cancer or breast cancer.

Embodiment 73 relates to a method of inhibiting a cyclin dependent kinase (CDK) in a cell with a compound, or pharmaceutically acceptable salt thereof, of any one of Embodiments 1 to 67, or any sub-embodiments thereof, or the pharmaceutical composition of Embodiment 68.

Embodiment 74 relates to a method of Embodiment 73, wherein the CDK is selected from CDK 2, CDK 4, CD6, or any combination thereof.

Embodiment 75 relates to a method of Embodiment 74, wherein the CDK is selected from CDK 2/4, CDK 2/6, CDK 4/6, and CDK 2/4/6.

Embodiment 76 relates to a method of Embodiment 75, wherein the CDK is CDK 2/4/6.

### Pharmaceutical Formulations

The compositions and methods described herein may be considered useful as pharmaceutical compositions for administration to a subject in need thereof. Pharmaceutical compositions may comprise at least a compound or pharmaceutically acceptable salt of Formula (I), (IA), (IAA), (IAAA), (IB), (IBB), (IC), (ICC), (ID), (IDD), (IE), or (IEE) described herein and one or more pharmaceutically acceptable carriers, diluents, excipients, stabilizers, dispersing agents, suspending agents, and/or thickening agents. In some embodiments, disclosed herein is a pharmaceutical composition comprising a compound or pharmaceutically acceptable salt described herein and a pharmaceutically acceptable excipient. In some embodiments, disclosed herein is a pharmaceutical composition comprising a compound or pharmaceutically acceptable salt of Formula (I), (IA), (IAA), (IAAA), (IB), (IBB), (IC), (ICC), (ID), (IDD), (IE), or (IEE) described herein and a pharmaceutically acceptable excipient.

Pharmaceutical compositions comprising a compound or pharmaceutically acceptable salt of Formula (I), (IA), (IAA), (IAAA), (IB), (IBB), (IC), (ICC), (ID), (IDD), (IE), or (IEE) may be formulated using one or more physiologically-acceptable carriers comprising excipients and auxiliaries. Formulation may be modified depending upon the route of administration chosen. Pharmaceutical compositions comprising a compound, salt or conjugate may be manufactured, for example, by lyophilizing the compound, salt or conjugate, mixing, dissolving, emulsifying, encapsulating or entrapping the conjugate. The pharmaceutical compositions may also include the compounds, salts or conjugates in a free-base form or pharmaceutically-acceptable salt form.

Additional embodiments of the pharmaceutical formulations of this disclosure include the following:

Embodiment 68 relates to a pharmaceutical composition comprising a compound or pharmaceutically acceptable salt of any one of Embodiments 1 to 67, or any sub-embodiments thereof, and a pharmaceutically acceptable excipient.

Methods for formulation of a compound or pharmaceutically acceptable salt of Formula (I), (IA), (IAA), (IAAA), (IB), (IBB), (IC), (ICC), (ID), (IDD), (IE), or (IEE) may include formulating any of the compounds, salts or conjugates with one or more inert, pharmaceutically-acceptable excipients or carriers to form a solid, semi-solid, or liquid composition. Solid compositions may include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates may be lyophilized or in powder form for reconstitution with a suitable vehicle, e.g., sterile pyrogen-free water, before use.

Pharmaceutical compositions comprising a compound or pharmaceutically acceptable salt of Formula (I), (IA), (IAA), (IAAA), (IB), (IBB), (IC), (ICC), (ID), (IDD), (IE), or (IEE) may comprise at least one active ingredient (e.g., a compound, salt or conjugate and other agents). The active ingredients may be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

The compositions and formulations may be sterilized. Sterilization may be accomplished by filtration through sterile filtration.

The compositions comprising a compound or pharmaceutically acceptable salt of Formula (I), (IA), (IAA), (IAAA), (IB), (IBB), (IC), (ICC), (ID), (IDD), (IE), or (IEE) may be formulated for administration as an injection. Non-limiting examples of formulations for injection may include a sterile suspension, solution or emulsion in oily or aqueous vehicles. Suitable oily vehicles may include, but are not limited to, lipophilic solvents or vehicles such as fatty oils or synthetic fatty acid esters, or liposomes. Aqueous injection suspensions may contain substances which increase the viscosity of the suspension. The suspension may also contain suitable stabilizers. Injections may be formulated for bolus injection or continuous infusion. Alternatively, the compositions may be lyophilized or in powder form for reconstitution with a suitable vehicle, e.g., sterile pyrogen-free water, before use.

For parenteral administration, a compound or pharmaceutically acceptable salt of Formula (I), (IA), (IAA), (IAAA), (IB), (IBB), (IC), (ICC), (ID), (IDD), (IE), or (IEE) may be formulated in a unit dosage injectable form (e.g., solution, suspension, emulsion) in association with a pharmaceutically acceptable parenteral vehicle. Such vehicles may be inherently non-toxic, and non-therapeutic. Vehicles may be water, saline, Ringer's solution, dextrose solution, and 5% human serum albumin. Non-aqueous vehicles such as fixed oils and ethyl oleate may also be used. Liposomes may be used as carriers. The vehicle may contain minor amounts of additives such as substances that enhance isotonicity and chemical stability (e.g., buffers and preservatives).

In one embodiment the invention relates to methods and compositions of Formula (I), (IA), (IAA), (IAAA), (IB), (IBB), (IC), (ICC), (ID), (IDD), (IE), or (IEE) formulated for oral delivery to a subject in need. In one embodiment a composition is formulated so as to deliver one or more pharmaceutically active agents to a subject through a mucosa layer in the mouth or esophagus. In another embodiment the composition is formulated to deliver one or more pharmaceutically active agents to a subject through a mucosa layer in the stomach and/or intestines.

In one embodiment compositions of Formula (I), (IA), (IAA), (IAAA), (IB), (IBB), (IC), (ICC), (ID), (IDD), (IE), or (IEE) are provided in modified release dosage forms. Suitable modified release dosage vehicles include, but are not limited to, hydrophilic or hydrophobic matrix devices, water-soluble separating layer coatings, enteric coatings, osmotic devices, multi-particulate devices, and combinations thereof. The compositions may also comprise non-release controlling excipients.

In another embodiment compositions of Formula (I), (IA), (IAA), (IAAA), (IB), (IBB), (IC), (ICC), (ID), (IDD), (IE), or (IEE) are provided in enteric coated dosage forms. These enteric coated dosage forms can also comprise non-release controlling excipients. In one embodiment the compositions are in the form of enteric-coated granules, as controlled-release capsules for oral administration. The compositions can further comprise cellulose, disodium hydrogen phosphate, hydroxypropyl cellulose, pyridazine, lactose, mannitol, or sodium lauryl sulfate. In another embodiment the compositions are in the form of enteric-coated pellets, as controlled-release capsules for oral administration. The compositions can further comprise glycerol monostearate 40-50, hydroxypropyl cellulose, pyridazine, magnesium stearate, methacrylic acid copolymer type C, polysorbate 80, sugar spheres, talc, or triethyl citrate.

In another embodiment the compositions of Formula (I), (IA), (IAA), (IAAA), (IB), (IBB), (IC), (ICC), (ID), (IDD), (IE), or (IEE) are enteric-coated controlled-release tablets for oral administration. The compositions can further comprise carnauba wax, crospovidone, diacetylated monoglycerides, ethylcellulose, hydroxypropyl cellulose, pyridazine phthalate, magnesium stearate, mannitol, sodium hydroxide, sodium stearyl fumarate, talc, titanium dioxide, or yellow ferric oxide.

Sustained-release preparations comprising a compound or pharmaceutically acceptable salt of Formula (I), (IA), (IAA), (IAAA), (IB), (IBB), (IC), (ICC), (ID), (IDD), (IE), or (IEE) may also be prepared. Examples of sustained-release preparations may include semipermeable matrices of solid hydrophobic polymers that may contain the compound, salt or conjugate, and these matrices may be in the form of shaped articles (e.g., films or microcapsules). Examples of sustained-release matrices may include polyesters, hydrogels (e.g., poly(2-hydroxyethyl-methacrylate), or poly(vinyl alcohol)), polylactides, copolymers of L-glutamic acid and y ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations comprising a compound or pharmaceutically acceptable salt of Formula (I), (IA), (IAA), (IAAA), (IB), (IBB), (IC), (ICC), (ID), (IDD), (IE), or (IEE) may be prepared for storage by mixing a compound, salt or conjugate with a pharmaceutically acceptable carrier, excipient, and/or a stabilizer. This formulation may be a lyophilized formulation or an aqueous solution. Acceptable carriers, excipients, and/or stabilizers may be nontoxic to recipients at the dosages and concentrations used. Acceptable carriers, excipients, and/or stabilizers may include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

In another embodiment the compositions of Formula (I), (IA), (IAA), (IAAA), (IB), (IBB), (IC), (ICC), (ID), (IDD), (IE), or (IEE) can further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

In another embodiment compositions of Formula (I), (IA), (IAA), (IAAA), (IB), (IBB), (IC), (ICC), (ID), (IDD), (IE), or (IEE) are provided in effervescent dosage forms. These effervescent dosage forms can also comprise non-release controlling excipients.

In another embodiment compositions of Formula (I), (IA), (IAA), (IAAA), (IB), (IBB), (IC), (ICC), (ID), (IDD), (IE), or (IEE) can be provided in a dosage form that has at least one component that can facilitate the immediate release of an active agent, and at least one component that can facilitate the controlled release of an active agent. In a further embodiment the dosage form can be capable of giving a discontinuous release of the compound in the form of at least two consecutive pulses separated in time from 0.1 up to 24 hours. The compositions can comprise one or more release controlling and non-release controlling excipients, such as those excipients suitable for a disruptable semi-permeable membrane and as swellable substances.

In another embodiment compositions of Formula (I), (IA), (IAA), (IAAA), (IB), (IBB), (IC), (ICC), (ID), (IDD), (IE), or (IEE) are provided in a dosage form for oral administration to a subject, which comprise one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In some embodiments, the compositions of (I), (IA), (IAA), (IAAA), (IB), (IBB), (IC), (ICC), (ID), (IDD), (IE), or (IEE) provided herein can be in unit-dosage forms or multiple-dosage forms. Unit-dosage forms, as used herein, refer to physically discrete units suitable for administration to human or non-human animal subjects and packaged individually. Each unit-dose can contain a predetermined quantity of an active ingredient(s) sufficient to produce the desired therapeutic effect, in association with the required pharmaceutical carriers or excipients. Examples of unit-dosage forms include, but are not limited to, ampoules, syringes, and individually packaged tablets and capsules. In some embodiments, unit-dosage forms may be administered in fractions or multiples thereof. A multiple-dosage form is a plurality of identical unit-dosage forms packaged in a single container, which can be administered in segregated unit-dosage form. Examples of multiple-dosage forms include, but are not limited to, vials, bottles of tablets or capsules, or bottles of pints or gallons. In another embodiment the multiple dosage forms comprise different pharmaceutically active agents.

In some embodiments, the compositions of (I), (IA), (IAA), (IAAA), (IB), (IBB), (IC), (ICC), (ID), (IDD), (IE), or (IEE) may also be formulated as a modified release dosage form, including immediate-, delayed-, extended-, prolonged-, sustained-, pulsatile-, controlled-, extended, accelerated- and fast-, targeted-, programmed-release, and gastric retention dosage forms. These dosage forms can be prepared according to known methods and techniques (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126, which are herein incorporated by reference in their entirety).

### Combination Therapies

Also contemplated herein are combination therapies, for example, co-administering a disclosed compound and an additional active agent, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually hours, days, weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration is accomplished, for example, by administering to the subject a single formulation or composition, (e.g., a tablet or capsule having a fixed ratio of each therapeutic agent or in multiple, single formulations (e.g., capsules) for each of the therapeutic agents. Sequential or substantially simultaneous administration of each therapeutic agent is affected by any appropriate route including, but not limited to, oral routes, intravenous routes, intramuscular routes, and direct absorption through mucous membrane tissues. The therapeutic agents are administered by the same route or by different routes. For example, a first therapeutic agent of the combination selected is administered by intravenous injection while the other therapeutic agents of the combination are administered orally. Alternatively, for example, all therapeutic agents are administered orally or all therapeutic agents are administered by intravenous injection.

The components of the combination are administered to a patient simultaneously or sequentially. It will be appreciated that the components are present in the same pharmaceutically acceptable carrier and, therefore, are administered simultaneously. Alternatively, the active ingredients are present in separate pharmaceutical carriers, such as, conventional oral dosage forms, that are administered either simultaneously or sequentially.

### Additional Embodiments

Embodiment 101. A compound, or a pharmaceutically acceptable salt or solvate thereof, having the structure of Formula (I):

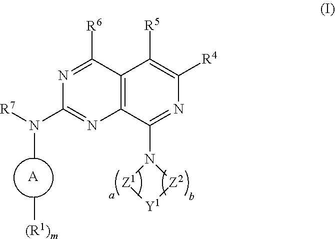

wherein,
A is a ring selected from optionally substituted carbocycle, optionally substituted 4- to 6-membered heterocycle, and optionally substituted isoindoline;
each of Z¹, Z², and Y¹ are independently selected from —C(R²)₂—, —C(O)—, —NR³—, —N(C(O)R²)—, —NS(O₂)R², —O—, —S—, —S(O)—, and —S(O)₂—;
each of a and b are independently selected from 1, 2, 3, and 4;
each R¹ is independently selected from halogen, —CN, —NO₂, optionally substituted alkyl, optionally substituted alkenyl, optionally substituted alkynyl, optionally substituted carbocycle, and optionally substituted heterocycle;
m is selected from 0 to 5;
each R² is independently selected from hydrogen, halogen, —CN, —OH, —O—C₁₋₄ alkyl, optionally substituted alkyl, optionally substituted cycloalkyl, and optionally substituted heterocycloalkyl, or two R² substituents come together to form an optionally substituted heterocycle or an optionally substituted carbocycle, or R² and R³ substituents come together to form an optionally substituted heterocycle;
each R³ is independently selected from hydrogen, optionally substituted alkyl, optionally substituted C₃₋₄ carbocycle, and optionally substituted 3- to 4-membered heterocycloalkyl;
R⁴ is selected from hydrogen, halogen, —CN, optionally substituted C₁₋₄ alkyl, optionally substituted C₃₋₄ carbocycle, and optionally substituted 3- to 4-membered heterocycloalkyl;
each of R⁵, R⁶, is independently selected from hydrogen, halogen, —CN, optionally substituted C₁₋₄ alkyl, optionally substituted C₃₋₄ carbocycle, and optionally substituted 3- to 4-membered heterocyclcoalkyl; and
R⁷ is selected from hydrogen and optionally substituted C₁₋₄ alkyl, and
wherein if A is optionally substituted phenyl, m is from 1 to 5 and at least one R¹ is a heterocycloalkyl,
wherein if A is optionally substituted pyridine or optionally substituted pyrimidine, R⁴ is selected from hydrogen, halogen, and —CN, and
wherein if A is an optionally substituted piperidine sulfonamide, then either (i) Y¹ is —C(R²)₂— and the two R² substituents come together to form a ring selected from an optionally substituted heterocycle and an optionally substituted carbocycle, or (ii) R⁴ is selected from hydrogen, halogen, and —CN.

Embodiment 102. The compound, or a pharmaceutically acceptable salt or solvate thereof, of embodiment 101 having the structure of Formula (IA):

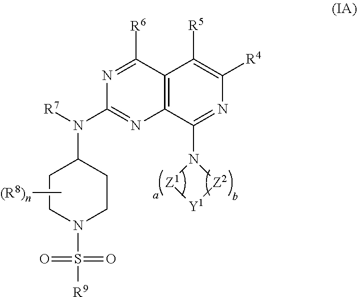

wherein,
R⁸ is selected from halogen, —CN, and optionally substituted C₁₋₄ alkyl;
R⁹ is selected from optionally substituted C₁₋₄ alkyl, optionally substituted C₃₋₆ carbocycle, and 3- to 6-membered heterocycloalkyl; and
n is selected from 0 to 9;
wherein either (i) Y¹ is —C(R²)₂— and the two R² substituents come together to form a ring selected from an optionally substituted heterocycle and an optionally substituted carbocycle, or (ii) R⁴ is selected from hydrogen, halogen, and —CN.

Embodiment 103. The compound or salt of embodiment 1 or 3, wherein Z¹ and Z² are independently selected from —C(R²)₂—, —NR³—, —O—, and —S—.

Embodiment 104. The compound or salt of embodiment 4, wherein Z¹ and Z² are independently —C(R²)₂—.

Embodiment 105. The compound or salt of embodiment 5, wherein each of a and b are independently selected from 1 and 2.

Embodiment 106. The compound or salt of any one of embodiments 1-6, wherein Y¹ is selected from —C(R²)₂—, —NR³—, —O—, and —S—.

Embodiment 107. The compound or salt of embodiment 7, wherein Y¹ is selected from —C(R²)₂— and —NR³.

Embodiment 108. The compound or salt of any one of embodiments 1-8, wherein each R² is independently selected from hydrogen, halogen, —CN, optionally substituted cycloalkyl, and optionally substituted heterocycloalkyl.

Embodiment 109. The compound or salt of embodiment 9, wherein each R² is independently selected from hydrogen, —CN, cyclopropyl, cyclobutyl, optionally substituted cycloalkyl, and optionally substituted heterocycloalkyl.

Embodiment 110. The compound, or a pharmaceutically acceptable salt or solvate thereof, of embodiment 10, wherein each R² is independently selected from hydrogen, —CN and cyclopropyl.

Embodiment 111. The compound or salt of embodiments 1-Error! Reference source not found., wherein each R³ is selected from cyclopropyl, cyclobutyl, optionally substituted oxetane, and optionally substituted azetidine.

Embodiment 112. The compound or salt of embodiment 12, wherein each R³ is selected from cyclopropyl and cyclobutyl.

Embodiment 113. The compound or salt of embodiment Error! Reference source not found., wherein R³ is cyclopropyl.

Embodiment 114. The compound, or a pharmaceutically acceptable salt or solvate thereof, of embodiment 1 or 3, having the structure of Formula (IAA):

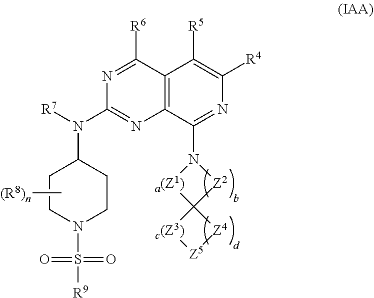

wherein,
R⁸ is selected from halogen, —CN, and optionally substituted C₁₋₄ alkyl;
R⁹ is selected from optionally substituted C₁₋₄ alkyl, optionally substituted C₃₋₆ carbocycle, and 3- to 6-membered heterocycloalkyl;
n is selected from 0 to 9;
each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond; and
each of a, b, c, and d are independently selected from 1, 2, 3, and 4.

Embodiment 115. The compound or salt of embodiment 13, wherein each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond.

Embodiment 116. The compound or salt of embodiment 14, wherein each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond.

Embodiment 117. The compound or salt of embodiment [0439], wherein each of Z¹, Z², Z³, Z⁴ and Z⁵ are independently selected from —C(R²)₂—, —NR³—, —O—, and —S(O)₂—, wherein Z⁵ is additionally selected from a bond.

Embodiment 118. The compound or salt of embodiments 13-15, wherein each R² is independently selected from hydrogen, halogen, optionally substituted cycloalkyl, and optionally substituted heterocycloalkyl, or two R² substituents come together to form an optionally substituted heterocycle or an optionally substituted carbocycle.

Embodiment 119. The compound or salt of embodiment 16, wherein each R² is selected from hydrogen, fluoro, or two R² substituents come together to form an optionally substituted heterocycle or an optionally substituted carbocycle.

Embodiment 120. The compound or salt of any one of embodiments 13-17, wherein R³ is selected from hydrogen, cyclopropyl, cyclobutyl, optionally substituted oxetane, and optionally substituted azetidine.

Embodiment 121. The compound or salt of embodiment Error! Reference source not found., wherein R³ is selected from hydrogen, —(CH₂)₂OMe, cyclopropyl, cyclobutyl, optionally substituted oxetane, and optionally substituted azetidine.

Embodiment 122. The compound or salt of embodiment Error! Reference source not found., wherein R³ is selected from —(CH₂)₂₀Me and cyclopropyl.

Embodiment 123. The compound or salt of any one of embodiments 13-18, wherein each a, b, c, and d are independently selected from 1 and 2.

Embodiment 124. The compound or salt of any one of embodiments 1-19, wherein the compound is selected from:

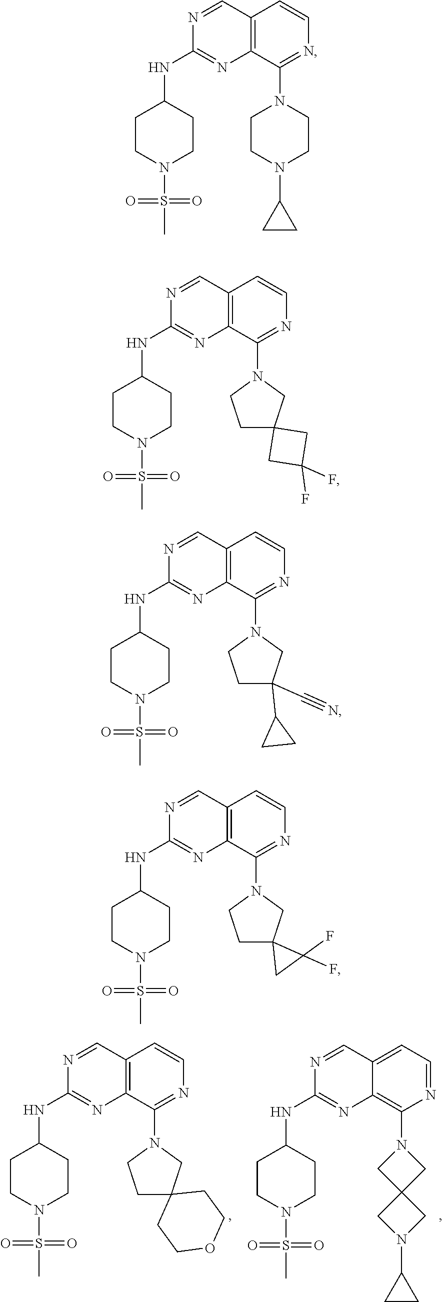

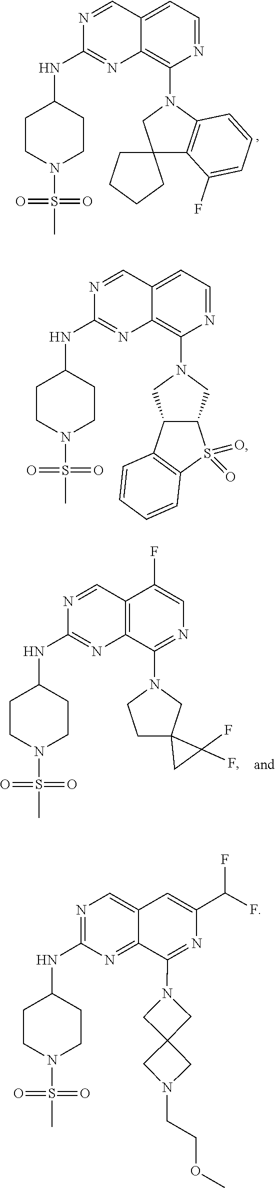

Embodiment 125. The compound or salt of embodiment [0447], wherein the compound is selected from:

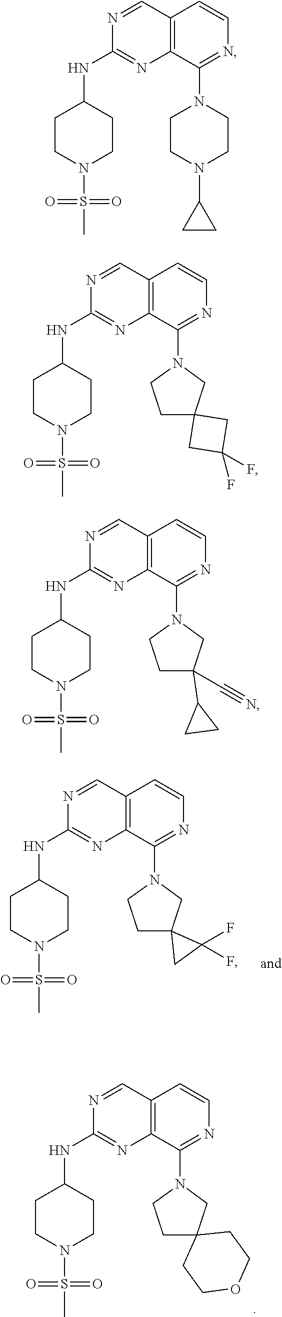

Embodiment 126. The compound, or a pharmaceutically acceptable salt or solvate thereof, of embodiment 1 having the structure of Formula (IB):

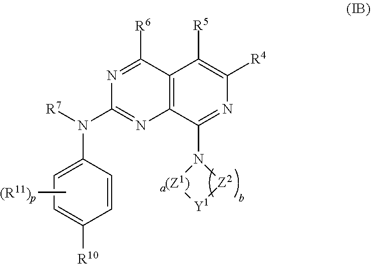

wherein,
R¹⁰ is optionally substituted heterocycloalkyl;
R¹¹ is selected from halogen, —CN, and optionally substituted C₁₋₄ alkyl; and
p is selected from 0 to 4.

Embodiment 127. The compound or salt of embodiment [0449], wherein Z¹ and Z² are independently selected from —C(R²)₂—, —NR³—, —O—, and —S—.

Embodiment 128. The compound or salt of embodiment [0450], wherein Z¹ and Z² are independently —C(R²)₂—.

Embodiment 129. The compound or salt of embodiment [0451], wherein each of a and b are independently selected from 1 and 2.

Embodiment 130. The compound or salt of any one of embodiments [0449]-[0452], wherein Y¹ is selected from —C(R²)₂—, —NR³—, —O—, and —S—.

Embodiment 131. The compound or salt of embodiment [0453], wherein Y¹ is —C(R²)₂—.

Embodiment 132. The compound or salt of any one of embodiments [0449]-[0454], wherein each R² is independently selected from hydrogen, halogen, —CN, optionally substituted cycloalkyl, and optionally substituted heterocycloalkyl.

Embodiment 133. The compound or salt of embodiment [0455], wherein each R² is independently selected from hydrogen, —CN, cyclopropyl, cyclobutyl, optionally substituted cycloalkyl, and optionally substituted heterocycloalkyl.

Embodiment 134. The compound or salt of embodiment [0456], wherein each R² is independently selected from hydrogen, —CN and cyclopropyl.

Embodiment 135. The compound or salt of any one of embodiments [0449]-[0457], wherein R³ is selected from cyclopropyl, cyclobutyl, optionally substituted oxetane, and optionally substituted azetidine.

Embodiment 136. The compound or salt of embodiment [0458], wherein R³ is selected from cyclopropyl and cyclobutyl.

Embodiment 137. The compound or salt of embodiment [0459], wherein R³ is cyclopropyl.

Embodiment 138. The compound, or a pharmaceutically acceptable salt or solvate thereof, of embodiment 1 or [0449], having the structure of Formula (IBB):

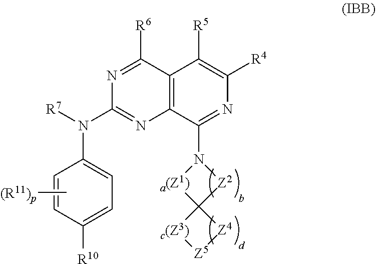

wherein,
R¹⁰ is optionally substituted heterocycloalkyl;
R¹¹ is selected from halogen, —CN, and optionally substituted C₁₋₄ alkyl;
p is selected from 0 to 4;
each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond; and
each of a, b, c, and d are independently selected from 1, 2, 3, and 4.

Embodiment 139. The compound or salt of embodiment [0461], wherein each of Z¹, Z², Z³, Z⁴ and Z⁵ are independently selected from —C(R²)₂—, —NR³—, —NS(O₂)R³, —O—, and —S(O)₂—, wherein Z⁵ is additionally selected from a bond.

Embodiment 140. The compound or salt embodiment [0462], wherein each of Z¹, Z², Z³, Z⁴ and Z⁵ are independently selected from —C(R²)₂—, —NS(O₂)R³, —O—, and —S(O)₂—, wherein Z⁵ is additionally selected from a bond.

Embodiment 141. The compound or salt of any one of embodiments [0461]-[0463], wherein each R² is independently selected from hydrogen, halogen, —CN, optionally substituted cycloalkyl, and optionally substituted heterocycloalkyl.

Embodiment 142. The compound or salt of embodiment [0464], wherein each R² is independently selected from hydrogen, fluoro, —CN, cyclopropyl, cyclobutyl, optionally substituted oxetane, and optionally substituted azetidine.

Embodiment 143 The compound or salt of embodiment [0465], wherein each R² is independently selected from hydrogen, fluoro, —CN, and cyclopropyl.

Embodiment 144. The compound or salt of any one of embodiments [0461]-[0466], wherein R³ is selected from hydrogen, methyl, ethyl, propyl, cyclopropyl, cyclobutyl, optionally substituted oxetane, and optionally substituted azetidine.

Embodiment 145. The compound or salt of embodiment [0467], wherein R³ is selected from hydrogen, methyl, ethyl, and propyl.

Embodiment 146. The compound, or a pharmaceutically acceptable salt or solvate thereof, of embodiment [0468], wherein R³ is methyl.

Embodiment 147. The compound or salt of embodiment [0461]-[0469], wherein each a, b, c, and d are independently selected from 1 and 2.

Embodiment 148. The compound or salt of any one of embodiments [0449]-[0470], wherein the compound is selected from:

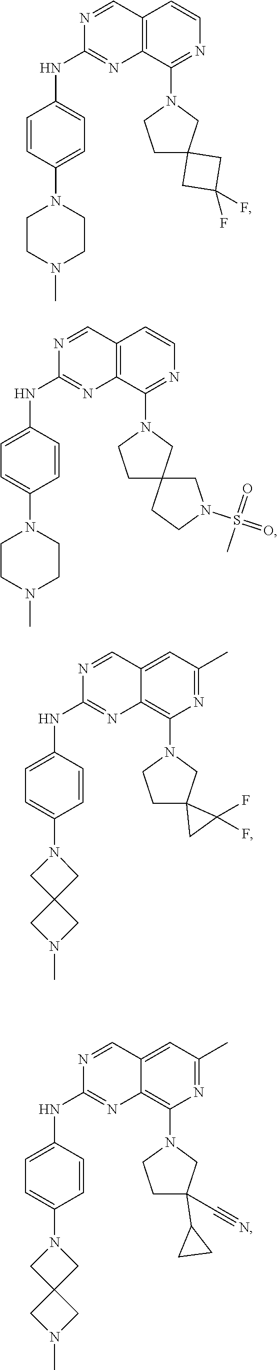

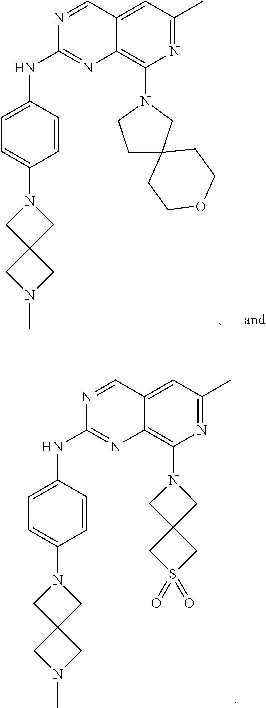 [SMILES: Cc1cc2cnc(Nc3ccc(N4CC5(CN(C)C5)C4)cc3)nc2c(N2CCC3(CCOCC3)C2)n1 ; Cc1cc2cnc(Nc3ccc(N4CC5(CN(C)C5)C4)cc3)nc2c(N2CC3(C2)CS(=O)(=O)C3)n1]

Embodiment 149. The compound, or a pharmaceutically acceptable salt or solvate thereof, of embodiment 1 having the structure of Formula (IC):

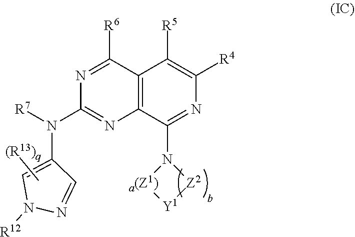

wherein,
R¹² is selected from an optionally substituted heterocycloalkyl and optionally substituted cycloalkyl; or R¹² and R¹³ come together to form an optionally substituted heterocycle;
R¹³ is selected from halogen, —CN, and optionally substituted C₁₋₄ alkyl; and
q is selected from 0 to 2.

Embodiment 150. The compound or salt of embodiment [0472], wherein Z¹ and Z² are independently selected from —C(R²)₂—, —NR³—, —O—, and —S—.

Embodiment 151. The compound or salt of embodiment [0473], wherein Z¹ and Z² are independently —C(R²)₂—.

Embodiment 152. The compound or salt of embodiment [0474], wherein each of a and b are independently selected from 1 and 2.

Embodiment 153. The compound or salt of any one of embodiments [0472]-[0475], wherein Y¹ is selected from —C(R²)₂—, —NR³—, —NS(O₂)R², —O—, and —S—.

Embodiment 154. The compound or salt of embodiment [0476], wherein Y¹ is selected from —C(R²)₂—, —NR₃, and —NS(O₂)R².

Embodiment 155. The compound or salt of any one of embodiments [0472]-[0477], wherein each R² is independently selected from hydrogen, halogen, —CN, optionally substituted cycloalkyl, and optionally substituted heterocycloalkyl.

Embodiment 156. The compound or salt of embodiment [0478], wherein each R² is selected from hydrogen, fluoro, —CN, cyclopropyl, cyclobutyl, optionally substituted cycloalkyl, and optionally substituted heterocycloalkyl.

Embodiment 157. The compound or salt of embodiment [0479], wherein each R² is selected from hydrogen, fluoro, —CN and cyclopropyl.

Embodiment 158. The compound or salt of any one of embodiments [0472]-[0480], wherein R³ is selected from optionally substituted alkyl.

Embodiment 159. The compound or salt of embodiment [0481], wherein R³ is methyl.

Embodiment 160. A pharmaceutically acceptable salt or solvate thereof, having the structure of Formula (ICC):

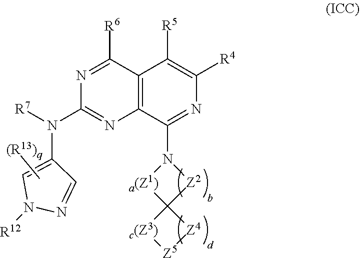

wherein,
R¹² is optionally substituted heterocycloalkyl; or R¹² and R¹³ come together to form an optionally substituted heterocycle;
R¹³ is selected from halogen, —CN, and optionally substituted C₁₋₄ alkyl; q is selected from 0 to 2; and
each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond; and
each of a, b, c, and d are independently selected from 1, 2, 3, and 4.

Embodiment 161. The compound or salt of embodiment [0483], wherein each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond.

Embodiment 162. The compound or salt of embodiment [0484], wherein each of Z¹, Z², Z³, Z⁴ and Z⁵ are independently selected from —C(R²)₂—, —NR³—, and —NS(O₂)R³, wherein Z⁵ is additionally selected from a bond.

Embodiment 163. The compound or salt of any one of embodiments [0483]-[0485], wherein each R² is independently selected from hydrogen, halogen, —CN, —OH, and optionally substituted alkyl.

Embodiment 164. The compound, or a pharmaceutically acceptable salt or solvate thereof, of embodiment [0486], wherein each R² is independently selected from hydrogen, fluoro, and —OH.

Embodiment 165. The compound or salt of any one of embodiments [0483]-[0487], wherein R³ is independently selected form hydrogen and optionally substituted alkyl.

Embodiment 166. The compound or salt of embodiment [0488], wherein R³ is independently selected form hydrogen, methyl, ethyl, and propyl

Embodiment 167. The compound or salt of embodiment [0489], wherein R³ is methyl.

Embodiment 168. The compound or salt of any one of embodiments [0472]-[0490], wherein R¹² is optionally substituted 5- to 6-membered heterocycle, or R¹² and R¹³ come together to form an optionally substituted heterocycle or an optionally substituted carbocycle.

Embodiment 169. The compound or salt of embodiment [0491], wherein R¹² is optionally substituted piperidine, optionally substituted azaspiro[3.3]heptane, or R¹² and R¹³ come together to form an optionally substituted heterocycle.

Embodiment 170. The compound or salt of any one of embodiments [0472]-[0492], wherein R¹³ and R¹² come together to form an optionally substituted heterocycle.

Embodiment 171. The compound or salt of any one of embodiments [0472]-[0493], wherein the compound is selected from:

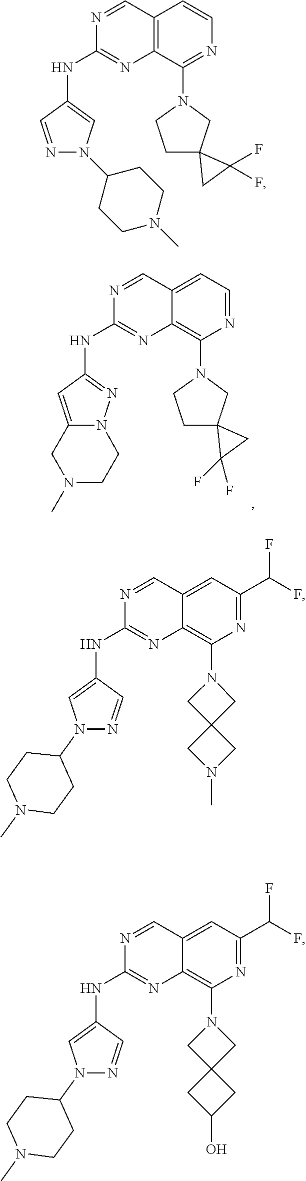

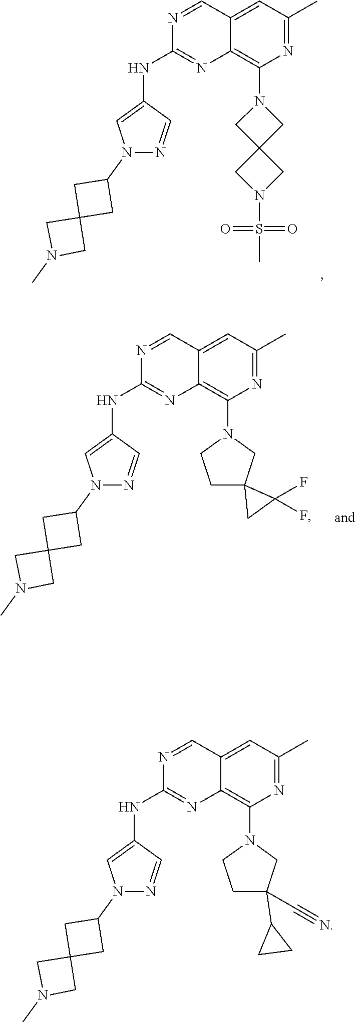

Embodiment 172. The compound or salt of embodiment 1, having the structure of Formula (ID):

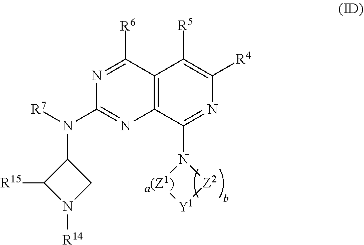

wherein,
R¹⁴ is selected from —SOR¹⁶—, and optionally substituted heterocycloalkyl;
R¹⁵ is selected from hydrogen, halogen, —CN, and optionally substituted C₁₋₄ alkyl; and
R¹⁶ is selected from optionally substituted C₁₋₄ alkyl, optionally substituted C₃₋₆ carbocycle, and optionally substituted 3- to 6-membered heterocycloalkyl.

Embodiment 173. The compound, or a pharmaceutically acceptable salt or solvate thereof, of embodiment 1 or [0495], having the structure of Formula (IDD):

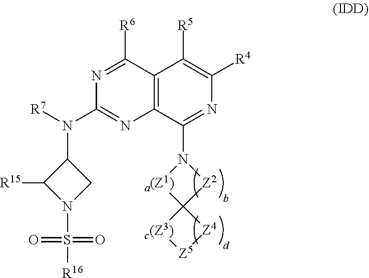

wherein,
   R¹⁶ is selected from optionally substituted C₁₋₄ alkyl, optionally substituted C₃₋₆ carbocycle, and optionally substituted 3- to 6-membered heterocycloalkyl.

Embodiment 174. A pharmaceutically acceptable salt or solvate thereof, having the structure of Formula (IE):

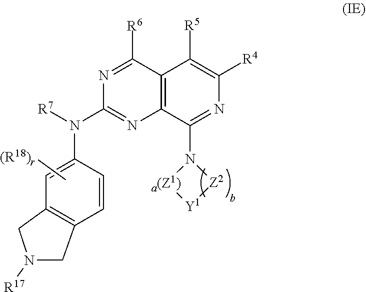

wherein,
R¹⁷ is selected from —SOR¹⁹—, optionally substituted alkyl, optionally substituted carbocycle, and optionally substituted heterocycloalkyl;
R¹⁸ is selected from halogen, —CN, and optionally substituted C₁₋₄ alkyl;
R¹⁹ is selected from optionally substituted C₁₋₄ alkyl, optionally substituted C₃₋₆ carbocycle, and optionally substituted 3- to 6-membered heterocycloalkyl; and
r is selected from 0 to 5.

Embodiment 175. The compound or salt of embodiment [0497], wherein Z¹ and Z² are independently selected from —C(R²)₂—, —NR³—, —O—, and —S—.

Embodiment 176. The compound or salt of embodiment [0498], wherein Z¹ and Z² are independently —C(R²)₂—.

Embodiment 177. The compound or salt of embodiment [0499], wherein each of a and b are independently selected from 1 and 2.

Embodiment 178. The compound or salt of any one of embodiments [0497]-[0500], wherein Y¹ is selected from —C(R²)₂—, —NR³—, —NS(O₂)R², —O—, and —S(O)₂—.

Embodiment 179. The compound or salt of any one of embodiments [0497]-[0501], wherein each R² is independently selected from hydrogen, halogen, —CN, optionally substituted cycloalkyl, and optionally substituted heterocycloalkyl.

Embodiment 180. The compound or salt of any one of embodiments [0497]-[0502], R³ is selected from optionally substituted alkyl.

Embodiment 181. The compound or salt of any one of embodiments [0497]-[0503], wherein r is 0.

Embodiment 182. The compound or salt of any one of embodiments [0497]-[0504], wherein R¹⁷ is selected from —SOR¹⁹—, optionally substituted alkyl, and optionally substituted 3- to 5 membered heterocycloalkyl.

Embodiment 183. The compound or salt of embodiment [0505], wherein R¹⁷ is selected from —SOR¹⁹—, methyl, and optionally substituted 4-membered heterocycloalkyl.

Embodiment 184. The compound, or a pharmaceutically acceptable salt or solvate thereof, of embodiment [0497], having the structure of Formula (IEE):

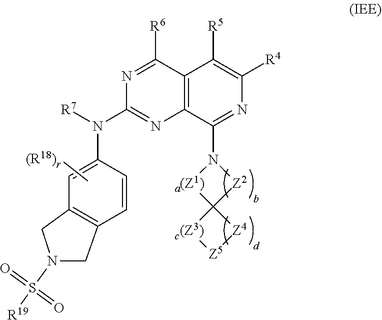

wherein,
R¹⁹ is selected from optionally substituted C₁₋₄ alkyl, optionally substituted C₃₋₆ carbocycle, and optionally substituted 3- to 6-membered heterocycloalkyl.

Embodiment 185. The compound or salt of embodiment [0507], wherein each of Z¹, Z², Z³, Z⁴ and Z⁵ are independently selected from —C(R²)₂—, —NR³—, —NS(O₂)R³, —O—, and —S(O)₂—, wherein Z⁵ is additionally selected from a bond.

Embodiment 186. The compound or salt of embodiment [0508], wherein each of Z¹, Z², Z³, Z⁴ and Z⁵ are independently selected from —C(R²)₂—, —NR³—, —NS(O₂)R³, and —S(O)₂—, wherein Z⁵ is additionally selected from a bond.

Embodiment 187. The compound or salt of any one of embodiments [0507]-[0509], wherein each R² is independently selected from hydrogen, halogen, —CN, —OH, and optionally substituted alkyl.

Embodiment 188. The compound or salt of embodiment [0510], wherein each R² is independently selected from hydrogen and fluoro.

Embodiment 189. The compound or salt of any one of embodiments [0507]-[0511], wherein R³ is optionally substituted alkyl.

Embodiment 190. The compound or salt of embodiment [0512], wherein R³ is methyl, ethyl, or propyl.

Embodiment 191. The compound or salt of embodiment [0513], wherein R³ is methyl.

Embodiment 192. The compound or salt of any one of embodiments [0507]-[0504], wherein R¹⁹ is optionally substituted alkyl.

Embodiment 193. The compound or salt of embodiment [0515], wherein R¹⁹ is methyl.

Embodiment 194. The compound or salt of any one of embodiments [0497]-[0516], wherein the compound is selected from:

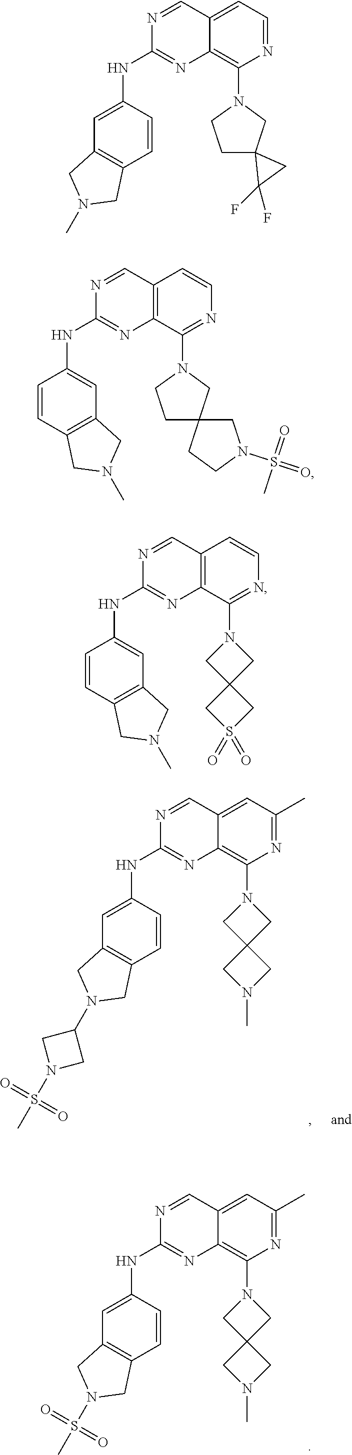

Embodiment 195. The compound or salt of embodiment 1, wherein A is selected from optionally substituted pyridine, optionally substituted azabicyclo[3.1.0]hexane, optionally substituted azetidine, and optionally substituted tetrahydroisoquinoline.

Embodiment 196. The compound or salt of embodiment 20, wherein A is substituted with —SO₂Me.

Embodiment 197. The compound or salt of embodiment 20 or 22, wherein m is selected from 0 to 1.

Embodiment 198. The compound or salt of any one of embodiments 20-23, wherein each R¹ is independently selected from optionally substituted alkyl, optionally substituted carbocycle, and optionally substituted heterocycle.

Embodiment 199. The compound or salt of embodiment 24, wherein each R¹ is independently selected from optionally substituted alkyl and optionally substituted heterocycle.

Embodiment 200. The compound or salt of embodiment 24, wherein each R¹ is independently selected from methyl, ethyl, and optionally substituted diazaspiro[3.3]heptane.

Embodiment 201. The compound or salt of any one of embodiments 1—Error! Reference source not found., [0449]-[0460], [0472]-[0482], [0495], [0497]-[0506], and 20—Error! Reference source not found, wherein Z¹ and Z² are independently selected from —C(R²)₂—, —NR³—, —O—, and —S—.

Embodiment 202. The compound or salt of embodiment Error! Reference source not found., wherein Z¹ and Z² are independently —C(R²)₂—.

Embodiment 203. The compound or salt of embodiments Error! Reference source not found. or Error! Reference source not found., wherein each of a and b are independently selected from 1 and 2.

Embodiment 204. The compound or salt of any one of embodiments 1—Error! Reference source not found., [0449]-[0460], [0472]-[0482], [0495], [0497]-[0506], and 20—Error! Reference source not found., wherein Y¹ is selected from —C(R²)₂—, —NR₃—, —O—, and —S—.

Embodiment 205. The compound or salt of embodiment Error! Reference source not found., wherein Y¹ is selected from —C(R²)₂— and —NR₃.

Embodiment 206. The compound or salt of embodiment Error! Reference source not found., wherein Y¹ is —C(R²)₂— and two R² substituents come together to form a ring selected from an optionally substituted heterocycle and an optionally substituted carbocycle.

Embodiment 207. The compound or salt of embodiment Error! Reference source not found., wherein Y¹ is —C(R²)₂— and the two R² substituents come together to form a ring selected from an optionally substituted heterocycle.

Embodiment 208. The compound or salt of any one of embodiments 1—Error! Reference source not found., [0449]-[0460], [0472]-[0482], [0495], [0497]-[0506], and 20—Error! Reference source not found., wherein the N containing heterocyclic ring depicted as

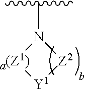

in Formula (I), (IB), (IC), (ID), and (IE) is selected from optionally substituted azetidine, optionally substituted pyrrolidine, optionally substituted piperidine, optionally substituted piperazine, optionally substituted morpholine, optionally substituted tetrahydrothienopyrroledioxide, and optionally substituted dihydroindole.

Embodiment 209. The compound or salt of embodiment 25, wherein the N containing heterocyclic ring depicted as

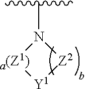

in Formula (I), (IB), (IC), (ID), and (IE) is selected from

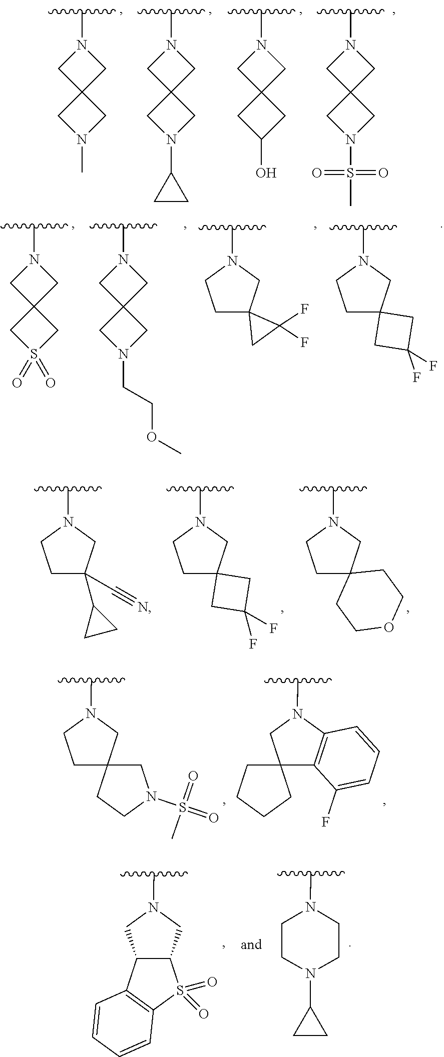

Embodiment 210. The compound or salt of any one of embodiments 20-Error! Reference source not found., wherein the compound is selected from:

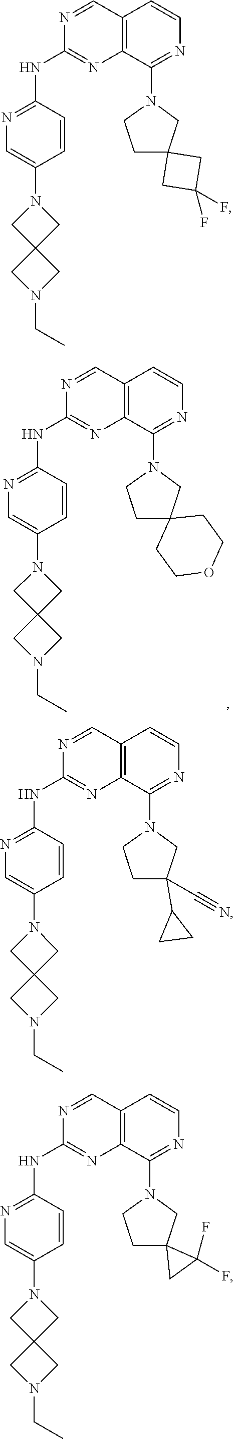

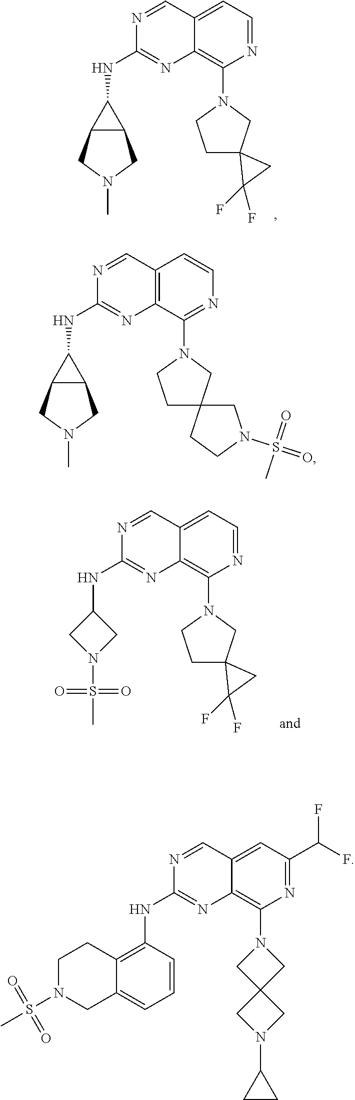

Embodiment 211. The compound or salt of any one of embodiments 1-19, [0449]-[0470], [0493], [0495]-[0516], or 20-109, wherein R⁴ is selected from hydrogen, halogen, optionally substituted C₁₋₂ alkyl, optionally substituted C₃₋₄ carbocycle, and optionally substituted 3- to 4 membered heterocycloalkyl.

Embodiment 212. The compound or salt of embodiment 27, wherein R⁴ is selected from hydrogen and optionally substituted C₁ alkyl.

Embodiment 213. The compound or salt of embodiment Error! Reference source not found., wherein R⁴ is selected from hydrogen, methyl, and —CHF₂.

Embodiment 214. The compound or salt of any one of embodiments 1-19, [0449]-[0470], [0472]-[0493], [0495]-[0516], or 20-26, wherein R⁴ is selected from hydrogen, halogen, and —CN.

Embodiment 215. The compound or salt of any one of embodiments 1-19, [0449]-[0470], [0472]-[0493], [0495]-[0516], or 20-26, wherein R⁴ is selected from hydrogen.

Embodiment 216. The compound or salt of any one of embodiments 1-19, [0449]-[0470], [0472]-[0493], [0495]-[0516], or 20-26, wherein R⁴ is not optionally substituted phenyl.

Embodiment 217. The compound or salt of any one of embodiments 1-19, [0449]-[0470], [0472]-[0493], [0495]-[0516], or 20-26, wherein R⁴ is not optionally substituted alkyl.

Embodiment 218. The compound or salt of any one of embodiments 1-19, [0449]-[0470], [0472]-[0493], [0495]-[0516], 20-Error! Reference source not found., or 27 to 117, wherein R⁵ is selected from hydrogen, halogen, optionally substituted C₁₋₂ alkyl, optionally substituted C₃₋₄ carbocycle, and optionally substituted 3- to 4 membered heterocycloalkyl.

Embodiment 219. The compound or salt of embodiment 29, wherein R⁵ is selected from hydrogen, halogen, and optionally substituted C₁₋₂ alkyl.

Embodiment 220. The compound or salt of embodiment Error! Reference source not found., wherein R⁵ is selected from hydrogen and fluoro.

Embodiment 221. The compound or salt of any one of embodiments 1-19, [0449]-[0470], [0472]-[0493], [0495]-[0516], 20-Error! Reference source not found., or 27-Error! Reference source not found., wherein R⁶ is selected from hydrogen, halogen, optionally substituted C₁₋₂ alkyl, optionally substituted C₃₋₄ carbocycle, and optionally substituted 3- to 4 membered heterocycloalkyl.

Embodiment 222. The compound or salt of embodiment 30, wherein R⁶ is selected from hydrogen, halogen, and optionally substituted C₁₋₂ alkyl.

Embodiment 223. The compound or salt of embodiment Error! Reference source not found., wherein R⁶ is hydrogen.

Embodiment 224. The compound or salt of any one of embodiments 1-19, [0449]-[0470], [0471]-[0493], [0495]-[0516], 20-Error! Reference source not found., or 27-Error! Reference source not found., wherein R⁷ is hydrogen.

Embodiment 225. A pharmaceutical composition comprising a compound or salt of any one of embodiments 1 to 31 and a pharmaceutically acceptable excipient.

Embodiment 226. A method of treating cancer, comprising administering to a subject in need thereof the pharmaceutical composition of embodiment 34.

Embodiment 227. The method of embodiment 35, wherein the cancer is a solid tumor.

Embodiment 228. The method of embodiment 35 or 36, wherein the cancer is selected from ovarian cancer, breast cancer, colon cancer, and brain cancer.

Embodiment 229. The method of embodiment 37, wherein the cancer is ovarian cancer or breast cancer.

Embodiment 230. A method of inhibiting a cyclin dependent kinase (CDK) in a cell with a compound or salt of any one of embodiments 1 to 31 or the pharmaceutical composition of embodiment 34.

Embodiment 231. The method of embodiment 38, wherein the CDK is selected from CDK 2, CDK 4, CD6, or any combination thereof.

Embodiment 232. The method of embodiment 39, wherein the CDK is selected from CDK 2/4, CDK 2/6, CDK 4/6, and CDK 2/4/6.

Embodiment 233. The method of embodiment 40, wherein the CDK is CDK 2/4/6.

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### General Synthetic Schemes 1-3

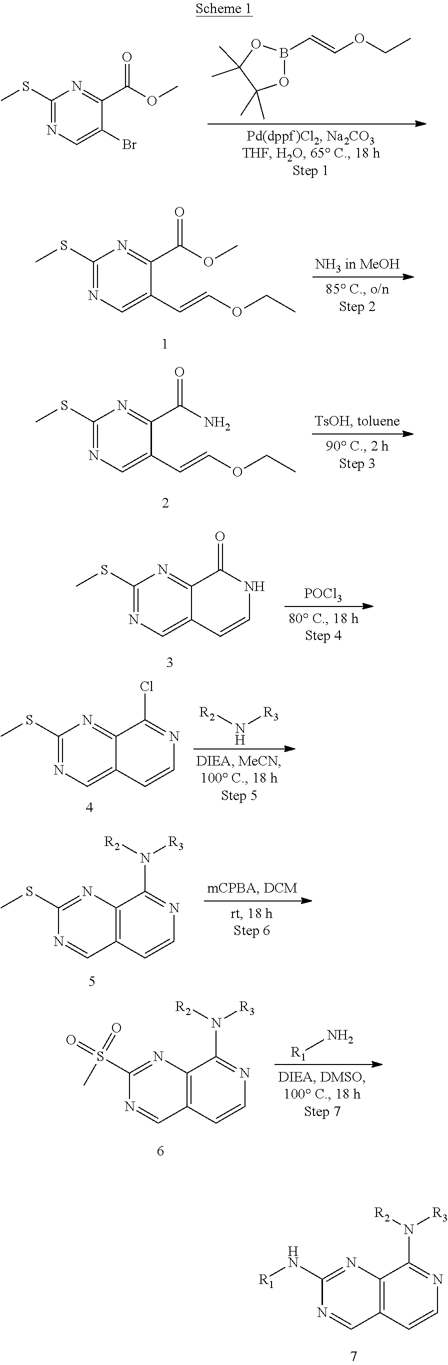

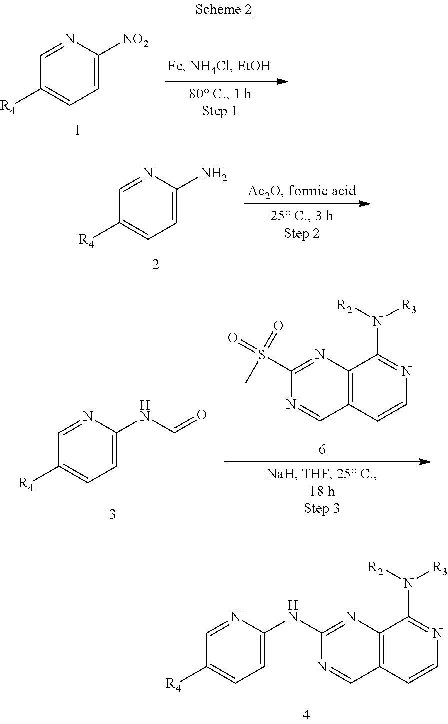

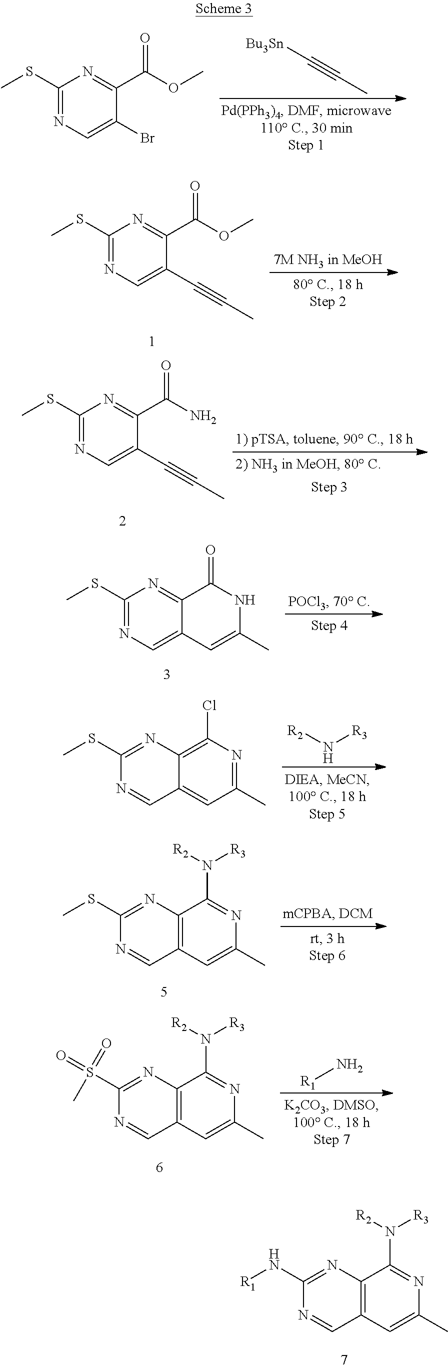

### INTERMEDIATES

### Intermediate 1: 8-chloro-2-(methylthio)pyrido [3,4-d]pyrimidine

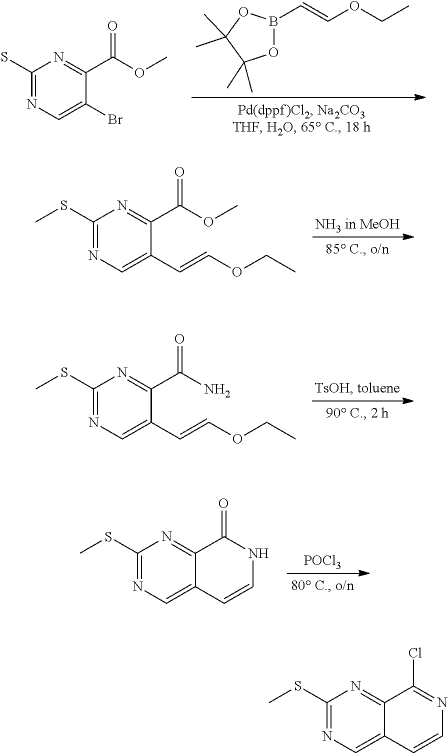

Step 1: methyl (E)-5-(2-ethoxyvinyl)-2-(methylthio)pyrimidine-4-carboxylate

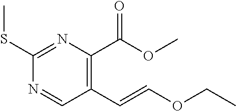 [SMILES: CCO/C=C/c1cnc(SC)nc1C(=O)OC]

To a stirred mixture of methyl 5-bromo-2-(methylsulfanyl) pyrimidine-4-carboxylate (7.8 g, 29.64 mmol) and 2-[(E)-2-ethoxyethenyl]-4,4,5,5-tetramethyl-1,3,2-dioxaborolane (9.45 g, 44.46 mmol) in tetrahydrofuran (60 mL) were added sodium carbonate (4.71 g, 44.46 mmol) in water (20 mL) and Pd(dppf)Cl₂ (1.08 g, 1.48 mmol) under a nitrogen atmosphere. The resulting mixture was heated to 65° C. and stirred for 18 hours. The reaction mixture was allowed to cool to room temperature, diluted with water (200 mL) and extracted with ethyl acetate (3×200 mL). The combined organic layers were washed with brine (200 mL), dried over anhydrous sodium sulfate, filtered, and concentrated under vacuum to afford the crude product. The residue was purified by silica gel column chromatography (ethyl acetate/petroleum ether, 85:15) to afford methyl (E)-5-(2-ethoxyvinyl)-2-(methylthio) pyrimidine-4-carboxylate (5.30 g, 70.3% yield).

LCMS (ESI) m/z=255 [M+H]⁺.

### Step 2: (E)-5-(2-ethoxyvinyl)-2-(methylthio)pyrimidine-4-carboxamide

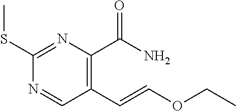 [SMILES: CCO/C=C/c1cnc(SC)nc1C(N)=O]

To a stirred mixture of methyl (E)-5-(2-ethoxyvinyl)-2-(methylthio) pyrimidine-4-carboxylate (4.8 g, 18.87 mmol) in methanol (5 mL) was added a solution of ammonium in methanol (7 M, 50 mL, 350 mmol). The resulting mixture was heated to 85° C. and stirred overnight in a sealed tube. The resulting mixture was allowed to cool to room temperature and concentrated under vacuum to afford the crude title product (4.2 g). The crude product was used for next step directly without further purification.

LCMS (ESI) m/z=240 [M+H]⁺.

### Step 3: 2-(methylthio)pyrido[3,4-d]pyrimidin-8(7H)-one

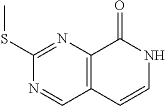

To a stirred mixture of (E)-5-(2-ethoxyvinyl)-2-(methylthio)pyrimidine-4-carboxamide (4.2 g, 17.55 mmol) in toluene (50 mL) was added p-toluene sulfonic acid (0.30 g, 1.75 mmol). The resulting mixture was heated to 90° C. and stirred for 2 hours. The reaction mixture was allowed to cool to room temperature and concentrated under vacuum to afford the crude product. The residue was diluted with water (200 mL) and extracted with ethyl acetate (3×200 mL). The combined organic layers were washed with brine (200 mL), dried over anhydrous sodium sulfate, filtered and concentrated under vacuum to afford the crude title product (4.4 g). The crude product was used for next step directly without further purification.

LCMS (ESI) m/z=194 [M+H]⁺.

### Step 4: 8-chloro-2-(methylthio)pyrido[3,4-d]pyrimidine

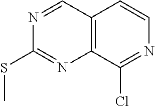

A mixture of 2-(methylthio)pyrido[3,4-d]pyrimidin-8(7H)-one (4.2 g, 21.73 mmol) in phosphorus oxychloride (150 mL) was heated to 80° C. and stirred overnight. The reaction mixture was allowed to cool to room temperature and concentrated under high vacuum. The residue was carefully quenched by addition of saturated aqueous sodium bicarbonate (500 mL) and extracted with ethyl acetate (3×500 mL). The combined organic layers were washed with brine (1000 mL), dried over anhydrous sodium sulfate, filtered, and concentrated under vacuum to afford the crude product. The residue was purified by silica gel column chromatography (petroleum ether/ethyl acetate, 2:1) to afford 8-chloro-2-(methylthio)pyrido [3,4-d]pyrimidine (3.70 g, 92.4% yield).

LCMS (ESI) m/z=212 [M+H]⁺.

### Intermediate 2: 6-methyl-2-(methylthio)pyrido[3,4-d]pyrimidin-8(7H)-one

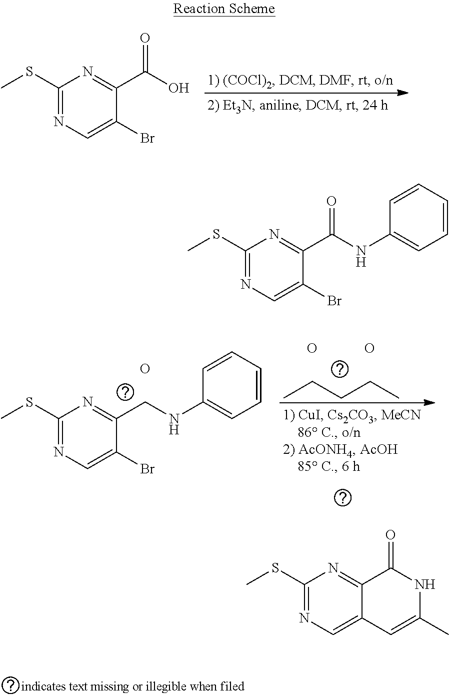

Detailed Procedure

### Step 1: 5-Bromo-2-(methylthio)-N-phenylpyrimidine-4-carboxamide

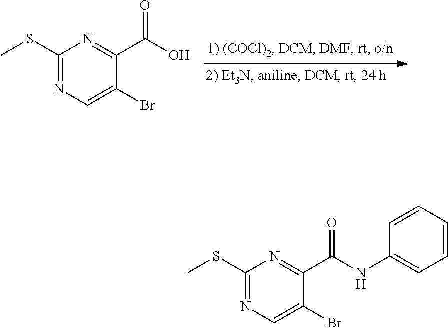 [SMILES: CSc1ncc(Br)c(C(=O)O)n1 ; CSc1ncc(Br)c(C(=O)Nc2ccccc2)n1]

Oxalyl dichloride (13.25 g, 104.4 mmol) was added to a solution of 5-bromo-2-(methylthio)pyrimidine-4-carboxylic acid (20 g, 80.3 mmol) in DCM (800 mL) at 0° C. A drop of DMF was added and the resulting mixture was stirred overnight at room temperature and concentrated under reduced pressure. The residue was dissolved in DCM (800 mL) and aniline (11.96 g, 128.5 mmol) and Et₃N (17.06 g, 168.6 mmol) were added at 0° C. The resulting mixture was stirred at room temperature for 24 h. The reaction was quenched by addition of aqueous HCl (0.5 N, 500 mL) and extracted with DCM (2×500 mL). The combined organic layers were washed with water (2×500 mL) and brine (500 mL), dried over anhydrous sodium sulfate and concentrated under reduced pressure to afford the crude product 5-bromo-2-(methylthio)-N-phenylpyrimidine-4-carboxamide (26 g crude). The crude product was used as such for next step.

LCMS (ESI-MS) m/z=324.0, 326.0 [M+H]⁺.

### Step 2: 6-Methyl-2-(methylthio)pyrido[3,4-d]pyrimidin-8(7H)-one

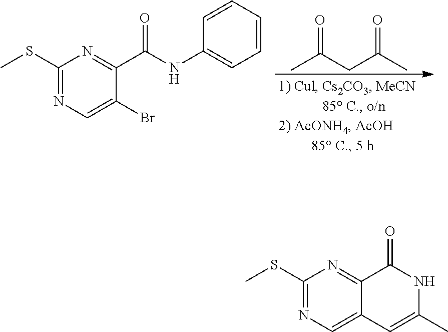

To a solution of 5-bromo-2-(methylthio)-N-phenylpyrimidine-4-carboxamide (26 g, 80.2 mmol) in ACN (270 mL) was added pentane-2,4-dione (16.06 g, 160.4 mmol), CuI (1.53 g, 8.02 mmol) and Cs₂CO₃ (52.26 g, 160.4 mmol). The resulting mixture was heated to 85° C. and stirred overnight. Ammonium acetate (101.8 g, 1.72 mol) and acetic acid (200 mL) were added and the resulting mixture was heated to 85° C. and stirred at for 5 h. After cooling to room temperature, the reaction mixture was concentrated under high vacuum until most of the liquid was evaporated. The residue was then neutralized by the addition of saturated aqueous NaOH (˜500 mL) at 0° C. followed by saturated aqueous NaHCO₃ at 0° C. until the pH value was adjusted to 6-7. The aqueous layer was extracted with DCM (2×1 L). The combined organic layers were washed with brine (2×500 mL), dried over anhydrous sodium sulfate and concentrated under reduced pressure to afford the crude product. The residue was purified by flash column chromatography eluting with 20% to 80% of ethyl acetate in petroleum ether followed by 0% to 10% MeOH in DCM to afford the desired product 6-methyl-2-(methylthio)pyrido[3,4-d]pyrimidin-8(7H)-one (5.8 g, 35% yield for 3 steps).

¹H NMR (400 MHz, DMSO-d₆) δ 11.9 (s, 1H), 9.07 (s, 1H), 6.31 (s, 1H), 2.58 (s, 3H), 2.22 (s, 3H).

LCMS (ESI-MS) m/z=208.0 [M+H]⁺.

### Intermediate 3: 8-chloro-6-methyl-2-(methylthio)pyrido[3,4-d]pyrimidine 8-Chloro-6-methyl-2-(methylthiolpyrido[3,4-d]pyrimidine

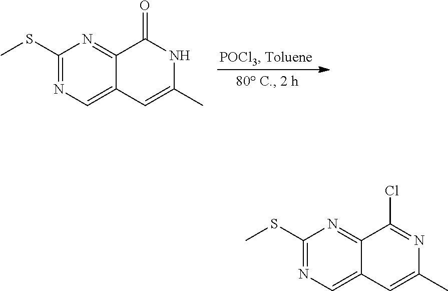

To a solution of 6-methyl-2-(methylthio)pyrido[3,4-d]pyrimidin-8(7H)-one (4 g, 19.30 mmol) in toluene (20 mL) was added POCl₃ (32.00 mL). The mixture was stirred at 80° C. for 2 h. The resulting mixture was concentrated under reduced pressure, quenched with water (200 mL) and extracted with ethyl acetate (3×200 mL). The combined organic layers were dried over anhydrous sodium sulfate, filtered and concentrated under reduced pressure. The crude product was purified by silica gel column chromatography, eluting with ethyl acetate/petroleum ether (0-20%) to afford 8-chloro-6-methyl-2-(methylthio)pyrido[3,4-d]pyrimidine (2.01 g, 46.4% yield).

¹H NMR (400 MHz, DMSO-d₆) δ 9.47 (s, 1H), 7.79 (d, J=0.6 Hz, 1H), 2.66 (s, 3H), 2.60 (s, 3H).

LCMS (ESI-MS) m/z=226.0 [M+H]⁺.

### Intermediate 4: 8-chloro-6-methyl-2-(methylsulfonyl)pyrido[3,4-d]pyrimidine and 8-chloro-6-methyl-2-(methylsulfinyl)pyrido[3,4-d]pyrimidine

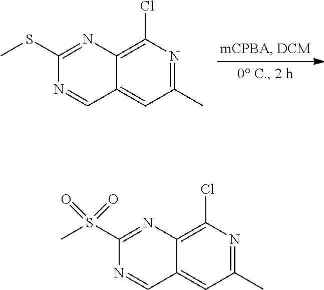

3-Chloroperoxybenzoic acid (6.88 g, 39.87 mmol) was added to 8-chloro-6-methyl-2-(methylthio)pyrido[3,4-d]pyrimidine (3.0 g, 13.29 mmol) in dichloromethane (50 mL) at 0° C. The reaction mixture was stirred for 30 minutes at 0° C., warmed to room temperature and stirred for 2 h. The reaction was quenched with saturated aqueous sodium bicarbonate (200 mL). The resulting mixture was extracted with dichloromethane (3×200 mL). The combined organic layers were dried over anhydrous sodium sulfate. After filtration, the filtrate was concentrated under reduced pressure. The residue was purified by column chromatography (EA in PE 0% to 100%) to afford crude 8-chloro-6-methyl-2-(methylsulfonyl)pyrido[3,4-d]pyrimidine and 8-chloro-6-methyl-2-(methylsulfinyl)pyrido[3,4-d]pyrimidine (1.07 g, 30.4% yield).

¹H NMR (400 MHz, DMSO-d₆) δ 9.99 (s, 1H), 8.09-8.06 (m, 1H), 3.54 (s, 3H), 2.81-2.66 (m, 3H).

LCMS (ESI-MS) m/z=257.9 [M+H]⁺.

### Intermediate 5: 8-bromo-6-methyl-N-(1-(methylsulfonyl)piperidin-4-yl)pyrido[3,4-d]pyrimidin-2-amine

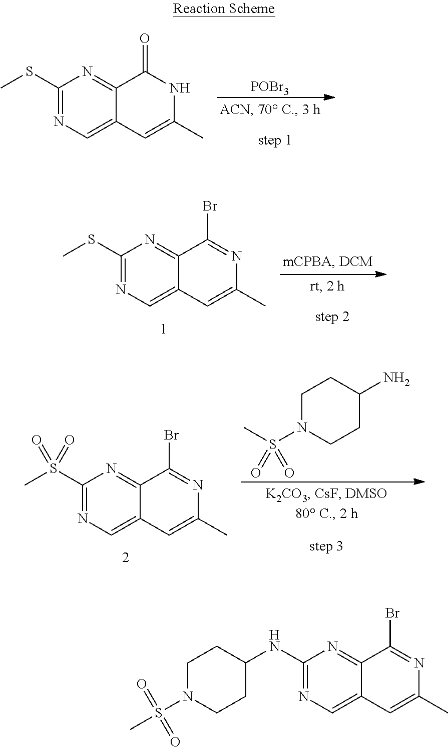

### Step 1: 8-Bromo-6-methyl-2-(methylthio)pyrido[3,4-d]pyrimidine

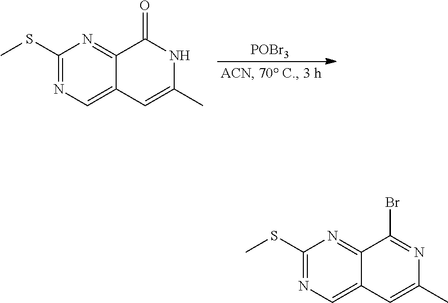

POBr3 (50 mL, 174.4 mmol) was added to 6-methyl-2-(methylthio)pyrido[3,4-d]pyrimidin-8(7H)-one (5 g, 24.12 mmol) in MeCN (50 mL). The mixture was stirred at 70° C. for 3 h and concentrated under reduced pressure. The residue was quenched with aqueous sodium bicarbonate at 0° C. and extracted with EA (3×200 mL). The combined organic layers were dried over anhydrous sodium sulfate. After filtration, the filtrate was concentrated under reduced pressure. The residue was purified by silica gel column chromatography, eluting with EA/PE (0-20%) to afford 8-bromo-6-methyl-2-(methylthio)pyrido[3,4-d]pyrimidine (2.2 g, 33.8% yield).

LCMS (ESI-MS) m/z=270.0, 272.0 [M+H]⁺.

### Step 2: 8-Bromo-6-methyl-2-(methylsulfonyl)pyrido[3,4-d]pyrimidine

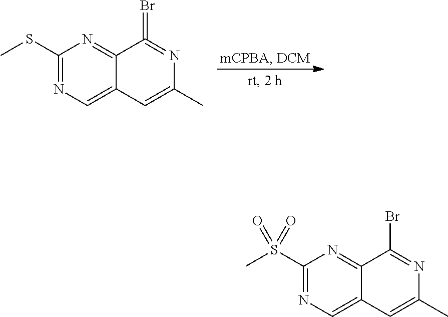

3-Chloroperoxybenzoic acid (2.81 g, 16.28 mmol) was added to 8-bromo-6-methyl-2-(methylthio)pyrido[3,4-d]pyrimidine (2.2 g, 8.14 mmol) in DCM (50 mL) at 0° C. The reaction mixture was stirred for 30 minutes at 0° C., then warmed to room temperature and stirred for 2 h. The reaction was quenched with saturated aqueous sodium bicarbonate (200 mL). The resulting mixture was extracted with DCM (3×200 mL). The combined organic layers were dried over anhydrous sodium sulfate. After filtration, the filtrate was concentrated under reduced pressure. The residue was purified by column chromatography (EA in PE from 0% to 100%) to afford crude 8-bromo-6-methyl-2-(methylsulfonyl)pyrido[3,4-d]pyrimidine (1.3 g, 52.8% yield).

LCMS (ESI-MS) m/z=302.0, 304.0 [M+H]⁺.

### Step 3: 8-Bromo-6-methyl-N-(1-(methylsulfonyl)piperidin-4-yl)pyrido[3,4-d]pyrimidin-2-amine

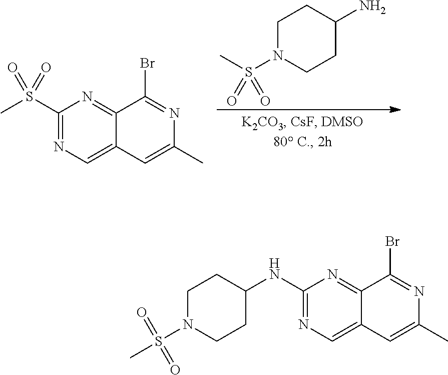

To a solution of 8-bromo-6-methyl-2-(methylsulfonyl)pyrido[3,4-d]pyrimidine (1.3 g, 4.30 mmol) in DMSO (10 mL) was added 1-(methylsulfonyl)piperidin-4-amine (0.77 g, 4.30 mmol), K₂CO₃ (1.19 g, 8.60 mmol) and CsF (1.31 g, 8.60 mmol). The reaction mixture was stirred for 2 h at 80° C. The reaction mixture was diluted with water (50 mL) and extracted with DCM (3×50 mL). The combined organic layers were washed with brine (2×40 mL), dried over anhydrous sodium sulfate and concentrated under reduced pressure. The residue was purified by silica gel column chromatography (EA/PE, 0-20%) to afford 8-bromo-6-methyl-N-(1-(methylsulfonyl)piperidin-4-yl)pyrido[3,4-d]pyrimidin-2-amine (206.1 mg, 10.8% yield).

¹H NMR (400 MHz, DMSO-d₆) δ 9.18 (s, 1H), 8.12-7.96 (m, 1H), 7.57-7.54 (m, 1H), 4.09-3.94 (m, 1H), 3.64-3.51 (m, 2H), 2.90 (s, 6H), 2.57-2.45 (m, 2H), 2.18-2.01 (m, 2H), 1.71-1.56 (m, 2H).

LCMS (ESI-MS) m/z=400.0, 402.0 [M+H]⁺.

### Intermediate 6: 8-chloro-6-methyl-N-(1-(methylsulfonyl)piperidin-4-yl)pyrido[3,4-d]pyrimidin-2-amine

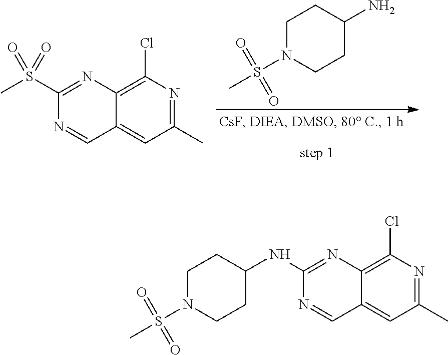

A mixture of 8-chloro-6-methyl-2-(methylsulfonyl)pyrido[3,4-d]pyrimidine (1 g, 3.88 mmol), 1-(methylsulfonyl)piperidin-4-amine (0.8 g, 4.26 mmol), DIEA (1.50 g, 11.64 mmol) and CsF (1.77 g, 11.64 mmol) in DMSO (5 mL) was stirred for 1 h at 80° C. The reaction mixture was diluted with water (100 mL) and extracted with CH₂Cl₂ (3×100 mL). The combined organic layers were dried over anhydrous Na₂SO₄. After filtration, the filtrate was concentrated under reduced pressure. The residue was purified by silica gel column chromatography(PE/EA, 1:1) to afford 8-chloro-6-methyl-N-(1-(methylsulfonyl)piperidin-4-yl)pyrido[3,4-d]pyrimidin-2-amine (505.8 mg, 33.3% yield).

LCMS (ESI-MS) m/z=356.0 [M+H]⁺.

### Intermediate 7: 8-chloro-6-cyclopropyl-2-methanesulfonylpyrido[3,4-d]pyrimidine

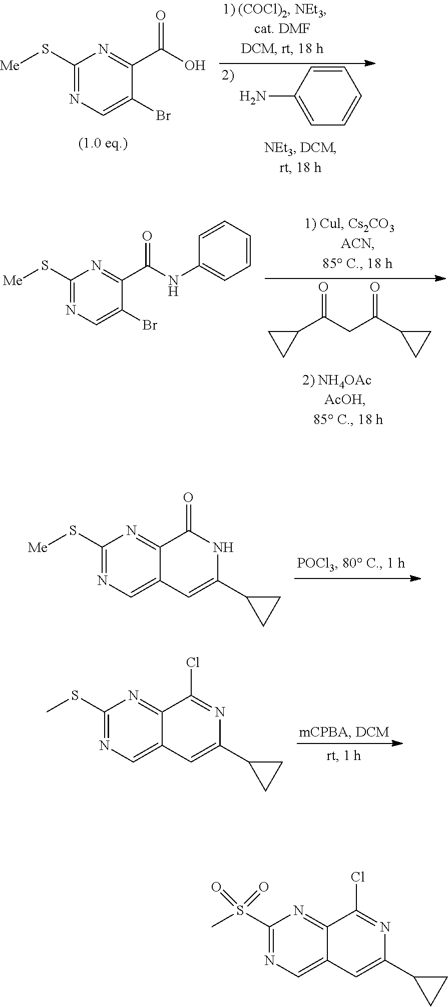

### Step 1: 5-bromo-2-(methylsulfanyl)-N-phenylpyrimidine-4-carboxamide

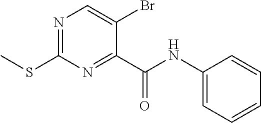 [SMILES: CSc1ncc(Br)c(C(=O)Nc2ccccc2)n1]

A solution of 5-bromo-2-(methylthio)pyrimidine-4-carboxylic acid (15.0 g, 60.222 mmol) in dichloromethane anhydrous (225.0 ml) was cooled down to 0° C. Oxalyl chloride (6.624 ml, 78.288 mmol) and one drop of dimethylformamide anhydrous were added under argon atmosphere and the reaction mixture was stirred at room temperature for 18 hours. Then, the reaction mixture was concentrated under reduced pressure and the residue was redissolved in dichloromethane anhydrous (225.0 ml) and cooled down to 0° C. Aniline (9.329 ml, 102.377 mmol) and triethylamine (18.467 ml, 132.488 mmol) were slowly added and the reaction mixture was stirred at room temperature for additional 18 hours. The reaction mixture was quenched with 0.5 M HCl aq. sol, and layers were separated. Aqueous layer was extracted with dichloromethane (3×200 ml) and the combined organic layers were dried over anhydrous MgSO4, filtered and concentrated under reduced pressure. Crude material was purified by automated flash column chromatography on silica gel (from hexane to hexane/EtOAc 4:1) to afford the title compound (17.34 g, 89% yield).

¹H NMR (300 MHz, Chloroform-d) δ 9.63 (bs, 1H), 8.86 (s, 1H), 7.77-7.71 (m, 2H), 7.42 (t, J=7.9 Hz, 2H), 7.21 (t, J=7.4 Hz, 1H), 2.66 (s, 3H).

UPLC (ESI) [M+H]⁺=323.70 and 325.70 (Br isotopic pattern).

### Step 2: 6-cyclopropyl-2-(methylsulfanyl)-7H,8H-pyrido[3,4-d]pyrimidin-8-one

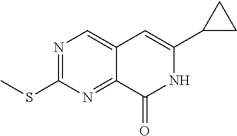 [SMILES: CSc1ncc2cc(C3CC3)[nH]c(=O)c2n1]

A solution of 5-bromo-2-(methylsulfanyl)-N-phenylpyrimidine-4-carboxamide (10.0 g, 30.845 mmol), 1,3-dicyclopropylpropane-1,3-dione (7.633 ml, 61.69 mmol), cesium carbonate (20.1 g, 61.69 mmol) and copper(I) iodide (0.587 g, 3.085 mmol) in acetonitrile anhydrous (88.13 ml) was stirred at 85° C. for 18 hours. Then, acetic acid (88.13 ml) and ammonium acetate (35.665 g, 462.677 mmol) were added and the reaction mixture was stirred at 85° C. for additional 18 hours. The reaction mixture was concentrated under reduced pressure to remove acetonitrile, and acetic acid-containing mixture was diluted with water (100 ml) and extracted with dichloromethane (3×100 ml). The combined organic layers were dried over anhydrous MgSO4, filtered and concentrated under reduced pressure. Crude material was purified by automated flash column chromatography on silica gel (from hexane/EtOAc 3:2 to EtOAc) to afford the title compound (4.76 g, 66% yield).

¹H NMR (300 MHz, DMSO-d₆) δ 12.01 (bs, 1H), 9.06 (s, 1H), 6.21 (s, 1H), 2.58 (s, 3H), 1.88 (tt, J=8.3, 5.1 Hz, 1H), 1.04-0.92 (m, 2H), 0.89-0.77 (m, 2H).

UPLC (ESI) [M+H]⁺=233.90.

### Step 3: 8-chloro-6-cyclopropyl-2-(methylsulfanyl)pyrido[3,4-d]pyrimidine

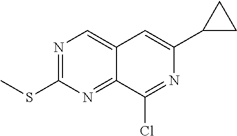 [SMILES: CSc1ncc2cc(C3CC3)nc(Cl)c2n1]

A solution of 6-cyclopropyl-2-(methylsulfanyl)-7H,8H-pyrido[3,4-d]pyrimidin-8-one (4.76 g, 20.404 mmol) in phosphorus oxychloride (32.428 ml, 346.864 mmol) was stirred at 80° C. for 1 hour. The reaction mixture was concentrated under reduced pressure and the residue was redissolved in ethyl acetate (50 ml) and quenched with NaHCO₃ sat. aq. solution. Layers were separated and aqueous layer was extracted with ethyl acetate (3×50 ml). The combined organic layers were dried over anhydrous MgSO4, filtered and concentrated under reduced pressure. Crude material was purified by automated flash column chromatography on silica gel (from hexane to hexane/EtOAc 7:3) to afford the title compound (4.58 g, 89% yield).

¹H NMR (300 MHz, DMSO-d₆) δ 9.48 (s, 1H), 7.87 (s, 1H), 2.66 (s, 3H), 2.38-2.19 (m, 1H), 1.14-1.02 (m, 2H), 0.97 (ddd, J=7.2, 5.1, 2.9 Hz, 2H).

UPLC (ESI) [M+H]⁺=251.85.

### Step 4: 8-chloro-6-cyclopropyl-2-methanesulfonylpyrido[3,4-d]pyrimidine

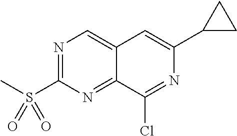 [SMILES: CS(=O)(=O)c1ncc2cc(C3CC3)nc(Cl)c2n1]

To a solution of 8-chloro-6-cyclopropyl-2-(methylsulfanyl)pyrido[3,4-d]pyrimidine (2.5 g, 9.931 mmol) in dichloromethane (125.0 ml), 3-chloroperbenzoic acid (8.569 g, 49.656 mmol) was added portionwise and the reaction mixture was stirred at room temperature for 1 hour. The reaction mixture was quenched by the slowly addition of a 10% Na₂S₂O₃ aq. solution (50 ml) and extracted with dichloromethane (3×70 ml). The combined organic layers were washed with NaHCO₃ sat. aq. solution, dried over anhydrous MgSO4, filtered and concentrated under reduced pressure. Crude material was purified by automated flash column chromatography on silica gel (from hexane/EtOAc 4:1 to hexane/EtOAc 2:3) to afford the title compound (2.54 g, 89% yield).

¹H NMR (300 MHz, DMSO-d₆) δ 9.94 (s, 1H), 8.12 (s, 1H), 3.52 (s, 3H), 2.42 (dq, J=8.4, 4.7, 4.1 Hz, 1H), 1.17 (dt, J=7.9, 3.0 Hz, 2H), 1.09-1.02 (m, 2H).

UPLC (ESI) [M+H]⁺=283.80.

### Intermediate 8: 8-chloro-6-cyclopropyl-N-(1-(methylsulfonyl)piperidin-4-yl)pyrido[3,4-d]pyrimidin-2-amine

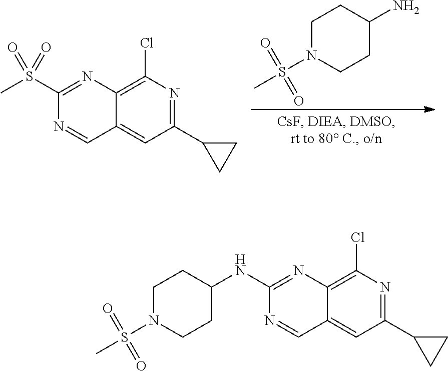 [SMILES: CS(=O)(=O)c1ncc2cc(C3CC3)nc(Cl)c2n1 ; CS(=O)(=O)N1CCC(N)CC1 ; CS(=O)(=O)N1CCC(Nc2ncc3cc(C4CC4)nc(Cl)c3n2)CC1]

A solution of 8-chloro-6-cyclopropyl-2-(methylsulfonyl)pyrido[3,4-d]pyrimidine (100 mg, 352 μmol) in dimethylsulfoxide anhydrous (1.8 mL) was charged with 1-(methylsulfonyl)piperidin-4-amine (69.1 mg, 388 μmol), cesium fluoride (161 mg, 1.06 mmol), and N-ethyl-N-isopropylpropan-2-amine (137 mg, 184 μL, 1.06 mmol). The reaction vial was capped and stirred at 30° C. for 72 hours. After, the crude mixture was diluted with water (50.0 mL) and extracted with dichloromethane (3×20.0 mL). The combined organic fractions washed with brine, dried over anhydrous magnesium sulfate, filtered, and the solvent was removed in vacuo. The crude material was purified by flash chromatography on silica gel (heptane/EtOAc 1:1) to afford the title compound (62.0 mg, 46% yield).

LCMS (ESI) [M+H]⁺=382.10

### Intermediate 9: 8-chloro-6-(difluoromethyl)-2-(methylsulfanyl)pyrido[3,4-d]pyrimidine

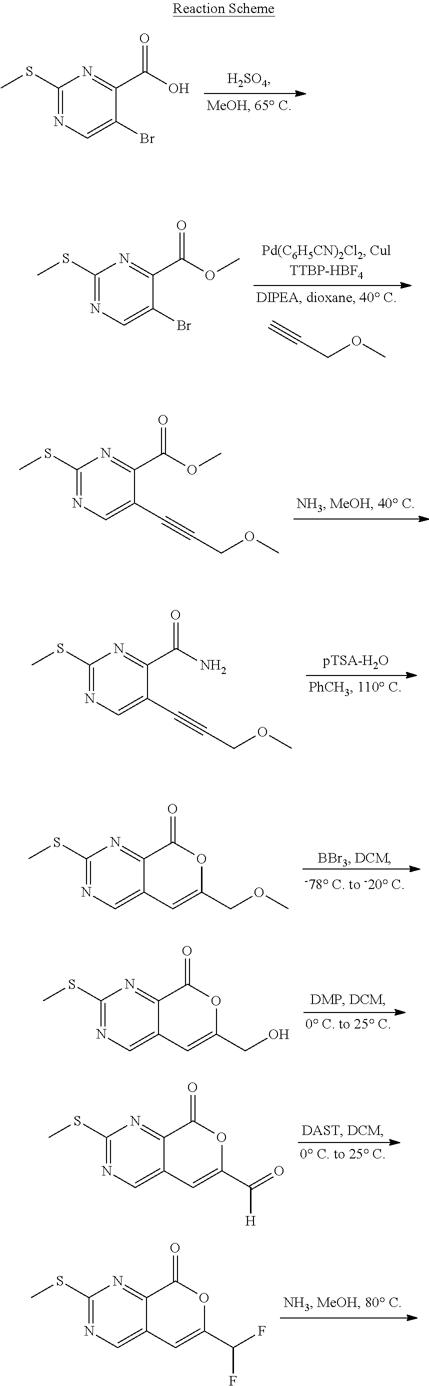

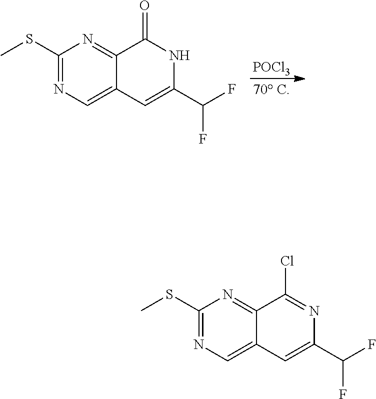 [SMILES: CSc1ncc2cc(C(F)F)[nH]c(=O)c2n1 ; "POCl3" ; CSc1ncc2cc(C(F)F)nc(Cl)c2n1]

### Detailed Procedure

### Step 1: methyl 5-bromo-2-(methylsulfanyl)pyrimidine-4-carboxylate

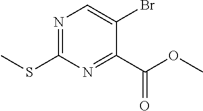

To a solution of 5-bromo-2-(methylthio)pyrimidine-4-carboxylic acid (15.0 g, 60.222 mmol) in methanol (600.0 ml), sulfuric acid (98%, 3.852 ml, 72.266 mmol) was slowly added. The reaction mixture was stirred at 65° C. for 16 hours. Then, the reaction mixture was poured onto ice water and extracted with dichloromethane (300 mL×3). The combined organic layers were washed with NaHCO₃ sat. aq. solution, dried over anhydrous MgSO4, filtered and concentrated under reduced pressure to provide the title compound (15.02 g, 95% yield). Crude material was used in the next step without further purification.

¹H NMR (300 MHz, Chloroform-d) δ 8.73 (s, 1H), 4.02 (s, 3H), 2.59 (s, 3H).

UPLC (ESI) [M+H]⁺=262.70 and 264.65 (Br isotopic pattern).

### Step 2: methyl 5-(3-methoxyprop-1-yn-1-yl)-2-(methylsulfanyl)pyrimidine-4-carboxylate

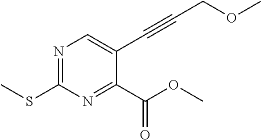

A solution of bis(benzonitrile)palladium chloride (0.729 g, 1.9 mmol), copper(I) iodide (0.362 g, 1.9 mmol) and tri-tert-butylphosphonium tetrafluoroborate (1.103 g, 3.801 mmol) in dioxane anhydrous (50.0 ml) was purged with argon for 10 minutes. Then, N,N-diisopropylethylamine (23.171 ml, 133.024 mmol) was added and the mixture was stirred for 5 min at room temperature. Next, methyl 5-bromo-2-(methylsulfanyl)pyrimidine-4-carboxylate (5.0 g, 19.003 mmol) and 3-methoxyprop-1-yne (4.814 ml, 57.01 mmol) were slowly added and the reaction mixture was stirred at 40° C. overnight. The reaction mixture was cooled down to room temperature, filtered through a pad of celite and washed with ethyl acetate (50 mL) and dichloromethane (50 mL). The filtrate was concentrated under reduced pressure and purified automated flash column chromatography on silica gel (from hexane to hexane/EtOAc 7:3) to provide the title compound (2.47 g, 52% yield).

¹H NMR (300 MHz, Chloroform-d) δ 8.73 (s, 1H), 4.39 (s, 2H), 4.01 (s, 3H), 3.50 (s, 3H), 2.63 (s, 3H).

UPLC (ESI) [M+H]⁺=252.80.

### Step 3: 5-(3-methoxyprop-1-yn-1-yl)-2-(methylsulfanyl)pyrimidine-4-carboxamide

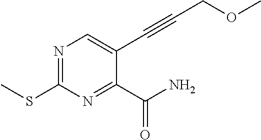

A solution of methyl 5-(3-methoxyprop-1-yn-1-yl)-2-(methylsulfanyl)pyrimidine-4-carboxylate (2.47 g, 9.79 mmol) in ammonia (7.0 N solution in methanol, 34.965 ml, 244.758 mmol) was stirred at 40° C. for 2 hours. The mixture was concentrated under reduced pressure to provide the crude title compound (2.28 g, 98% yield). The crude product was used for the next step without further purification.

¹H NMR (300 MHz, Chloroform-d) δ 8.78 (s, 1H), 7.55 (s, 1H), 5.58 (s, 1H), 4.44 (s, 2H), 3.53 (s, 3H), 2.63 (s, 3H).

UPLC (ESI) [M+H]⁺=237.85.

### Step 4: 6-(methoxymethyl)-2-(methylsulfanyl)-8H-pyrano[3,4-d]pyrimidin-8-one

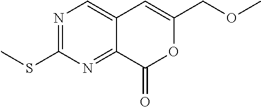

To a solution of 5-(3-methoxyprop-1-yn-1-yl)-2-(methylsulfanyl)pyrimidine-4-carboxamide (1.580 g, 6.660 mmol) in toluene (28.56 ml) was added p-toluenesolfonic acid monohydrate (0.916 g, 4.815 mmol). The reaction mixture was stirred at 110° C. for 48 hours. The reaction mixture was concentrated under reduced pressure and purified by flash column chromatography on silica gel (from hexane/EtOAc 7:3 to hexane/EtOAc 3:7) to provide the title compound (0.685 g, 43% yield).

¹H NMR (300 MHz, Chloroform-d) δ 8.85 (s, 1H), 6.53 (t, J=1.2 Hz, 1H), 4.31 (d, J=1.2 Hz, 2H), 3.53 (s, 3H), 2.70 (s, 3H).

### Step 5: 6-(hydroxymethyl)-2-(methylsulfanyl)-8H-pyrano[3,4-d]pyrimidin-8-one

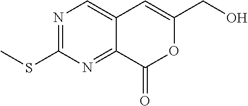

A solution of 6-(methoxymethyl)-2-(methylsulfanyl)-8H-pyrano[3,4-d]pyrimidin-8-one (1.275 g, 5.373 mmol) in dichloromethane anhydrous (44.78 ml) was cooled down to −78° C. Then, boron tribromide (1.0 M solution in dichloromethane, 32.247 ml, 32.247 mmol) was added dropwise via addition funnel and the reaction mixture was stirred at the same temperature for 20 minutes. Next, the reaction mixture was warmed up to −20° C. and stirred for an additional 2 hours and 30 minutes. The reaction mixture was quenched by the dropwise addition of methanol followed by NaHCO₃ sat. aq. solution (until gas release stopped). The mixture was extracted with dichloromethane (50 mL×3). The combined organic layers were dried over anhydrous MgSO4, filtered, concentrated under reduced pressure and purified by flash column chromatography on silica gel (from hexane/EtOAc 7:3 to hexane/EtOAc 1:9) to provide the title compound (0.787 g, 59% yield).

¹H NMR (300 MHz, DMSO-d₆) δ 9.19 (s, 1H), 6.74 (d, J=1.2 Hz, 1H), 5.74 (t, J=6.0 Hz, 1H), 4.30 (dd, J=6.0, 1.2 Hz, 2H), 2.61 (s, 3H).

### Step 6: 2-(methylsulfanyl)-8-oxo-8H-pyrano[3,4-d]pyrimidine-6-carbaldehyde

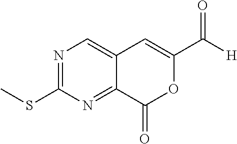

A solution of 6-(hydroxymethyl)-2-(methylsulfanyl)-8H-pyrano[3,4-d]pyrimidin-8-one (0.787 g, 3.173 mmol) in dichloromethane anhydrous (15.74 ml) was cooled down to 0° C. Then, Dess-Martin periodinane (2.692 g, 6.347 mmol) was added portionwise. The reaction mixture was allowed to warm up to room temperature and stirred for 30 minutes. The solid was filtered off and the filtrate was washed with 0.5 M NaOH aq. solution, dried over anhydrous MgSO4, filtered and concentrated under reduced pressure. The crude material was purified by flash column chromatography on silica gel (from hexane/EtOAc 8:2 to hexane/EtOAc 4:6) to provide the title compound (0.738 g, 100% yield).

¹H NMR (300 MHz, DMSO-d₆) δ 9.58 (s, 1H), 9.35 (s, 1H), 7.78 (s, 1H), 2.65 (s, 3H).

### Step 7: 6-(difluoromethyl)-2-(methylsulfanyl)-8H-pyrano[3,4-d]pyrimidin-8-one

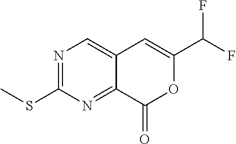 [SMILES: CSc1ncc2cc(C(F)F)oc(=O)c2n1]

A solution of 2-(methylsulfanyl)-8-oxo-8H-pyrano[3,4-d]pyrimidine-6-carbaldehyde (0.738 g, 3.167 mmol) in dichloromethane (22.13 ml) was cooled down to 0° C. Then, DAST (0.418 ml, 3.167 mmol) was added dropwise and the reaction mixture was allowed to warm up to room temperature and stirred for 1 hour. The reaction mixture was quenched by the dropwise addition of a 10% Na₂S₂O₃ aq. solution (10 mL). Layers were separated and aqueous layer was extracted with dichloromethane (15 mL×3). The combined organic layers were dried over anhydrous MgSO4, filtered and concentrated under reduced pressure to provide the title compound (0.784 g, 92% yield). Crude material was used in the next step without further purification.

¹H NMR (300 MHz, DMSO-d₆) δ 9.24 (s, 1H), 7.25 (d, J=1.6 Hz, 1H), 6.94 (t, J=52.7 Hz, 1H), 2.63 (s, 3H).

UPLC (ESI) [M+H]⁺=244.85.

### Step 8: 6-(difluoromethyl)-2-(methylsulfanyl)-7H,8H-pyrido[3,4-d]pyrimidin-8-one

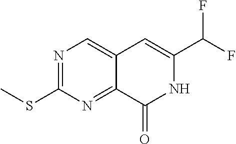 [SMILES: CSc1ncc2cc(C(F)F)[nH]c(=O)c2n1]

A solution of 6-(difluoromethyl)-2-(methylsulfanyl)-8H-pyrano[3,4-d]pyrimidin-8-one (0.734 g, 2.703 mmol) in ammonia (7.0 N solution in methanol, 21.242 ml, 148.691 mmol) was stirred at 80° C. for 16 hours. The volatiles were removed under reduced pressure to provide the crude title compound (0.704 g, 96% yield) as a dark solid. The crude product was used for the next step without further purification.

¹H NMR (300 MHz, DMSO-d₆) δ 9.28 (s, 1H), 6.95 (d, J=1.6 Hz, 1H), 6.88 (t, J=53.8 Hz, 1H), 2.62 (s, 3H).

UPLC (ESI) [M+H]⁺=243.75.

### Step 9: 8-chloro-6-(difluoromethyl)-2-(methylsulfanyl)pyrido[3,4-d]pyrimidine

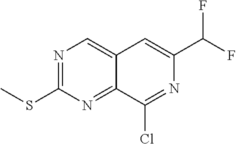 [SMILES: CSc1ncc2cc(C(F)F)nc(Cl)c2n1]

A solution of 6-(difluoromethyl)-2-(methylsulfanyl)-7H,8H-pyrido[3,4-d]pyrimidin-8-one (0.704 g, 2.605 mmol) in phosphorus oxychloride (7.306 ml, 78.148 mmol) was stirred at 70° C. for 3 hours. The reaction mixture was concentrated under reduced pressure and the residue was redissolved in ethyl acetate (15 mL) and washed with sat. aq. NaHCO₃(15 mL). The aqueous layer was extracted with ethyl acetate (15 mL×3) and the combined organic layers were dried over anhydrous MgSO4, filtered and concentrated under reduced pressure. Crude material was purified by flash column chromatography on silica gel (from hexane to hexane/EtOAc 7:3) to provide the title compound (0.277 g, 41% yield).

¹H NMR (300 MHz, DMSO-d₆) δ 9.69 (s, 1H), 8.37 (s, 1H), 7.17 (t, J=54.5 Hz, 1H), 2.71 (s, 3H).

UPLC (ESI) [M+H]⁺=261.80.

### Intermediate 10: N-(5-(6-ethyl-2,6-diazaspiro[3.3]heptan-2-yl) pyridin-2-yl) formamide

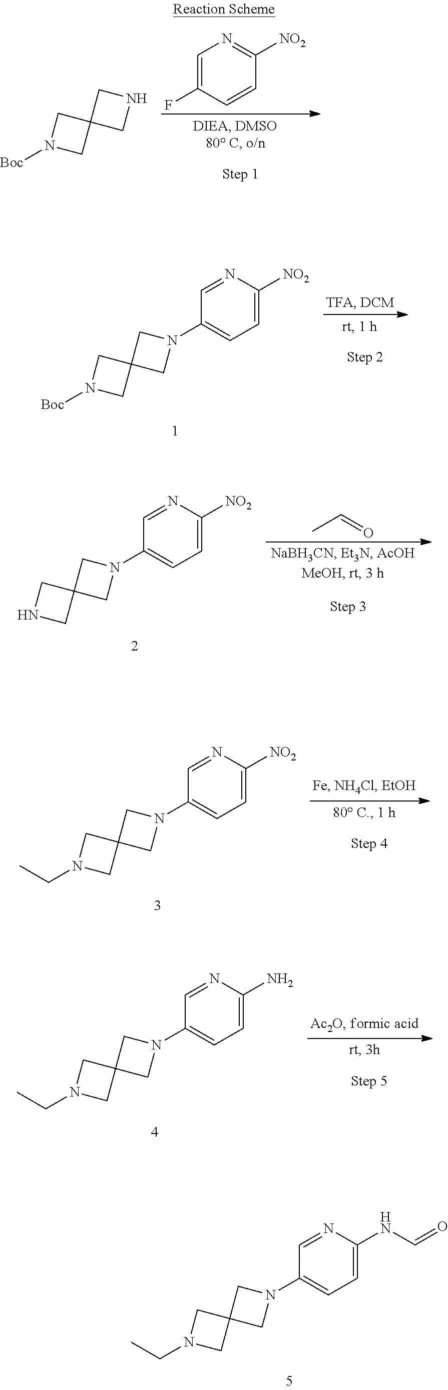

### Step 1: tert-butyl 6-(6-nitropyridin-3-yl)-2,6-diazaspiro[3.3]heptane-2-carboxylate

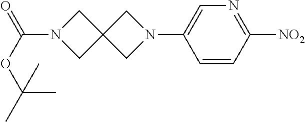 [SMILES: CC(C)(C)OC(=O)N1CC2(C1)CN(c1ccc([N+](=O)[O-])nc1)C2]

To a stirred mixture of tert-butyl 2,6-diazaspiro[3.3]heptane-2-carboxylate (5 g, 25.21 mmol) and 5-fluoro-2-nitropyridine (5.37 g, 37.82 mmol) in dimethyl sulfoxide (30 mL) was N,N-diisopropylethylamine (9.78 g, 75.65 mmol). The resulting mixture was heated to 80° C. and stirred for 3 hours. The reaction mixture was allowed to cool to room temperature, diluted with water (500 mL) and extracted with ethyl acetate (3×500 mL). The combined organic layers were washed with brine (500 mL), dried over anhydrous sodium sulfate, and filtered. The filtrate was concentrated under vacuum to afford the crude product. The residue was purified by trituration with petroleum ether/ethyl acetate (5:1,100 mL) to afford tert-butyl 6-(6-nitropyridin-3-yl)-2,6-diazaspiro[3.3]heptane-2-carboxylate (6.78 g, 83.7% yield).

LCMS (ESI) m/z=321 [M+H]⁺.

### Step 2: 2-(6-nitropyridin-3-yl)-2,6-diazaspiro [3.3] heptane

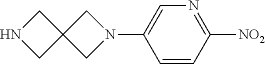 [SMILES: O=[N+]([O-])c1ccc(N2CC3(CNC3)C2)cn1]

To a stirred mixture of tert-butyl 6-(6-nitropyridin-3-yl)-2,6-diazaspiro[3.3]heptane-2-carboxylate (6.78 g, 21.16 mmol) in dichloromethane (80 mL) was added trifluoroacetic acid (16 mL). The resulting mixture was stirred for 1 hour at room temperature and concentrated under vacuum. The residue was diluted with dichloromethane (100 mL) and concentrated under vacuum again to afford crude 2-(6-nitropyridin-3-yl)-2,6-diazaspiro[3.3]heptane trifluoroacetic acid salt (6 g). The crude product was used for next step directly without further purification.

LCMS (ESI) m/z=221 [M+H]⁺.

### Step 3: 2-ethyl-6-(6-nitropyridin-3-yl)-2,6-diazaspiro[3.3]heptane

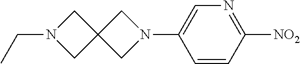 [SMILES: CCN1CC2(C1)CN(c1ccc([N+](=O)[O-])nc1)C2]

A solution of 2-(6-nitropyridin-3-yl)-2,6-diazaspiro[3.3]heptane trifluoroacetic acid salt (6 g, 18.9 mmol) in methanol (100 mL) was treated with triethylamine (5.73 g, 56.7 mmol) for 10 minutes followed by the addition of acetaldehyde (4.16 g, 94.5 mmol), acetic acid (0.23 mL, 4.08 mmol) and sodium cyanoborohydride (2.51 g, 39.8 mmol). The resulting mixture was stirred for 3 hours at room temperature and concentrated under vacuum. The residue was diluted with water (500 mL) and extracted with ethyl acetate (3×500 mL). The combined organic layers were washed with brine (1000 mL), dried over anhydrous sodium sulfate, and filtered. The filtrate was concentrated under reduced pressure to afford the crude product. The residue was purified by trituration with dichloromethane (100 mL) to afford 2-ethyl-6-(6-nitropyridin-3-yl)-2,6-diazaspiro[3.3]heptane (4 g, 58.9% yield) as an orange solid.

LCMS (ESI) m/z=249 [M+H]⁺.

### Step 4: 5-(6-ethyl-2,6-diazaspiro[3.3]heptan-2-yl) pyridin-2-amine

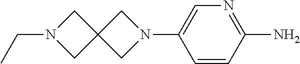 [SMILES: CCN1CC2(C1)CN(c1ccc(N)nc1)C2]

A solution of 2-ethyl-6-(6-nitropyridin-3-yl)-2,6-diazaspiro[3.3]heptane (4 g, 16.11 mmol), ammonium chloride (4.31 g, 80.55 mmol), iron powder (9.00 g, 161.100 mmol) and water (20 mL) in ethanol (60 mL) was stirred for 1 hour at 80° C. The resulting mixture was filtered and the filter cake was washed with ethanol (100 mL). The filtrate was concentrated under vacuum to afford the crude product. The residue was purified by reversed-phase flash chromatography (C18 silica gel, acetonitrile/water (with 10 mmol/L NH₄HCO₃) gradient) to afford 5-(6-ethyl-2,6-diazaspiro[3.3]heptan-2-yl)pyridin-2-amine (2 g, 56.6% yield).

LCMS (ESI) m/z=219 [M+H]⁺.

### Step 5: N-(5-(6-ethyl-2,6-diazaspiro[3.3]heptan-2-yl) pyridin-2-yl) formamide

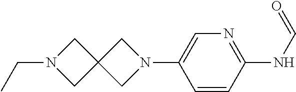 [SMILES: CCN1CC2(C1)CN(c1ccc(NC=O)nc1)C2]

A solution of acetic anhydride (2 mL) in formic acid (4 mL) was stirred for 1 hour at room temperature followed by the addition of 5-(6-ethyl-2,6-diazaspiro[3.3]heptan-2-yl)pyridin-2-amine (400 mg, 1.83 mmol) in portions at room temperature. The resulting mixture was stirred for 3 hours at room temperature. The reaction mixture was neutralized to PH=7 with saturated aqueous sodium bicarbonate (200 mL) and extracted with ethyl acetate (3×100 mL). The combined organic layers were washed with brine, dried over anhydrous sodium sulfate, and filtered. The filtrate was concentrated under vacuum and the residue was purified by preparative reverse phase HPLC (acetonitrile/water (with 10 mM NH₄HCO₃ and 0.1% NH₃·H₂O) gradient) to afford the title compound (70 mg, 15.3% yield).

LCMS (ESI) m/z=247 [M+H]⁺.

### Intermediate 12: 1-(cyclopropylsulfonyl)piperidin-4-amine

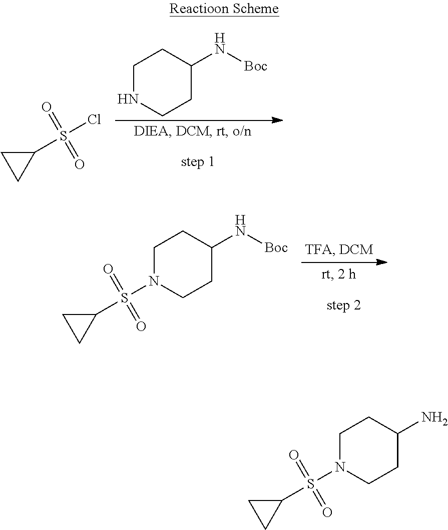

Detailed Procedure

### Step 1: Tert-butyl (1-(cyclopropylsulfonyl)piperidin-4-yl)carbamate

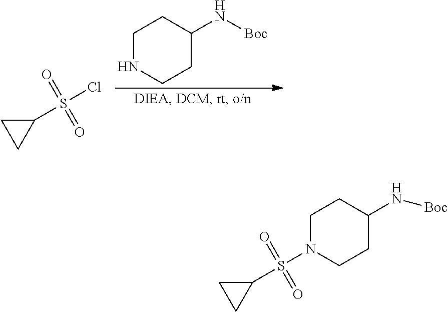

To a solution of cyclopropanesulfonyl chloride (7.02 g, 49.93 mmol) and DIEA (19.36 g, 149.79 mmol) in DCM (100 mL) was added tert-butyl N-(piperidin-4-yl)carbamate (10 g, 49.93 mmol) dropwise at 0° C. The resulting mixture was stirred overnight at room temperature. The resulting mixture was diluted with water (500 mL). The aqueous solution was extracted with CH₂Cl₂ (3×500 mL). The combined organic layers were dried over anhydrous Na₂SO₄. After filtration, the filtrate was concentrated under reduced pressure. The residue was purified by trituration with EA (100 mL) to afford tert-butyl (1-(cyclopropylsulfonyl)piperidin-4-yl)carbamate (10 g, 59.2% yield).

LCMS (ESI-MS) m/z=249.1 [M+H-56]⁺.

### Step 2: 1-(cyclopropylsulfonyl)piperidin-4-amine

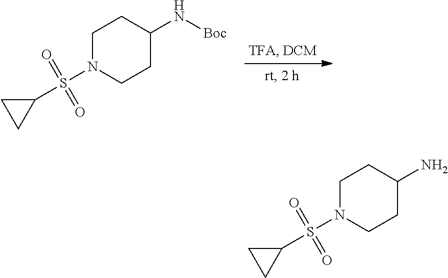 [SMILES: CC(C)(C)OC(=O)NC1CCN(S(=O)(=O)C2CC2)CC1 ; NC1CCN(S(=O)(=O)C2CC2)CC1]

A solution of tert-butyl (1-(cyclopropylsulfonyl)piperidin-4-yl)carbamate (10 g, 32.87 mmol) in TFA (15 mL) and DCM (45 mL) was stirred for 2 h at room temperature. The resulting mixture was concentrated under reduced pressure to afford 1-(cyclopropylsulfonyl)piperidin-4-amine (8 g).

LCMS (ESI-MS) m/z=205.1 [M+H]⁺.

### Intermediate 13: 8-chloro-N-(1-(cyclopropylsulfonyl)piperidin-4-yl)-6-methylpyrido[3,4-d]pyrimidin-2-amine

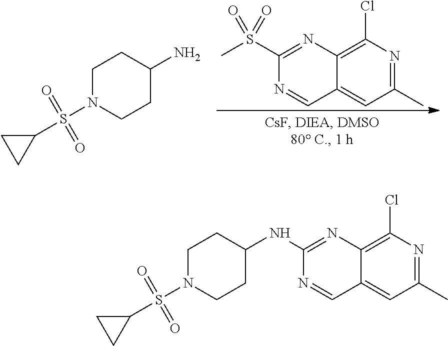

A solution of 8-chloro-6-methyl-2-(methylsulfonyl)pyrido[3,4-d]pyrimidine (1 g, 3.88 mmol), 1-(cyclopropanesulfonyl)piperidin-4-amine (1.59 g, 7.76 mmol), DIEA (1.50 g, 11.64 mmol) and CsF (1.77 g, 11.64 mmol) in DMSO (10 mL) was stirred for 1 h at 80° C. The reaction mixture was diluted with water (200 mL) and extracted with EA (3×200 mL). The combined organic layers were dried over anhydrous Na₂SO₄. After filtration, the filtrate was concentrated under reduced pressure. The residue was purified by silica gel column chromatography (PE/EA, 1:1) to afford 8-chloro-N-(1-(cyclopropylsulfonyl)piperidin-4-yl)-6-methylpyrido[3,4-d]pyrimidin-2-amine (505.8 mg, 33.3% yield).

¹H NMR (400 MHz, DMSO-d₆) δ 9.22 (s, 1H), 8.08 (d, J=7.4 Hz, 1H), 7.56 (s, 1H), 4.03 (s, 1H), 3.69-3.57 (m, 2H), 3.09-2.92 (m, 2H), 2.69-2.55 (m, 1H), 2.17-1.89 (m, 2H), 1.71-1.55 (m, 2H), 1.42-1.30 (m, 1H), 1.29-1.13 (m, 2H), 1.05-0.97 (m, 2H), 0.97-0.91 (m, 2H).

LCMS (ESI-MS) m/z=382.2 [M+H]⁺.

### Intermediate 14: 1-((1-methyl-1H-pyrazol-4-yl)sulfonyl)piperidin-4-amine

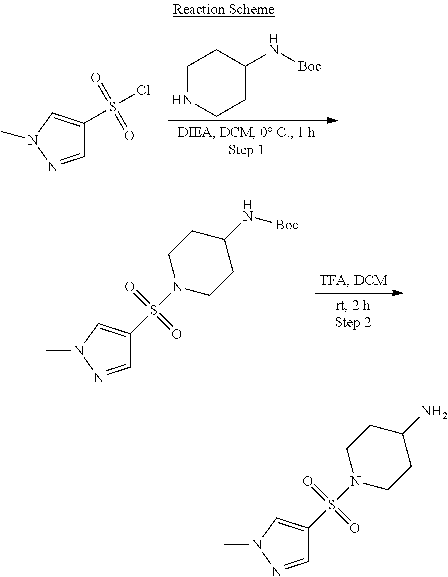

### Step 1: Tert-butyl (1-((1-methyl-1H-pyrazol-4-yl)sulfonyl)piperidin-4-yl)carbamate

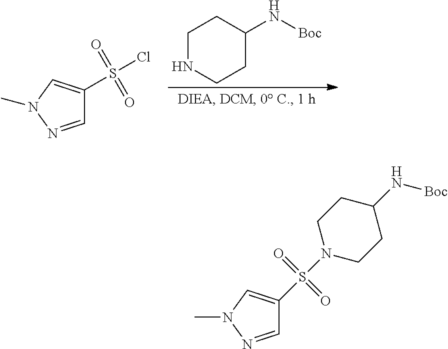

To a stirred mixture of 1-methyl-1H-pyrazole-4-sulfonyl chloride (7 g, 38.75 mmol) and DIEA (15.03 g, 116.27 mmol) in DCM (70 mL) was added tert-butyl piperidin-4-ylcarbamate (7.76 g, 38.75 mmol) dropwise at 0° C. The resulting mixture was stirred for 1 h at 0° C. The resulting mixture was diluted with water (500 mL). The aqueous solution was extracted with CH₂Cl₂ (3×500 mL). The combined organic layers were dried over anhydrous Na₂SO₄. After filtration, the filtrate was concentrated under reduced pressure. The residue was purified by silica gel column chromatography (CH₂Cl₂/MeOH, 99:1) to afford tert-butyl (1-((1-methyl-1H-pyrazol-4-yl)sulfonyl)piperidin-4-yl)carbamate (9 g, 64.1% yield).

LCMS (ESI-MS) m/z=289.2 [M+H-56]⁺.

### Step 2: 1-((1-methyl-1H-pyrazol-4-yl)sulfonyl)piperidin-4-amine

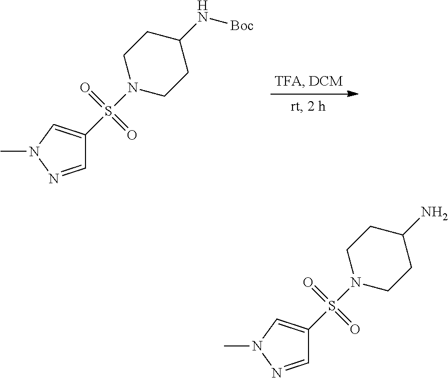 [SMILES: Cn1cc(S(=O)(=O)N2CCC(NC(=O)OC(C)(C)C)CC2)cn1 ; Cn1cc(S(=O)(=O)N2CCC(N)CC2)cn1]

A solution of tert-butyl (1-((1-methyl-1H-pyrazol-4-yl)sulfonyl)piperidin-4-yl)carbamate (6 g, 17.42 mmol) in TFA (12 mL) and DCM (36 mL) was stirred for 2 h at room temperature. The resulting mixture was concentrated under reduced pressure to afford 1-(1-methylpyrazol-4-ylsulfonyl)piperidin-4-amine (6.5 g crude).

LCMS (ESI-MS) m/z=245.2 [M+H]⁺.

### Intermediate 15: 8-chloro-6-methyl-N-(1-((1-methyl-1H-pyrazol-4-yl)sulfonyl)piperidin-4-yl)pyrido[3,4-d]pyrimidin-2-amine

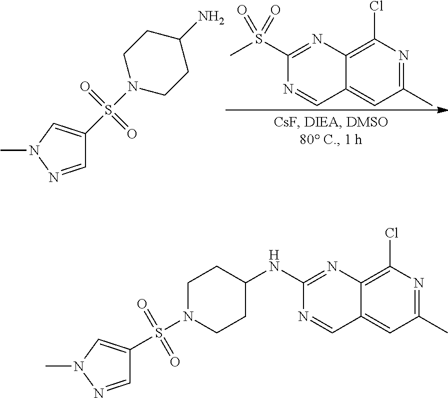

A solution of 1-(1-methylpyrazol-4-ylsulfonyl)piperidin-4-amine (6.07 g, 24.83 mmol), 8-chloro-6-methyl-2-(methylsulfonyl)pyrido[3,4-d]pyrimidine (4 g, 15.52 mmol), DIEA (6.02 g, 46.56 mmol) and CsF (7.07 g, 46.56 mmol) in DMSO (20 mL) was stirred for 1 h at 80° C. The reaction mixture was diluted with water (500 mL) and then extracted with EA (3×500 mL). The combined organic layers were dried over anhydrous Na₂SO₄. After filtration, the filtrate was concentrated under reduced pressure. The crude product was purified by silica gel column chromatography (PE/EA, 1:4) to afford 8-chloro-6-methyl-N-(1-((1-methyl-1H-pyrazol-4-yl)sulfonyl)piperidin-4-yl)pyrido[3,4-d]pyrimidin-2-amine (2.5 g, 37.4% yield).

LCMS (ESI-MS) m/z=422.0 [M+H]⁺.

### Intermediate 16: 8-chloro-N-(6-fluoro-2-(methylsulfonyl)isoindolin-5-yl)-6-methylpyrido[3,4-d]pyrimidin-2-amine

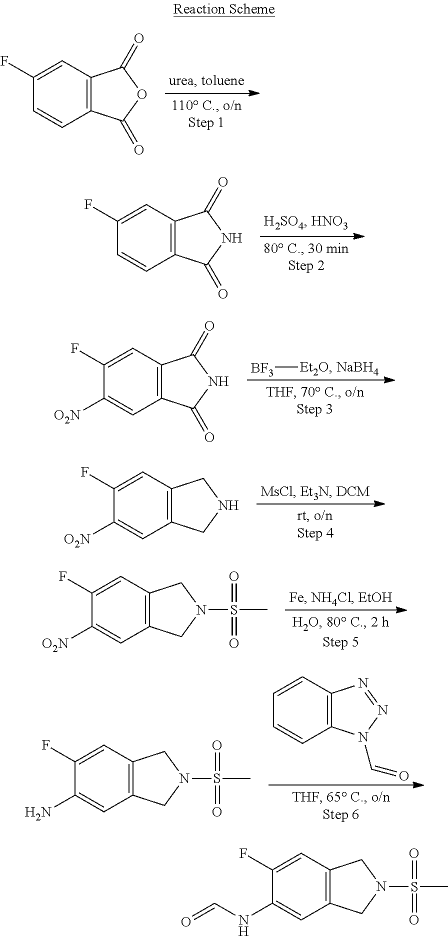

### Step 1; 5-fluoroisnindoline-1,3-dione

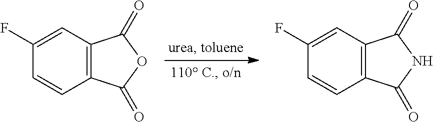 [SMILES: O=C1OC(=O)c2cc(F)ccc21 ; O=C1NC(=O)c2cc(F)ccc21]

To a solution of 5-fluoro-2-benzofuran-1,3-dione (10 g, 60.20 mmol) in toluene (30 mL) was added urea (4.34 g, 72.24 mmol) in portions at 110° C. The resulting mixture was stirred for 18 h at 110° C. The mixture was allowed to cool down to room temperature. The residue was purified by trituration with H₂O (100 mL). This resulted in 5-fluoroisoindoline-1,3-dione (9 g, 38.9% yield).

LCMS (ESI-MS) m/z=166.0 [M+H]⁺.

### Step 2: 5-fluoro-6-nitroisoindoline-1,3-dione

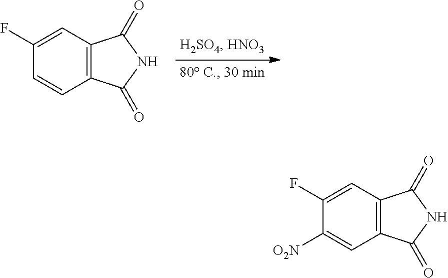 [SMILES: O=C1NC(=O)c2cc(F)ccc21 ; O=C1NC(=O)c2cc([N+](=O)[O-])c(F)cc21]

To a solution of 5-fluoroisoindoline-1,3-dione (9 g, 54.50 mmol) in H₂SO₄ (90 mL) were added HNO₃ (9 mL) in portions at 0° C. The resulting mixture was stirred for additional 30 min at 80° C. The mixture was allowed to cool down to 0° C. The reaction was quenched with cold water at 0° C. The resulting mixture was stirred for an additional 30 min at 0° C. The precipitated solids were collected by filtration and washed with H₂O (3×500 mL) to afford the desired product 5-fluoro-6-nitroisoindoline-1,3-dione (4 g, 34.9% yield).

LCMS (ESI-MS) m/z=211.0 [M+H]⁺.

### Step 3: 5-fluoro-6-nitroisoindoline

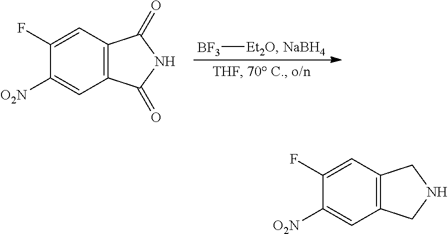

To a solution of 5-fluoro-6-nitroisoindoline-1,3-dione (5 g, 23.79 mmol) in THF (217.5 mL) were added NaBH₄ (0.87 g, 22.99 mmol) and BF3-Et₂O (35.56 g, 250.57 mmol) in portions at −10° C. The resulting mixture was stirred for 18 h at 70° C. under a nitrogen atmosphere and concentrated under vacuum to afford crude5-fluoro-6-nitroisoindoline (1.5 g crude).

LCMS (ESI-MS) m/z=183.0 [M+H]⁺.

### Step 4: 5-fluoro-2-(methylsulfonyl)-6-nitroisoindoline

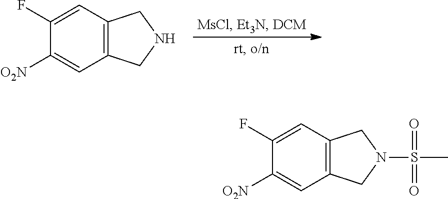

To a solution of 5-fluoro-6-nitroisoindoline (1.5 g, 8.23 mmol) in DCM (10 mL) was added Et₃N (4.50 mL, 32.36 mmol) over 3 min at 0° C. under a nitrogen atmosphere. MsCl (1.71 g, 14.90 mmol) was then added in portions at 0° C. The resulting mixture was stirred overnight at room temperature. The aqueous layer was extracted with EtOAc (2×200 mL). The resulting mixture was washed with saturated aq. sodium chloride solution (2×200 mL). The combined organic layers were dried over anhydrous Na₂SO₄. After filtration, the filtrate was concentrated under vacuum. The residue was purified by silica gel column chromatography, eluting with PE/EA and CH₂Cl₂/MeOH (5:1) to afford 5-fluoro-2-(methylsulfonyl)-6-nitroisoindoline (500 mg, 23.3% yield).

LCMS (ESI-MS) m/z=261.0 [M+H]⁺.

### Step 5: 6-fluoro-2-(methylsulfonyl)isoindolin-5-amine

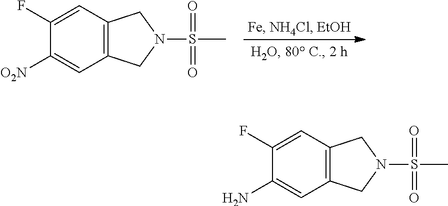

To a solution of 5-fluoro-2-(methylsulfonyl)-6-nitroisoindoline (500 mg, 1.92 mmol) in EtOH (12 mL) and H₂O (4 mL) were added Fe (1.07 g, 19.21 mmol) and NH₄Cl (411.08 mg, 7.68 mmol) in portions at 80° C. The resulting mixture was stirred for 2 h at 80° C. under a nitrogen atmosphere and concentrated under vacuum. The residue was purified by silica gel column chromatography, eluting with PE/EA (1:1) to afford 6-fluoro-2-(methylsulfonyl)isoindolin-5-amine (280 mg, 63.3% yield).

LCMS (ESI-MS) m/z=231.0 [M+H]⁺.

### Step 6: N-(6-fluoro-2-(methylsulfonyl)isoindolin-5-yl)formamide

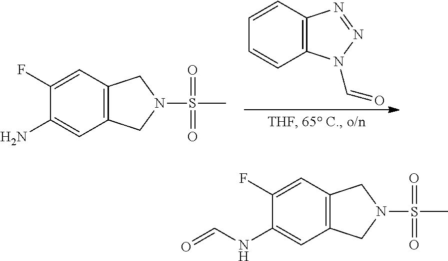 [SMILES: CS(=O)(=O)N1Cc2cc(N)c(F)cc2C1 ; O=Cn1nnc2ccccc21 ; CS(=O)(=O)N1Cc2cc(F)c(NC=O)cc2C1]

To a solution of 6-fluoro-2-(methylsulfonyl)isoindolin-5-amine (110 mg, 0.47 mmol) in THF (2 mL) was added 1,2,3-benzotriazole-1-carbaldehyde (70.29 mg, 0.47 mmol). The resulting mixture was stirred overnight at 65° C. under a nitrogen atmosphere and allowed to cool down to room temperature. The mixture was concentrated under reduced pressure and purified by silica gel column chromatography, eluting with PE/EA (1:1) to afford N-(6-fluoro-2-(methylsulfonyl)isoindolin-5-yl)formamide (60 mg, 48.6% yield).

LCMS (ESI-MS) m/z=259.0 [M+H]⁺.

### Step 7: 8-chloro-N-(6-fluoro-2-(methylsulfonyl)isoindolin-5-yl)-6-methylpyrido[3,4-d]pyrimidin-2-amine

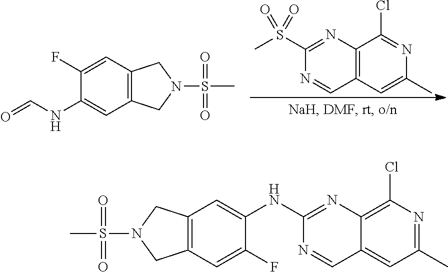

To a solution of N-(6-fluoro-2-(methylsulfonyl)isoindolin-5-yl)formamide (55 mg, 0.21 mmol) in dimethylformamide (2 mL) was added sodium hydride (25.55 mg, 1.06 mmol, 60% in mineral oil). The resulting mixture was stirred for 1 h at 0° C. under a nitrogen atmosphere. 8-chloro-6-methyl-2-(methylsulfonyl)pyrido[3,4-d]pyrimidine (54.88 mg, 0.21 mmol) was added and the resulting mixture was stirred overnight at room temperature. The reaction was quenched by the addition of a saturated aq. NH₄Cl solution (60 mL) at room temperature, diluted with EtOAc (500 mL) and washed with a saturated aq. sodium chloride solution (3×500 mL). The combined organic layers were dried over anhydrous Na₂SO₄. After filtration, the filtrate was concentrated under reduced pressure to give 8-chloro-N-(6-fluoro-2-(methylsulfonyl)isoindolin-5-yl)-6-methylpyrido[3,4-d]pyrimidin-2-amine (60 mg, 69.1% yield).

LCMS (ESI-MS) m/z=408.1 [M+H]⁺.

### Intermediate 17: 1-(2-methyl-2-azaspiro[3.3]heptan-6-yl)-1H-pyrazol-4-amine

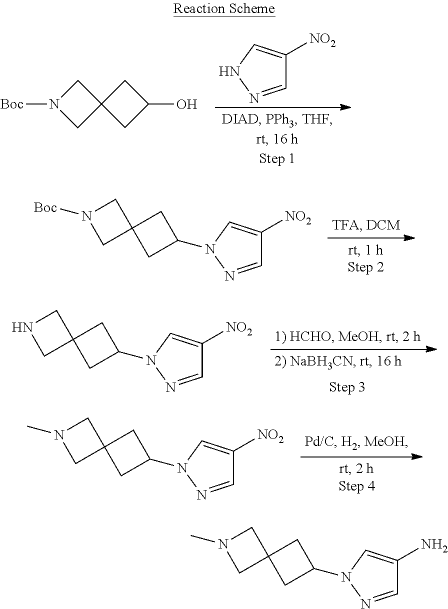

### Detailed Procedure

### Step 1: Tert-butyl 6-(4-nitro-1H-pyrazol-1-yl)-2-azaspiro[3.3]heptane-2-carboxylate

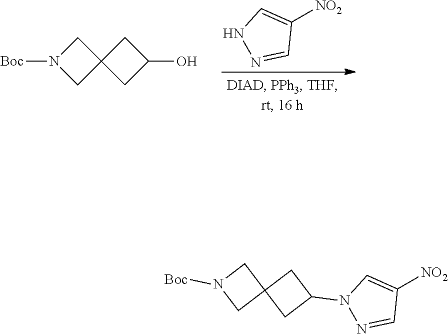 [SMILES: CC(C)(C)OC(=O)N1CC2(CC(O)C2)C1 ; O=[N+]([O-])c1cn[nH]c1 ; CC(C)(C)OC(=O)N1CC2(CC(n3cc([N+](=O)[O-])cn3)C2)C1]

To a solution of tert-butyl 6-hydroxy-2-azaspiro[3.3]heptane-2-carboxylate (10 g, 46.88 mmol) in THF (100 mL) was added 4-nitropyrazole (5.30 g, 46.88 mmol), DIAD (9.48 g, 46.88 mmol) and PPh₃ (12.30 g, 46.88 mmol). The resulting mixture was stirred at room temperature for 16 h. The reaction mixture was concentrated under vacuum and purified by silica gel column eluting with DCM/MeOH=10:1 to afford the title product (8 g, 49.8% yield).

LCMS (ESI-MS) m/z=309.1 [M+H]⁺.

### Step 2: 6-(4-nitro-1H-pyrazol-1-yl)-2-azaspiro[3.3]heptane

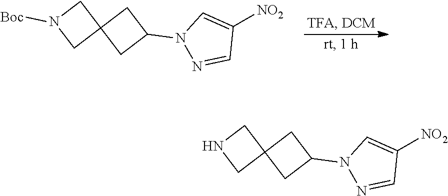 [SMILES: CC(C)(C)OC(=O)N1CC2(CC(n3cc([N+](=O)[O-])cn3)C2)C1 ; O=[N+]([O-])c1cnn(C2CC3(CNC3)C2)c1]

A mixture of tert-butyl 6-(4-nitropyrazol-1-yl)-2-azaspiro[3.3]heptane-2-carboxylate (8 g, 25.94 mmo), TFA (3 mL) and DCM (6 mL) was stirred at room temperature for 1 h. The reaction mixture was concentrated under vacuum to afford 6-(4-nitro-1H-pyrazol-1-yl)-2-azaspiro[3.3]heptane (5 g, 83.3% yield).

LCMS (ESI-MS) m/z=209.1 [M+H]⁺.

### Step 3: 2-methyl-6-(4-nitro-1H-pyrazol-1-yl)-2-azaspiro[3.3]heptane

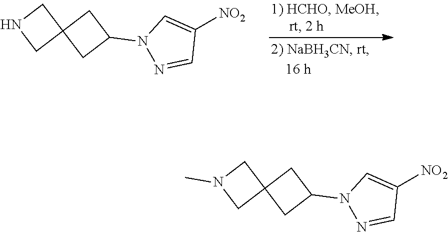 [SMILES: O=[N+]([O-])c1cnn(C2CC3(CNC3)C2)c1 ; CN1CC2(CC(n3cc([N+](=O)[O-])cn3)C2)C1]

A mixture of 6-(4-nitropyrazol-1-yl)-2-azaspiro[3.3]heptane (4.5 g, 21.61 mmol) and HCHO (1.95 g, 64.83 mmol) in MeOH (50 mL) was stirred at room temperature for 2 h. NaBH₃CN (2.72 g, 43.22 mmol) was added and the resulting mixture was stirred for 16 h at room temperature. The mixture was purified by silica gel column chromatography, eluting with DCM/MeOH=10:1 to afford 2-methyl-6-(4-nitro-1H-pyrazol-1-yl)-2-azaspiro[3.3]heptane (3.5 g, 58.3% yield).

LCMS (ESI-MS) m/z=223.1 [M+H]⁺.

### Step 4: 1-(2-methyl-2-azaspiro[3.3]heptan-6-yl)-1H-pyrazol-4-amine

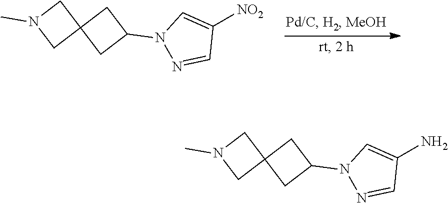 [SMILES: CN1CC2(CC(n3cc([N+](=O)[O-])cn3)C2)C1 ; CN1CC2(CC(n3cc(N)cn3)C2)C1]

Pd/C (350 mg, 10% on carbon) was added to a solution of 2-methyl-6-(4-nitropyrazol-1-yl)-2-azaspiro [3.3]heptane (3.5 g, 15.74 mmol) in MeOH (50 mL) under a nitrogen atmosphere. The resulting mixture was stirred at room temperature for 2 h under hydrogen atmosphere. The reaction mixture was filtered and the filtrate was concentrated under reduced pressure to afford crude 1-(2-methyl-2-azaspiro[3.3]heptan-6-yl)-1H-pyrazol-4-amine (3 g, 99.1% yield).

LCMS (ESI-MS) m/z=193.1 [M+H]⁺.

### Intermediate 18: 2-(methylsulfonyl) isoindolin-5-amine

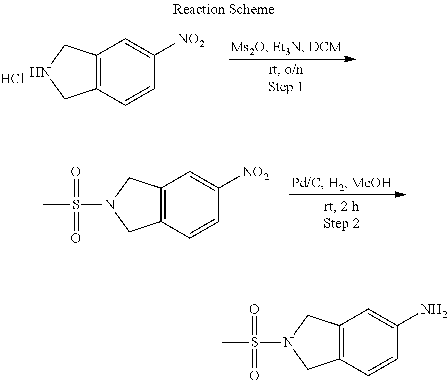 [SMILES: Cl ; O=[N+]([O-])c1ccc2c(c1)CNC2 ; CS(=O)(=O)N1Cc2ccc([N+](=O)[O-])cc2C1 ; CS(=O)(=O)N1Cc2ccc(N)cc2C1]

### Detailed Procedure

### Step 1: 2-(methylsulfonyl)-5-nitroisoindoline

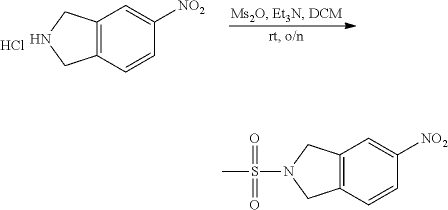 [SMILES: Cl ; O=[N+]([O-])c1ccc2c(c1)CNC2 ; CS(=O)(=O)N1Cc2ccc([N+](=O)[O-])cc2C1]

Methanesulfonyl methanesulfonate (1326.39 mg, 7.61 mmol) was added to a stirred solution of 5-nitro-2,3-dihydro-1H-isoindole (500 mg, 3.04 mmol) and Et₃N (462.31 mg, 4.56 mmol) in DCM (10 mL) at 0° C. The resulting mixture was stirred overnight at room temperature. The reaction was quenched by the addition of water (100 mL) at 0° C. The mixture was extracted with CH₂Cl₂ (3×150 mL). The combined organic layers were dried over anhydrous Na₂SO₄. After filtration, the filtrate was concentrated under reduced pressure to afford crude2-(methylsulfonyl)-5-nitroisoindoline (500 mg, 67.7% yield).

No mass signal.

### Step 2: 2-(methylsulfonyl)isoindolin-5-amine

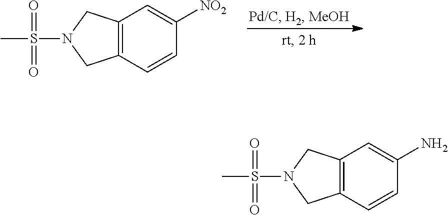 [SMILES: CS(=O)(=O)N1Cc2ccc([N+](=O)[O-])cc2C1 ; CS(=O)(=O)N1Cc2ccc(N)cc2C1]

Pd/C (219.6 mg, 2.06 mmol, 10% on carbon) was added to a solution of 2-(methylsulfonyl)-5-nitroisoindoline (500 mg, 2.06 mmol) in MeOH (10 mL) under a nitrogen atmosphere. The resulting mixture was stirred for 2 h at room temperature under a hydrogen atmosphere. The reaction mixture was filtered. The filter cake was washed with MeOH (4×100 mL) and the combined filtrate was concentrated under reduced pressure to afford crude 2-(methylsulfonyl) isoindolin-5-amine (400 mg, 91.3% yield).

LCMS (ESI-MS) m/z=213.1 [M+H]⁺.

### Intermediate 19: 2-(1-(methylsulfonyl)azetidin-3-yl)-5-nitroisoindoline

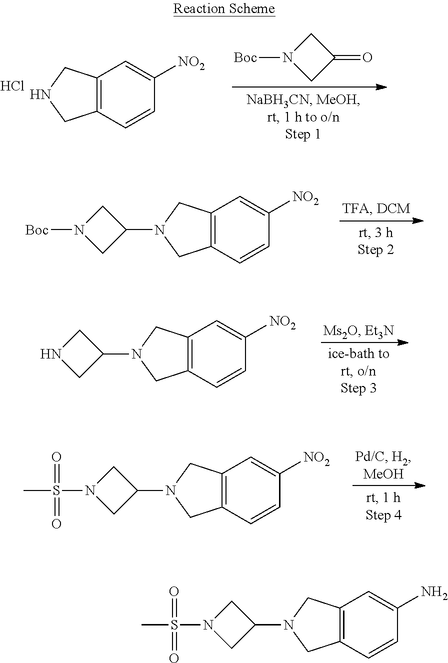

### Detailed Procedure

### Step 1: Tert-butyl 3-(5-nitroisoindolin-2-yl)azetidine-1-carboxylate

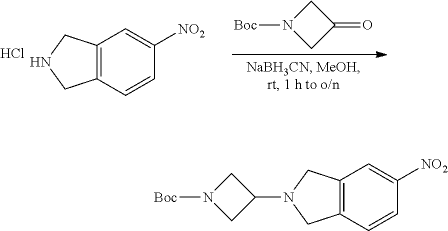 [SMILES: Cl ; O=[N+]([O-])c1ccc2c(c1)CNC2 ; CC(C)(C)OC(=O)N1CC(=O)C1 ; CC(C)(C)OC(=O)N1CC(N2Cc3ccc([N+](=O)[O-])cc3C2)C1]

A mixture of 5-nitro-2,3-dihydro-1H-isoindole hydrochloride (1.8 g, 8.97 mmol) and tert-butyl 3-oxoazetidine-1-carboxylate (1.54 g, 8.97 mmol) in MeOH (40 mL) was stirred for 1 h at room temperature. NaBH₃CN (1.13 g, 17.94 mmol) was added and the resulting mixture was stirred overnight at room temperature. The reaction mixture was concentrated and quenched with water (100 mL). The mixture was extracted with EA (3×100 mL). The combined organic layers were dried over anhydrous Na₂SO₄. After filtration, the filtrate was concentrated under reduced pressure. The crude product was purified by silica gel column chromatography, eluting with EA/PE (30%) to afford tert-butyl 3-(5-nitroisoindolin-2-yl)azetidine-1-carboxylate (2.4 g, 80.8% yield).

LCMS (ESI-MS) m/z=320.2 [M+H]⁺.

### Step 2: 2-(azetidin-3-yl)-5-nitroisoindoline

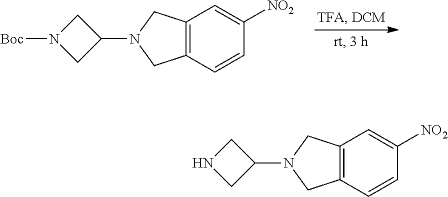 [SMILES: CC(C)(C)OC(=O)N1CC(N2Cc3ccc([N+](=O)[O-])cc3C2)C1 ; O=[N+]([O-])c1ccc2c(c1)CN(C1CNC1)C2]

A mixture of tert-butyl 3-(5-nitroisoindolin-2-yl)azetidine-1-carboxylate (2.4 g, 7.51 mmol) in TFA (4 mL) and DCM (12 mL) was stirred for 3 h at room temperature. The reaction mixture was concentrated purified by silica gel column chromatography, eluting with DCM (0.5% Et₃N)/MeOH (1:1) to afford 2-(azetidin-3-yl)-5-nitroisoindoline (850 mg, 51.6% yield).

LCMS (ESI-MS) m/z=220.1 [M+H]⁺.

### Step 3: 2-(1-(methylsulfonyl)azetidin-3-yl)-5-nitroisoindoline

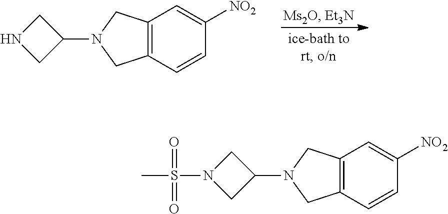 [SMILES: O=[N+]([O-])c1ccc2c(c1)CN(C1CNC1)C2 ; CS(=O)(=O)N1CC(N2Cc3ccc([N+](=O)[O-])cc3C2)C1]

Methanesulfonic anhydride (699.16 mg, 4.01 mmol) was added to a cooled to 0° C. mixture of 2-(azetidin-3-yl)-5-nitroisoindoline (800 mg, 3.64 mmol) and Et₃N (1107 mg, 10.94 mmol) in DCM (12 mL). The reaction mixture was stirred at room temperature overnight and quenched with water (60 mL). The resulting mixture was extracted with DCM (3×60 mL). The combined organic layers were dried over anhydrous Na₂SO₄. After filtration, the filtrate was concentrated under reduced pressure and purified by silica gel column chromatography, eluting with PE/EA (61%) to afford 2-(1-(methylsulfonyl)azetidin-3-yl)-5-nitroisoindoline (850 mg, 66.6% yield).

LCMS (ESI-MS) m/z=298.1 [M+H]⁺.

### Step 4: 2-(1-(methylsulfonyl)azetidin-3-yl)isoindolin-5-amine

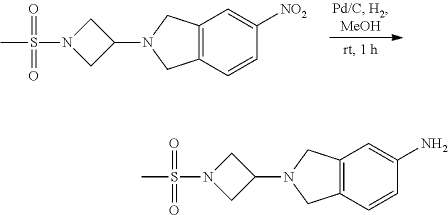 [SMILES: CS(=O)(=O)N1CC(N2Cc3ccc([N+](=O)[O-])cc3C2)C1 ; CS(=O)(=O)N1CC(N2Cc3ccc(N)cc3C2)C1]

Pd/C (143.17 mg, 1.34 mmol) was added to a mixture of 2-(1-(methylsulfonyl)azetidin-3-yl)-5-nitroisoindoline (800 mg, 2.69 mmol) in MeOH (15 mL) under a nitrogen atmosphere, the reaction mixture was stirred for 1 h at room temperature under a hydrogen atmosphere. The mixture was filtered and the filter cake was washed with MeOH (2×50 mL). The filtrate was concentrated under reduced pressure to afford crude 2-(1-(methylsulfonyl)azetidin-3-yl)isoindolin-5-amine (600 mg, 65.3% yield).

LCMS (ESI-MS) m/z=268.1 [M+H]⁺.

### Intermediate 20: 2-(3-(4-amino-1H-pyrazol-1-yl)azetidin-1-yl)acetonitrile

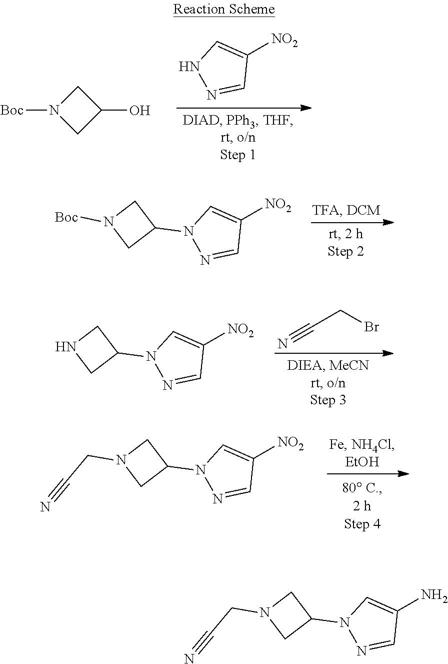

### Detailed Procedure

### Step 1: Tert-butyl 3-(4-nitro-1H-pyrazol-1-yl)azetidine-1-carboxylate

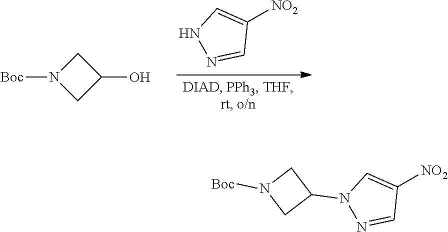 [SMILES: CC(C)(C)OC(=O)N1CC(O)C1 ; O=[N+]([O-])c1cn[nH]c1 ; CC(C)(C)OC(=O)N1CC(n2cc([N+](=O)[O-])cn2)C1]

To a solution of tert-butyl 3-hydroxyazetidine-1-carboxylate (3 g, 17.32 mmol) and 4-nitropyrazole (1.96 g, 17.32 mmol) in THF (20 mL) was added PPh₃ (6.81 g, 25.98 mmol) in portions at 0° C. and DIAD (5.25 g, 25.98 mmol) at room temperature. The resulting mixture was stirred overnight at room temperature under a nitrogen atmosphere. The resulting mixture was concentrated under vacuum and purified by silica gel column chromatography (PE/EA, 5:1) to afford tert-butyl 3-(4-nitro-1H-pyrazol-1-yl)azetidine-1-carboxylate (5 g).

LCMS (ESI-MS) m/z=269.1[M+H]⁺.

### Step 2: 1-(azetidin-3-yl)-4-nitro-1H-pyrazole

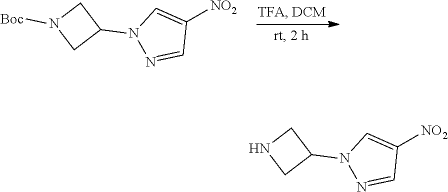 [SMILES: CC(C)(C)OC(=O)N1CC(n2cc([N+](=O)[O-])cn2)C1 ; O=[N+]([O-])c1cnn(C2CNC2)c1]

A mixture of tert-butyl 3-(4-nitro-TH-pyrazol-1-yl)azetidine-1-carboxylate (6 g, 22.36 mmol) and TFA (20 mL) in DCM (60 mL) was stirred for 2 h at room temperature. The resulting mixture was concentrated under vacuum to afford the crude 1-(azetidin-3-yl)-4-nitro-TH-pyrazole (4 g).

LCMS (ESI-MS) m/z=169.1[M+H]⁺.

### Step 3: 2-(3-(4-nitro-1H-pyrazol-1-yl)azetidin-1-yl)acetonitrile

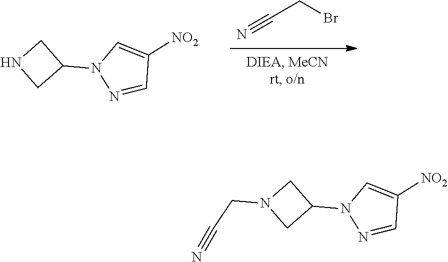

To a solution of 1-(azetidin-3-yl)-4-nitro-TH-pyrazole (1.5 g, 8.92 mmol) in MeCN (50 mL) was added 2-bromoacetonitrile (1.60 g, 13.38 mmol) and N,N-diisopropylethylamine (3.46 g, 26.76 mmol). The resulting mixture was stirred overnight at room temperature. The reaction mixture was concentrated under reduced pressure and purified by silica gel column chromatography (PE/EA, 1:1) to afford the desired product 2-(3-(4-nitro-TH-pyrazol-1-yl)azetidin-1-yl)acetonitrile (300 mg, 16.23%).

LCMS (ESI-MS) m/z=208.1[M+H]⁺.

### Step 4: 2-(3-(4-amino-1H-pyrazol-1-yl)azetidin-1-yl)acetonitrile

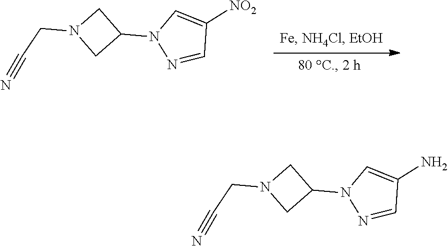 [SMILES: N#CCN1CC(n2cc([N+](=O)[O-])cn2)C1 ; N#CCN1CC(n2cc(N)cn2)C1]

To a solution of 2-(3-(4-nitro-1H-pyrazol-1-yl)azetidin-1-yl)acetonitrile (200 mg, 0.96 mmol) and NH₄Cl (206.53 mg, 3.86 mmol) in water (3 mL) and EtOH (9 mL) was added Fe (539.06 mg, 9.65 mmol) in portions at 80° C. The resulting mixture was stirred for 2 h at 80° C. under a nitrogen atmosphere. The reaction mixture was filtered and the filtrate was concentrated, diluted with water (30 mL) and extracted with EA (3×30 mL). The combined organic layers were dried over anhydrous Na₂SO₄, filtered and concentrated under vacuum to afford crude 2-(3-(4-amino-1H-pyrazol-1-yl)azetidin-1-yl)acetonitrile (120 mg, 70.2% yield).

LCMS (ESI-MS) m/z=178.1[M+H]⁺.

### Intermediate 21: 1-((1-(dimethylamino)cyclopropyl)methyl)-1H-pyrazol-4-amine

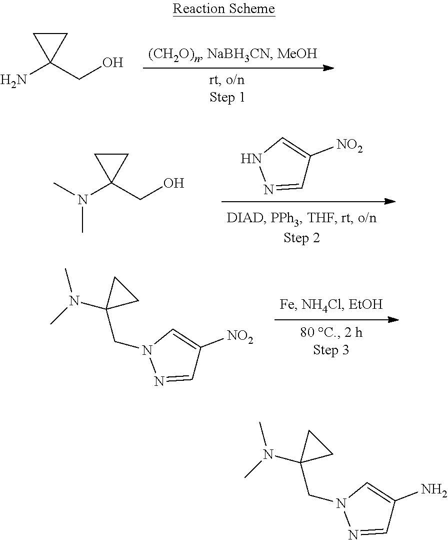

### Detailed Procedure

### Step 1: (1-(dimethylamino)cyclopropyl)methanol

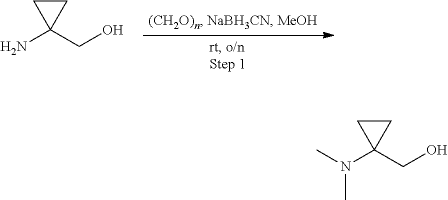

NaBH₃CN (2.89 g, 45.9 mmol) was added to a solution of (1-aminocyclopropyl)methanol (2 g, 22.9 mmol) and paraformaldehyde (6.20 g, 68.8 mmol) in MeOH (20 mL). The resulting mixture was stirred overnight at room temperature. The reaction mixture was concentrated under vacuum and purified by silica gel column chromatography, eluting with MeOH in DCM (0% to 20%). The fractions with the desired mass signal were combined and concentrated under vacuum to afford the desired product (1-(dimethylamino)cyclopropyl)methanol (600 mg, 22.7% yield).

LCMS (ESI-MS) m/z=116.1 [M+H]⁺.

### Step 2:N,N-dimethyl-1-((4-nitro-1H-pyrazol-1-yl)methyl)cyclopropan-1-amine

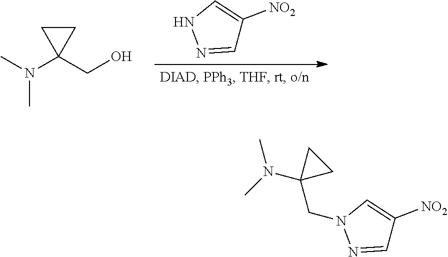

DIAD (1.4 g, 7.16 mmol) was added to a solution of (1-(dimethylamino)cyclopropyl)methanol (550 mg, 4.77 mmol), 4-nitropyrazole (648 mg, 5.73 mmol) and PPh₃ (1.8 g, 7.16 mmol) in THF (6 mL) under a nitrogen atmosphere. The resulting mixture was stirred overnight at room temperature. The reaction mixture was concentrated under vacuum and purified by silica gel column chromatography, eluting with EA in PE (0% to 30%). The fractions with desired mass signal were combined and concentrated under vacuum to afford the desired product N,N-dimethyl-1-((4-nitro-1H-pyrazol-1-yl)methyl)cyclopropan-1-amine (400 mg, 39.8% yield).

LCMS (ESI-MS) m/z=211.1 [M+H]⁺.

### Step 3:1-((1-(dimethylamino)cyclopropyl)methyl)-1H-pyrazol-4-amine

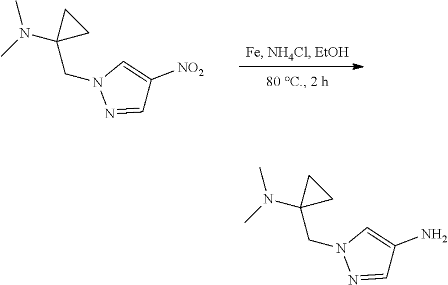

Fe (1.0 g, 19.0 mmol) was added to a solution of N,N-dimethyl-1-((4-nitro-1H-pyrazol-1-yl)methyl)cyclopropan-1-amine (400 mg, 1.90 mmol) and NH₄Cl (407 mg, 7.61 mmol) in a mixture of EtOH (3 mL) and H₂O (1 mL). The resulting mixture was heated to 80° C. and stirred for 2 h. After cooling to room temperature, the resulting mixture was filtered and the filter cake was washed with ethanol (2×10 mL). The filtrate was concentrated under reduced pressure to afford crude 1-((1-(dimethylamino)cyclopropyl)methyl)-1H-pyrazol-4-amine (300 mg, 87.5% yield).

LCMS (ESI-MS) m/z=181.1 [M+H]⁺.

### Intermediate 22: 2-cyclopropylisoindolin-5-amine

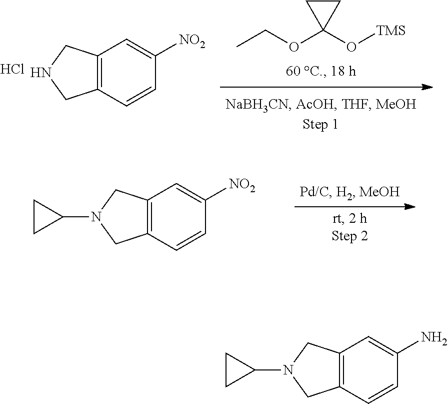

### Detailed Procedure

### Step 1: 2-cyclopropyl-5-nitroisoindoline

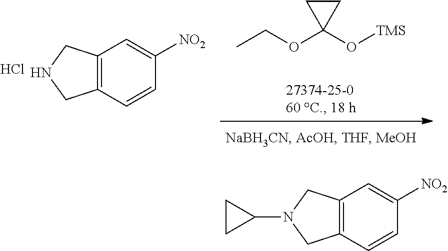

To a solution of 5-nitro-2,3-dihydro-1H-isoindole (2.6 g, 15.8 mmol), AcOH (1.90 g, 31.6 mmol) and (1-ethoxycyclopropoxy)trimethylsilane (11.04 g, 63.3 mmol) in THF (100 mL) and MeOH (10 mL) was added NaBH₃CN (1.49 g, 23.7 mmol) at 20° C. The resulting mixture was stirred for 18 h at 60° C. The reaction mixture was cooled to room temperature and quenched with 1N HCl and extracted with EtOAc. The aqueous layer was basified to PH=10 with solid K₂CO₃ and extracted with DCM. The combined organic phase was washed with water and brine, dried over Na₂SO₄ and concentrated to afford 2-cyclopropyl-5-nitroisoindoline (2 g, 49.47%).

LCMS (ESI-MS) m/z=205.1 [M+H]⁺.

### Step 2: 2-cyclopropylisoindolin-5-amine

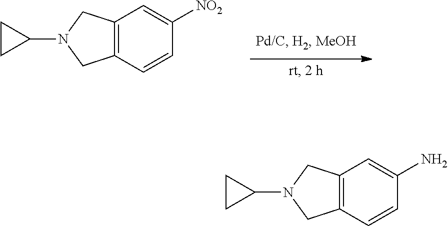 [SMILES: O=[N+]([O-])c1ccc2c(c1)CN(C1CC1)C2 ; Nc1ccc2c(c1)CN(C1CC1)C2]

Pd/C (14.59 mg, 0.14 mmol) was added to the solution of 2-cyclopropyl-5-nitroisoindoline (350 mg, 1.7 mmol) in MeOH (5 mL) under a nitrogen atmosphere. The resulting mixture was stirred for 2 h at room temperature under a hydrogen atmosphere. The reaction mixture was filtered and the filter cake was washed with MeOH (4×10 mL). The combined filtrate was concentrated under reduced pressure to afford crude product 2-cyclopropylisoindolin-5-amine (270 mg, 88.6% yield).

LCMS (ESI-MS) m/z=175.1 [M+H]⁺.

### Intermediate 23: 1-((cycloheptylmethyl)sulfonyl)piperidin-4-amine

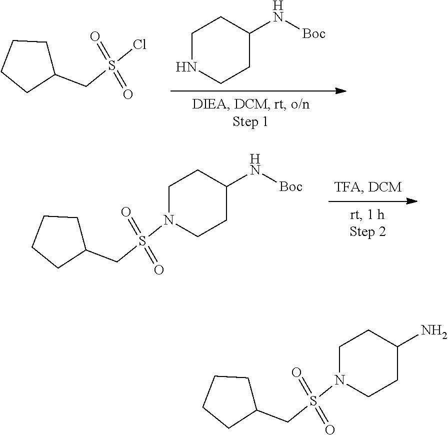 [SMILES: O=S(=O)(Cl)CC1CCCC1 ; CC(C)(C)OC(=O)NC1CCNCC1 ; CC(C)(C)OC(=O)NC1CCN(S(=O)(=O)CC2CCCC2)CC1 ; NC1CCN(S(=O)(=O)CC2CCCC2)CC1]

### Step 1: Tert-butyl (1-((cyclopentylmethyl)sulfonyl)piperidin-4-yl)carbamate

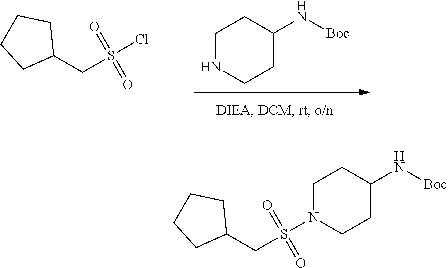 [SMILES: O=S(=O)(Cl)CC1CCCC1 ; CC(C)(C)OC(=O)NC1CCNCC1 ; CC(C)(C)OC(=O)NC1CCN(S(=O)(=O)CC2CCCC2)CC1]

To a solution of cyclopentylmethanesulfonyl chloride (470 mg, 2.57 mmol) and DIEA (831.41 mg, 6.43 mmol) in DCM (10 mL) was added tert-butyl N-(piperidin-4-yl)carbamate (429.45 mg, 2.14 mmol) dropwise at 0° C. The resulting mixture was stirred overnight at room temperature. The resulting mixture was diluted with water (200 mL) and extracted with CH₂Cl₂ (3×250 mL). The combined organic layers were dried over anhydrous Na₂SO₄. After filtration, the filtrate was concentrated under reduced pressure to afford tert-butyl (1-((cyclopentylmethyl)sulfonyl)piperidin-4-yl)carbamate (800 mg, 96.9% yield).

LCMS (ESI-MS) m/z=347.2 [M+H]⁺.

### Step 2: 1-((cyclopentylmethyl)sulfonyl)piperidin-4-amine

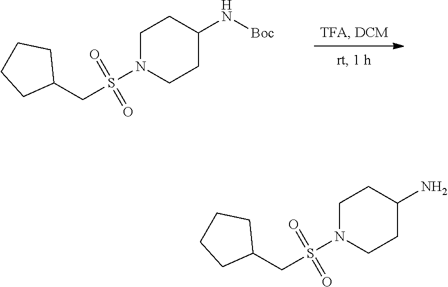 [SMILES: CC(C)(C)OC(=O)NC1CCN(S(=O)(=O)CC2CCCC2)CC1 ; NC1CCN(S(=O)(=O)CC2CCCC2)CC1]

A solution of tert-butyl (1-((cyclopentylmethyl)sulfonyl)piperidin-4-yl)carbamate (400 mg, 1.15 mmol) and TFA (3 mL) in DCM (10 mL) was stirred for 1 h at room temperature. The resulting mixture was concentrated under reduced pressure to afford 1-((cyclopentylmethyl)sulfonyl)piperidin-4-amine (350 mg crude).

LCMS (ESI-MS) m/z=247.1 [M+H]⁺. 8-chloro-N-(1-((cyclopentylmethyl)sulfonyl)piperidin-4-yl)-6-methylpyrido[3,4-d]pyrimidin-2-amine

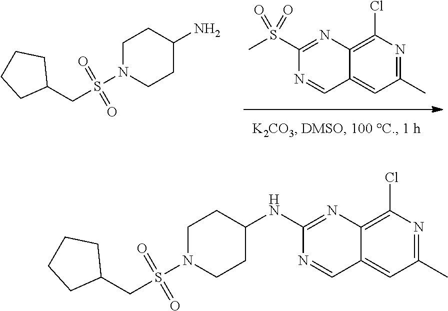

A mixture of 1-cyclopentylmethanesulfonylpiperidin-4-amine (350 mg, 1.42 mmol), 8-chloro-2-methanesulfonyl-6-methylpyrido[3,4-d]pyrimidine (366.1 mg, 1.42 mmol) and K₂CO₃ (588.98 mg, 4.26 mmol) in DMSO (3 mL) was stirred for 1 h at 100° C. The reaction mixture was diluted with water (150 mL) and extracted with DCM (3×150 mL). The combined organic layers were dried over anhydrous Na₂SO₄, filtered and concentrated under vacuum to afford crude 8-chloro-N-(1-((cyclopentylmethyl)sulfonyl) piperidin-4-yl)-6-methylpyrido[3,4-d]pyrimidin-2-amine (300 mg).

LCMS (ESI-MS) m/z=424.1 [M+H]⁺.

### Intermediate 24: 1-cyclopropylcyclobutane-1,3-diol

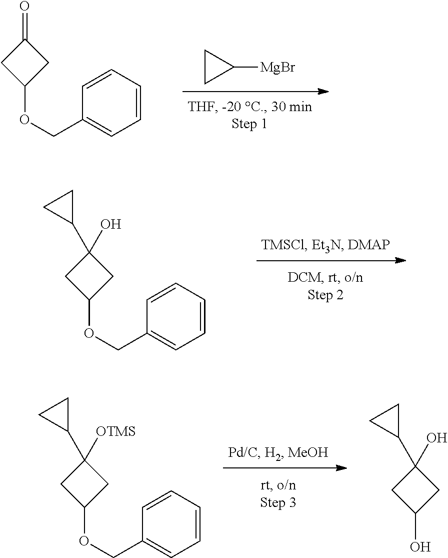

### Step 1: 3-(benzyloxy)-1-cyclopropylcyclobutan-1-ol

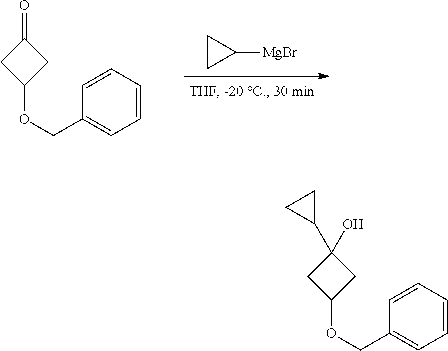

To a stirred solution of 3-(benzyloxy)-1-cyclopropylcyclobutan-1-ol (3 g, 17.0 mmol) in THF (30 mL) was added 1M cyclopropylmagnesium bromide in THF (20.43 mL, 20.4 mmol) dropwise at −20° C. under a nitrogen atmosphere. The mixture was stirred at −20° C. for 30 minutes. The reaction was quenched by the addition of sat. aq. NH₄Cl (30 mL) at 0° C. The resulting mixture was extracted with EA (3×30 mL), dried over anhydrous Na₂SO₄. After filtration, the filtrate was concentrated under reduced pressure. The residue was purified by silica gel column chromatography, eluting with PE/EA (3:1) to afford 3-(benzyloxy)-1-cyclopropylcyclobutan-1-ol (1.6 g, 43.0% yield).

LCMS (ESI-MS) m/z=219.1 [M+H]⁺.

### Step 2: (3-(benzyloxy)-1-cyclopropylcyclobutoxy)trimethylsilane

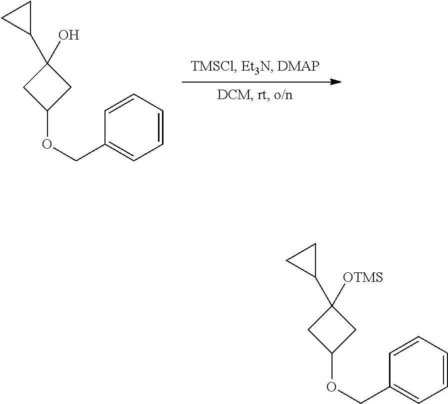

Et₃N (1.95 g, 19.2 mmol) was added to a mixture of 3-(benzyloxy)-1-cyclopropylcyclobutan-1-ol (1.4 g, 6.4 mmol), TMSCl (1.05 g, 9.6 mmol) and DMAP (0.08 g, 0.64 mmol) in DCM (20 mL). The mixture was stirred at room temperature overnight. The reaction was quenched by the addition of sat. aq. NH₄Cl (30 mL) at room temperature. The resulting mixture was extracted with DCM (3×30 mL) and dried over anhydrous Na₂SO₄. After filtration, the filtrate was concentrated under reduced pressure. The residue was purified by silica gel column chromatography, eluting with PE/EA (8:1) to afford (3-(benzyloxy)-1-cyclopropylcy-clobutoxy)trimethylsilane (700 mg, 37.5% yield).

LCMS (ESI-MS) m/z=291.2 [M+H]⁺.

### Step 3: 1-cyclopropylcyclobutane-1,3-diol

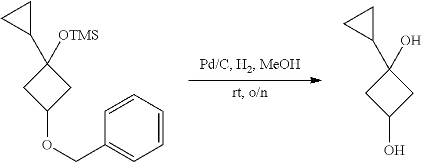

Pd/C (50 mg, 0.47 mmol) was added to (3-(benzyloxy)-1-cyclopropylcyclobutoxy)trimethylsilane (200 mg, 0.69 mmol) in MeOH (5 mL) under a nitrogen atmosphere. The mixture was stirred at room temperature overnight under a hydrogen atmosphere. The resulting mixture was filtered and the filter cake was washed with MeOH (3×30 mL). The filtrate was concentrated under reduced pressure. The residue was purified by Prep-TLC (PE/EA 1:2) to afford 1-cyclopropylcyclobutane-1,3-diol (50 mg, 56.6% yield).

LCMS (ESI-MS) m/z=129.1 [M+H]⁺.

### Intermediate 25: 1-(difluoromethyl)-3-hydroxycyclobutyl benzoate

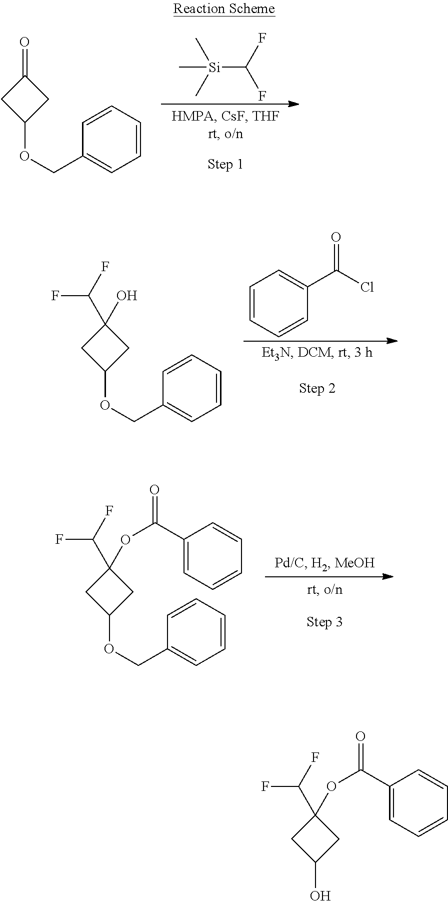

### Detailed Procedure

### Step 1: 3-(Benzyloxy)-1-(difluoromethyl)cyclobutan-1-ol

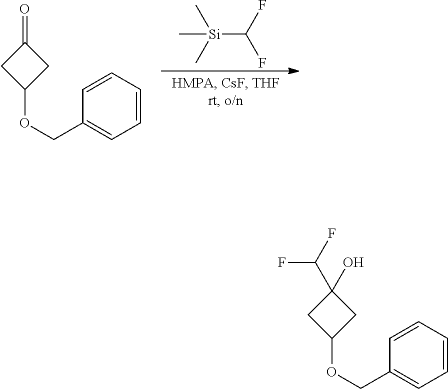

To a solution of 3-(benzyloxy)cyclobutan-1-one (10 g, 56.74 mmol) in THF (100 mL) was added (difluoromethyl)trimethylsilane (8.46 g, 68.09 mmol), HMPA (101.70 g, 567.49 mmol) and CsF (8.62 g, 56.74 mmol) at room temperature under a nitrogen atmosphere. The resulting mixture was stirred overnight at room temperature. The reaction mixture was diluted with water (500 mL) and extracted with EA (2×500 mL). The combined organic layers were dried over anhydrous Na₂SO₄. After filtration, the filtrate was concentrated under reduced pressure. The residue was purified by silica gel column chromatography (PE/EA, 5:1) to afford 3-(benzyloxy)-1-(difluoromethyl)cyclobutan-1-ol (3.5 g, 27.0% yield) as colorless oil.

LCMS (ESI-MS) m/z=229.1 [M+H]⁺.

### Step 2: 3-(benzyloxy)-1-(difluoromethyl)cyclobutyl benzoate

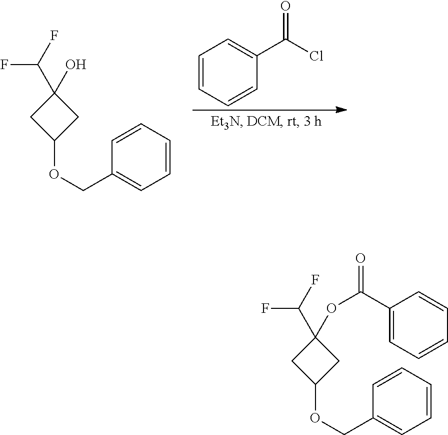 [SMILES: OC1(C(F)F)CC(OCc2ccccc2)C1 ; O=C(Cl)c1ccccc1 ; O=C(OC1(C(F)F)CC(OCc2ccccc2)C1)c1ccccc1]

A solution of 3-(benzyloxy)-1-(difluoromethyl)cyclobutan-1-ol (3.4 g, 14.89 mmol) in DCM (30 mL) was treated with Et₃N (4.52 g, 44.69 mmol) for 3 min at 0° C. Benzoyl chloride (2.30 g, 16.38 mmol) was then added in portions at 0° C. The resulting mixture was stirred 3 h at room temperature. The reaction mixture was diluted with water (200 mL) and extracted with DCM (3×200 mL). The combined organic layers were washed with saturated sodium chloride solution (2×200 mL) and dried over anhydrous Na₂SO₄. After filtration, the filtrate was concentrated under reduced pressure. The residue was purified by silica gel column chromatography (PE/EA, 1:1) to afford 3-(benzyloxy)-1-(difluoromethyl)cyclobutyl benzoate (1 g, 20.2% yield) as colorless oil.

LCMS (ESI-MS) m/z=333.1 [M+H]⁺.

### Step 3: 1-(difluoromethyl)-3-hydroxycyclobutyl benzoate

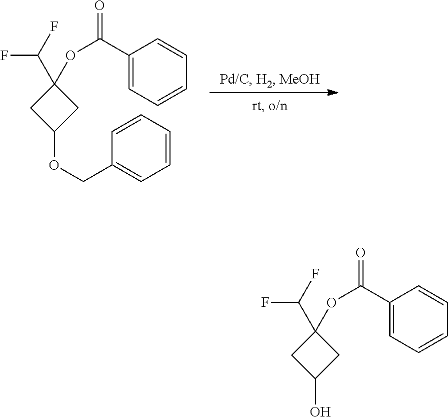 [SMILES: O=C(OC1(C(F)F)CC(OCc2ccccc2)C1)c1ccccc1 ; O=C(OC1(C(F)F)CC(O)C1)c1ccccc1]

Pd/C (200 mg, 10% on carbon) was added to a solution of 3-(benzyloxy)-1-(difluoromethyl)cyclobutyl benzoate (800 mg, 2.40 mmol) in MeOH (10 mL) under a nitrogen atmosphere, the resulting mixture was stirred overnight at room temperature under a hydrogen atmosphere. The reaction mixture was filtered and the filtrate was concentrated under reduced pressure to afford crude product 1-(difluoromethyl)-3-hydroxycyclobutyl benzoate (583.1 mg, 90.0% yield) as a colorless oil.

¹H NMR (400 MHz, Chloroform-d) δ 8.12-8.03 (m, 2H), 7.63-7.59 (m, 1H), 7.49-7.45 (m, 2H), 6.46-6.17 (m, 1H), 4.28-4.21 (m, 1H), 3.26-3.14 (m, 3H), 2.48-2.43 (m, 2H).

LCMS (ESI-MS) m/z=243.0 [M+H]⁺.

### Intermediate 26: Tert-butyl 2-hydroxy-5-oxa-8-azaspiro[3.5]nonane-8-carboxylate

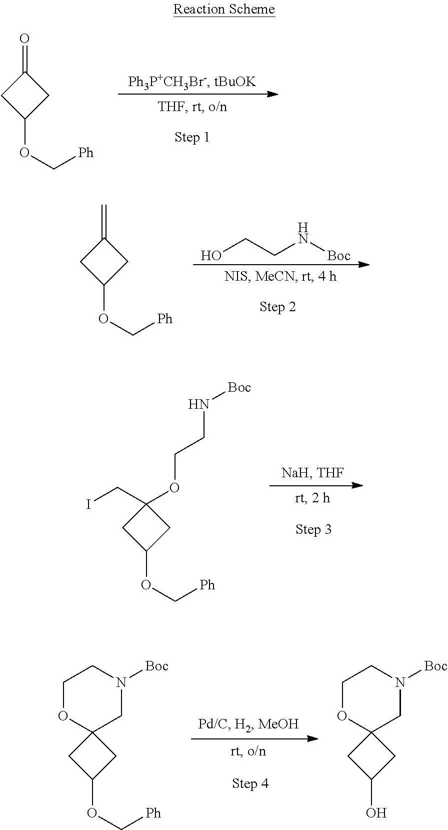 [SMILES: O=C1CC(OCc2ccccc2)C1 ; C=C1CC(OCc2ccccc2)C1 ; CC(C)(C)OC(=O)NCCO ; CC(C)(C)OC(=O)NCCOC1(CI)CC(OCc2ccccc2)C1 ; CC(C)(C)OC(=O)N1CCOC2(CC(OCc3ccccc3)C2)C1 ; CC(C)(C)OC(=O)N1CCOC2(CC(O)C2)C1]

Detailed Procedure

### Step 1: ((3-methylenecyclobutoxy)methyl)benzene

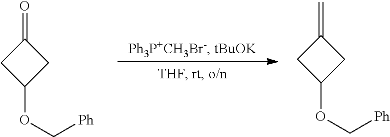

To a stirred mixture of methyltriphenylphosphanium bromide (3.24 g, 90.79 mmol) in THF (100 mL) was added t-BuOK (1.14 g, 102.14 mmol) at room temperature. The reaction mixture was stirred for 3 h at room temperature. 3-(Benzyloxy)cyclobutan-1-one (10 g, 56.74 mmol) was added and the reaction was stirred overnight at room temperature. The reaction mixture was diluted with water (300 mL) and extracted with ether (3×300 mL). The combined organic layers were dried and concentrated. The residue was purified by silica gel column chromatography (PE/EA, 96:4) to afford ((3-methylenecyclobutoxy)methyl)benzene (1.8 g, 18.2% yield) as a colorless oil.

LCMS (ESI-MS) m/z=175.1 [M+H]⁺.

### Step 2: Tert-butyl (2-(3-(benzyloxy)-1-(iodomethyl)cyclobutoxy)ethyl)carbamate

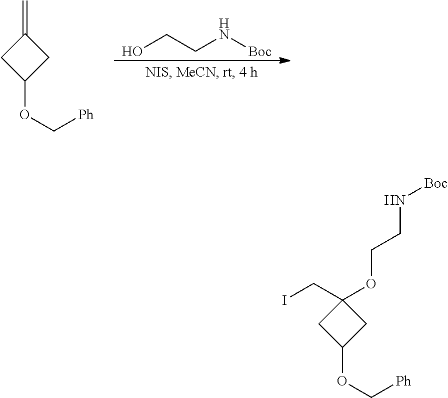 [SMILES: C=C1CC(OCc2ccccc2)C1 ; CC(C)(C)OC(=O)NCCO ; CC(C)(C)OC(=O)NCCOC1(CI)CC(OCc2ccccc2)C1]

To a solution of tert-butyl N-(2-hydroxyethyl)carbamate (7.77 g, 48.20 mmol) and ((3-methylenecyclobutoxy)methyl)benzene (7 g, 40.17 mmol) in MeCN (70 mL) was added 1-iodo-5-pyrrolidinedione (1.08 g, 48.20 mmol), the mixture was stirred at room temperature for 4 h. The reaction mixture was diluted with water (100 mL) and extracted with ether (3×100 mL). The combined organic layers were dried and concentrated. The residue was purified by silica gel column chromatography (PE/EA, 85:15) to afford tert-butyl (2-(3-(benzyloxy)-1-(iodomethyl)cyclobutoxy)ethyl)carbamate (2 g, 41.9% yield) as a colorless oil.

LCMS (ESI-MS) m/z=462.1 [M+H]⁺.

### Step 3: Tert-butyl 2-(benzyloxy)-5-oxa-8-azaspiro[3.5]nonane-8-carboxylate

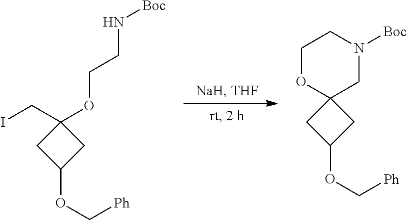 [SMILES: CC(C)(C)OC(=O)NCCOC1(CI)CC(OCc2ccccc2)C1 ; CC(C)(C)OC(=O)N1CCOC2(CC(OCc3ccccc3)C2)C1]

To a cooled to 0° C. solution of tert-butyl (2-(3-(benzyloxy)-1-(iodomethyl)cyclobutoxy)ethyl)carbamate (7.2 g, 15.60 mmol) in anhydrous THF (70 mL) was added NaH (0.75 g, 31.21 mmol, 60% in mineral oil). The mixture was stirred at room temperature for 2 h. The reaction was quenched with aq. NH₄Cl (100 mL) and extracted with EA (3×200 mL). The combined organic layers were dried and concentrated under reduced pressure. The residue was purified by silica gel chromatography (EA/PE, 20:80) to afford tert-butyl 2-(benzyloxy)-5-oxa-8-azaspiro[3.5]nonane-8-carboxylate (3 g, 57.7% yield) as a colorless oil.

LCMS (ESI-MS) m/z=334.1 [M+H]⁺.

### Step 4: Tert-butyl 2-hydroxy-5-oxa-8-azaspiro[3.5]nonane-8-carboxylate

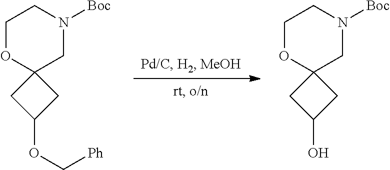 [SMILES: CC(C)(C)OC(=O)N1CCOC2(CC(OCc3ccccc3)C2)C1 ; CC(C)(C)OC(=O)N1CCOC2(CC(O)C2)C1]

A solution of Pd/C (2.87 g, 26.99 mmol, 10% on carbon) and tert-butyl 2-(benzyloxy)-5-oxa-8-azaspiro[3.5]nonane-8-carboxylate (3 g, 8.99 mmol) in MeOH (30 mL) was stirred at room temperature overnight under a hydrogen atmosphere. The reaction was filtered, and the filtrate was concentrated under reduced pressure. The crude product was purified by silica gel column chromatography (PE/EA, 70:30) to afford tert-butyl 2-hydroxy-5-oxa-8-azaspiro[3.5]nonane-8-carboxylate (2 g, 91.4% yield) as a colorless oil.

¹H NMR (400 MHz, DMSO-d₆) δ 5.08 (dd, J=41.6, 5.7 Hz, 1H), 4.26-3.70 (m, 1H), 3.45 (q, J=4.7 Hz, 2H), 3.33 (d, J=23.4 Hz, 1H), 3.28-3.19 (m, 2H), 3.19-3.11 (m, 1H), 2.33-2.26 (m, 1H), 2.25-2.17 (m, 1H), 1.80-1.66 (m, 2H), 1.40 (s, 9H).

### Intermediate 27: Tert-butyl 8,8-difluoro-2-hydroxy-6-azaspiro[3.4]octane-6-carboxylate

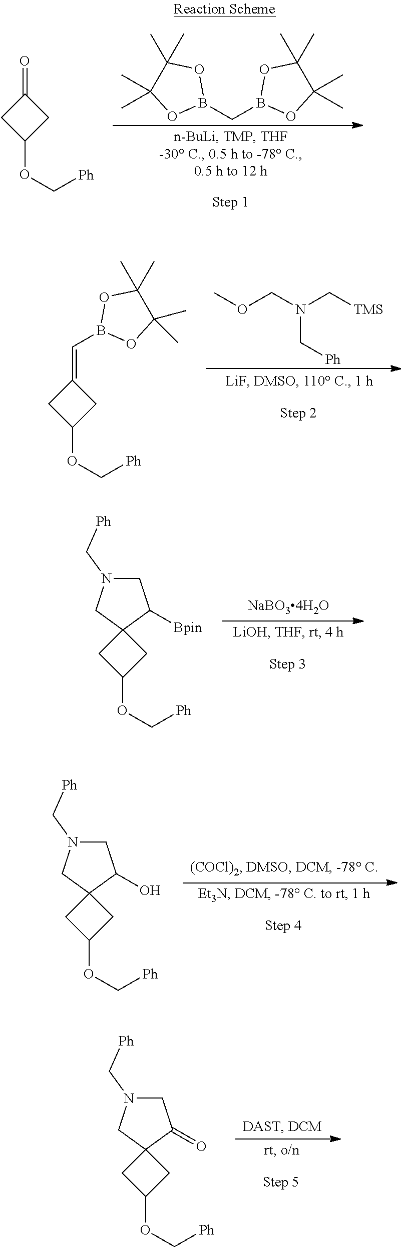

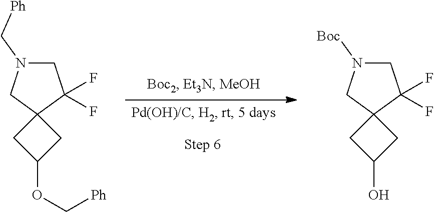 [SMILES: FC1(F)CN(Cc2ccccc2)CC12CC(OCc1ccccc1)C2 ; CC(C)(C)OC(=O)N1CC(F)(F)C2(CC(O)C2)C1]

### Step 1: 2-((3-(benzyloxy)cyclobutylidene)methyl)-4,4,5,5-tetramethyl-1,3,2-dioxaborolane

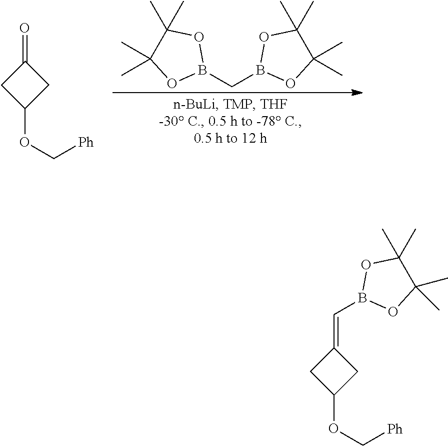

To a cooled to −30° C. solution of 2,2,6,6-tetramethylpiperidine (9.62 g, 68.09 mmol) in dry THF (100 mL) was added n-BuLi (2.5 M, 27.2 mL) dropwise under an N₂ atmosphere. The mixture was stirred at −30° C. for 0.5 h. The reaction was then cooled to −78° C. and a solution of bis(4,4,5,5-tetramethyl-1,3,2-dioxaborolan-2-yl)methane (15.21 g, 56.74 mmol) in 50 mL of dry THF was added dropwise. The reaction mixture was stirred at −78° C. for 0.5 h and a solution of 3-(benzyloxy)cyclobutan-1-one (10 g, 56.74 mmol) in 50 mL of dry THF was added dropwise. The reaction mixture was then warmed to 20° C. and stirred for an additional 12 h. The reaction mixture was slowly poured into 20 mL of saturated aqueous NH₄Cl at 0° C. and after stirring for 1 h, the solution was diluted with H₂O (200 mL) and extracted with EtOAc (400 mL×3). The organic phase was dried over Na₂SO₄, filtered and concentrated under reduced pressure to afford 2-((3-(benzyloxy)cyclobutylidene)methyl)-4,4,5,5-tetramethyl-1,3,2-dioxaborolane (13.7 g crude) which was used without further purification.

LCMS (ESI-MS) m/z=301.1 [M+H]⁺.

### Step 2: 6-benzyl-2-(benzyloxy)-8-(4,4,5,5-tetramethyl-1,3,2-dioxaborolan-2-yl)-6-azaspiro[3.4]octane

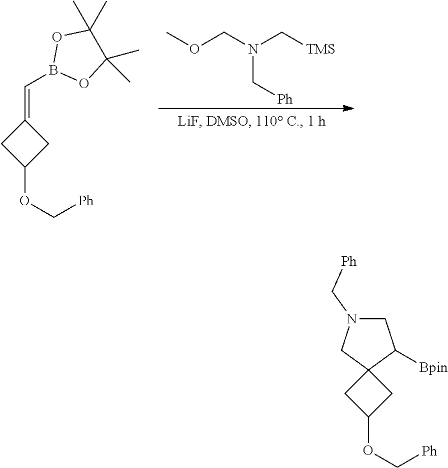

A solution of 2-((3-(benzyloxy)cyclobutylidene)methyl)-4,4,5,5-tetramethyl-1,3,2-dioxaborolane (13.7 g crude), N-benzyl-1-methoxy-N-((trimethylsilyl)methyl)methanamine (13.00 g, 54.76 mmol) and LiF (3.55 g, 136.90 mmol) in DMSO (200 mL) was stirred at 110° C. for 1 h. The reaction mixture was diluted with H₂O (200 mL) and extracted with EA (1000 mL). The combined organic layers were washed with brine, dried over anhydrous Na₂SO₄, filtered and concentrated under reduced pressure to afford crude 6-benzyl-2-(benzyloxy)-8-(4,4,5,5-tetramethyl-1,3,2-dioxaborolan-2-yl)-6-azaspiro[3.4]octane (20 g) as a colorless oil which was used for the next step without further purification.

LCMS (ESI-MS) m/z=434.2 [M+H]⁺.

### Step 3: 6-benzyl-2-(benzyloxy)-6-azaspiro[3.4]octan-8-ol

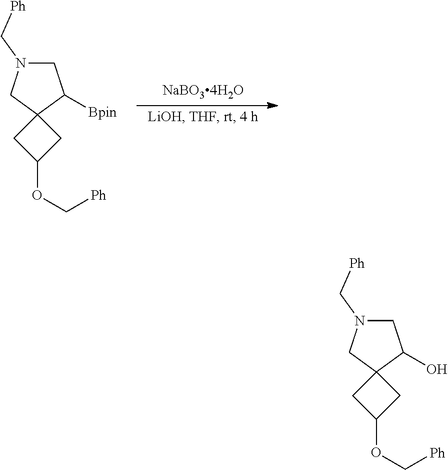

A solution of 6-benzyl-2-(benzyloxy)-8-(4,4,5,5-tetramethyl-1,3,2-dioxaborolan-2-yl)-6-azaspiro[3.4]octane (20 g crude), sodium perborate (4.53 g, 55.37 mmol) and LiOH (3.32 g, 138.44 mmol) in THF (50 mL) and H₂O (200 mL) was stirred at room temperature for 4 h. The reaction mixture was diluted with H₂O (200 mL) and extracted with EA (3×500 mL). The combined organic layers were washed with brine, dried over anhydrous Na₂SO₄, filtered and concentrated under reduced pressure. The crude product was purified by silica gel column chromatography (MeOH/DCM, 3:97) to obtain 6-benzyl-2-(benzyloxy)-6-azaspiro[3.4]octan-8-ol (10 g, 67.0%) as colorless oil.

LCMS (ESI-MS) m/z=324.1 [M+H]⁺.

### Step 4: 6-benzyl-2-(benzyloxy)-6-azaspiro[3.4]octan-8-one

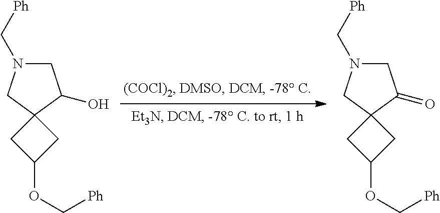 [SMILES: OC1CN(Cc2ccccc2)CC12CC(OCc1ccccc1)C2 ; O=C1CN(Cc2ccccc2)CC12CC(OCc1ccccc1)C2]

To a cooled to −78° C. solution of oxalyl chloride (7.85 g, 61.8 mmol) in DCM (100 mL) was added dropwise a solution of DMSO (4.83 g, 61.8 mmol) in DCM (20 mL) under a nitrogen atmosphere. The mixture was stirred at −78° C. for 20 min. A solution of 6-benzyl-2-(benzyloxy)-6-azaspiro[3.4]octan-8-ol (10 g, 30.9 mmol) in DCM (20 mL) was then added dropwise and the mixture stirred for 20 min. Et₃N (12.5 g, 123 mmol) was added dropwise and the mixture stirred for 20 min. The reaction mixture was diluted with water (200 mL) and extracted with DCM (3×200 mL). The combined organic layers were washed with brine, dried over anhydrous Na₂SO₄, filtered and concentrated under reduced pressure. The residue was purified by silica gel column chromatography (EA in PE, 0 to 10%). The fractions with the desired mass signal were combined and concentrated under reduced pressure to afford 6-benzyl-2-(benzyloxy)-6-azaspiro[3.4]octan-8-one (5.8 g, 58.4% yield).

LCMS (ESI-MS) m/z=322.2 [M+H]⁺.

### Step 5: 6-benzyl-2-(benzyloxy)-8,8-difluoro-6-azaspiro[3.4]octane

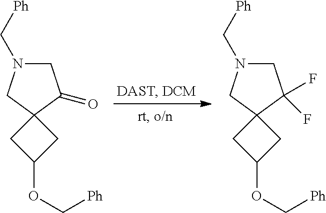 [SMILES: O=C1CN(Cc2ccccc2)CC12CC(OCc1ccccc1)C2 ; FC1(F)CN(Cc2ccccc2)CC12CC(OCc1ccccc1)C2]

DAST (8.73 g, 54.1 mmol) was added to a solution of 6-benzyl-2-(benzyloxy)-6-azaspiro[3.4]octan-8-one (5.8 g, 18.0 mmol) in DCM (60 mL) at 0° C. The resulting mixture was stirred overnight at room temperature. The reaction mixture was concentrated under reduced pressure. The residue was purified by silica gel column chromatography (EA in PE, 0% to 10%). The fractions with the desired mass signal were combined and concentrated under reduced pressure to afford 6-benzyl-2-(benzyloxy)-8,8-difluoro-6-azaspiro[3.4]octane (1.2 g, 19.4% yield).

LCMS (ESI-MS) m/z=344.2 [M+H]⁺.

### Step 6: Tert-butyl 8,8-difluoro-2-hydroxy-6-azaspiro[3.4]octane-6-carboxylate

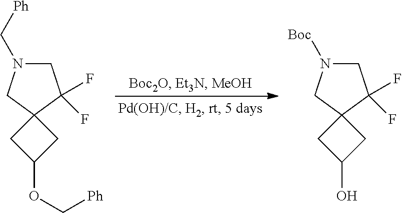 [SMILES: FC1(F)CN(Cc2ccccc2)CC12CC(OCc1ccccc1)C2 ; CC(C)(C)OC(=O)N1CC(F)(F)C2(CC(O)C2)C1]

Pd(OH)₂/C (0.49 g, 3.49 mmol) was added to a solution of 6-benzyl-2-(benzyloxy)-8,8-difluoro-6-azaspiro[3.4]octane (1.2 g, 3.49 mmol), Boc₂O (0.92 g, 4.19 mmol) and Et₃N (1.06 g, 10.48 mmol) in MeOH (120 mL). The resulting mixture was stirred 5 days at room temperature under a H₂ atmosphere. The reaction mixture was filtered and the filtrate was concentrated under reduced pressure. The residue was purified by silica gel column chromatography (MeOH in DCM, 0% to 5%). The fractions with the desired mass signal were combined, concentrated under reduced pressure and lyophilized to afford the tert-butyl 8,8-difluoro-2-hydroxy-6-azaspiro[3.4]octane-6-carboxylate (500 mg, 54.4% yield).

¹H NMR (400 MHz, DMSO-d₆) δ 5.3-5.06 (m, 1H), 4.20-4.00 (m, 1H), 3.69-3.50 (m, 3H), 3.47-3.39 (m, 2H), 2.14-2.10 (m, 1H), 2.04-1.96 (m, 1H), 1.93-1.85 (m, 1H), 1.40 (s, 9H).

### Intermediate 28: 6-benzyl 2-(tert-butyl) 8-(difluoromethyl)-2,6-diazaspiro[3.4]octane-2,6-dicarboxylate

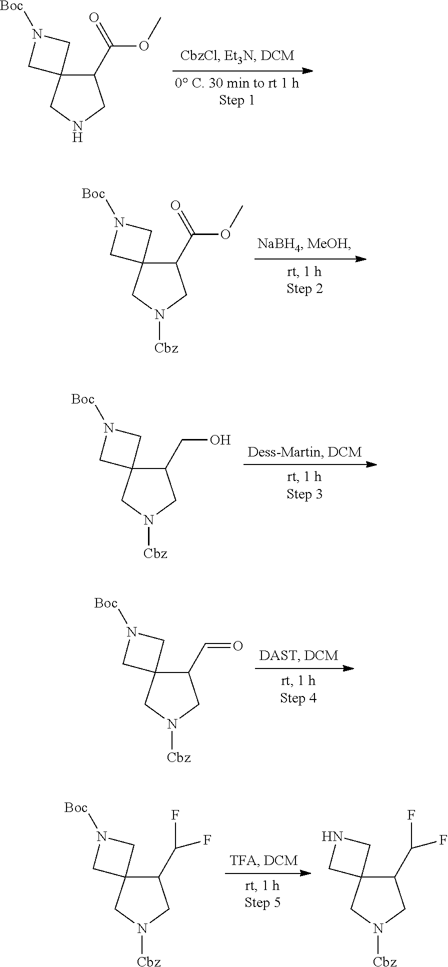 [SMILES: COC(=O)C1CNCC12CN(C(=O)OC(C)(C)C)C2 ; COC(=O)C1CN(C(=O)OCc2ccccc2)CC12CN(C(=O)OC(C)(C)C)C2 ; CC(C)(C)OC(=O)N1CC2(CN(C(=O)OCc3ccccc3)CC2CO)C1 ; CC(C)(C)OC(=O)N1CC2(CN(C(=O)OCc3ccccc3)CC2C=O)C1 ; CC(C)(C)OC(=O)N1CC2(CN(C(=O)OCc3ccccc3)CC2C(F)F)C1 ; O=C(OCc1ccccc1)N1CC(C(F)F)C2(CNC2)C1]

### Step 1: 6-benzyl 2-(tert-butyl) 8-methyl 2,6-diazaspiro[3.4]octane-2,6,8-tricarboxylate

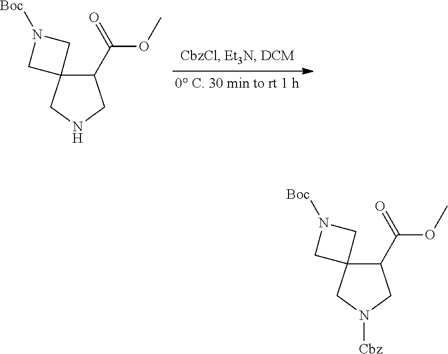 [SMILES: COC(=O)C1CNCC12CN(C(=O)OC(C)(C)C)C2 ; COC(=O)C1CN(C(=O)OCc2ccccc2)CC12CN(C(=O)OC(C)(C)C)C2]

CbzCl (1.14 g, 6.65 mmol) and Et₃N (0.90 g, 8.87 mmol) were added to a cooled to 0° C. solution of 2-(tert-butyl) 8-methyl 2,6-diazaspiro[3.4]octane-2,8-dicarboxylate (1.2 g, 4.43 mmol) in DCM (20 mL). The reaction mixture was stirred for 30 minutes, then warmed to room temperature and stirred for 1 h. The reaction was quenched with water (200 mL). The resulting mixture was extracted with CH₂Cl₂ (3×200 mL). The combined organic layers were dried over anhydrous Na₂SO₄. After filtration, the filtrate was concentrated under reduced pressure. The residue was purified by silica gel column chromatography (PE/EA, 1:1) to afford 6-benzyl 2-(tert-butyl) 8-methyl 2,6-diazaspiro[3.4]octane-2,6,8-tricarboxylate (1.5 g, 83.6% yield).

LCMS (ESI-MS) m/z=305.1 [M+H-100]⁺.

### Step 2: 6-benzyl 2-(tert-butyl) 8-(hydroxymethyl)-2,6-diazaspiro[3.4]octane-2,6-dicarboxylate

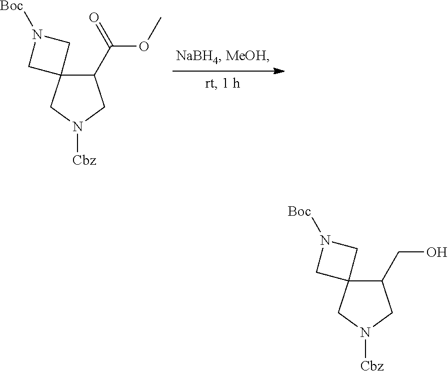 [SMILES: COC(=O)C1CN(C(=O)OCc2ccccc2)CC12CN(C(=O)OC(C)(C)C)C2 ; CC(C)(C)OC(=O)N1CC2(CN(C(=O)OCc3ccccc3)CC2CO)C1]

NaBH₄ (74.83 mg, 1.97 mmol) was added to a solution of 6-benzyl 2-(tert-butyl) 8-methyl 2,6-diazaspiro[3.4]octane-2,6,8-tricarboxylate (200 mg, 0.49 mmol) in MeOH (3 mL). The reaction mixture was stirred for 1 h at room temperature and quenched with water (20 mL). The resulting mixture was extracted with EA (3×20 mL). The combined organic layers were dried over anhydrous Na₂SO₄. After filtration, the filtrate was concentrated under reduced pressure and used as such for the next step.

LCMS (ESI-MS) m/z=277.1 [M+H-100]⁺.

### Step 3: 6-benzyl 2-(tert-butyl) 8-formyl-2,6-diazaspiro[3.4]octane-2,6-dicarboxylate

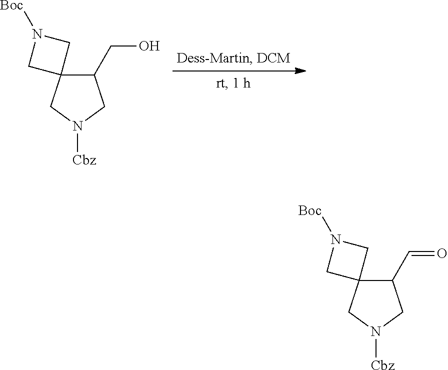 [SMILES: CC(C)(C)OC(=O)N1CC2(CN(C(=O)OCc3ccccc3)CC2CO)C1 ; CC(C)(C)OC(=O)N1CC2(CN(C(=O)OCc3ccccc3)CC2C=O)C1]

Dess-Martin (581.37 mg, 1.37 mmol) was added to a mixture of 6-benzyl 2-(tert-butyl) 8-(hydroxymethyl)-2,6-diazaspiro[3.4]octane-2,6-dicarboxylate (430 mg, 1.14 mmol) in DCM (8 mL). The resulting mixture was stirred for 1 h at room temperature and filtered. The filter cake was washed with CH₂Cl₂ (30 mL) and the filtrate was concentrated under reduced pressure. The residue was purified by Prep-TLC (PE/EA, 1:3) to afford 6-benzyl 2-(tert-butyl) 8-formyl-2,6-diazaspiro[3.4]octane-2,6-dicarboxylate (240 mg, 56.1% yield).

LCMS (ESI-MS) m/z=275.1 [M+H-100]⁺.

### Step 4: 6-benzyl 2-(tert-butyl) 8-(difluoromethyl)-2,6-diazaspiro[3.4]octane-2,6-dicarboxylate

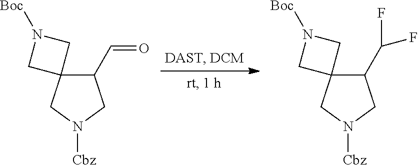 [SMILES: CC(C)(C)OC(=O)N1CC2(CN(C(=O)OCc3ccccc3)CC2C=O)C1 ; CC(C)(C)OC(=O)N1CC2(CN(C(=O)OCc3ccccc3)CC2C(F)F)C1]

DAST (206.63 mg, 1.28 mmol) was added to a mixture of 6-benzyl 2-(tert-butyl) 8-formyl-2,6-diazaspiro[3.4]octane-2,6-dicarboxylate (240 mg, 0.64 mmol) in DCM (5 mL). The mixture was stirred for 1 h at room temperature and quenched with water (20 mL). The resulting mixture was extracted with CH₂Cl₂ (2×20 mL). The combined organic layers were dried over anhydrous Na₂SO₄. After filtration, the filtrate was concentrated under reduced pressure. The residue was purified by Prep-TLC (PE/EA, 1:3) to afford 6-benzyl 2-(tert-butyl) 8-(difluoromethyl)-2,6-diazaspiro[3.4]octane-2,6-dicarboxylate (160 mg, 63.0% yield).

LCMS (ESI-MS) m/z=297.1 [M+H-100]⁺.

### Step 5: Benzyl 8-(difluoromethyl)-2,6-diazaspiro[3.4]octane-6-carboxylate

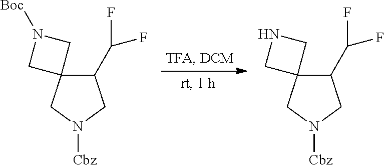 [SMILES: CC(C)(C)OC(=O)N1CC2(CN(C(=O)OCc3ccccc3)CC2C(F)F)C1 ; O=C(OCc1ccccc1)N1CC(C(F)F)C2(CNC2)C1]

A solution of 6-benzyl 2-(tert-butyl) 8-(difluoromethyl)-2,6-diazaspiro[3.4]octane-2,6-dicarboxylate (130 mg, 0.32 mmol) in TFA (0.3 mL) and DCM (0.9 mL) was stirred for 1 h at room temperature. The reaction mixture was concentrated under reduced pressure and used as such for the next step.

LCMS (ESI-MS) m/z=297.1 [M+H]⁺.

### EXAMPLE COMPOUNDS

### Example 2: 8-(2,2-difluoro-6-azaspiro[3.4]octan-6-yl)-N-(1-(methylsulfonyl)piperidin-4-yl)pyrido[3,4-d]pyrimidin-2-amine

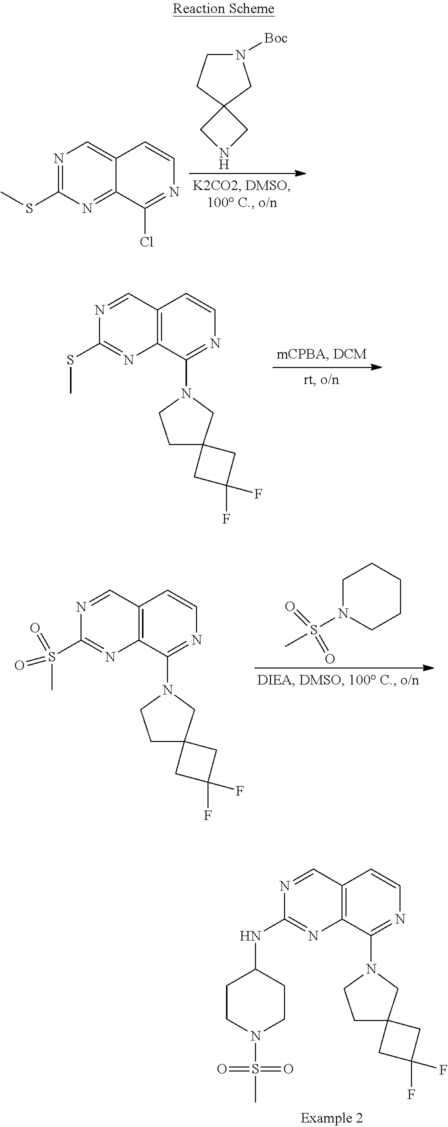

### Detailed Procedure

### Step 1: 8-(2,2-difluoro-6-azaspiro [3.4] octan-6-yl)-2-(methylthio)pyrido[3,4-d]pyrimidine

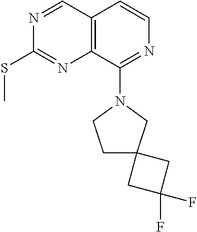 [SMILES: CSc1ncc2ccnc(N3CCC4(C3)CC(F)(F)C4)c2n1]

To a stirred mixture of 8-chloro-2-(methylthio)pyrido[3,4-d]pyrimidine (900 mg, 4.25 mmol) and 2,2-difluoro-6-azaspiro[3.4]octane hydrochloride (780.4 mg, 4.25 mmol) in acetonitrile (10 mL) was added N,N-diisopropylethylamine (1.65 g, 12.75 mmol). The resulting mixture was heated to 100° C. and stirred overnight. The reaction mixture was allowed to cool to room temperature and concentrated under vacuum. The residue was diluted with water (100 mL) and extracted with ethyl acetate (3×100 mL). The combined organic layers were washed with brine, dried over anhydrous sodium sulfate and filtered. The filtrate was concentrated under vacuum to afford the crude product 8-(2,2-difluoro-6-azaspiro[3.4]octan-6-yl)-2-(methylthio)pyrido[3,4-d]pyrimidine (880 mg). The crude product was used for next step directly without further purification.

LCMS (ESI) m/z=323 [M+H]⁺.

### Step 2: 8-(2,2-difluoro-6-azaspiro[3.4]octan-6-yl)-2-(methylsulfonyl)pyrido[3,4-d]pyrimidine

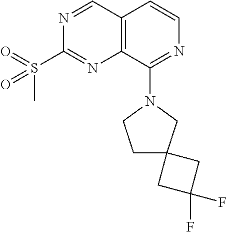 [SMILES: CS(=O)(=O)c1ncc2ccnc(N3CCC4(C3)CC(F)(F)C4)c2n1]

To a stirred solution of 8-(2,2-difluoro-6-azaspiro[3.4]octan-6-yl)-2-(methylthio)pyrido[3,4-d]pyrimidine (780 mg, 2.42 mmol) in dichloromethane (20 mL) was added 3-chloroperoxybenzoic acid (1.04 g, 6.05 mmol). The resulting mixture was stirred overnight at room temperature and concentrated under vacuum to afford the crude 8-(2,2-difluoro-6-azaspiro[3.4]octan-6-yl)-2-(methylsulfonyl)pyrido[3,4-d]pyrimidine (910 mg), the crude product was used for next step directly without further purification. LCMS (ESI) m/z=355 [M+H]⁺.

### Step 3: 8-(2,2-difluoro-6-azaspiro[3.4]octan-6-yl)-N-(1-(methylsulfonyl)piperidin-4-yl)pyrido[3,4-d]pyrimidin-2-amine

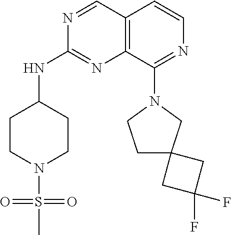 [SMILES: CS(=O)(=O)N1CCC(Nc2ncc3ccnc(N4CCC5(C4)CC(F)(F)C5)c3n2)CC1]

To a stirred mixture of 8-(2,2-difluoro-6-azaspiro[3.4]octan-6-yl)-2-(methylsulfonyl)pyrido[3,4-d]pyrimidine (150 mg, 0.42 mmol) and 1-(methylsulfonyl)piperidin-4-amine (75.45 mg, 0.42 mmol) in dimethyl sulfoxide (2 mL) were added N,N-diisopropylethylamine (164.12 mg, 1.26 mmol). The resulting mixture was heated to 100° C. and stirred overnight. The reaction mixture was allowed to cool to room temperature, diluted with water (20 mL) and extracted with ethyl acetate (3×20 mL). The combined organic layers were washed with brine (50 mL), dried with anhydrous sodium sulfate, filtered and the filtrate was concentrated under vacuum to afford the crude product. The crude product was purified by preparative reverse phase HPLC (acetonitrile/water (with 10 mM NH₄HCO₃ and 0.1% NH₃·H₂O) gradient) to afford the title compound (48.9 mg, 23.8% yield).

¹H NMR (400 MHz, DMSO-d₆) δ 8.99 (s, 1H), 7.73 (d, J=5.6 Hz, 1H), 7.52 (s, 1H), 6.81 (d, J=5.2 Hz, 1H), 4.10 (S, 2H), 3.89 (S, 3H), 3.59 (d, J=12.4 Hz, 2H), 2.93-2.86 (m, 5H), 2.75-2.56 (m, 4H), 2.13-1.96 (m, 4H), 1.69-1.56 (m, 2H).

LCMS (ESI) m/z=453 [M+H]⁺.

### Example 6: 8-(2,2-difluoro-6-azaspiro[3.4]octan-6-yl)-N-(5-(6-ethyl-2,6-diazaspiro[3.3]heptan-2-yl)pyridin-2-yl)pyrido[3,4-d]pyrimidine-2-amine

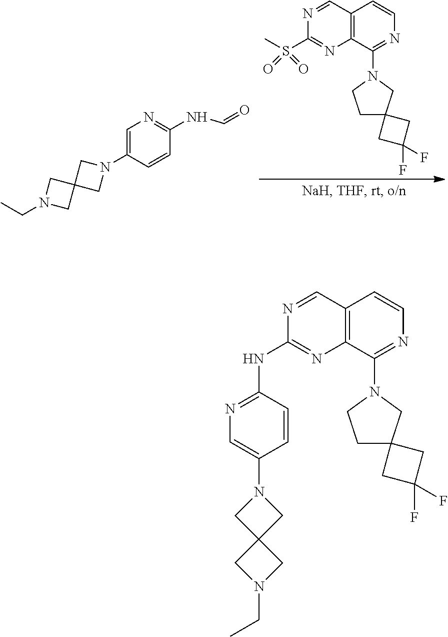 [SMILES: CCN1CC2(C1)CN(c1ccc(NC=O)nc1)C2 ; CS(=O)(=O)c1ncc2ccnc(N3CCC4(C3)CC(F)(F)C4)c2n1 ; CCN1CC2(C1)CN(c1ccc(Nc3ncc4ccnc(N5CCC6(C5)CC(F)(F)C6)c4n3)nc1)C2]

To a stirred mixture of N-(5-(6-ethyl-2,6-diazaspiro[3.3]heptan-2-yl)pyridin-2-yl)formamide (50 mg, 0.20 mmol) in tetrahydrofuran (2 mL) was added sodium hydride (60% in mineral oil, 24 mg, 0.60 mmol) at 0° C. The resulting mixture was stirred for 15 minutes at 0° C. Then 8-(2,2-difluoro-6-azaspiro[3.4]octan-6-yl)-2-(methylsulfonyl)pyrido[3,4-d]pyrimidine (78 mg, 0.22 mmol) was added to the mixture and the resulting mixture was warmed to room temperature and stirred overnight. The reaction mixture was quenched by addition of water (50 mL) and extracted with dichloromethane (3×50 mL). The combined organic layers were washed with brine (100 mL), dried over anhydrous sodium sulfate, and filtered. The filtrate was concentrated under vacuum and the residue was purified by preparative reverse phase HPLC (acetonitrile/water (with 10 mM NH₄HCO₃ and 0.1%₀NH₃·H₂O) gradient) to afford the title compound (12.7 mg, 10.0% yield).

¹H NMR (400 MHz, DMSO-d₆) δ (ppm) 9.13 (s, 1H), 7.94-7.62 (m, 3H), 7.14-6.97 (m, 2H), 4.39 (s, 4H), 4.12-4.18 (m, 6H), 3.98-3.92 (m, 2H), 3.27-3.25 (m, 2H), 2.67-2.61 (m, 4H), 2.16-2.13 (m, 2H), 1.42-1.34 (m, 1H), 1.31-1.23 (m, 3H).

LCMS (ESI) m/z=493 [M+H]⁺.

### Example 6: 8-(2,2-difluoro-6-azaspiro[3.4]octan-6-yl)-N-(5-(6-ethyl-2,6-diazaspiro[3.3]heptan-2-yl)pyridin-2-yl)pyrido[3,4-d]pyrimidin-2-amine

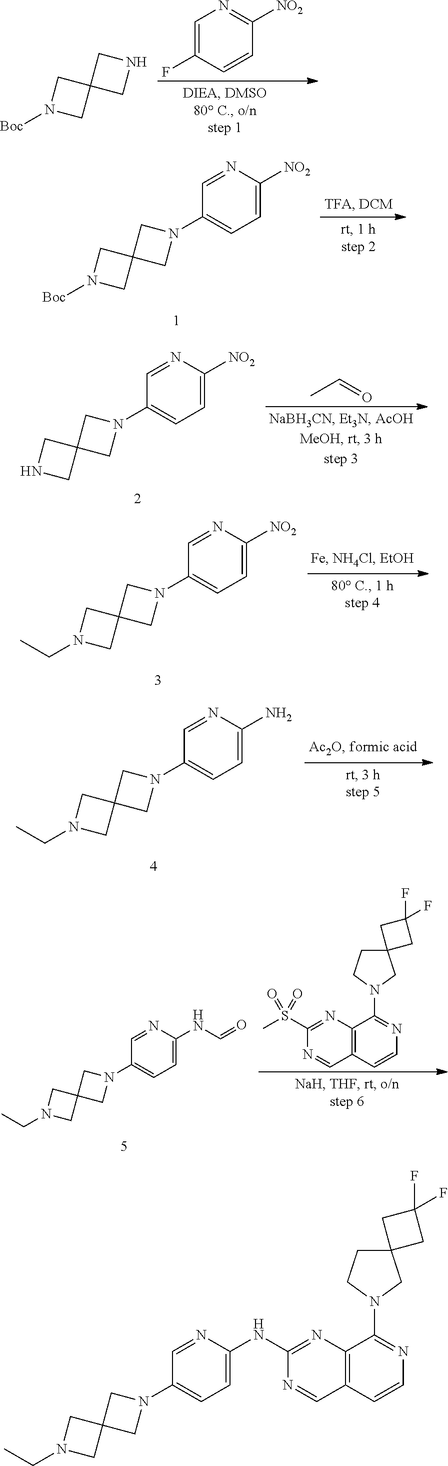

### Step 1: tert-butyl 6-(6-nitropyridin-3-yl)-2,6-diazaspiro[3.31heptane, 2-carboxylate

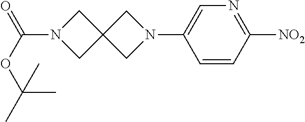 [SMILES: CC(C)(C)OC(=O)N1CC2(C1)CN(c1ccc([N+](=O)[O-])nc1)C2]

To a stirred mixture of tert-butyl 2,6-diazaspiro[3.3]heptane-2-carboxylate (5 g, 25.21 mmol) and 5-fluoro-2-nitropyridine (5.37 g, 37.82 mmol) in dimethyl sulfoxide (30 mL) was N,N-diisopropylethylamine (9.78 g, 75.65 mmol). The resulting mixture was heated to 80° C. and stirred for 3 hours. The reaction mixture was allowed to cool to room temperature, diluted with water (500 mL) and extracted with ethyl acetate (3×500 mL). The combined organic layers were washed with brine (500 mL), dried over anhydrous sodium sulfate, and filtered. The filtrate was concentrated under vacuum to afford the crude product. The residue was purified by trituration with petroleum ether/ethyl acetate (5:1, 100 mL) to afford tert-butyl 6-(6-nitropyridin-3-yl)-2,6-diazaspiro[3.3]heptane-2-carboxylate (6.78 g, 83.7% yield) as a yellow solid.

LCMS (ESI) m/z=321 [M+H]⁺.

### Step 2: 2-(6-nitropyridin-3-yl)-2,6-diazaspiro [3.3] heptane

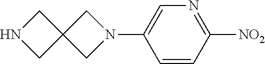 [SMILES: O=[N+]([O-])c1ccc(N2CC3(CNC3)C2)cn1]

To a stirred mixture of tert-butyl 6-(6-nitropyridin-3-yl)-2,6-diazaspiro[3.3]heptane-2-carboxylate (6.78 g, 21.16 mmol) in dichloromethane (80 mL) was added trifluoroacetic acid (16 mL). The resulting mixture was stirred for 1 hour at room temperature and concentrated under vacuum. The residue was diluted with dichloromethane (100 mL) and concentrated under vacuum again to afford crude 2-(6-nitropyridin-3-yl)-2,6-diazaspiro[3.3]heptane trifluoroacetic acid salt (6 g) as a brown yellow solid. The crude product was used for next step directly without further purification.

LCMS (ESI) m/z=221 [M+H]⁺.

### Step 3: 2-ethyl-6-(6-nitropyridin-3-yl)-2,6-diazaspiro[3.31heptane

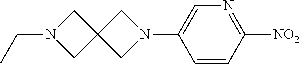 [SMILES: CCN1CC2(C1)CN(c1ccc([N+](=O)[O-])nc1)C2]

A solution of 2-(6-nitropyridin-3-yl)-2,6-diazaspiro[3.3]heptane trifluoroacetic acid salt (6 g, 18.9 mmol) in methanol (100 mL) was treated with triethylamine (5.73 g, 56.7 mmol) for 10 minutes followed by the addition of acetaldehyde (4.16 g, 94.5 mmol), acetic acid (0.23 mL, 4.08 mmol) and sodium cyanoborohydride (2.51 g, 39.8 mmol). The resulting mixture was stirred for 3 hours at room temperature and concentrated under vacuum. The residue was diluted with water (500 mL) and extracted with ethyl acetate (3×500 mL). The combined organic layers were washed with brine (1000 mL), dried over anhydrous sodium sulfate, and filtered. The filtrate was concentrated under reduced pressure to afford the crude product. The residue was purified by trituration with dichloromethane (100 mL) to afford 2-ethyl-6-(6-nitropyridin-3-yl)-2,6-diazaspiro[3.3]heptane (4 g, 58.9% yield) as an orange solid. LCMS (ESI) m/z=249 [M+H]⁺.

### Step 4: 5-(6-ethyl-2,6-diazaspiro[3.3heptan-2-yl) pyridin-2-amine

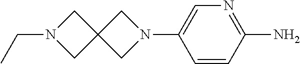 [SMILES: CCN1CC2(C1)CN(c1ccc(N)nc1)C2]

A solution of 2-ethyl-6-(6-nitropyridin-3-yl)-2,6-diazaspiro[3.3]heptane (4 g, 16.11 mmol), ammonium chloride (4.31 g, 80.55 mmol), iron powder (9.00 g, 161.100 mmol) and water (20 mL) in ethanol (60 mL) was stirred for 1 hour at 80° C. The resulting mixture was filtered and the filter cake was washed with ethanol (100 mL). The filtrate was concentrated under vacuum to afford the crude product. The residue was purified by reversed-phase flash chromatography (C18 silica gel, acetonitrile/water (with 10 mmol/L NH₄HCO₃) gradient) to afford 5-(6-ethyl-2,6-diazaspiro[3.3]heptan-2-yl)pyridin-2-amine (2 g, 56.6% yield) as a black solid.

LCMS (ESI) m/z=219 [M+H]⁺.

### Step 5: N-(5-(6-ethyl-2,6-diazaspiro[3.3]heptan-2-yl) pyridin-2-yl) formamide

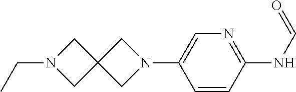 [SMILES: CCN1CC2(C1)CN(c1ccc(NC=O)nc1)C2]

A solution of acetic anhydride (2 mL) in formic acid (4 mL) was stirred for 1 hour at room temperature followed by the addition of 5-(6-ethyl-2,6-diazaspiro[3.3]heptan-2-yl)pyridin-2-amine (400 mg, 1.83 mmol) in portions at room temperature. The resulting mixture was stirred for 3 hours at room temperature. The reaction mixture was neutralized to PH=7 with saturated aqueous sodium bicarbonate (200 mL) and extracted with ethyl acetate (3×100 mL). The combined organic layers were washed with brine, dried over anhydrous sodium sulfate, and filtered. The filtrate was concentrated under vacuum and the residue was purified by preparative reverse phase HPLC (acetonitrile/water (with 10 mM NH₄HCO₃ and 0.1% NH₃·H₂O) gradient) to afford the title compound (70 mg, 15.3% yield) as an off-white solid. LCMS (ESI) m/z=247 [M+H]⁺.

### Step 6: 8-(2,2-difluoro-6-azaspiro[3.4]octan-6-yl)-N-(5-(6-ethyl-2,6-diazaspiro[3.3]heptan-2-yl)pyridin-2-yl) pyrido[3,4-d]pyrimidin-2-amine

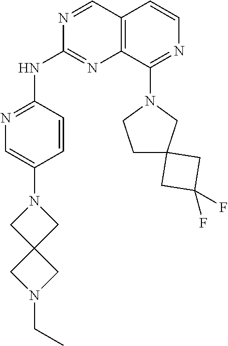 [SMILES: CCN1CC2(C1)CN(c1ccc(Nc3ncc4ccnc(N5CCC6(C5)CC(F)(F)C6)c4n3)nc1)C2]

To a stirred mixture of N-(5-(6-ethyl-2,6-diazaspiro[3.3]heptan-2-yl)pyridin-2-yl)formamide (50 mg, 0.20 mmol) in tetrahydrofuran (2 mL) was added sodium hydride (60% in mineral oil, 24 mg, 0.60 mmol) at 0° C. The resulting mixture was stirred for 15 minutes at 0° C. Then 8-(2,2-difluoro-6-azaspiro[3.4]octan-6-yl)-2-(methylsulfonyl)pyrido[3,4-d]pyrimidine (78 mg, 0.22 mmol) was added to the mixture and the resulting mixture was warmed to room temperature and stirred overnight. The reaction mixture was quenched by addition of water (50 mL) and extracted with dichloromethane (3×50 mL). The combined organic layers were washed with brine (100 mL), dried over anhydrous sodium sulfate, and filtered. The filtrate was concentrated under vacuum and the residue was purified by preparative reverse phase HPLC (acetonitrile/water (with 10 mM NH₄HCO₃ and 0.1% NH₃·H₂O) gradient) to afford the title compound (12.7 mg, 10.0% yield) as an orange solid.

LCMS (ESI) m/z=493 [M+H]⁺. ¹H NMR (400 MHz, DMSO-d₆) δ (ppm) 9.13 (s, 1H), 7.94-7.62 (m, 3H), 7.14-6.97 (m, 2H), 4.39 (s, 4H), 4.12-4.18 (m, 6H), 3.98-3.92 (m, 2H), 3.27-3.25 (m, 2H), 2.67-2.61 (m, 4H), 2.16-2.13 (m, 2H), 1.42-1.34 (m, 1H), 1.31-1.23 (m, 3H).

### Example 11: 8-(8,8-difluoro-2-(methyl-d₃)-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine

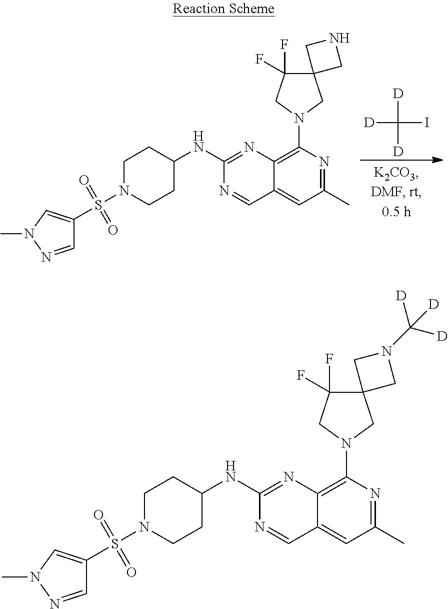

Iodomethane-d₃ (9.51 mg, 0.06 mmol) was added to a mixture of 8-(8,8-difluoro-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine (70 mg, 0.13 mmol) and K₂CO₃ (36.3 mg, 0.26 mmol) in DMF (1 mL). The reaction mixture was stirred for 30 minutes at room temperature, quenched by addition of water (5 mL) and extracted with DCM (3×5 mL). The combined organic layers were washed with brine (10 mL), dried over anhydrous Na₂SO₄, filtered and the filtrate was concentrated under reduced pressure to afford the crude product. The residue was purified by Prep-TLC (MeOH/DCM=1:10) to afford the desired product 8-(8,8-difluoro-2-(methyl-d₃)-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine (23.6 mg, 32.0% yield).

¹H NMR (400 MHz, DMSO-d₆) δ 8.96 (s, 1H), 8.34 (s, 1H), 7.77 (s, 1H), 7.53 (s, 1H), 6.76 (s, 1H), 4.33-4.24 (m, 2H), 4.23-4.15 (m, 1H), 3.92 (s, 3H), 3.70 (s, 1H), 3.60-3.50 (m, 2H), 3.31-3.25 (m, 2H), 3.18-2.99 (m, 2H), 2.49-2.40 (m, 3H), 2.33 (s, 3H), 2.08-1.97 (m, 2H), 1.70-1.54 (m, 2H).

LCMS (ESI-MS) m/z=551.3 [M+H]⁺.

### Example 12: 8-(8,8-difluoro-2-(methyl-d₃)-2,6-diazaspiro[3.4]octan-6-yl)-6-methyl-N-(1-(methylsulfonyl)piperidin-4-yl)pyrido [3,4-d]pyrimidin-2-amine

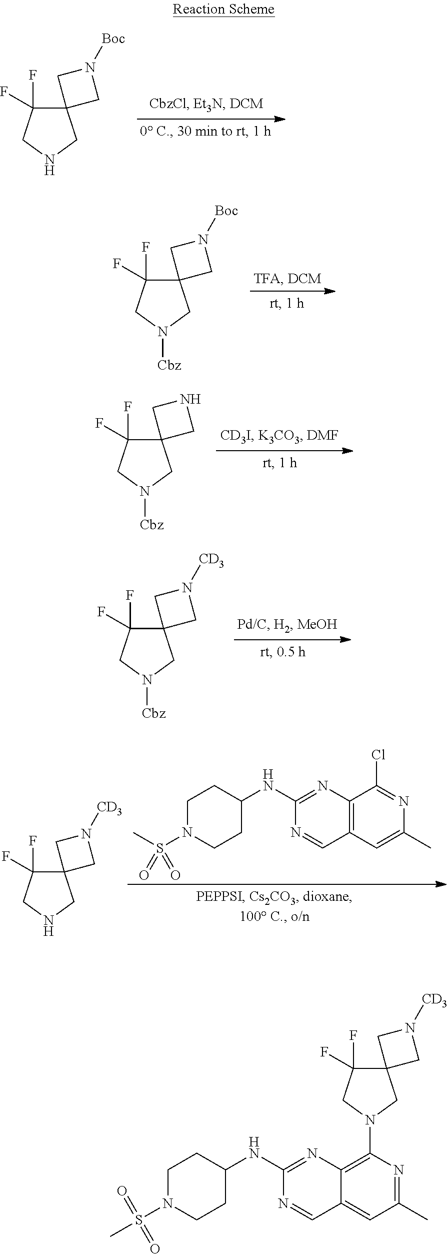

### Step 1: 6-benzyl 2-(tert-butyl) 8,8-difluoro-2,6-diazaspiro[3.4]octane-2,6-dicarboxylate

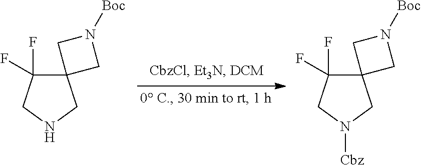 [SMILES: CC(C)(C)OC(=O)N1CC2(CNCC2(F)F)C1 ; CC(C)(C)OC(=O)N1CC2(CN(C(=O)OCc3ccccc3)CC2(F)F)C1]

Benzyl chloroformate (412 mg, 2.42 mmol) was added to a cooled to 0° C. solution of

tert-butyl 8,8-difluoro-2,6-diazaspiro[3.4]octane-2-carboxylate (500 mg, 2.01 mmol) and Et₃N (408 mg, 4.03 mmol) in DCM (6 mL). The resulting mixture was stirred for 30 minutes at 0° C. and then warmed to room temperature and stirred for another hour. The resulting mixture was quenched by addition of water (15 mL) and extracted with DCM (3×15 mL). The combined organic layers were washed with brine (50 mL), dried over anhydrous Na₂SO₄, filtered and the filtrate was concentrated under vacuum. The residue was purified by Prep-TLC (PE/EA, 2:1) to afford the desired product 6-benzyl 2-(tert-butyl) 8,8-difluoro-2,6-diazaspiro[3.4]octane-2,6-dicarboxylate (700 mg, 90.9% yield).

LCMS (ESI-MS) m/z=383.2 [M+H]⁺.

### Step 2: benzyl 8,8-difluoro-2,6-diazaspiro[3.4]octane-6-carboxylate

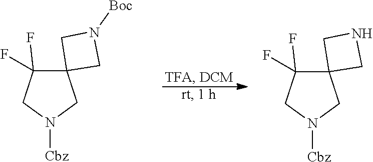 [SMILES: CC(C)(C)OC(=O)N1CC2(CN(C(=O)OCc3ccccc3)CC2(F)F)C1 ; O=C(OCc1ccccc1)N1CC(F)(F)C2(CNC2)C1]

TFA (2 mL) was added to a stirred mixture of 6-benzyl 2-(tert-butyl) 8,8-difluoro-2,6-diazaspiro[3.4]octane-2,6-dicarboxylate (500 mg, 1.31 mmol) in DCM (6 mL). The resulting mixture was stirred for 1 hour at room temperature and concentrated under high vacuum to afford the crude product (350 mg). The crude product was used for the next step without further purification.

LCMS (ESI-MS) m/z=283.2 [M+H]⁺.

### Step 3: Benzyl 8,8-difluoro-2-(methyl-d₃)-2,6-diazaspiro[3.4]octane-6-carboxylate

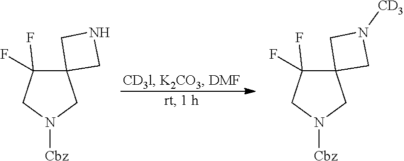 [SMILES: O=C(OCc1ccccc1)N1CC(F)(F)C2(CNC2)C1 ; [2H]C([2H])([2H])N1CC2(CN(C(=O)OCc3ccccc3)CC2(F)F)C1]

Iodomethane-d₃ (64.2 mg, 0.44 mmol) was slowly added to a mixture of benzyl 8,8-difluoro-2,6-diazaspiro [3.4]octane-6-carboxylate (250 mg, 0.89 mmol) and K₂CO₃ (245 mg, 1.77 mmol) in DMF (3 mL). The resulting mixture was stirred for 1 hour at room temperature, quenched with water (8 mL) and extracted with DCM (3×8 mL). The combined organic layers were washed with brine (15 mL), dried over anhydrous Na₂SO₄, filtered and the filtrate was concentrated under vacuum. The residue was purified by Prep-TLC (PE/EA, 1:1) to afford the desired product benzyl 8,8-difluoro-2-(methyl-d₃)-2,6-diazaspiro[3.4]octane-6-carboxylate (90 mg, 33.9% yield).

LCMS (ESI-MS) m/z=300.3 [M+H]⁺.

### Step 4: 8,8-difluoro-2-(methyl-d₃)-2,6-diazaspiro[3.4]octane

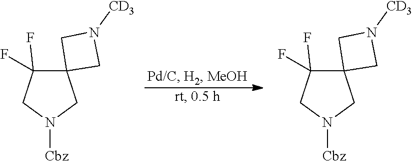 [SMILES: [2H]C([2H])([2H])N1CC2(CN(C(=O)OCc3ccccc3)CC2(F)F)C1 ; [2H]C([2H])([2H])N1CC2(CN(C(=O)OCc3ccccc3)CC2(F)F)C1]

Pd/C (10% on carbon, 10 mg) was added to the mixture of benzyl 8,8-difluoro-2-(methyl-d₃)-2,6-diazaspiro[3.4]octane-6-carboxylate (70 mg, 0.23 mmol) in MeOH (4 mL) under a nitrogen atmosphere. The resulting mixture was stirred for 0.5 hour at room temperature under a hydrogen atmosphere. The reaction mixture was filtered, the filter cake was washed with MeOH (20 mL). The filtrate was concentrated under reduced pressure to afford the crude product 8,8-difluoro-2-(methyl-d₃)-2,6-diazaspiro[3.4]octane (60 mg) as a colorless oil. The crude product was used for the next step without further purification.

LCMS (ESI-MS) m/z=166.2 [M+H]⁺.

### Step 5: 8-(8,8-difluoro-2-(methyl-d3)-2,6-diazaspiro[3.4]octan-6-yl)-6-methyl-N-(1-(methylsulfonyl)piperidin-4-yl)pyrido [3,4-d]pyrimidin-2-amine

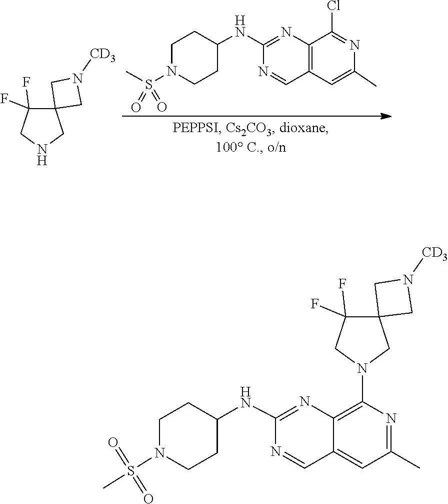

Pd-PEPPSI-iHeptCl 3-chloropyridine (13.7 mg, 0.014 mmol) was added to a mixture of 8-chloro-6-methyl-N-(1-(methylsulfonyl)piperidin-4-yl)pyrido[3,4-d]pyrimidin-2-amine (50 mg, 0.141 mmol), 8,8-difluoro-2-(methyl-d3)-2,6-diazaspiro[3.4]octane (23.2 mg, 0.141 mmol) and Cs₂CO₃ (91.6 mg, 0.28 mmol) in 1,4-dioxane (1 mL) under a nitrogen atmosphere. The resulting mixture was heated to 100° C. and stirred overnight under a nitrogen atmosphere. After cooling to room temperature, the resulting mixture was filtered and the filter cake was washed with DCM (10 mL). The filtrate was concentrated under reduced pressure. The residue was purified by Prep-TLC (DCM/MeOH, 10:1). The product was further purified by preparative RP-HPLC to afford the desired product 8-(8,8-difluoro-2-(methyl-d3)-2,6-diazaspiro[3.4]octan-6-yl)-6-methyl-N-(1-(methylsulfonyl) piperidin-4-yl)pyrido [3,4-d]pyrimidin-2-amine (2.7 mg, 3.79% yield).

¹H NMR (400 MHz, DMSO-d₆) δ 8.98 (s, 1H), 7.54 (s, 1H), 6.78 (s, 1H), 4.40-4.22 (m, 4H), 3.90-3.79 (m, 1H), 3.61 (d, J=11.6 Hz, 3H), 3.26-3.15 (m, 3H), 2.97-2.84 (m, 5H), 2.34 (s, 3H), 2.13-1.98 (m, 2H), 1.69-1.55 (m, 2H).

LCMS (ESI-MS) m/z=485.3 [M+H]⁺.

### Example 39: N-(6-fluoro-2-(methylsulfonyl)isoindolin-5-yl)-6-methyl-8-(2,6-diazaspiro [3.4]octan-2-yl)pyrido[3,4-d]pyrimidin-2-amine

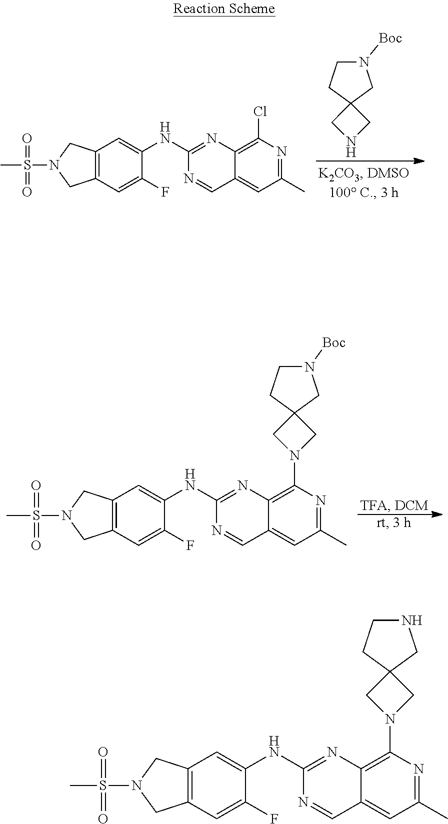

### Detailed Procedure

### Step 1: Tert-butyl 2-(2-((6-fluoro-2-(methylsulfonyl)isoindolin-5-yl)amino)-6-methylpyrido[3,4-d]pyrimidin-8-yl)-2,6-diazaspiro[3.4]octane-6-carboxylate

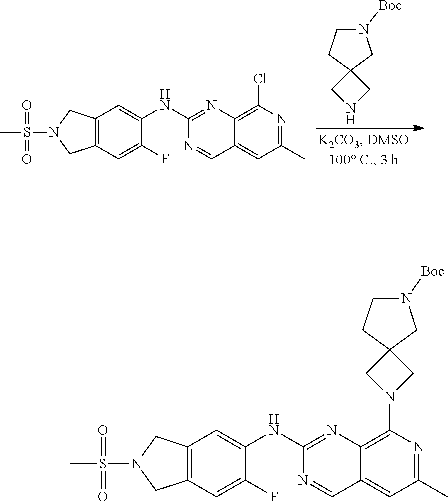

To a solution of 8-chloro-N-(6-fluoro-2-(methylsulfonyl)isoindolin-5-yl)-6-methylpyrido[3,4-d]pyrimidin-2-amine (65 mg, 0.16 mmol) in dimethyl sulfoxide (5 mL) was added tert-butyl 2,6-diazaspiro[3.4]octane-6-carboxylate (67.67 mg, 0.31 mmol) and K₂CO₃(66.56 mg, 0.47 mmol). The resulting mixture was stirred for 3 h at 100° C. under a nitrogen atmosphere. The mixture was allowed to cool down to room temperature, diluted with EA (200 mL) and washed with a saturated aq. sodium chloride solution (3×200 mL). The combined organic layers were dried over anhydrous Na₂SO₄. After filtration, the filtrate was concentrated under reduced pressure to give tert-butyl 2-(2-((6-fluoro-2-(methylsulfonyl)isoindolin-5-yl)amino)-6-methylpyrido[3,4-d]pyrimidin-8-yl)-2,6-diazaspiro[3.4]octane-6-carboxylate (60 mg, 64.5% yield).

LCMS (ESI-MS) m/z=584.2 [M+H]⁺.

### Step 2: N-(6-fluoro-2-(methylsulfonyl)isoindolin-5-yl)-6-methyl-8-(2,6-diazaspiro [3.4]octan-2-yl)pyrido[3,4-d]pyrimidin-2-amine

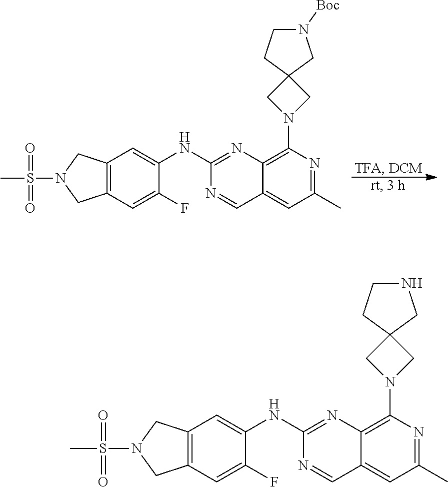

To a solution of tert-butyl 2-(2-((6-fluoro-2-(methylsulfonyl)isoindolin-5-yl)amino)-6-methylpyrido[3,4-d]pyrimidin-8-yl)-2,6-diazaspiro[3.4]octane-6-carboxylate (55 mg, 0.09 mmol) in DCM (2 mL) was added TFA (0.2 mL). The resulting mixture was stirred for 3 h at room temperature under a nitrogen atmosphere. The crude product (55 mg) was purified by preparative RP-HPLC to afford N-(6-fluoro-2-(methylsulfonyl)isoindolin-5-yl)-6-methyl-8-(2,6-diazaspiro [3,4]octan-2-yl)pyrido[3,4-d]pyrimidin-2-amine (17.8 mg, 37.70% yield).

¹H NMR (400 MHz, DMSO-d₆) δ 9.38 (s, 1H), 9.18-9.09 (m, 1H), 7.82-7.55 (m, 1H), 7.32-7.21 (m, 1H), 6.81-6.75 (m, 1H), 4.70-4.60 (m, 5H), 4.15 (s, 4H), 3.10-2.98 (m, 5H), 2.95-2.88 (m, 2H), 2.35 (s, 3H), 2.01-1.95 (m, 2H).

LCMS (ESI-MS) m/z=484.2 [M+H]⁺

### Example 25: N-(1-(cyclopropylsulfonyl)piperidin-4-yl)-6-methyl-8-(2,6-diazaspiro[3.4]octan-2-yl)pyrido[3,4-d]pyrimidin-2-amine

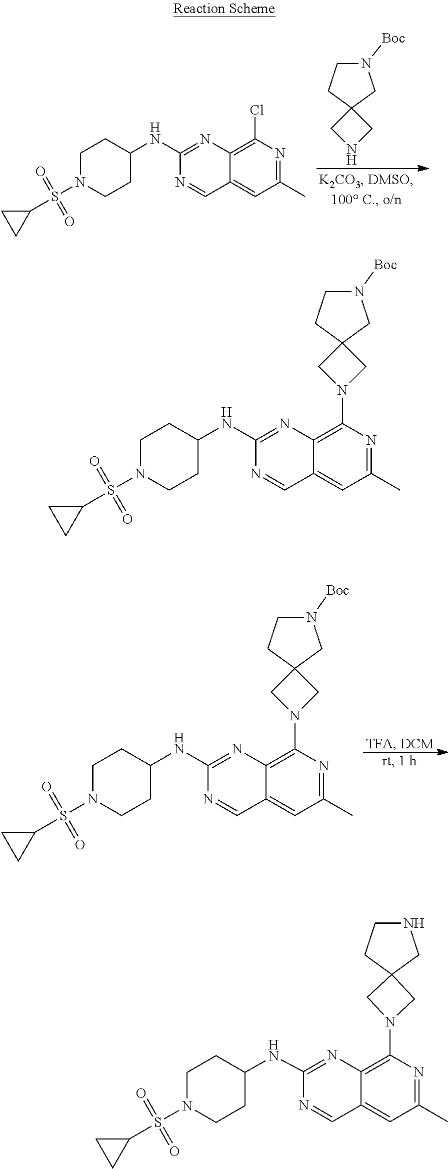 [SMILES: Cc1cc2cnc(NC3CCN(S(=O)(=O)C4CC4)CC3)nc2c(Cl)n1 ; CC(C)(C)OC(=O)N1CCC2(CNC2)C1 ; Cc1cc2cnc(NC3CCN(S(=O)(=O)C4CC4)CC3)nc2c(N2CC3(CCN(C(=O)OC(C)(C)C)C3)C2)n1 ; Cc1cc2cnc(NC3CCN(S(=O)(=O)C4CC4)CC3)nc2c(N2CC3(CCN(C(=O)OC(C)(C)C)C3)C2)n1 ; Cc1cc2cnc(NC3CCN(S(=O)(=O)C4CC4)CC3)nc2c(N2CC3(CCNC3)C2)n1]

### Step 1: Tert-butyl 2-(2-((1-(cyclopropylsulfonyl)piperidin-4-yl)amino)-6-methylpyrido[3,4-d]pyrimidin-8-yl)-2,6-diazaspiro[3.4]octane-6-carboxylate

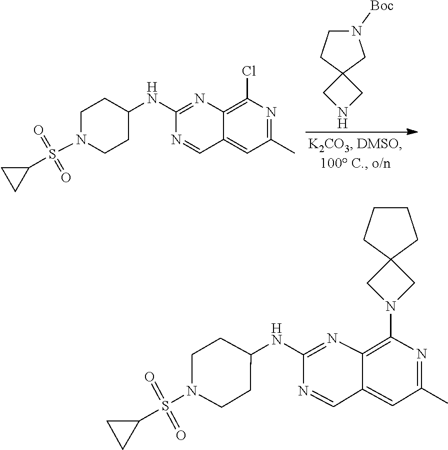 [SMILES: Cc1cc2cnc(NC3CCN(S(=O)(=O)C4CC4)CC3)nc2c(Cl)n1 ; CC(C)(C)OC(=O)N1CCC2(CNC2)C1 ; Cc1cc2cnc(NC3CCN(S(=O)(=O)C4CC4)CC3)nc2c(N2CC3(CCCC3)C2)n1]

A solution of 8-chloro-N-(1-(cyclopropylsulfonyl)piperidin-4-yl)-6-methylpyrido[3,4-d]pyrimidin-2-amine (100 mg, 0.26 mmol), tert-butyl 2,6-diazaspiro[3.4]octane-6-carboxylate (55.59 mg, 0.26 mmol) and K₂CO₃ (108.57 mg, 0.78 mmol) in DMSO (1 mL) was stirred overnight at 100. The resulting mixture was diluted with water (20 mL) and extracted with CH₂Cl₂ (3×20 mL). The combined organic layers were dried over anhydrous Na₂SO₄. After filtration, the filtrate was concentrated under reduced pressure. The residue was purified by Prep-TLC (DCM/MeOH, 10:1) to afford tert-butyl 2-(2-((1-(cyclopropylsulfonyl) piperidin-4-yl)amino)-6-methylpyrido[3,4-d]pyrimidin-8-yl)-2,6-diazaspiro[3.4]octane-6-carboxylate (130 mg, 80.11% yield).

LCMS (ESI-MS) m/z=558.3 [M+H]⁺.

### Step 2: N-(1-(cyclopropylsulfonyl)piperidin-4-yl)-6-methyl-8-(2,6-diazaspiro[3.4]octan-2-yl)pyrido[3,4-d]pyrimidin-2-amine

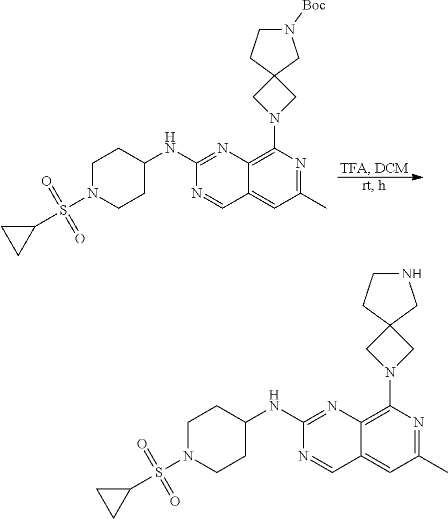 [SMILES: Cc1cc2cnc(NC3CCN(S(=O)(=O)C4CC4)CC3)nc2c(N2CC3(CCN(C(=O)OC(C)(C)C)C3)C2)n1 ; Cc1cc2cnc(NC3CCN(S(=O)(=O)C4CC4)CC3)nc2c(N2CC3(CCNC3)C2)n1]

A solution of tert-butyl 2-(2-((1-(cyclopropylsulfonyl) piperidin-4-yl)amino)-6-methylpyrido[3,4-d] pyrimidin-8-yl)-2,6-diazaspiro[3.4]octane-6-carboxylate (120 mg, 0.21 mmol) in TFA (1 mL) and DCM (3 mL) was stirred for 1 h at room temperature. The reaction mixture was concentrated and purified by Prep-TLC (EA). The product was further purified by preparative RP-HPLC to afford N-(1-(cyclopropylsulfonyl)piperidin-4-yl)-6-methyl-8-(2,6-diazaspiro[3.4]octan-2-yl)pyrido[3,4-d]pyrimidin-2-amine (50.1 mg, 49.56% yield).

¹H NMR (400 MHz, DMSO-d₆) δ 8.93 (d, J=2.7 Hz, 1H), 7.45 (s, 1H), 6.67 (d, J=6.0 Hz, 1H), 4.25 (s, 4H), 3.81 (s, 1H), 3.63 (d, J=12.1 Hz, 2H), 3.47 (s, 1H), 3.07-2.78 (m, 5H), 2.68-2.54 (m, 2H), 2.30 (s, 3H), 2.17-2.07 (m, 1H), 2.06-1.92 (m, 3H), 1.66-1.48 (m, 2H), 1.07-0.86 (m, 4H).

LCMS (ESI-MS) m/z=458.3 [M+H]⁺.

### Example 46: 1-(cyclopropanesulfonyl)-N-(8-{2,6-diazaspiro[3.41octan-2-yl}-6-(difluoromethyl)pyrido[3,4-d]pyrimidin-2-yl)piperidin-4-amine

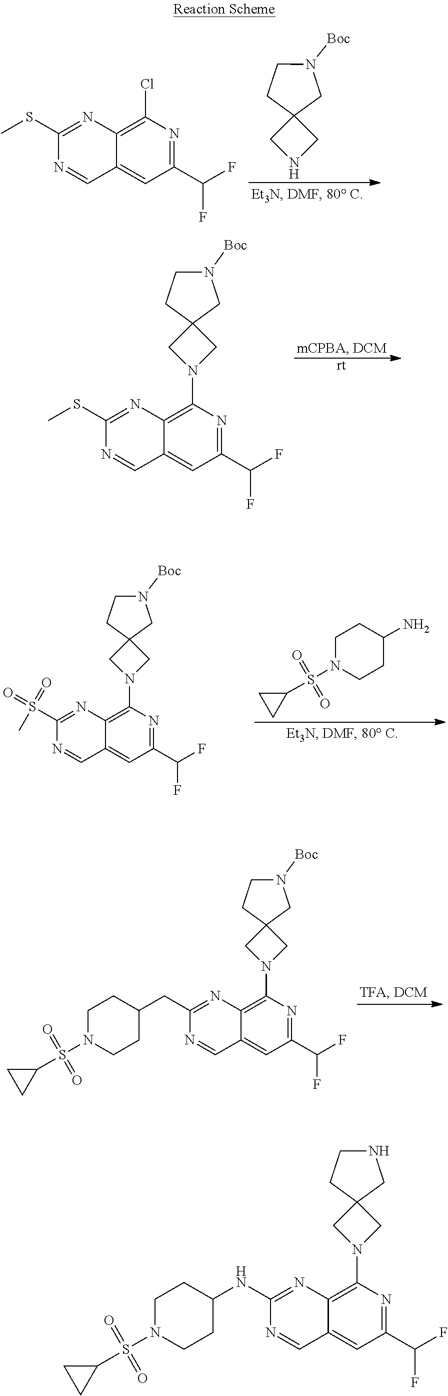

### Step 1: Tert-butyl 2-[6-(difluoromethyl)-2-(methylsulfanyl)pyrido[3,4-d]pyrimidin-8-yl]-2,6-diazaspiro[3.4]octane-6-carboxylate

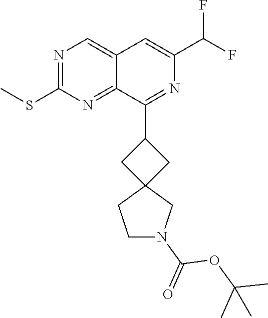 [SMILES: CSc1ncc2cc(C(F)F)nc(C3CC4(CCN(C(=O)OC(C)(C)C)C4)C3)c2n1]

A solution of tert-butyl 2,6-diazaspiro[3.4]octane-6-carboxylate (0.225 g, 1.059 mmol) and triethylamine anhydrous (0.738 ml, 5.293 mmol) in dimethylformamide (5.54 ml) was stirred at room temperature for 5 minutes. Then, 8-chloro-6-(difluoromethyl)-2-(methylsulfanyl)pyrido[3,4-d]pyrimidine (0.277 g, 1.059 mmol) was added and the reaction mixture was stirred at 80° C. for 18 h. The reaction mixture was diluted with water (10 mL) and dichloromethane (10 mL) and the layers were separated. The aqueous layer was extracted with dichloromethane (10 mL×3) and the combined organic layers were washed with K₂CO₃ sat. aq. solution, dried over anhydrous MgSO4, filtered and concentrated under reduced pressure. The crude material was purified by flash column chromatography on silica gel (from hexane to hexane/EtOAc 6:4) to provide the title compound (0.389 g, 84% yield).

¹H NMR (300 MHz, Chloroform-d) δ 9.07 (s, 1H), 7.14 (s, 1H), 6.53 (t, J=55.8 Hz, 1H), 3.68-3.56 (m, 2H), 3.56-3.35 (m, 2H), 2.66 (s, 3H), 2.19 (t, J=7.1 Hz, 2H), 1.58 (s, 4H), 1.50 (s, 9H).

UPLC (ESI) [M+H]⁺=438.45.

### Step 2: Tert-butyl 2-[6-(difluoromethyl)-2-methanesulfonylpyrido[3,4-d]pyrimidin-8-yl]-2,6-diazaspiro[3.4]octane-6-carboxylate

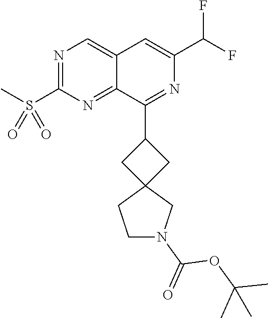 [SMILES: CC(C)(C)OC(=O)N1CCC2(CC(c3nc(C(F)F)cc4cnc(S(C)(=O)=O)nc34)C2)C1]

To a solution of tert-butyl 2-[6-(difluoromethyl)-2-(methylsulfanyl)pyrido[3,4-d]pyrimidin-8-yl]-2,6-diazaspiro[3.4]octane-6-carboxylate (0.389 g, 0.890 mmol) in dichloromethane (11.68 ml), was added portionwise 3-chloroperbenzoic acid (0.768 g, 4.450 mmol). The reaction mixture was stirred at room temperature for 90 minutes, quenched by the dropwise addition of a 10% Na₂S₂O₃ aq. solution (10 mL) and extracted with dichloromethane (10 mL×3). The combined organic layers were washed with NaHCO₃ sat. aq. solution, dried over anhydrous MgSO4, filtered and concentrated under reduced pressure. The crude material was purified by flash column chromatography on silica gel (from hexane/EtOAc 9:1 to hexane/EtOAc 4:6) to provide the title compound (0.326 g, 74%).

¹H NMR (300 MHz, Chloroform-d) δ 9.42 (s, 1H), 7.27 (s, 1H), 6.55 (t, J=55.4 Hz, 1H), 3.63 (s, 2H), 3.57-3.43 (m, 2H), 3.42 (s, 3H), 2.31-2.15 (m, 2H), 1.68-1.57 (m, 3H), 1.57-1.40 (m, 10H).

UPLC (ESI) [M+H]⁺=470.00.

### Step 3: Tert-butyl 2-(2-{[1-(cyclopropanesulfonyl)piperidin-4-yl]amino}-6-(difluoromethyl) pyrido[3,4-d]pyrimidin-8-yl)-2,6-diazaspiro[3.4]octane-6-carboxylate

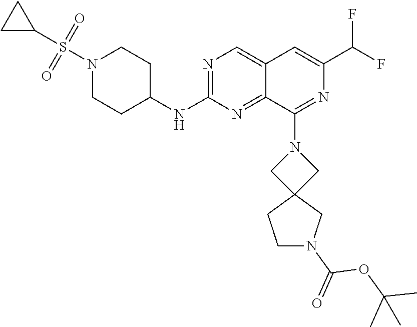 [SMILES: CC(C)(C)OC(=O)N1CCC2(C1)CN(c1nc(C(F)F)cc3cnc(NC4CCN(S(=O)(=O)C5CC5)CC4)nc13)C2]

A solution of 1-(cyclopropanesulfonyl)piperidin-4-amine hydrochloride (0.051 g, 0.202 mmol) and triethylamine anhydrous (0.141 ml, 1.012 mmol) in dimethylformamide (2.0 ml) was stirred at room temperature for 5 minutes. Then, tert-butyl 2-[6-(difluoromethyl)-2-methanesulfonylpyrido[3,4-d]pyrimidin-8-yl]-2,6-diazaspiro[3.4]octane-6-carboxylate (0.1 g, 0.202 mmol) was added and the reaction mixture was stirred at 80° C. for 24 h. The reaction mixture was concentrated under reduced pressure and purified by flash column chromatography on silica gel (from hexane to hexane/EtOAc 4:6) to provide the title compound (0.079 g, 53% yield).

¹H NMR (300 MHz, Chloroform-d) δ 8.92 (s, 1H), 7.09 (s, 1H), 6.53 (t, J=56.1 Hz, 1H), 5.33 (s, 1H), 4.01 (s, 1H), 3.81 (d, J=12.5 Hz, 2H), 3.61 (d, J=9.7 Hz, 2H), 3.47 (s, 2H), 3.09 (t, J=11.3 Hz, 2H), 2.33 (tt, J=8.3, 4.9 Hz, 1H), 2.20 (q, J=9.4, 7.1 Hz, 4H), 1.86-1.54 (m, 6H), 1.50 (s, 9H), 1.26-1.15 (m, 2H), 1.05 (d, J=6.8 Hz, 2H).

UPLC (ESI) [M+H]⁺=594.50.

### Step 4: 1-(cyclopropanesulfonyl)-N-(8-{2,6-diazaspiro[3.4]octan-2-yl}-6-(difluoromethyl) pyrido[3,4-d]pyrimidin-2-yl)piperidin-4-amine

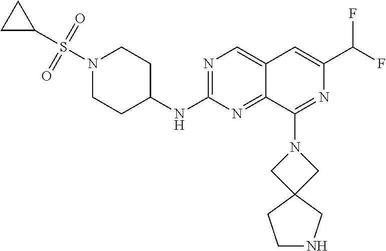 [SMILES: O=S(=O)(C1CC1)N1CCC(Nc2ncc3cc(C(F)F)nc(N4CC5(CCNC5)C4)c3n2)CC1]

A solution of tert-butyl 2-(2-{[1-(cyclopropanesulfonyl) piperidin-4-yl]amino}-6-(difluoromethyl)pyrido[3,4-d]pyrimidin-8-yl)-2,6-diazaspiro[3.4]octane-6-carboxylate (0.079 g, 0.107 mmol) in dichloromethane anhydrous (3.97 ml) was cooled down to 0° C. Then, trifluoroacetic acid (0.79 ml) was added dropwise and the reaction mixture was stirred at 0° C. for 2 h. Volatiles were removed under reduced pressure and the crude material was purified by preparative HPLC (0.1% formic acid) to provide the title compound (0.043 g, 80% yield).

¹H NMR (400 MHz, DMSO-d₆) δ 9.12 (s, 1H), 8.80 (s, 1H), 7.88 (s, 1H), 7.19 (s, 1H), 6.75 (t, J=55.5 Hz, 1H), 4.37 (s, 4H), 3.87 (s, 1H), 3.63 (dd, J=12.5, 4.5 Hz, 2H), 3.36 (s, 2H), 3.22-3.14 (m, 2H), 3.11-3.01 (m, 2H), 2.60 (tt, J=7.8, 4.9 Hz, 1H), 2.21 (t, J=7.2 Hz, 2H), 2.02 (d, J=12.8 Hz, 2H), 1.63 (q, J=11.3 Hz, 2H), 1.01 (dt, J=7.9, 2.8 Hz, 2H), 0.98-0.92 (m, 2H).

LCMS (ESI) [M+H]⁺=494.3.

### Example 51: N-(1-((cyclopentylmethyl)sulfonyl)piperidin-4-yl)-6-methyl-8-(2,6-diazaspiro[3.4]octan-2-yl)pyrido[3,4-d]pyrimidin-2-amine

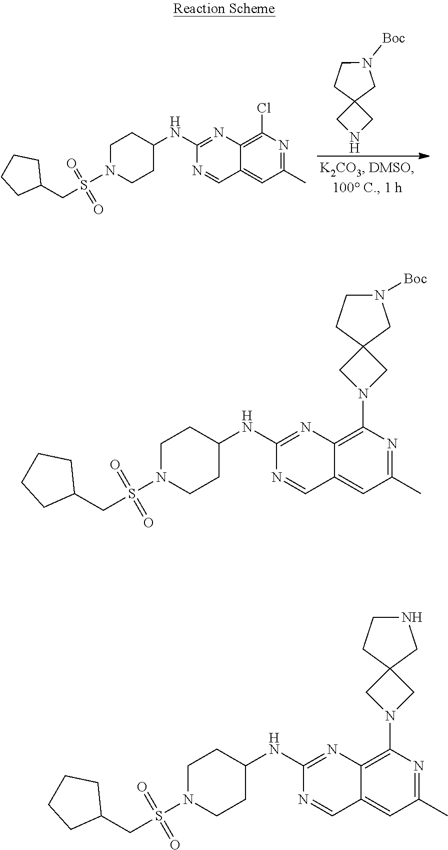 [SMILES: Cc1cc2cnc(NC3CCN(S(=O)(=O)CC4CCCC4)CC3)nc2c(Cl)n1 ; CC(C)(C)OC(=O)N1CCC2(CNC2)C1 ; Cc1cc2cnc(NC3CCN(S(=O)(=O)CC4CCCC4)CC3)nc2c(N2CC3(CCN(C(=O)OC(C)(C)C)C3)C2)n1 ; Cc1cc2cnc(NC3CCN(S(=O)(=O)CC4CCCC4)CC3)nc2c(N2CC3(CCNC3)C2)n1]

### Step 1: Tert-butyl 2-(2-((1-((cyclopentylmethyl)sulfonyl)piperidin-4-yl)amino)-6-methylpyrido[3,4-d] pyrimidin-8-yl)-2,6-diazaspiro[3.4]octane-6-carboxylate

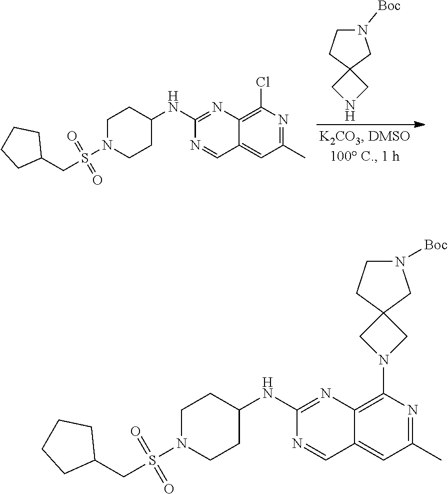 [SMILES: Cc1cc2cnc(NC3CCN(S(=O)(=O)CC4CCCC4)CC3)nc2c(Cl)n1 ; CC(C)(C)OC(=O)N1CCC2(CNC2)C1 ; Cc1cc2cnc(NC3CCN(S(=O)(=O)CC4CCCC4)CC3)nc2c(N2CC3(CCN(C(=O)OC(C)(C)C)C3)C2)n1]

A mixture of 8-chloro-N-(1-((cyclopentylmethyl)sulfonyl)piperidin-4-yl)-6-methylpyrido[3,4-d]pyrimidin-2-amine (300 mg, 0.70 mmol), K₂CO₃ (293.39 mg, 2.12 mmol) and tert-butyl 2,6-diazaspiro[3.4]octane-6-carboxylate (150.22 mg, 0.70 mmol) in DMSO (5 mL) was stirred for 1 h at 100° C. The reaction mixture was diluted with water (250 mL) and extracted with DCM (3×250 mL). The combined organic layers were dried over anhydrous Na₂SO₄, filtered and concentrated under vacuum. The crude product was purified by Prep-TLC (EA) to afford tert-butyl 2-(2-((1-((cyclopentylmethyl)sulfonyl) piperidin-4-yl)amino)-6-methylpyrido[3,4-d]pyrimidin-8-yl)-2,6-diazaspiro[3.4]octane-6-carboxylate (150 mg, 31.81% yield).

LCMS (ESI-MS) m/z=600.3 [M+H]⁺.

### Step 2: N-(1-((cyclopentylmethyl)sulfonyl)piperidin-4-yl)-6-methyl-8-(2,6-diazaspiro[3.4]octan-2-yl) pyrido[3,4-d]pyrimidin-2-amine

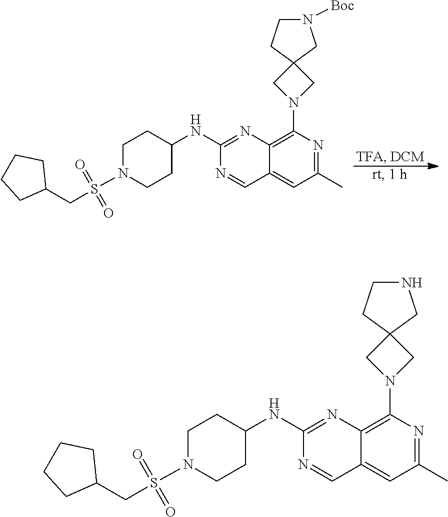 [SMILES: Cc1cc2cnc(NC3CCN(S(=O)(=O)CC4CCCC4)CC3)nc2c(N2CC3(CCN(C(=O)OC(C)(C)C)C3)C2)n1 ; Cc1cc2cnc(NC3CCN(S(=O)(=O)CC4CCCC4)CC3)nc2c(N2CC3(CCNC3)C2)n1]

A mixture of tert-butyl 2-(2-((1-((cyclopentylmethyl)sulfonyl)piperidin-4-yl)amino)-6-methylpyrido[3,4-d]pyrimidin-8-yl)-2,6-diazaspiro[3.4]octane-6-carboxylate (130 mg, 0.21 mmol) and TFA (1 mL) in DCM (3 mL) was stirred for 1 h at room temperature. The residue was purified by Prep-TLC (EA) followed by preparative RP-HPLC to afford N-(1-((cyclopentylmethyl)sulfonyl)piperidin-4-yl)-6-methyl-8-(2,6-diazaspiro[3.4]octan-2-yl)pyrido[3,4-d]pyrimidin-2-amine (39.5 mg, 34.4% yield).

¹H NMR (400 MHz, DMSO-d₆) δ 8.92 (s, 1H), 7.40 (s, 1H), 6.66 (d, J=7.9 Hz, 1H), 4.24 (s, 4H), 3.82 (s, 1H), 3.63-3.55 (m, 2H), 3.06 (d, J=6.9 Hz, 2H), 2.94 (d, J=14.2 Hz, 4H), 2.84-2.77 (m, 2H), 2.30 (s, 3H), 2.26-2.15 (m, 1H), 2.03-1.91 (m, 4H), 1.90-1.81 (m, 2H), 1.67-1.48 (m, 6H), 1.35-1.21 (m, 3H).

LCMS (ESI-MS) m/z=500.3 [M+H]⁺.

### Example 88: 1-cyclopropyl-3-(6-methyl-2-((1-(methylsulfonyl)piperidin-4-yl)amino)pyrido[3,4-d]pyrimidin-8-yl)cyclobutan-1-ol

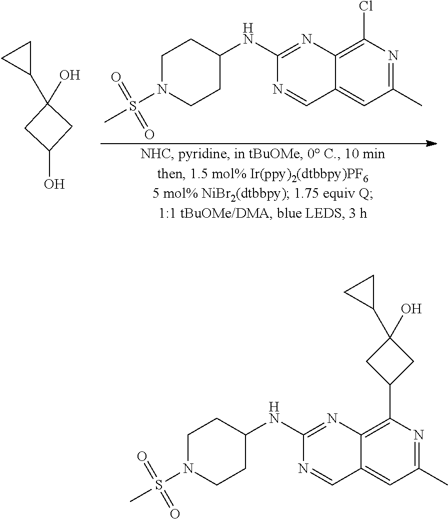

An over-dried 20 mL vial was charged with 1-cyclopropylcyclobutane-1,3-diol (43 mg, 0.49 mmol) and NHC (177 mg, 0.45 mmol). After the vial was vacuumed and refilled with nitrogen atmosphere, METB (4 mL) was added and the reaction stirred at room temperature for 5 minutes. A mixture of pyridine (81 mg, 1.03 mmol) in METB (1 mL) was added and the mixture stirred at room temperature for 10 minutes. Another over-dried 40 mL vial was charged with 8-chloro-6-methyl-N-(1-(methylsulfonyl)piperidin-4-yl)pyrido[3,4-d]pyrimidin-2-amine (100 mg, 0.28 mmol), NHC (177.72 mg, 0.45 mmol), Ir(ppy)₂(dtbbpy)PF₆ (3.85 mg, 0.004 mmol), NiBr₂(dtbbpy) (6.84 mg, 0.014 mmol) and 1-azabicyclo[2.2.2]octane (54.68 mg, 0.49 mmol) in DMA (5 mL) under a nitrogen atmosphere. The mixture was stirred at 800 rpm for 3 h under a 450 nm blue LEDs. The resulting mixture was concentrated under vacuum. The residue was purified by preparative RP-HPLC to afford 1-cyclopropyl-3-(6-methyl-2-((1-(methylsulfonyl)piperidin-4-yl)amino)pyrido[3,4-d]pyrimidin-8-yl)cyclobutan-1-ol (2.1 mg, 1.54%).

¹H NMR (400 MHz, Chloroform-d) δ 8.97-8.95 (m, 2H), 7.29-7.26 (m, 1H), 4.63-4.60 (m, 1H), 4.10-3.83 (m, 3H), 2.99-2.93 (m, 4H), 2.89-2.87 (m, 4H), 2.72-2.70 (m, 3H), 2.54-2.20 (m, 4H), 1.84-1.78 (m, 3H), 0.47-0.41 (m, 4H).

LCMS (ESI-MS) m/z=432.2 [M+H]⁺.

### Example 60: 6-cyclopropyl-8-(8,8-difluoro-2,6-diazaspiro[3.4]octan-6-yl)-N-(1-(methylsulfonyl)piperidin-4-yl)pyrido[3,4-d]pyrimidin-2-amine

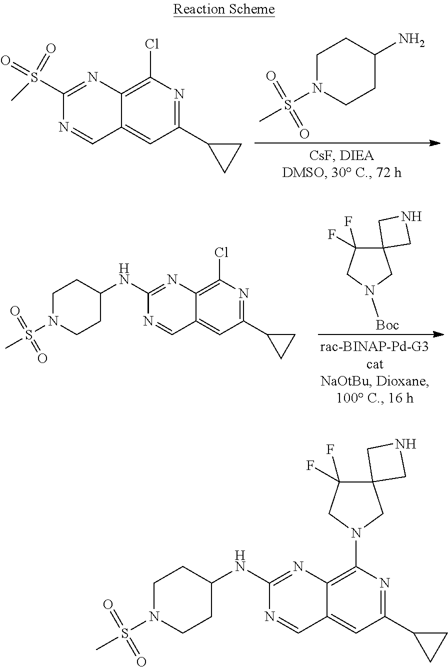

### Detailed Procedure

### Step 1: 8-chloro-6-cyclopropyl-N-(1-(methylsulfonyl)piperidin-4-yl)pyrido[3,4-d]pyrimidin-2-amine

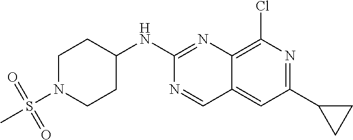 [SMILES: CS(=O)(=O)N1CCC(Nc2ncc3cc(C4CC4)nc(Cl)c3n2)CC1]

A solution of 8-chloro-6-cyclopropyl-2-(methylsulfonyl)pyrido[3,4-d]pyrimidine (100 mg, 352 μmol) in anhydrous dimethylsulfoxide (1.8 mL) was charged with 1-(methylsulfonyl)piperidin-4-amine (69.1 mg, 388 μmol), cesium fluoride (161 mg, 1.06 mmol), and N-ethyl-N-isopropylpropan-2-amine (137 mg, 184 μL, 1.06 mmol). The reaction vial was capped and stirred at 30° C. for 72 h. The crude mixture was diluted with water (50.0 mL) and extracted with dichloromethane (3×20.0 mL). The combined organic layers were washed with brine, dried over anhydrous magnesium sulfate, filtered, and concentrated under reduced pressure. The residue was purified by flash chromatography on silica gel (heptane/EtOAc, 1:1) to afford the title compound (62.0 mg, 46% yield).

LCMS (ESI) [M+H]⁺=382.10

### Step 2: 6-cyclopropyl-8-(8,8-difluoro-2,6-diazaspiro[3.4]octan-6-yl)-N-(1-(methylsulfonyl)piperidin-4-yl)pyrido[3,4-d]pyrimidin-2-amine

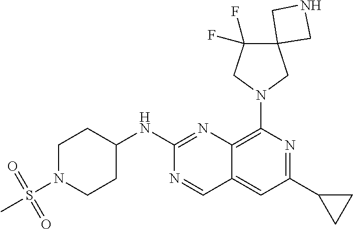 [SMILES: CS(=O)(=O)N1CCC(Nc2ncc3cc(C4CC4)nc(N4CC(F)(F)C5(CNC5)C4)c3n2)CC1]

To a solution of 8-chloro-6-cyclopropyl-N-(1-(methylsulfonyl)piperidin-4-yl)pyrido[3,4-d]pyrimidin-2-amine (22.0 mg, 58.0 μmol) in anhydrous dioxane (290 μL) was added tert-butyl-8,8-difluoro-2,6-diazaspiro[3.4]octane-2-carboxylate (17.0 mg, 69.0 μmol), sodium tert-butoxide (17.0 mg, 170 μmol), and [2′-(amino-κN)[1,1′-biphenyl]-2-yl-κC][[2′-(diphenylphosphino)[1,1′-binaphthalen]-2-yl]diphenylphosphine-κP](methanesulfonato-κO)-palladium (5.7 mg, 5.8 μmol). The reaction vial was capped and stirred at 100° C. for 16 h. The mixture was cooled to rt, diluted with ethyl acetate (30.0 mL) and water (20.0 mL), extracted (3×20.0 mL ethyl acetate), washed with brine, dried over anhydrous MgSO4, filtered, and concentrated to dryness.

To the crude material was added trifluoroacetic acid (800 μL) at 0° C. After 10 minutes, the reaction was allowed to warm to rt and continuously stirred until completion of reaction as determined by LC-MS. Afterwards, the reaction was concentrated to dryness. The resulting solid was dissolved in dichloromethane (10 mL), neutralized with NaHCO₃ sat. aq. solution, and extracted with dichloromethane (3×10.0 mL). The combined organic phases were concentrated to dryness and purified by preparative RP-HPLC to provide the title compound (14.0 mg, 48% yield).

¹H NMR (DMSO-d₆, 499 MHz) δ 8.98 (s, 1H), 7.4-7.6 (m, 1H), 6.8-6.9 (m, 1H), 4.9-5.0 (m, 1H), 4.2-4.4 (m, 4H), 3.80 (d, 2H, J=8.5 Hz), 3.60 (d, 2H, J=12.3 Hz), 3.46 (d, 1H, J=8.5 Hz), 2.8-3.0 (m, 5H), 2.04 (d, 2H, J=12.3 Hz), 1.9-2.0 (m, 2H), 1.5-1.7 (m, 3H), 0.92 (m, 2H), 0.7-0.8 (m, 2H).

LCMS (ESI) [M+H]⁺=494.30

### Example 62: N-(1-(cyclopropylsulfonyl)piperidin-4-yl)-8-(8,8-difluoro-2,6-diazaspiro[3.4]octan-6-yl)-6-methylpyrido[3,4-d]pyrimidin-2-amine

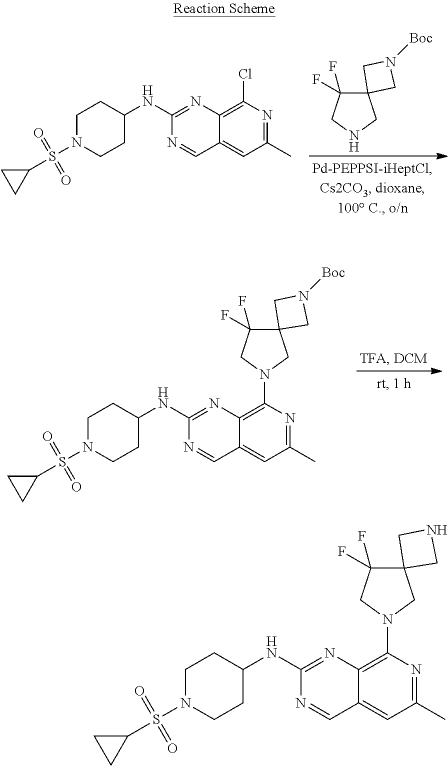 [SMILES: Cc1cc2cnc(NC3CCN(S(=O)(=O)C4CC4)CC3)nc2c(Cl)n1 ; CC(C)(C)OC(=O)N1CC2(CNCC2(F)F)C1 ; Cc1cc2cnc(NC3CCN(S(=O)(=O)C4CC4)CC3)nc2c(N2CC(F)(F)C3(CN(C(=O)OC(C)(C)C)C3)C2)n1 ; Cc1cc2cnc(NC3CCN(S(=O)(=O)C4CC4)CC3)nc2c(N2CC(F)(F)C3(CNC3)C2)n1]

### Detailed Procedure

### Step 1: Tert-butyl 6-(2-((1-(cyclopropylsulfonyl)piperidin-4-yl)amino)-6-methylpyrido[3,4-d]pyrimidin-8-yl)-8,8-difluoro-2,6-diazaspiro[3.4]octane-2-carboxylate

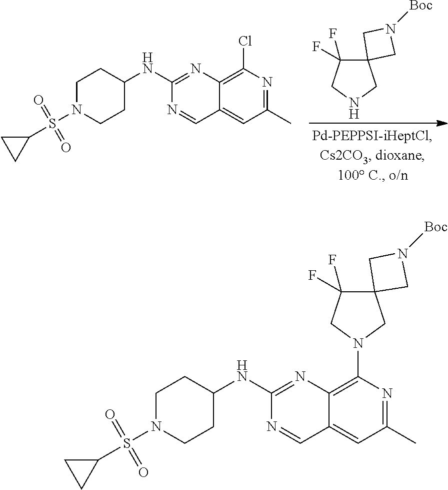 [SMILES: Cc1cc2cnc(NC3CCN(S(=O)(=O)C4CC4)CC3)nc2c(Cl)n1 ; CC(C)(C)OC(=O)N1CC2(CNCC2(F)F)C1 ; Cc1cc2cnc(NC3CCN(S(=O)(=O)C4CC4)CC3)nc2c(N2CC(F)(F)C3(CN(C(=O)OC(C)(C)C)C3)C2)n1]

Pd-PEPPSI-iHeptCl 3-chloropyridine (255 mg, 0.26 mmol) was added to a mixture of 8-chloro-N-(1-(cyclopropylsulfonyl)piperidin-4-yl)-6-methylpyrido[3,4-d]pyrimidin-2-amine (2 g, 5.23 mmol), tert-butyl 8,8-difluoro-2,6-diazaspiro[3.4]octane-2-carboxylate (1.30 g, 5.23 mmol) and Cs₂CO₃ (3.41 g, 10.47 mmol) in dioxane (30 mL) under a nitrogen atmosphere. The resulting mixture was stirred overnight at 100° C. The resulting mixture was filtered and the filter cake was washed with DCM (50 mL). The filtrate was concentrated under reduced pressure. The residue was purified by silica gel column chromatography (CH₂Cl₂/MeOH, 3:97) to afford tert-butyl 6-(2-((1-(cyclopropylsulfonyl)piperidin-4-yl)amino)-6-methylpyrido[3,4-d]pyrimidin-8-yl)-8,8-difluoro-2,6-diazaspiro[3.4]octane-2-carboxylate (2.8 g, 70.60% yield).

LCMS (ESI-MS) m/z=594.3 [M+H]⁺.

### Step 2: N-(1-(cyclopropylsulfonyl)piperidin-4-yl)-8-(8,8-difluoro-2,6-diazaspiro[3.4]octan-6-yl)-6-methylpyrido[3,4-d]pyrimidin-2-amine

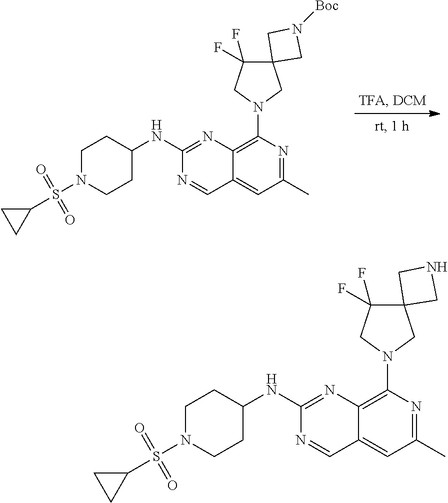 [SMILES: Cc1cc2cnc(NC3CCN(S(=O)(=O)C4CC4)CC3)nc2c(N2CC(F)(F)C3(CN(C(=O)OC(C)(C)C)C3)C2)n1 ; Cc1cc2cnc(NC3CCN(S(=O)(=O)C4CC4)CC3)nc2c(N2CC(F)(F)C3(CNC3)C2)n1]

A solution of tert-butyl 6-(2-((1-(cyclopropylsulfonyl)piperidin-4-yl)amino)-6-methylpyrido[3,4-d]pyrimidin-8-yl)-8,8-difluoro-2,6-diazaspiro[3.4]octane-2-carboxylate (2.6 g, 4.37 mmol) in TFA (5 mL) and DCM (15 mL) was stirred for 1 h at room temperature. The reaction mixture was concentrated and the residue was purified by silica gel column chromatography (CH₂Cl₂/MeOH, 90:10) to afford N-(1-(cyclopropylsulfonyl)piperidin-4-yl)-8-(8,8-difluoro-2,6-diazaspiro[3.4]octan-6-yl)-6-methylpyrido[3,4-d]pyrimidin-2-amine (2.0686 g, 91.97% yield).

¹H NMR (400 MHz, DMSO-d₆) δ 9.20 (s, 1H), 9.00 (s, 1H), 7.60 (s, 1H), 6.82 (s, 1H), 4.58-4.41 (m, 2H), 4.38-4.32 (m, 2H), 4.31-4.21 (m, 2H), 4.19-4.09 (m, 2H), 3.87 (s, 1H), 3.72-3.57 (m, 2H), 3.09-2.97 (m, 2H), 2.63-2.54 (m, 1H), 2.36 (s, 3H), 2.10-1.97 (m, 2H), 1.69-1.52 (m, 2H), 1.07-0.91 (m, 4H).

LCMS (ESI-MS) m/z=494.2 [M+H]⁺.

### Example 89: 8-(8,8-difluoro-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine

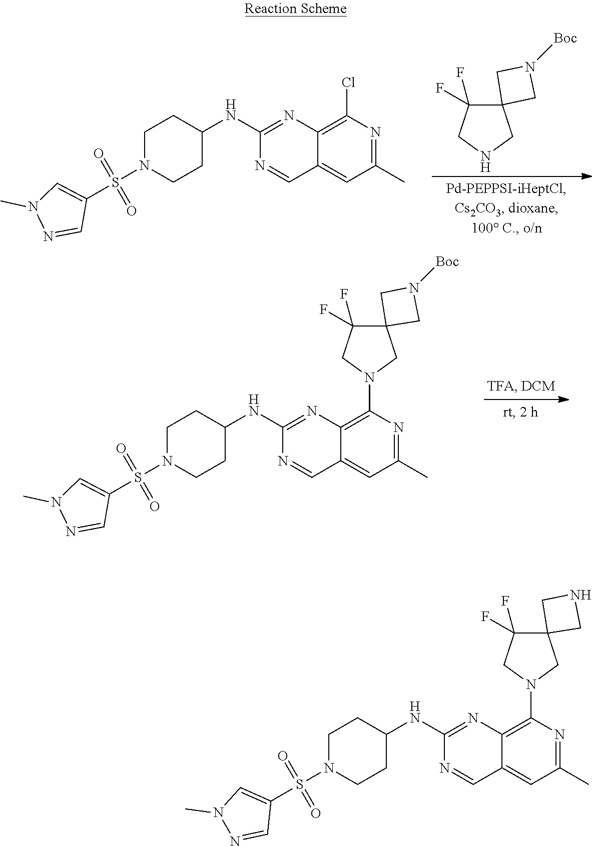

### Step 1: Tert-butyl 8,8-difluoro-6-(6-methyl-2-((1-((1-methyl-1H-pyrazol-4-yl)sulfonyl)piperidin-4-yl)amino)pyrido[3,4-d]pyrimidin-8-yl)-2,6-diazaspiro[3.4]octane-2-carboxylate

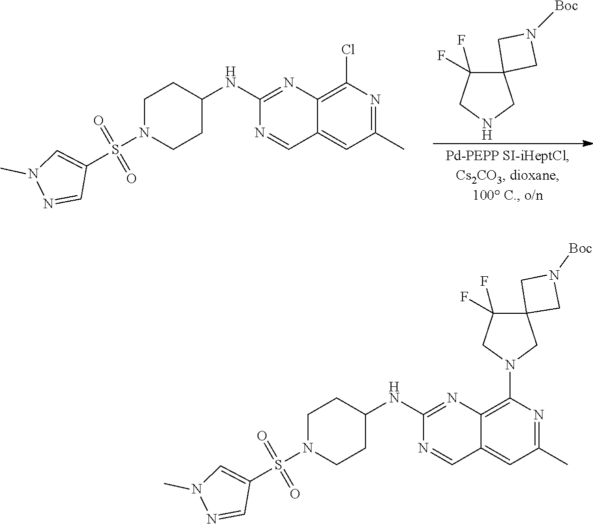

A solution of 8-chloro-6-methyl-N-(1-((1-methyl-1H-pyrazol-4-yl)sulfonyl)piperidin-4-yl)pyrido[3,4-d]pyrimidin-2-amine (500 mg, 1.18 mmol), tert-butyl 8,8-difluoro-2,6-diazaspiro[3.4]octane-2-carboxylate (293.83 mg, 1.18 mmol), Cs₂CO₃ (1158.40 mg, 3.55 mmol) and Pd-PEPPSI-iHeptCl 3-chloropyridine (115.41 mg, 0.11 mmol) in dioxane (10 mL) was stirred overnight at 100° C. under a nitrogen atmosphere. The reaction mixture was diluted with water (100 mL) and extracted with EA (3×100 mL). The combined organic layers were dried over anhydrous Na₂SO₄. After filtration, the filtrate was concentrated under reduced pressure. The residue was purified by silica gel column chromatography (PE/EA, 1:1) to afford tert-butyl 8,8-difluoro-6-(6-methyl-2-((1-((1-methyl-1H-pyrazol-4-yl)sulfonyl)piperidin-4-yl)amino)pyrido[3,4-d] pyrimidin-8-yl)-2,6-diazaspiro[3.4]octane-2-carboxylate (600 mg, 75.9% yield).

LCMS (ESI-MS) m/z=634.3 [M+H]⁺.

### Step 2: 8-(8,8-difluoro-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine

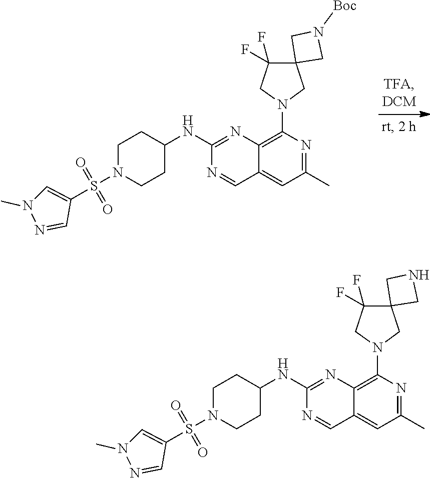

A solution of tert-butyl 8,8-difluoro-6-(6-methyl-2-((1-((1-methyl-1H-pyrazol-4-yl)sulfonyl)piperidin-4-yl)amino)pyrido[3,4-d]pyrimidin-8-yl)-2,6-diazaspiro[3.4]octane-2-carboxylate (3 g, 4.73 mmol) in TFA (5 mL) and DCM (15 mL) was stirred for 2 h at room temperature. The resulting mixture was concentrated under reduced pressure. The residue was purified by silica gel column chromatography (CH₂Cl₂/MeOH, 10:1) to afford the TFA salt of 8-(8,8-difluoro-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine (2.3177 g, 91.7% yield).

¹H NMR (400 MHz, DMSO-d₆) δ 9.09 (s, 2H), 8.98 (s, 1H), 8.34 (s, 1H), 7.78 (s, 1H), 7.56 (s, 1H), 6.81 (s, 1H), 4.42 (s, 2H), 4.29 (s, 2H), 4.22 (d, J=11.6 Hz, 2H), 4.09 (d, J=11.5 Hz, 2H), 3.92 (s, 3H), 3.72 (s, 1H), 3.49 (d, J=11.3 Hz, 2H), 2.53 (s, 2H), 2.35 (s, 3H), 2.02 (d, J=12.2 Hz, 2H), 1.69-1.57 (m, 2H).

LCMS (ESI-MS) m/z=534.2 [M+H]⁺.

### Example 97: 8-(8,8-difluoro-2-methyl-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine

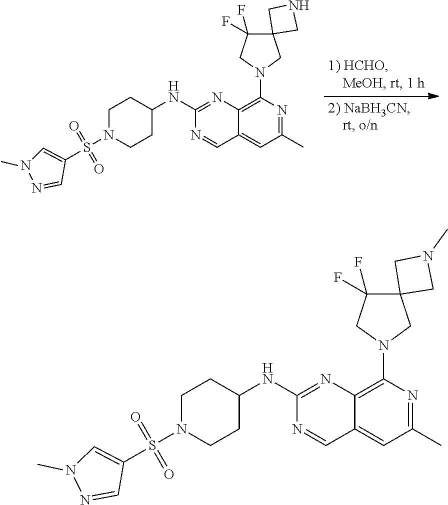

A mixture of 8-(8,8-difluoro-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine (250 mg, 0.46 mmol) and formaldehyde (28.14 mg, 0.93 mmol) in MeOH (10 mL) was stirred for 1 h at room temperature. NaBH₃CN (117.77 mg, 1.87 mmol) was added and the resulting mixture was stirred overnight at room temperature. The mixture was concentrated under reduced pressure and the residue purified by silica gel column chromatography (CH₂Cl₂/MeOH, 10:1) to afford 8-(8,8-difluoro-2-methyl-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine (96.2 mg, 37.1% yield).

¹H NMR (400 MHz, DMSO-d₆) δ 8.96 (s, 1H), 8.34 (s, 1H), 7.77 (d, J=0.6 Hz, 1H), 7.53 (s, 1H), 6.76 (d, J=0.9 Hz, 1H), 4.28 (s, 2H), 4.23 (s, 1H), 3.92 (s, 3H), 3.70 (s, 1H), 3.55 (d, J=11.6 Hz, 2H), 3.39 (d, J=7.8 Hz, 2H), 3.18 (s, 2H), 2.45 (d, J=11.4 Hz, 1H), 2.33 (s, 3H), 2.26 (s, 3H), 2.08 (s, 2H), 2.06-1.99 (m, 2H), 1.69-1.59 (m, 2H).

LCMS (ESI-MS) m/z=548.4 [M+H]⁺.

### Example 69: (1-methyl-3-(6-methyl-2-((1-(methylsulfonyl)piperidin-4-yl)amino)pyrido[3,4-d]pyrimidin-8-yl)cyclobutyl)methanol

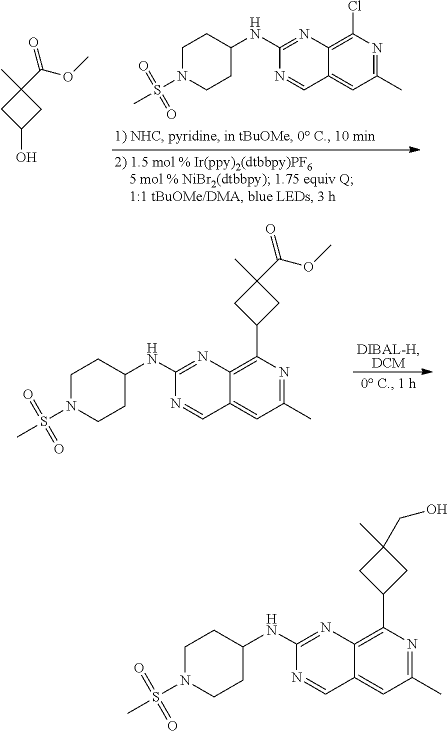

### Step 1: Methyl 1-methyl-3-(6-methyl-2-((1-(methylsulfonyl)piperidin-4-yl)amino)pyrido[3,4-d]pyrimidin-8-yl)cyclobutane-1-carboxylate

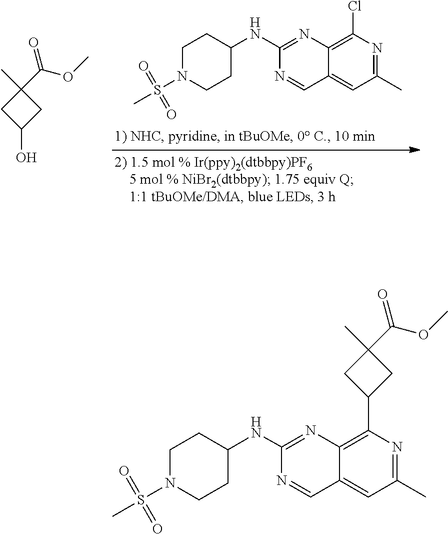

An oven-dried 20 mL vial was charged with methyl 3-hydroxy-1-methylcyclobutane-1-carboxylate (100 mg, 0.69 mmol) and 5,7-di-tert-butyl-3-phenylbenzo[d]oxazol-3-ium tetrafluoroborate (250 mg, 0.63 mmol). Under nitrogen, tert-butyl methyl ether (4 mL) was added and the reaction stirred at room temperature for 5 minutes. A mixture of pyridine (50.16 mg, 0.63 mmol) in tert-butyl methyl ether (1 mL) was added and the mixture stirred at room temperature for 10 minutes (mixture A). Another oven-dried 40 mL vial was charged with 8-chloro-6-methyl-N-(1-(methylsulfonyl)piperidin-4-yl)pyrido[3,4-d]pyrimidin-2-amine (141.04 mg, 0.39 mmol), NiBr₂(dtbbpy) (14.66 mg, 0.03 mmol), Ir(ppy)₂(dtbbpy)PF₆ (5.43 mg, 0.006 mmol), phatalamide (9.65 mg, 0.02 mmol) and 1-azabicyclo[2.2.2]octane (77.1 mg, 0.69 mmol). DMA (5 mL) was added under nitrogen (mixture B). The mixture A was added to the mixture B under a nitrogen atmosphere and the resulting mixture was stirred and irradiated with a 450 nm LED lamp under a fan for 3 h. The residue was dissolved in water (20 mL) and extracted with EA (3×20 mL). The combined organic layers were dried over anhydrous Na₂SO₄. After filtration, the filtrate was concentrated under reduced pressure. The residue was purified by Prep-TLC (PE/EA, 1:2) to afford crude methyl 1-methyl-3-(6-methyl-2-((1-(methylsulfonyl)piperidin-4-yl)amino)pyrido[3,4-d]pyrimidin-8-yl)cyclobutane-1-carboxylate (90 mg).

LCMS (ESI-MS) m/z=448.2 [M+H]⁺.

### Step 2: (1-methyl-3-(6-methyl-2-((1-(methylsulfonyl)piperidin-4-yl)amino)pyrido[3,4-d]pyrimidin-8-yl)cyclobutyl)methanol

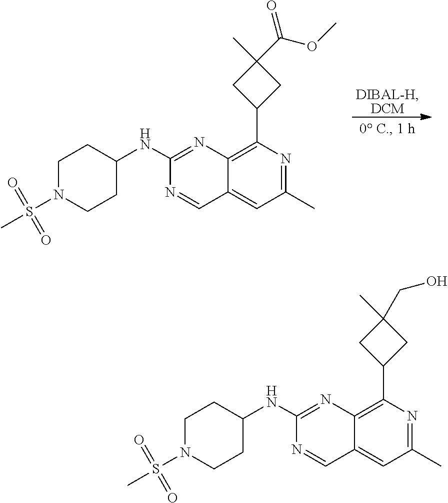

DIBAL-H (47.67 mg, 0.336 mmol) was added to a solution of methyl 1-methyl-3-(6-methyl-2-((1-(methylsulfonyl)piperidin-4-yl)amino)pyrido[3,4-d]pyrimidin-8-yl)cyclobutane-1-carboxylate (50 mg, 0.112 mmol) in DCM (1 mL) at 0° C. The resulting mixture was stirred for 1 h at 0° C. and quenched with water (20 mL) at 0° C. The resulting mixture was extracted with DCM (3×20 mL) and dried over anhydrous Na₂SO₄. After filtration, the filtrate was concentrated under reduced pressure. The residue was purified by Prep-TLC (EA=100%) to afford (1-methyl-3-(6-methyl-2-((1-(methylsulfonyl)piperidin-4-yl)amino)pyrido[3,4-d]pyrimidin-8-yl)cyclobutyl)methanol (1.1 mg, 2.33% yield).

¹H NMR (400 MHz, CDCl3) δ 8.95 (s, 1H), 7.22-7.11 (m, 1H), 5.31 (s, 1H), 4.65-4.51 (m, 1H), 4.41-4.26 (m, 1H), 4.18-3.99 (m, 1H), 3.87-3.68 (m, 3H), 3.52 (s, 1H), 3.11-2.93 (m, 2H), 2.93-2.78 (m, 4H), 2.76-2.68 (m, 2H), 2.44-2.16 (m, 5H), 1.87-1.60 (m, 5H), 1.19 (s, 1H).

LCMS (ESI-MS) m/z=420.2 [M+H]⁺.

### Example 70: 6-cyclopropyl-N-(1-(methylsulfonyl)piperidin-4-yl)-8-(5-oxa-8-azaspiro[3.5]nonan-2-yl)pyrido[3,4-d]pyrimidin-2-amine

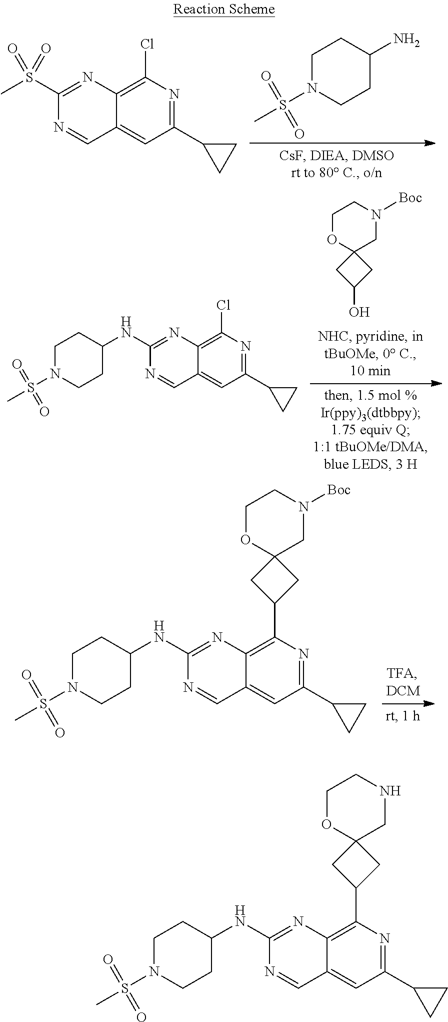

### Detailed Procedure

### Step 1: 8-chloro-6-cyclopropyl-N-(1-(methylsulfonyl)piperidin-4-yl)pyrido[3,4-d]pyrimidin-2-amine

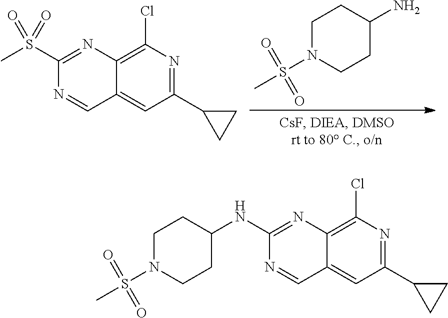

DIEA (137 mg, 1.05 mmol) was added to a mixture of 8-chloro-6-cyclopropyl-2-(methylsulfonyl)pyrido[3,4-d]pyrimidine (100 mg, 0.35 mmol), 1-(methylsulfonyl)piperidin-4-amine (62.8 mg, 0.35 mmol) and CsF (161 mg, 1.05 mmol) in DMSO (2 mL) at room temperature. The resulting mixture was stirred overnight at 80° C. The residue was dissolved in water (20 mL) and extracted with EA (3×20 mL). The combined organic layers were washed with brine (3×20 mL), dried over anhydrous Na₂SO₄. After filtration, the filtrate was concentrated under reduced pressure. The residue was purified by Prep-TLC (PE/EA, 1:2) to afford crude 8-chloro-6-cyclopropyl-N-(1-(methylsulfonyl)piperidin-4-yl)pyrido[3,4-d]pyrimidin-2-amine (80 mg).

LCMS (ESI-MS) m/z=382.2 [M+H]⁺.

### Step 2: Tert-butyl 2-(6-cyclopropyl-2-((1-(methylsulfonyl)piperidin-4-yl)amino)pyrido[3,4-d]pyrimidin-8-yl)-5-oxa-8-azaspiro[3.5]nonane-8-carboxylate

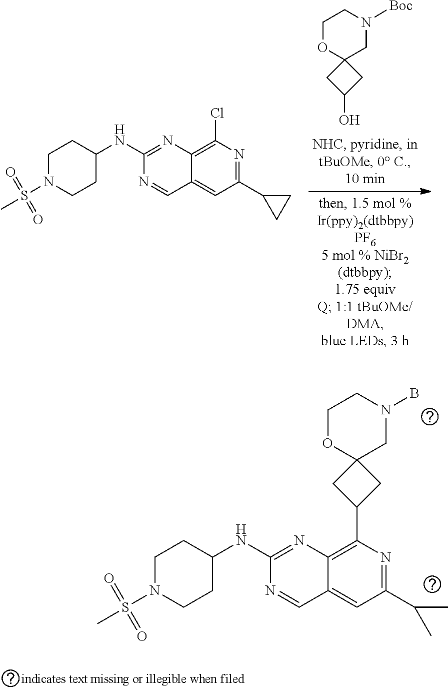

An oven-dried 20 mL vial was charged with tert-butyl 2-hydroxy-5-oxa-8-azaspiro[3.5]nonane-8-carboxylate (55.7 mg, 0.229 mmol) and 5,7-di-tert-butyl-3-phenylbenzo[d]oxazol-3-ium tetrafluoroborate (82.8 mg, 0.21 mmol). Under nitrogen, tert-butyl methyl ether (4 mL) was added and the reaction stirred at room temperature for 5 minutes. A mixture of pyridine (16.57 mg, 0.21 mmol) in tert-butyl methyl ether (1 mL) was added and the mixture stirred at room temperature for 10 minutes (mixture A). Another oven-dried 40 mL vial was charged with 8-chloro-6-cyclopropyl-N-(1-(methylsulfonyl)piperidin-4-yl)pyrido[3,4-d]pyrimidin-2-amine (50 mg, 0.13 mmol), NiBr₂(dtbbpy) (4.88 mg, 0.01 mmol), Ir(ppy)₂(dtbbpy)PF₆ (1.79 mg, 0.002 mmol), 1-azabicyclo[2.2.2]octane (25.89 mg, 0.23 mmol) and quinuclidone (25.48 mg, 0.229 mmol). DMA (5 mL) was added under nitrogen (mixture B). The mixture A was added to the mixture B under a nitrogen atmosphere and the resulting mixture was stirred and irradiated with a 450 nm LED lamp under a fan for 3 h. The residue was dissolved in water (20 mL) and extracted with EA (3×20 mL). The combined organic layers were dried over anhydrous Na₂SO₄. After filtration, the filtrate was concentrated under reduced pressure. The residue was purified by Prep-TLC (PE/EA, 1:2) to afford crude tert-butyl 2-(6-cyclopropyl-2-((1-(methylsulfonyl)piperidin-4-yl)amino)pyrido[3,4-d]pyrimidin-8-yl)-5-oxa-8-azaspiro[3.5]nonane-8-carboxylate (50 mg).

LCMS (ESI-MS) m/z=573.2 [M+H]⁺.

### Step 3: 6-cyclopropyl-N-(1-(methylsulfonyl)piperidin-4-yl)-8-(5-oxa-8-azaspiro[3.5]nonan-2-yl)pyrido[3,4-d]pyrimidin-2-amine

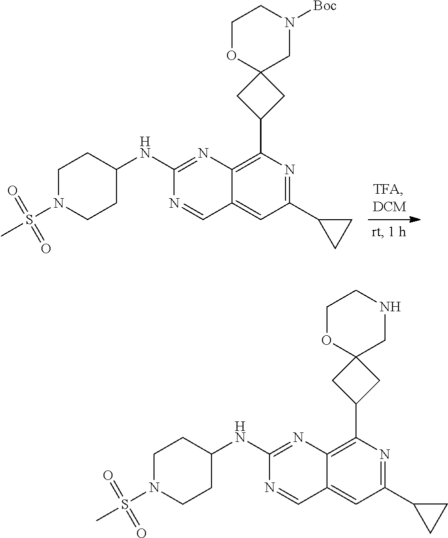

TFA (0.1 mL) was added to a solution of tert-butyl 2-(6-cyclopropyl-2-((1-(methylsulfonyl)piperidin-4-yl)amino)pyrido[3,4-d]pyrimidin-8-yl)-5-oxa-8-azaspiro[3.5]nonane-8-carboxylate (50 mg, 0.087 mmol) in DCM (2 mL). The resulting mixture was stirred for 1 h at room temperature. The reaction mixture was concentrated under reduced pressure and the residue was purified by preparative RP-HPLC to afford 6-cyclopropyl-N-(1-(methylsulfonyl)piperidin-4-yl)-8-(5-oxa-8-azaspiro[3.5]nonan-2-yl)pyrido[3,4-d]pyrimidin-2-amine (1.0 mg, 2.30% yield).

¹H NMR (400 MHz, CDCl₃) δ 8.93 (s, 1H), 7.16 (s, 1H), 5.30-5.20 (m, 1H), 4.12-3.91 (m, 2H), 3.87-3.67 (m, 4H), 3.19-2.98 (m, 4H), 2.96-2.80 (m, 5H), 2.61-2.49 (m, 4H), 2.34-2.20 (m, 3H), 2.17-2.00 (m, 1H), 1.83-1.65 (m, 2H), 1.15-1.05 (m, 2H), 1.02-0.93 (m, 2H).

LCMS (ESI-MS) m/z=473.2[M+H]⁺.

### Example 93: 1-(difluoromethyl)-3-(6-methyl-2-((1-(methylsulfonyl)piperidin-4-yl)amino) pyrido[3,4-d]pyrimidin-8-yl)cyclobutan-1-ol

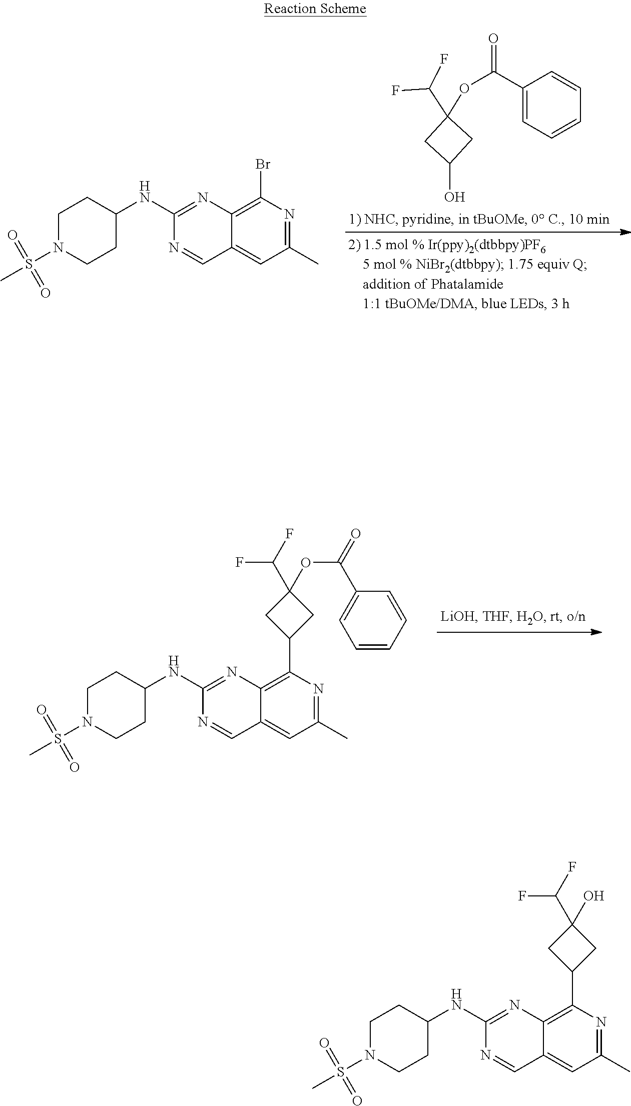

### Detailed Procedure

### Step 1: 1-(difluoromethyl)-3-(6-methyl-2-((1-(methylsulfonyl)piperidin-4-yl)amino)pyrido[3,4-d]pyrimidin-8-yl)cyclobutyl benzoate

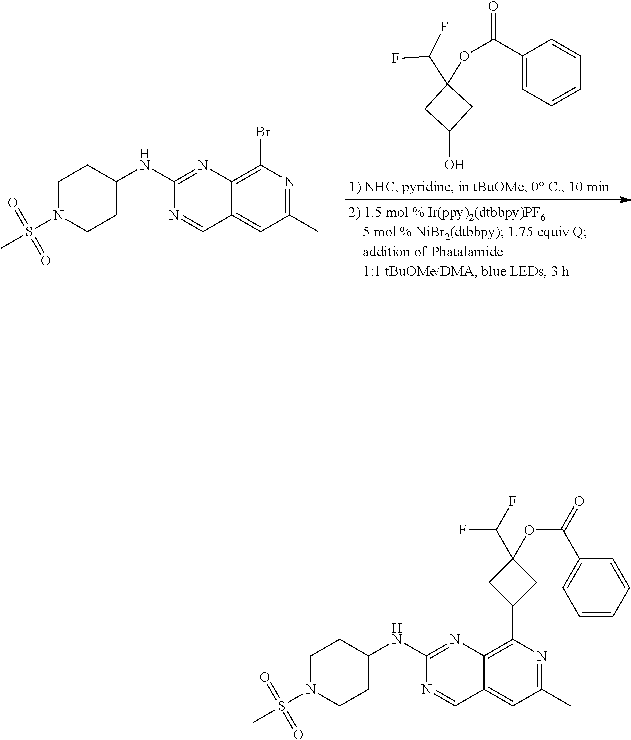

An oven-dried 20 mL vial was charged with 1-(difluoromethyl)-3-hydroxycyclobutyl benzoate (106 mg, 0.44 mmol) and 5,7-di-tert-butyl-3-phenylbenzo[d]oxazol-3-ium tetrafluoroborate (158 mg, 0.40 mmol). Under nitrogen, tert-butyl methyl ether (4 mL) was added and the reaction stirred at room temperature for 5 minutes. A mixture of pyridine (31.6 mg, 0.40 mmol) in tert-butyl methyl ether (1 mL) and the mixture stirred at room temperature for 10 minutes (mixture A). Another oven-dried 40 mL vial was charged with 8-bromo-6-methyl-N-(1-(methylsulfonyl)piperidin-4-yl)pyrido[3,4-d]pyrimidin-2-amine (100 mg, 0.25 mmol), Ir(ppy)₂(dtbbpy)PF₆ (3.43 mg, 0.004 mmol), NiBr₂(dtbbpy) (9.78 mg, 0.02 mmol), phthalimide (8.27 mg, 0.056 mmol) and 1-azabicyclo[2.2.2]octane (48.6 mg, 0.44 mmol). DMA (5 mL) was added under nitrogen (mixture B). The mixture A was added to the mixture B under a nitrogen atmosphere and the resulting mixture was stirred and irradiated with a 450 nm LED lamp under a fan for 3 h. The residue was dissolved in water (20 mL) and extracted with EA (3×20 mL). The combined organic layers were dried over anhydrous Na₂SO₄. After filtration, the filtrate was concentrated under reduced pressure. The residue was purified by Prep-TLC (PE/EA, 1:2) to afford crude 1-(difluoromethyl)-3-(6-methyl-2-((1-(methylsulfonyl)piperidin-4-yl)amino)pyrido[3,4-d]pyrimidin-8-yl)cyclobutyl benzoate (50 mg, 36.68% yield).

LCMS (ESI-MS) m/z=546.2 [M+H]⁺.

### Step 2: 1-(difluoromethyl)-3-(6-methyl-2-((1-(methylsulfonyl)piperidin-4-yl)amino)pyrido[3,4-d]pyrimidin-8-yl)cyclobutan-1-ol

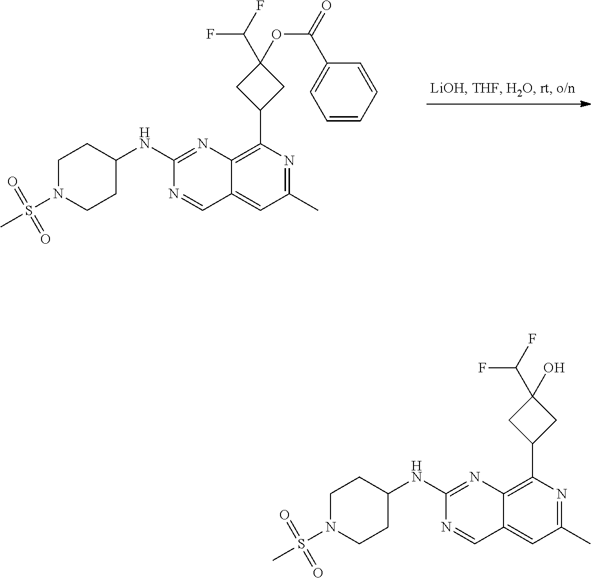

LiOH (10.9 mg, 0.46 mmol) was added to a solution of 1-(difluoromethyl)-3-(6-methyl-2-((1-(methylsulfonyl)piperidin-4-yl)amino)pyrido[3,4-d]pyrimidin-8-yl)cyclobutyl benzoate (50 mg, 0.09 mmol,) in THF (3 mL) and H₂O (1 mL). The resulting mixture was stirred overnight at room temperature. The reaction mixture was diluted with H₂O (10 mL) and extracted with EA (3×20 mL). The combined organic layers were washed with brine (3×20 mL), dried over anhydrous Na₂SO₄ and concentrated under reduced pressure. The residue purified by preparative RP-HPLC to afford 1-(difluoromethyl)-3-(6-methyl-2-((1-(methylsulfonyl)piperidin-4-yl)amino) pyrido[3,4-d]pyrimidin-8-yl)cyclobutan-1-ol (5.8 mg, 13.36%).

¹H NMR (400 MHz, DMSO-d₆) δ 9.16 (s, 1H), 7.70 (s, 1H), 7.37 (s, 1H), 6.06-5.85 (m, 2H), 4.08-3.86 (m, 2H), 3.60-3.50 (m, 2H), 2.96-2.83 (m, 5H), 2.72-2.62 (m, 2H), 2.59-2.52 (m, 4H), 2.14-2.01 (m, 3H), 1.67-1.55 (m, 2H).

LCMS (ESI-MS) m/z=442.1[M+H]⁺.

### Example 99: 8-(8,8-difluoro-6-azaspiro[3.4]octan-2-yl)-6-methyl-N-(1-(methylsulfonyl)piperidin-4-yl)pyrido[3,4-d]pyrimidin-2-amine

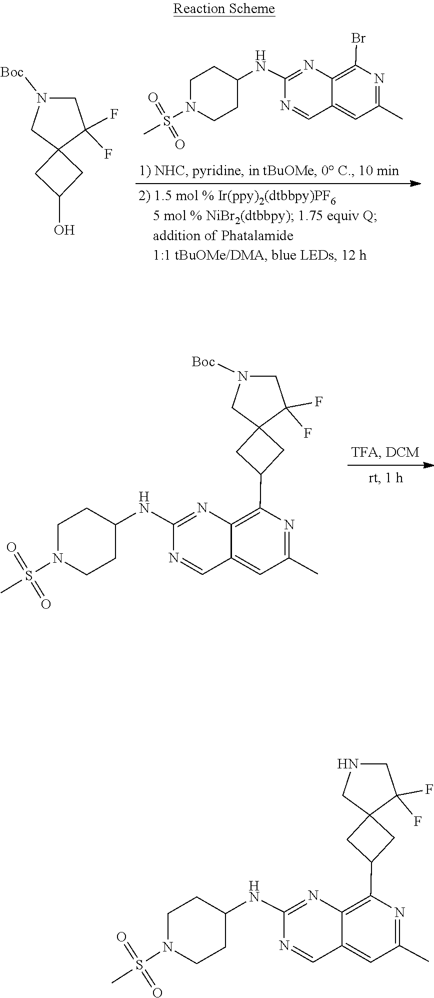

### Step 1: Tert-butyl 8,8-difluoro-2-(6-methyl-2-((1-(methylsulfonyl)piperidin-4-yl)amino)pyrido[3,4-d]pyrimidin-8-yl)-6-azaspiro[3.4]octane-6-carboxylate

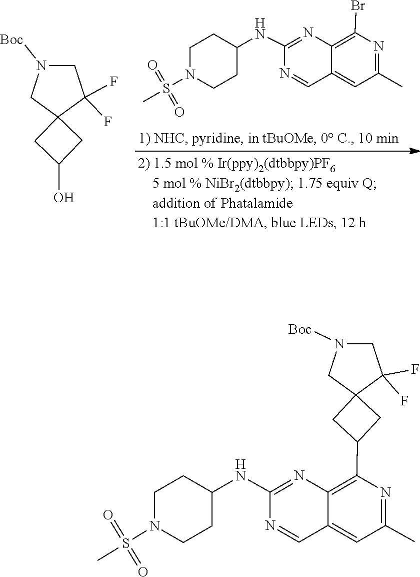

An oven-dried 20 mL vial was charged with tert-butyl 8,8-difluoro-2-hydroxy-6-azaspiro[3.4]octane-6-carboxylate (207 mg, 0.78 mmol) and 5,7-di-tert-butyl-3-phenylbenzo[d]oxazol-3-ium tetrafluoroborate (284 mg, 0.72 mmol). Under nitrogen, tert-butyl methyl ether (4 mL) was added and the reaction stirred at room temperature for 5 minutes. A mixture of pyridine (56.9 mg, 0.72 mmol) in tert-butyl methyl ether (1 mL) was added and the mixture was stirred at room temperature for 10 minutes (mixture A). Another oven-dried 40 mL vial was charged with 8-bromo-6-methyl-N-(1-(methylsulfonyl)piperidin-4-yl)pyrido[3,4-d]pyrimidin-2-amine (180 mg, 0.45 mmol), Ir(ppy)₂(dtbbpy)PF₆ (6.16 mg, 0.007 mmol), NiBr₂(dtbbpy) (16.42 mg, 0.034 mmol), phthalimide (26.46 mg, 0.18 mmol) and 1-azabicyclo[2.2.2]octane (87.49 mg, 0.78 mmol). DMA (5 mL) was added under nitrogen (mixture B). The mixture A was added to the mixture B under a nitrogen atmosphere and the resulting mixture was stirred and irradiated with a 450 nm LED lamp under a fan for 3 h. The residue was dissolved in water (20 mL) and extracted with EA (3×20 mL). The combined organic layers were dried over anhydrous Na₂SO₄. After filtration, the filtrate was concentrated under reduced pressure. The residue was purified by Prep-TLC (PE/EA, 1:2) to afford tert-butyl 8,8-difluoro-2-(6-methyl-2-((1-(methylsulfonyl)piperidin-4-yl)amino)pyrido[3,4-d]pyrimidin-8-yl)-6-azaspiro[3.4]octane-6-carboxylate (20 mg, 7.85% yield).

LCMS (ESI-MS) m/z=567.2 [M+H]⁺.

### Step 2: 8-(8,8-difluoro-6-azaspiro[3.4]octan-2-yl)-6-methyl-N-(1-(methylsulfonyl)piperidin-4-yl)pyrido[3,4-d]pyrimidin-2-amine

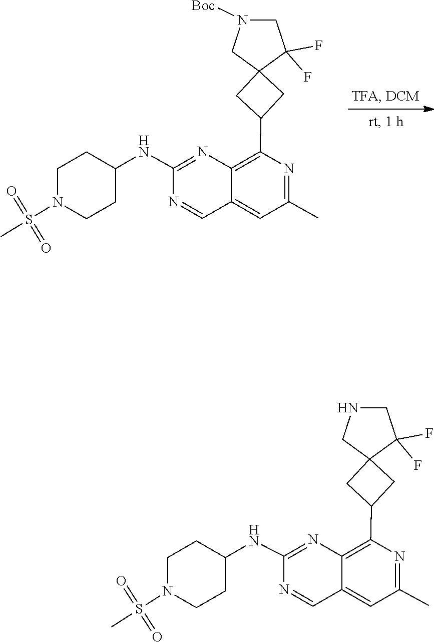 [SMILES: Cc1cc2cnc(NC3CCN(S(C)(=O)=O)CC3)nc2c(C2CC3(C2)CN(C(=O)OC(C)(C)C)CC3(F)F)n1 ; Cc1cc2cnc(NC3CCN(S(C)(=O)=O)CC3)nc2c(C2CC3(CNCC3(F)F)C2)n1]

TFA (0.23 mL) was added to a solution of tert-butyl 8,8-difluoro-2-(6-methyl-2-((1-(methylsulfonyl)piperidin-4-yl)amino)pyrido[3,4-d]pyrimidin-8-yl)-6-azaspiro[3.4]octane-6-carboxylate (20 mg, 0.035 mmol) in DCM (3 mL). The resulting mixture was stirred for 1 h at room temperature and concentrated under reduced pressure. The residue was purified by Prep-TLC (DCM/MeOH, 10:1) to afford 8-(8,8-difluoro-6-azaspiro[3.4]octan-2-yl)-6-methyl-N-(1-(methylsulfonyl)piperidin-4-yl)pyrido[3,4-d]pyrimidin-2-amine (1.0 mg, 5.65% yield).

¹H NMR (400 MHz, CD₃OD) δ 8.04 (s, 1H), 7.32 (s, 1H), 4.10-3.99 (m, 1H), 3.80-3.70 (m, 2H), 3.24-3.13 (m, 2H), 3.09-2.96 (m, 2H), 2.91-2.88 (m, 4H), 2.84-2.77 (m, 2H), 2.61 (s, 3H), 2.52-2.44 (m, 1H), 2.36-2.15 (m, 4H), 1.84-1.66 (m, 2H), 0.94-0.83 (m, 2H).

LCMS (ESI-MS) m/z=467.3 [M+H]⁺.

### Example 100: 8-(8-(difluoromethyl)-2,6-diazaspiro[3.4]octan-2-yl)-6-methyl-N-(1-(methylsulfonyl)piperidin-4-yl)pyrido[3,4-d]pyrimidin-2-amine

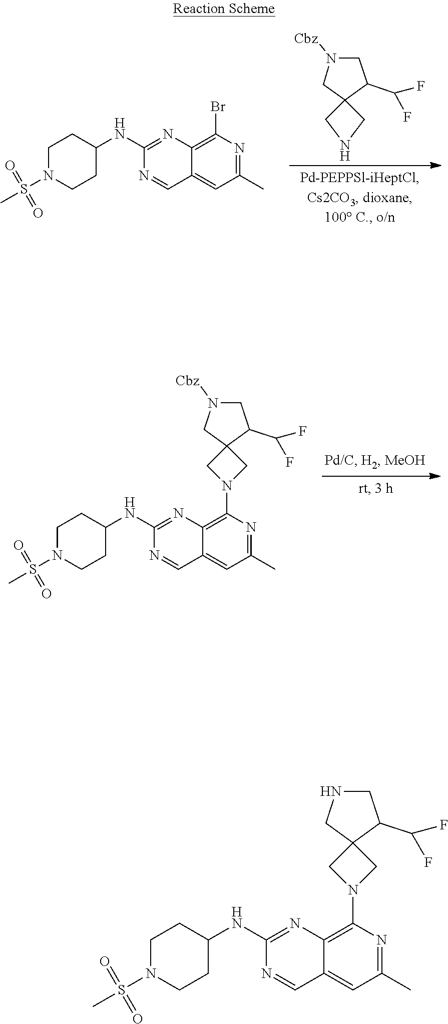

### Detailed Procedure

### Step 1: Benzyl 8-(difluoromethyl)-2-(6-methyl-2-((1-(methylsulfonyl)piperidin-4-yl)amino)pyrido[3,4-d]pyrimidin-8-yl)-2,6-diazaspiro[3.4]octane-6-carboxylate

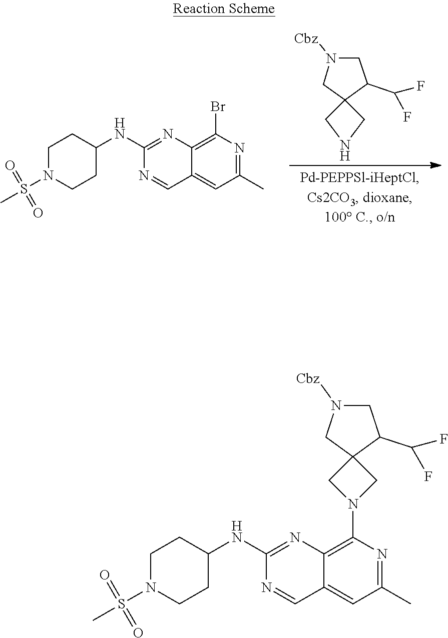

Pd-PEPPSI-iHeptCl 3-chloropyridine (31.55 mg, 0.03 mmol) was added to a mixture of benzyl 8-(difluoromethyl)-2,6-diazaspiro[3.4]octane-6-carboxylate (96 mg, 0.32 mmol), 8-bromo-6-methyl-N-(1-(methylsulfonyl)piperidin-4-yl)pyrido[3,4-d]pyrimidin-2-amine (129.69 mg, 0.32 mmol) and Cs₂CO₃ (211.12 mg, 0.64 mmol) in dioxane (3 mL) under a nitrogen atmosphere. The reaction mixture was stirred overnight at 100° C. under a nitrogen atmosphere. The resulting mixture was filtered and the filter cake was washed with DCM (20 mL). The filtrate was concentrated under reduced pressure. The residue was purified by Prep-TLC (CH₂Cl₂/MeOH, 10:1) to afford benzyl 8-(difluoromethyl)-2-(6-methyl-2-((1-(methylsulfonyl)piperidin-4-yl)amino)pyrido[3,4-d]pyrimidin-8-yl)-2,6-diazaspiro[3.4]octane-6-carboxylate (150 mg, 58.43% yield).

LCMS (ESI-MS) m/z=616.3 [M+H]⁺.

### Step 2: 8-(8-(difluoromethyl)-2,6-diazaspiro[3.4]octan-2-yl)-6-methyl-N-(1-(methylsulfonyl)piperidin-4-yl)pyrido[3,4-d]pyrimidin-2-amine

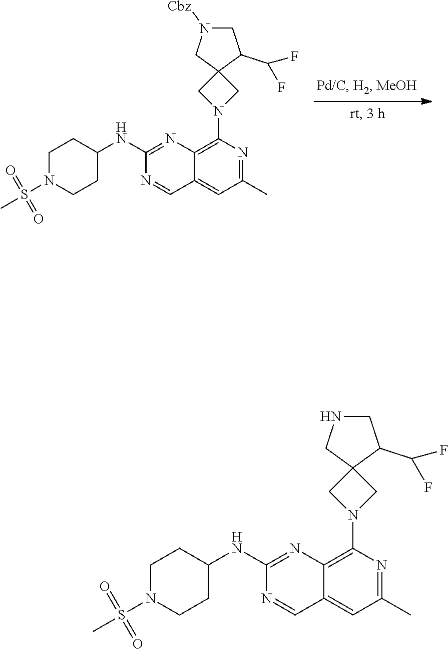 [SMILES: Cc1cc2cnc(NC3CCN(S(C)(=O)=O)CC3)nc2c(N2CC3(CN(C(=O)OCc4ccccc4)CC3C(F)F)C2)n1 ; Cc1cc2cnc(NC3CCN(S(C)(=O)=O)CC3)nc2c(N2CC3(CNCC3C(F)F)C2)n1]

Pd/C (116.67 mg, 0.11 mmol, 10% on carbon) was added to a solution of benzyl 8-(difluoromethyl)-2-(6-methyl-2-((1-(methylsulfonyl)piperidin-4-yl)amino)pyrido[3,4-d]pyrimidin-8-yl)-2,6-diazaspiro[3.4]octane-6-carboxylate (135 mg, 0.21 mmol) in MeOH (5 mL) under a nitrogen atmosphere. The reaction mixture was stirred for 3 h at room temperature under a hydrogen atmosphere. The resulting mixture was filtered and the filter cake was washed with MeOH (20 mL). The filtrate was concentrated under reduced pressure. The residue was purified by Prep-TLC (CH₂Cl₂/MeOH, 10:1). The product was further purified by preparative RP-HPLC to afford 8-(8-(difluoromethyl)-2,6-diazaspiro[3.4]octan-2-yl)-6-methyl-N-(1-(methylsulfonyl)piperidin-4-yl)pyrido[3,4-d]pyrimidin-2-amine (14.6 mg, 13.25% yield).

¹H NMR (400 MHz, Chloroform-d) δ 9.04-8.68 (m, 1H), 7.45-7.11 (m, 1H), 6.64 (d, J=4.1 Hz, 1H), 6.03 (t, J=55.8 Hz, 1H), 5.32-5.06 (m, 1H), 4.92-4.68 (m, 1H), 4.34 (s, 3H), 4.10-3.91 (m, 1H), 3.89-3.73 (m, 2H), 3.51-3.07 (m, 4H), 3.05-2.92 (m, 2H), 2.91-2.78 (m, 3H), 2.73-2.56 (m, 1H), 2.56-2.35 (m, 3H), 2.22 (s, 2H), 1.83-1.65 (m, 2H).

LCMS (ESI-MS) m/z=482.4 [M+H]⁺.

### Examples 103 and 104: (R)-8-(8-fluoro-6-methyl-2,6-diazaspiro[3.4]octan-2-yl)-6-methyl-N-(1-(methylsulfonyl)piperidin-4-yl)pyrido[3,4-d]pyrimidin-2-amine and (S)-8-(8-fluoro-6-methyl-2,6-diazaspiro[3.4]octan-2-yl)-6-methyl-N-(1-(methylsulfonyl)piperidin-4-yl)pyrido[3,4-d]pyrimidin-2-amine

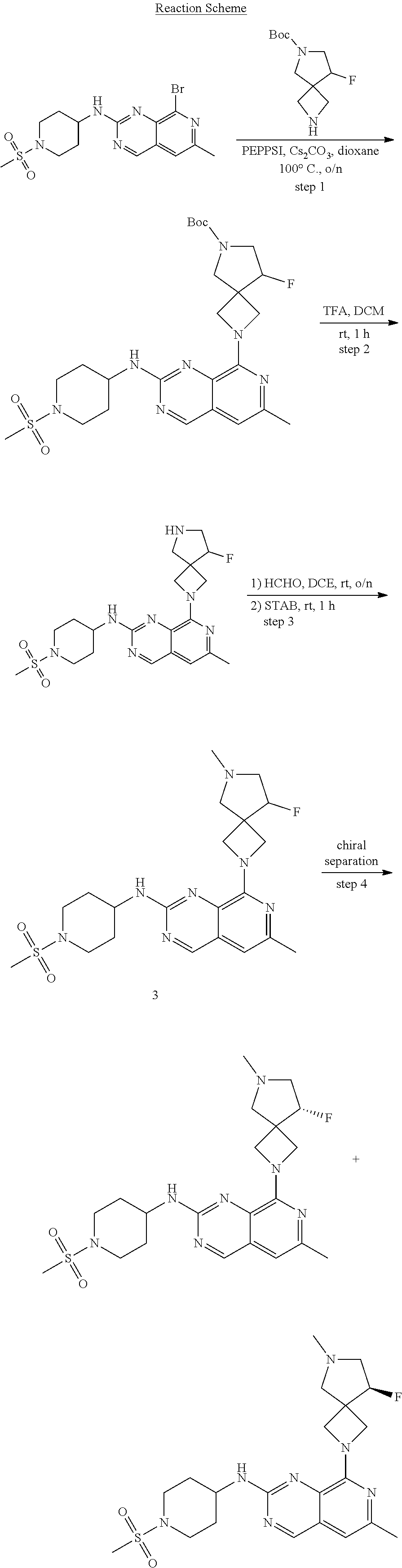

### Detailed Procedure

### Step 1: Tert-butyl8-fluoro-2-(6-methyl-2-((1-(methylsulfonyl)piperidin-4-yl)amino)pyrido[3,4-d]pyrimidin-8-yl)-2,6-diazaspiro[3.4]octane-6-carboxylate

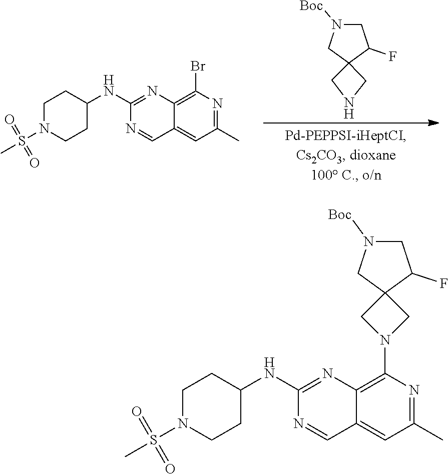

To a solution of 8-bromo-6-methyl-N-(1-(methylsulfonyl)piperidin-4-yl)pyrido[3,4-d]pyrimidin-2-amine (180 mg, 0.45 mmol) in dioxane (1 mL, 11.80 mmol) was added tert-butyl 8-fluoro-2,6-diazaspiro[3.4] octane-6-carboxylate (103.55 mg, 0.45 mmol), Pd-PEPPSI-iHeptCl 3-chloropyridine (43.79 mg, 0.04 mmol) and Cs₂CO₃ (293.02 mg, 0.90 mmol) under a nitrogen atmosphere. The resulting mixture was stirred overnight at 100° C. under a nitrogen atmosphere. The mixture was filtered and the filter cake was washed with DCM (20 mL). The filtrate was concentrated under reduced pressure. The residue was purified by silica gel column chromatography (PE/EA, 1:9) to afford tert-butyl8-fluoro-2-(6-methyl-2-((1-(methylsulfonyl)piperidin-4-yl)amino)pyrido[3,4-d]pyrimidin-8-yl)-2,6-diazaspiro[3.4]octane-6-carboxylate (180 mg, 58.26% yield).

LCMS (ESI-MS) m/z=550.2 [M+H]⁺.

### Step 2: 8-(8-fluoro-2,6-diazaspiro[3.4]octan-2-yl)-6-methyl-N-(1-(methylsulfonyl)piperidin-4-yl)pyrido[3,4-d]pyrimidin-2-amine

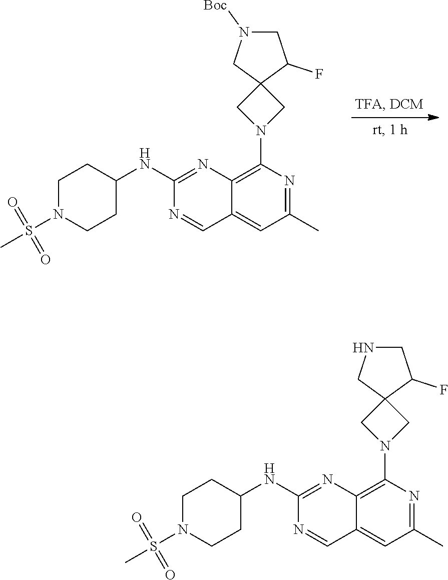 [SMILES: Cc1cc2cnc(NC3CCN(S(C)(=O)=O)CC3)nc2c(N2CC3(CN(C(=O)OC(C)(C)C)CC3F)C2)n1 ; Cc1cc2cnc(NC3CCN(S(C)(=O)=O)CC3)nc2c(N2CC3(CNCC3F)C2)n1]

A solution of tert-butyl8-fluoro-2-(6-methyl-2-((1-(methylsulfonyl)piperidin-4-yl)amino)pyrido[3,4-d]pyrimidin-8-yl)-2,6-diazaspiro[3.4]octane-6-carboxylate (180 mg, 0.327 mmol) in TFA (0.6 mL) and DCM (2 mL) was stirred at room temperature for 1 h. The reaction mixture was concentrated under reduced pressure to afford crude 8-(8-fluoro-2,6-diazaspiro[3.4]octan-2-yl)-6-methyl-N-(1-(methylsulfonyl)piperidin-4-yl)pyrido[3,4-d]pyrimidin-2-amine (145 mg, 78.80% yield).

LCMS (ESI-MS) m/z=450.2 [M+H]⁺.

### Step 3: 8-(8-fluoro-6-methyl-2,6-diazaspiro[3.4]octan-2-yl)-6-methyl-N-(1-(methylsulfonyl)piperidin-4-yl)pyrido[3,4-d]pyrimidin-2-amine

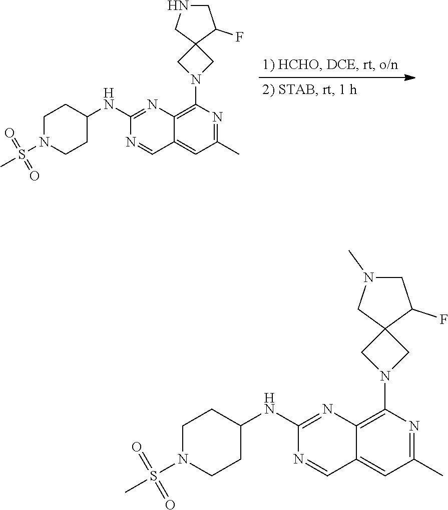 [SMILES: Cc1cc2cnc(NC3CCN(S(C)(=O)=O)CC3)nc2c(N2CC3(CNCC3F)C2)n1 ; Cc1cc2cnc(NC3CCN(S(C)(=O)=O)CC3)nc2c(N2CC3(CN(C)CC3F)C2)n1]

A mixture of 8-(8-fluoro-2,6-diazaspiro[3.4]octan-2-yl)-6-methyl-N-(1-(methylsulfonyl)piperidin-4-yl) pyrido[3,4-d]pyrimidin-2-amine (135 mg, 0.300 mmol) and HCHO (10.82 mg, 0.36 mmol) in DCE (1.5 mL) was stirred overnight at room temperature. STAB (95.47 mg, 0.45 mmol) was added and the resulting mixture was stirred for 1 h at room temperature. The mixture was purified by silica gel column chromatography (DCM/MeOH, 10:1) to afford racemic 8-(8-fluoro-6-methyl-2,6-diazaspiro[3.4]octan-2-yl)-6-methyl-N-(1-(methylsulfonyl)piperidin-4-yl)pyrido[3,4-d]pyrimidin-2-amine 10.2 mg, 7.09%).

LCMS (ESI-MS) m/z=464.2 [M+H]⁺.

### Step 4: (R)-8-(8-fluoro-6-methyl-2,6-diazaspiro[3.4]octan-2-yl)-6-methyl-N-(1-(methylsulfonyl)piperidin-4-yl)pyrido[3,4-d]pyrimidin-2-amine and (S)-8-(8-fluoro-6-methyl-2,6-diazaspiro[3.4]octan-2-yl)-6-methyl-N-(1-(methylsulfonyl)piperidin-4-yl)pyrido[3,4-d]pyrimidin-2-amine

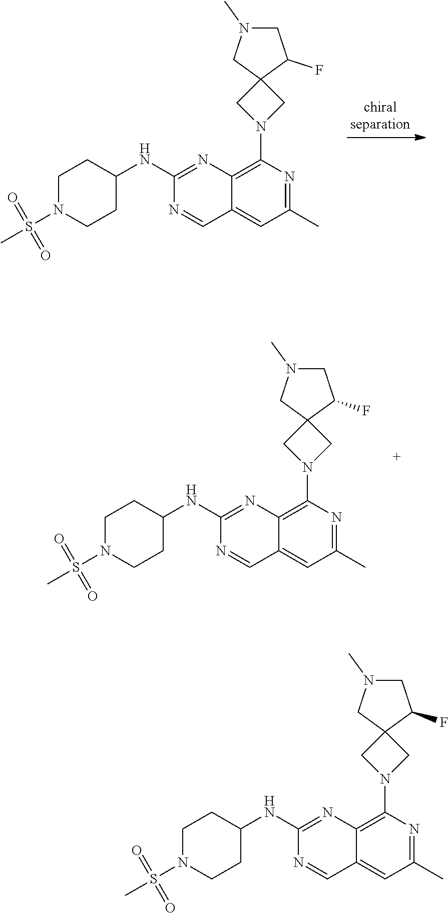 [SMILES: Cc1cc2cnc(NC3CCN(S(C)(=O)=O)CC3)nc2c(N2CC3(CN(C)CC3F)C2)n1 ; Cc1cc2cnc(NC3CCN(S(C)(=O)=O)CC3)nc2c(N2CC3(CN(C)C[C@@H]3F)C2)n1 ; Cc1cc2cnc(NC3CCN(S(C)(=O)=O)CC3)nc2c(N2CC3(CN(C)C[C@H]3F)C2)n1]

Racemic 8-(8-fluoro-6-methyl-2,6-diazaspiro[3.4]octan-2-yl)-6-methyl-N-(1-(methylsulfonyl) piperidin-4-yl)pyrido[3,4-d]pyrimidin-2-amine was separated by Prep-Chiral-HPLC using the following conditions:

### Column: CHIRAL Cellulose-SB, 4.6*100 mm, 3 μm.

### Mobile Phase: Hex (0.1% DEA)/(EtOH/DCM, 1:1), 75:25.

The desired fractions were combined and lyophilized to afford the title products as single enantiomers.

### First Eluting Peak:

17.4 mg, 99.8% ee, 7.09% yield.

¹H NMR (400 MHz, DMSO-d₆) δ 8.94 (s, 1H), 7.45-7.41 (m, 1H), 6.70 (s, 1H), 5.30-5.15 (m, 1H), 4.54-4.17 (m, 3H), 3.82 (s, 1H), 3.57-3.54 (m, 2H), 2.96-2.88 (m, 5H), 2.84-2.82 (m, 2H), 2.67-2.50 (m, 3H), 2.49-2.28 (m, 6H), 2.12-1.95 (m, 2H), 1.71-1.52 (m, 2H).

LCMS (ESI-MS) m/z=464.2 [M+H]⁺.

### Second Eluting Peak:

14.4 mg, 99.6% ee, 9.85% yield ¹H NMR (400 MHz, DMSO-d₆) δ 8.94 (s, 1H), 7.49-7.43 (m, 1H), 6.70 (s, 1H), 5.30-5.15 (m, 1H), 4.53-4.13 (m, 3H), 3.82 (s, 1H), 3.58-3.56 (m, 2H), 3.01-2.82 (m, 7H), 2.67-2.63 (m, 3H), 2.50-2.28 (m, 6H), 2.02-1.99 (m, 2H), 1.71-1.59 (m, 2H).

LCMS (ESI-MS) m/z=464.2 [M+H]⁺.

In some embodiments, compounds of the disclosure are below in Table 1.

TABLE 1

| Com- | | | Mass |
|---|---|---|---|
| pound | | | Spec. |
| No. | Structure | Name | M + H/1 |
| 1 | 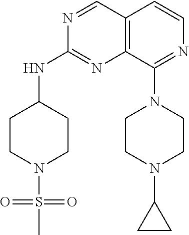 [SMILES: CS(=O)(=O)N1CCC(Nc2ncc3ccnc(N4CCN(C5CC5)CC4)c3n2)CC1] | 8-(4-cyclopropylpiperazin-1-yl)-N-(1- (methylsulfonyl)piperidin-4-yl)pyrido[3,4- d]pyrimidin-2-amine | 432.2 |
| 2 | 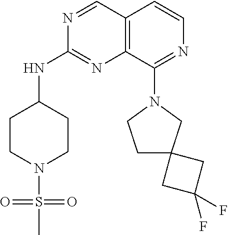 [SMILES: CS(=O)(=O)N1CCC(Nc2ncc3ccnc(N4CCC5(C4)CC(F)(F)C5)c3n2)CC1] | 8-(2,2-difluoro-6-azaspiro[3.4]octan-6-yl)-N-(1- (methylsulfonyl)piperidin-4-yl)pyrido[3,4- d]pyrimidin-2-amine | 453 |
| 3 | 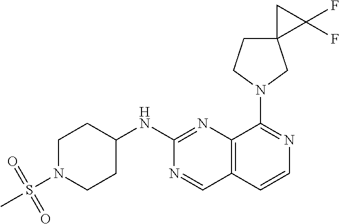 [SMILES: CS(=O)(=O)N1CCC(Nc2ncc3ccnc(N4CCC5(C4)CC5(F)F)c3n2)CC1] | 8-(1,1-difluoro-5-azaspiro[2.4]heptan-5-yl)-N-(1- (methylsulfonyl)piperidin-4-yl)pyrido[3,4- d]pyrimidin-2-amine | 439.2 |
| 4 | 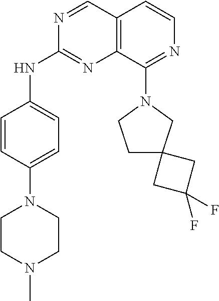 [SMILES: CN1CCN(c2ccc(Nc3ncc4ccnc(N5CCC6(C5)CC(F)(F)C6)c4n3)cc2)CC1] | 8-(2,2-difluoro-6-azaspiro[3.4]octan-6-yl)-N-(4-(4- methylpiperazin-1-yl)phenyl)pyrido[3,4- d]pyrimidin-2-amine | 466.2 |
| 5 | 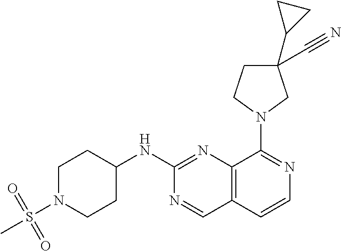 [SMILES: CS(=O)(=O)N1CCC(Nc2ncc3ccnc(N4CCC(C#N)(C5CC5)C4)c3n2)CC1] | 3-cyclopropyl-1-(2-((1-(methylsulfonyl)piperidin- 4-yl)amino)pyrido[3,4-d]pyrimidin-8- yl)pyrrolidine-3-carbonitrile | 442.2 |
| 6 | 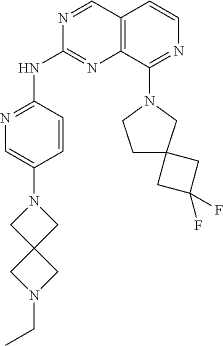 [SMILES: CCN1CC2(C1)CN(c1ccc(Nc3ncc4ccnc(N5CCC6(C5)CC(F)(F)C6)c4n3)nc1)C2] | 8-(2,2-difluoro-6-azaspiro[3.4]octan-6-yl)-N-(5-(6- ethyl-2,6-diazaspiro[3.3]heptan-2-yl)pyridin-2- yl)pyrido[3,4-d]pyrimidin-2-amine | 493 |
| 7 | 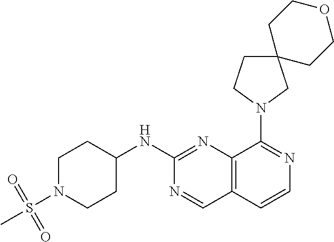 [SMILES: CS(=O)(=O)N1CCC(Nc2ncc3ccnc(N4CCC5(CCOCC5)C4)c3n2)CC1] | N-(1-(methylsulfonyl)piperidin-4-yl)-8-(8-oxa-2- azaspiro[4.5]decan-2-yl)pyrido[3,4-d]pyrimidin-2- amine | 447.2 |
| 8 | 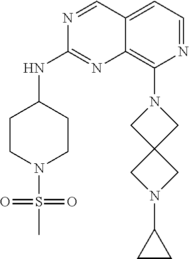 [SMILES: CS(=O)(=O)N1CCC(Nc2ncc3ccnc(N4CC5(C4)CN(C4CC4)C5)c3n2)CC1] | 8-(6-cyclopropyl-2,6-diazaspiro[3.3]heptan-2-yl)- N-(1-(methylsulfonyl)piperidin-4-yl)pyrido[3,4- d]pyrimidin-2-amine | 444.2 |
| 9 | 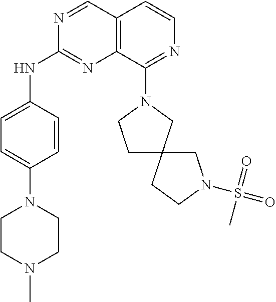 [SMILES: CN1CCN(c2ccc(Nc3ncc4ccnc(N5CCC6(CCN(S(C)(=O)=O)C6)C5)c4n3)cc2)CC1] | N-(4-(4-methylpiperazin-1-yl)phenyl)-8-(7- (methylsulfonyl)-2,7-diazaspiro[4.4]nonan-2- yl)pyrido[3,4-d]pyrimidin-2-amine | 523.3 |
| 10 | 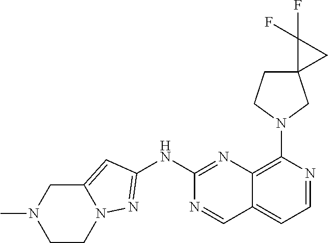 [SMILES: CN1CCn2nc(Nc3ncc4ccnc(N5CCC6(C5)CC6(F)F)c4n3)cc2C1] | 8-(1,1-difluoro-5-azaspiro[2.4]heptan-5-yl)-N-(5- methyl-4,5,6,7-tetrahydropyrazolo[1,5-a]pyrazin- 2-yl)pyrido[3,4-d]pyrimidin-2-amine | 413.1 |
| 11 | 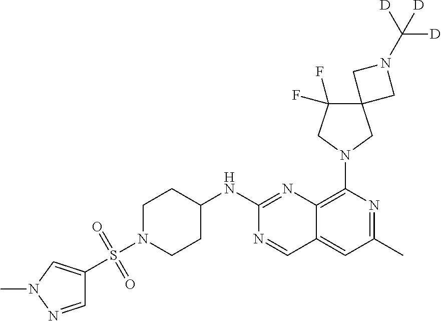 [SMILES: [2H]C([2H])([2H])N1CC2(CN(c3nc(C)cc4cnc(NC5CCN(S(=O)(=O)c6cnn(C)c6)CC5)nc34)CC2(F)F)C1] | 8-(8,8-difluoro-2-(methyl-d3)-2,6- diazaspiro[3.4]octan-6-yl)-6-methyl-N-(1-((1- methyl-1H-pyrazol-4-yl)sulfonyl)piperidin-4- yl)pyrido[3,4-d]pyrimidin-2-amine | 551.3 |
| 12 | 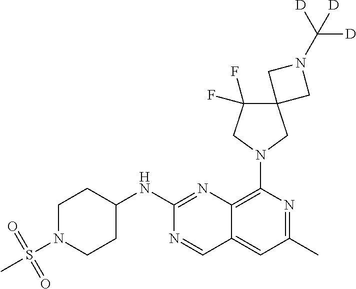 [SMILES: [2H]C([2H])([2H])N1CC2(CN(c3nc(C)cc4cnc(NC5CCN(S(C)(=O)=O)CC5)nc34)CC2(F)F)C1] | 8-(8,8-difluoro-2-(methyl-d3)-2,6- diazaspiro[3.4]octan-6-yl)-6-methyl-N-(1- (methylsulfonyl)piperidin-4-yl)pyrido[3,4- d]pyrimidin-2-amine | 485.3 |
| 13 | 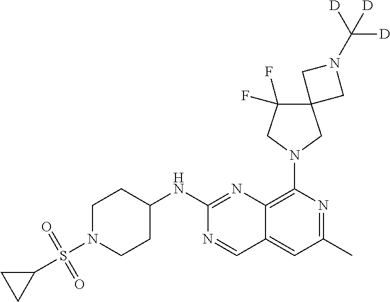 [SMILES: [2H]C([2H])([2H])N1CC2(CN(c3nc(C)cc4cnc(NC5CCN(S(=O)(=O)C6CC6)CC5)nc34)CC2(F)F)C1] | N-(1-(cyclopropylsulfonyl)piperidin-4-yl)-8-(8,8- difluoro-2-(methyl-d3)-2,6-diazaspiro[3.4]octan-6- yl)-6-methylpyrido[3,4-d]pyrimidin-2-amine | 511.3 |
| 14 | 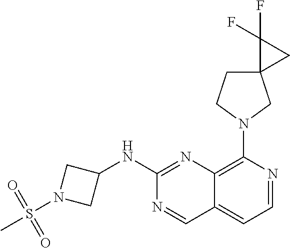 [SMILES: CS(=O)(=O)N1CC(Nc2ncc3ccnc(N4CCC5(C4)CC5(F)F)c3n2)C1] | 8-(1,1-difluoro-5-azaspiro[2.4]heptan-5-yl)-N-(1- (methylsulfonyl)azetidin-3-yl)pyrido[3,4- d]pyrimidin-2-amine | 411.1 |
| 15 | 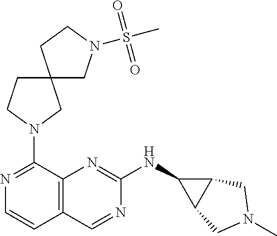 [SMILES: CN1C[C@@H]2[C@H](C1)[C@H]2Nc1ncc2ccnc(N3CCC4(CCN(S(C)(=O)=O)C4)C3)c2n1] | N-((1R,5S,6s)-3-methyl-3-azabicyclo[3.1.0]hexan- 6-yl)-8-(7-(methylsulfonyl)-2,7- diazaspiro[4.4]nonan-2-yl)pyrido[3,4-d]pyrimidin- 2-amine | 444.1 |
| 16 | 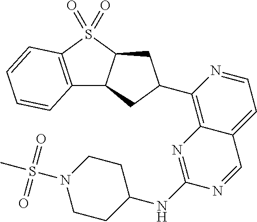 | (3aR,8bR)-2-(2-((1-(methylsulfonyl)piperidin-4- yl)amino)pyrido[3,4-d]pyrimidin-8-yl)-2,3,3a,8b- tetrahydro-1H-benzo[4,5]thieno[2,3-c]pyrrole 4,4- dioxide | 515 |
| 17 | 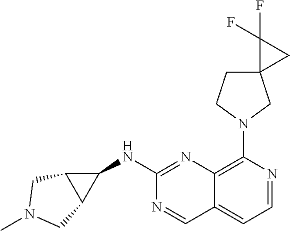 [SMILES: CN1C[C@@H]2[C@H](C1)[C@H]2Nc1ncc2ccnc(N3CCC4(C3)CC4(F)F)c2n1] | 8-(1,1-difluoro-5-azaspiro[2.4]heptan-5-yl)-N- ((1R,5S,6s)-3-methyl-3-azabicyclo[3.1.0]hexan-6- yl)pyrido[3,4-d]pyrimidin-2-amine | 373.1 |
| 18 | 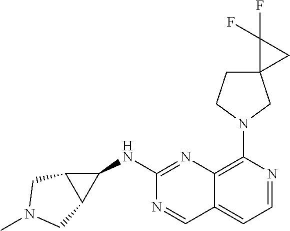 [SMILES: CN1C[C@@H]2[C@H](C1)[C@H]2Nc1ncc2ccnc(N3CCC4(C3)CC4(F)F)c2n1] | N-(5-(6-ethyl-2,6-diazaspiro[3.3]heptan-2- yl)pyridin-2-yl)-8-(8-oxa-2-azaspiro[4.5]decan-2- yl)pyrido[3,4-d]pyrimidin-2-amine | 487.2 |
| 19 | 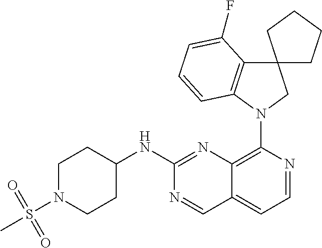 [SMILES: CS(=O)(=O)N1CCC(Nc2ncc3ccnc(N4CC5(CCCC5)c5c(F)cccc54)c3n2)CC1] | 8-(4′-fluorospiro[cyclopentane-1,3′-indolin]-1′-yl)- N-(1-(methylsulfonyl)piperidin-4-yl)pyrido[3,4- d]pyrimidin-2-amine | 497.1 |
| 20 | 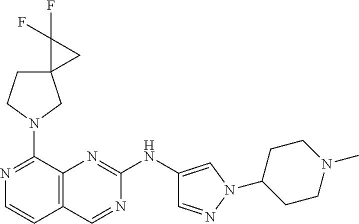 [SMILES: CN1CCC(n2cc(Nc3ncc4ccnc(N5CCC6(C5)CC6(F)F)c4n3)cn2)CC1] | 8-(1,1-difluoro-5-azaspiro[2.4]heptan-5-yl)-N-(1- (1-methylpiperidin-4-yl)-1H-pyrazol-4- yl)pyrido[3,4-d]pyrimidin-2-amine | 441.2 |
| 21 | 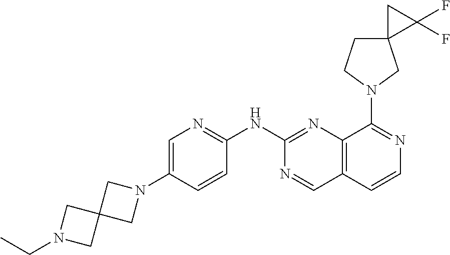 [SMILES: CCN1CC2(C1)CN(c1ccc(Nc3ncc4ccnc(N5CCC6(C5)CC6(F)F)c4n3)nc1)C2] | 8-(1,1-difluoro-5-azaspiro[2.4]heptan-5-yl)-N-(5- (6-ethyl-2,6-diazaspiro[3.3]heptan-2-yl)pyridin-2- yl)pyrido[3,4-d]pyrimidin-2-amine | 479.2 |
| 22 | 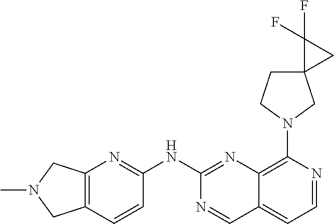 [SMILES: CN1Cc2ccc(Nc3ncc4ccnc(N5CCC6(C5)CC6(F)F)c4n3)nc2C1] | 8-(1,1-difluoro-5-azaspiro[2.4]heptan-5-yl)-N-(2- methylisoindolin-5-yl)pyrido[3,4-d]pyrimidin-2- amine | 409.2 |
| 23 | 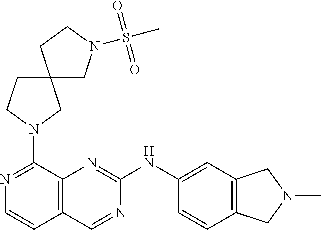 [SMILES: CN1Cc2ccc(Nc3ncc4ccnc(N5CCC6(CCN(S(C)(=O)=O)C6)C5)c4n3)cc2C1] | N-(2-methylisoindolin-5-yl)-8-(7- (methylsulfonyl)-2,7-diazaspiro[4.4]nonan-2- yl)pyrido[3,4-d]pyrimidin-2-amine | 480.2 |
| 24 | 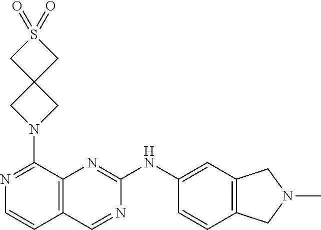 [SMILES: CN1Cc2ccc(Nc3ncc4ccnc(N5CC6(C5)CS(=O)(=O)C6)c4n3)cc2C1] | 6-(2-((2-methylisoindolin-5-yl)amino)pyrido[3,4- d]pyrimidin-8-yl)-2-thia-6- azaspiro[3.3]heptane 2,2-dioxide | 423.1 |
| 25 | 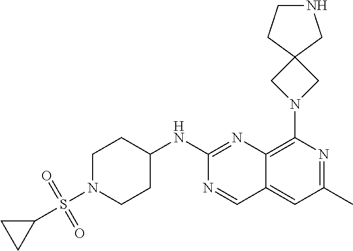 [SMILES: Cc1cc2cnc(NC3CCN(S(=O)(=O)C4CC4)CC3)nc2c(N2CC3(CCNC3)C2)n1] | N-(1-(cyclopropylsulfonyl)piperidin-4-yl)-6- methyl-8-(2,6-diazaspiro[3.4]octan-2- yl)pyrido[3,4-d]pyrimidin-2-amine | 458.3 |
| 26 | 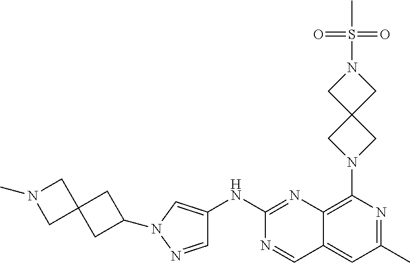 | 6-methyl-N-(1-(2-methyl-2-azaspiro[3.3]heptan-6- yl)-1H-pyrazol-4-yl)-8-(6-(methylsulfonyl)-2,6- diazaspiro[3.3]heptan-2-yl)pyrido[3,4-d]pyrimidin- 2-amine | 510.1 |
| 27 | 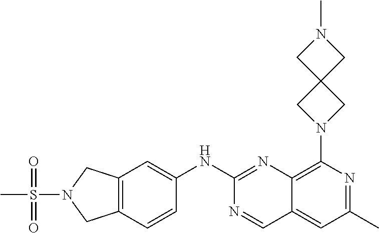 | 6-methyl-8-(6-methyl-2,6-diazaspiro[3.3]heptan-2- yl)-N-(2-(methylsulfonyl)isoindolin-5- yl)pyrido[3,4-d]pyrimidin-2-amine | 466.2 |
| 28 | 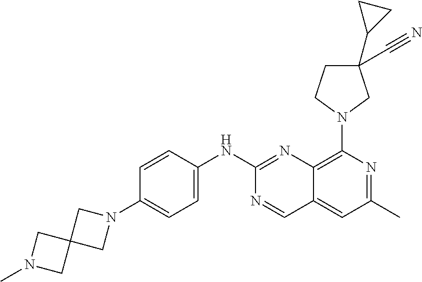 [SMILES: Cc1cc2cnc(Nc3ccc(N4CC5(CN(C)C5)C4)cc3)nc2c(N2CCC(C#N)(C3CC3)C2)n1] | 3-cyclopropyl-1-(6-methyl-2-((4-(6-methyl-2,6- diazaspiro[3.3]heptan-2- yl)phenyl)amino)pyrido[3,4-d]pyrimidin-8- yl)pyrrolidine-3-carbonitrile | 481.3 |
| 29 | 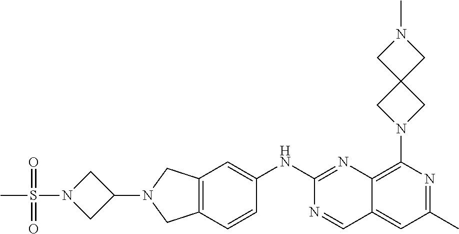 | 6-methyl-8-(6-methyl-2,6-diazaspiro[3.3]heptan-2- yl)-N-(2-(1-(methylsulfonyl)azetidin-3- yl)isoindolin-5-yl)pyrido[3,4-d]pyrimidin-2-amine | 521.3 |
| 30 | 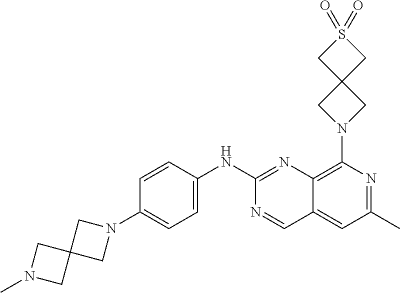 | 6-(6-methyl-2-((4-(6-methyl-2,6- diazaspiro[3.3]heptan-2- yl)phenyl)amino)pyrido[3,4-d]pyrimidin-8-yl)-2- thia-6-azaspiro[3.3]heptane 2,2-dioxide | 492.1 |
| 31 | 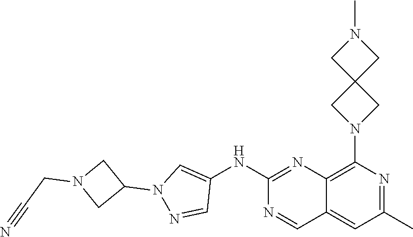 | 2-(3-(4-((6-methyl-8-(6-methyl-2,6- diazaspiro[3.3]heptan-2-yl)pyrido[3,4-d]pyrimidin- 2-yl)amino)-1H-pyrazol-1-yl)azetidin-1- yl)acetonitrile | 431.2 |
| 32 | 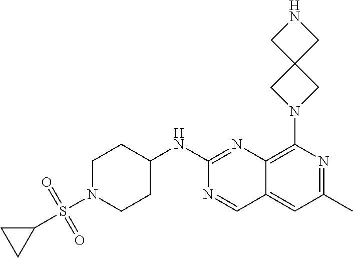 [SMILES: Cc1cc2cnc(NC3CCN(S(=O)(=O)C4CC4)CC3)nc2c(N2CC3(CNC3)C2)n1] | N-(1-(cyclopropylsulfonyl)piperidin-4-yl)-6- methyl-8-(2,6-diazaspiro[3.3]heptan-2- yl)pyrido[3,4-d]pyrimidin-2-amine | 444.2 |
| 33 | 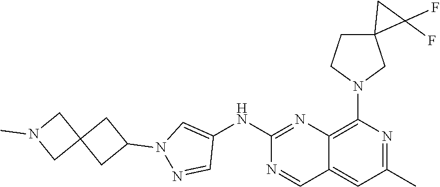 [SMILES: Cc1cc2cnc(Nc3cnn(C4CC5(C4)CN(C)C5)c3)nc2c(N2CCC3(C2)CC3(F)F)n1] | 8-(1,1-difluoro-5-azaspiro[2.4]heptan-5-yl)-6- methyl-N-(1-(2-methyl-2-azaspiro[3.3]heptan-6- yl)-1H-pyrazol-4-yl)pyrido[3,4-d]pyrimidin-2- amine | 467.2 |
| 34 | 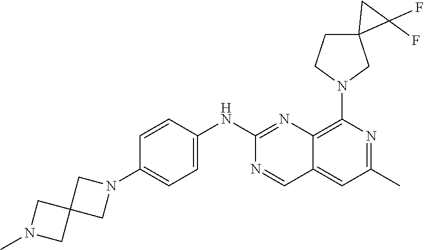 [SMILES: Cc1cc2cnc(Nc3ccc(N4CC5(CN(C)C5)C4)cc3)nc2c(N2CCC3(C2)CC3(F)F)n1] | 8-(1,1-difluoro-5-azaspiro[2.4]heptan-5-yl)-6- methyl-N-(4-(6-methyl-2,6-diazaspiro[3.3]heptan- 2-yl)phenyl)pyrido[3,4-d]pyrimidin-2-amine | 478.2 |
| 35 | 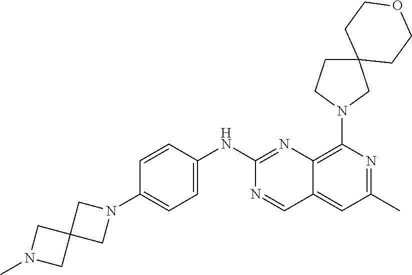 [SMILES: Cc1cc2cnc(Nc3ccc(N4CC5(CN(C)C5)C4)cc3)nc2c(N2CCC3(CCOCC3)C2)n1] | 6-methyl-N-(4-(6-methyl-2,6- diazaspiro[3.3]heptan-2-yl)phenyl)-8-(8-oxa-2- azaspiro[4.5]decan-2-yl)pyrido[3,4-d]pyrimidin-2- amine | 486.2 |
| 36 | 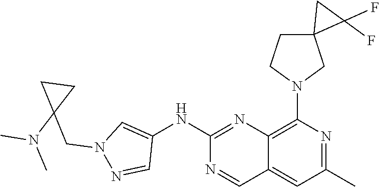 [SMILES: Cc1cc2cnc(Nc3cnn(CC4(N(C)C)CC4)c3)nc2c(N2CCC3(C2)CC3(F)F)n1] | 8-(1,1-difluoro-5-azaspiro[2.4]heptan-5-yl)-N-(1- ((1-(dimethylamino)cyclopropyl)methyl)-1H- pyrazol-4-yl)-6-methylpyrido[3,4-d]pyrimidin-2- amine | 455.1 |
| 37 | 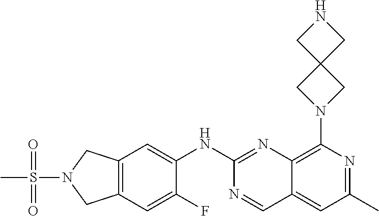 | N-(6-fluoro-2-(methylsulfonyl)isoindolin-5-yl)-6- methyl-8-(2,6-diazaspiro[3.3]heptan-2- yl)pyrido[3,4-d]pyrimidin-2-amine | 470.2 |
| 38 | 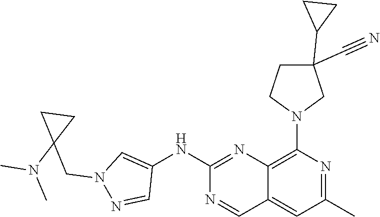 [SMILES: Cc1cc2cnc(Nc3cnn(CC4(N(C)C)CC4)c3)nc2c(N2CCC(C#N)(C3CC3)C2)n1] | 3-cyclopropyl-1-(2-((1-((1- (dimethylamino)cyclopropyl)methyl)-1H-pyrazol- 4-yl)amino)-6-methylpyrido[3,4-d]pyrimidin-8- yl)pyrrolidine-3-carbonitrile | 458.2 |
| 39 | 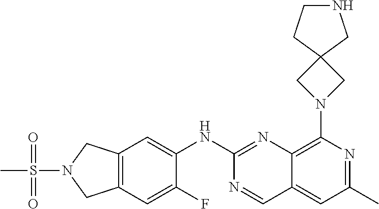 | N-(6-fluoro-2-(methylsulfonyl)isoindolin-5-yl)-6- methyl-8-(2,6-diazaspiro[3.4]octan-2- yl)pyrido[3,4-d]pyrimidin-2-amine | 484.2 |
| 40 | 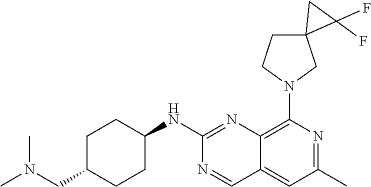 [SMILES: Cc1cc2cnc(N[C@H]3CC[C@H](CN(C)C)CC3)nc2c(N2CCC3(C2)CC3(F)F)n1] | 8-(1,1-difluoro-5-azaspiro[2.4]heptan-5-yl)-N- ((1r,4r)-4-((dimethylamino)methyl)cyclohexyl)-6- methylpyrido[3,4-d]pyrimidin-2-amine | 431.2 |
| 41 | 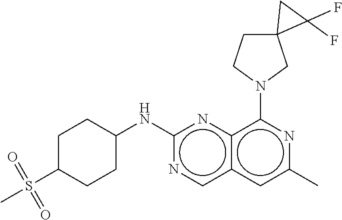 | 8-(1,1-difluoro-5-azaspiro[2.4]heptan-5-yl)-6- methyl-N-(1-(methylsulfonyl)piperidin-4- yl)pyrido[3,4-d]pyrimidin-2-amine | 453.3 |
| 42 | 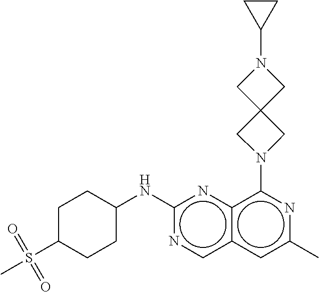 | 8-(6-cyclopropyl-2,6-diazaspiro[3.3]heptan-2-yl)- 6-methyl-N-(1-(methylsulfonyl)piperidin-4- yl)pyrido[3,4-d]pyrimidin-2-amine | 458.3 |
| 43 | 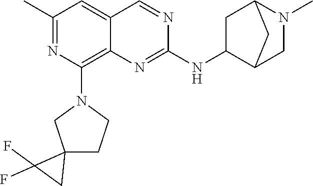 [SMILES: Cc1cc2cnc(NC3CC4CC3CN4C)nc2c(N2CCC3(C2)CC3(F)F)n1] | 8-(1,1-difluoro-5-azaspiro[2.4]heptan-5-yl)-6- methyl-N-(2-methyl-2-azabicyclo[2.2.1]heptan-5- yl)pyrido[3,4-d]pyrimidin-2-amine | 401.2 |
| 44 | 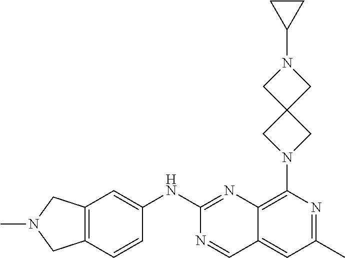 | 8-(6-cyclopropyl-2,6-diazaspiro[3.3]heptan-2-yl)- 6-methyl-N-(2-methylisoindolin-5-yl)pyrido[3,4- d]pyrimidin-2-amine | 428.3 |
| 45 | 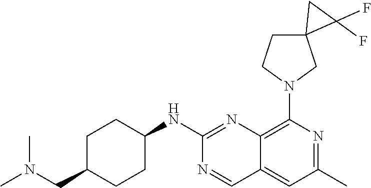 [SMILES: Cc1cc2cnc(N[C@H]3CC[C@@H](CN(C)C)CC3)nc2c(N2CCC3(C2)CC3(F)F)n1] | 8-(1,1-difluoro-5-azaspiro[2.4]heptan-5-yl)-N- ((1s,4s)-4-((dimethylamino)methyl)cyclohexyl)-6- methylpyrido[3,4-d]pyrimidin-2-amine | 431.2 |
| 46 | 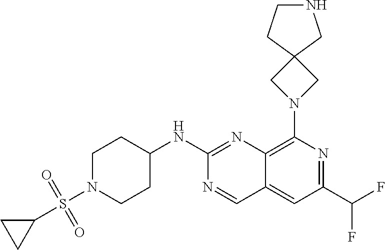 [SMILES: O=S(=O)(C1CC1)N1CCC(Nc2ncc3cc(C(F)F)nc(N4CC5(CCNC5)C4)c3n2)CC1] | 1-(cyclopropanesulfonyl)-N-(8-{2,6- diazaspiro[3.4]octan-2-yl}-6- (difluoromethyl)pyrido[3,4-d]pyrimidin-2- yl)piperidin-4-amine | 494.3 |
| 47 | 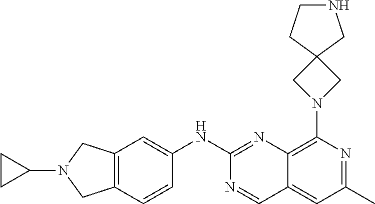 [SMILES: Cc1cc2cnc(Nc3ccc4c(c3)CN(C3CC3)C4)nc2c(N2CC3(CCNC3)C2)n1] | N-(2-cyclopropylisoindolin-5-yl)-6-methyl-8-(2,6- diazaspiro[3.4]octan-2-yl)pyrido[3,4-d]pyrimidin- 2-amine | 428.3 |
| 48 | 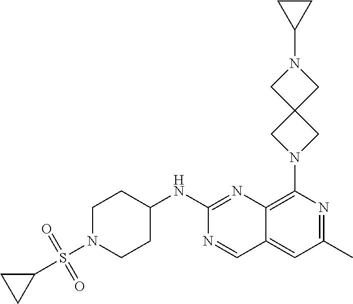 [SMILES: Cc1cc2cnc(NC3CCN(S(=O)(=O)C4CC4)CC3)nc2c(N2CC3(C2)CN(C2CC2)C3)n1] | 8-(6-cyclopropyl-2,6-diazaspiro[3.3]heptan-2-yl)- N-(1-(cyclopropylsulfonyl)piperidin-4-yl)-6- methylpyrido[3,4-d]pyrimidin-2-amine | 484.2 |
| 49 | 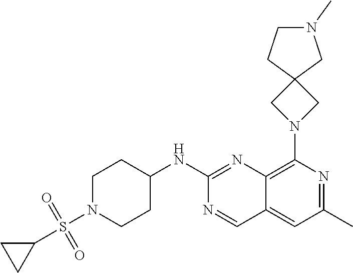 [SMILES: Cc1cc2cnc(NC3CCN(S(=O)(=O)C4CC4)CC3)nc2c(N2CC3(CCN(C)C3)C2)n1] | N-(1-(cyclopropylsulfonyl)piperidin-4-yl)-6- methyl-8-(6-methyl-2,6-diazaspiro[3.4]octan-2- yl)pyrido[3,4-d]pyrimidin-2-amine | 472.2 |
| 50 | 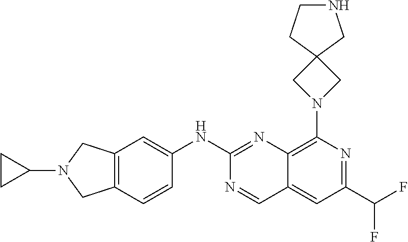 [SMILES: FC(F)c1cc2cnc(Nc3ccc4c(c3)CN(C3CC3)C4)nc2c(N2CC3(CCNC3)C2)n1] | N-(2-cyclopropylisoindolin-5-yl)-6- (difluoromethyl)-8-(2,6-diazaspiro[3.4]octan-2- yl)pyrido[3,4-d]pyrimidin-2-amine | 464.2 |
| 51 | 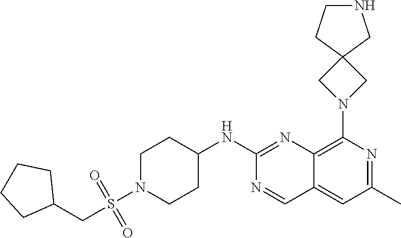 [SMILES: Cc1cc2cnc(NC3CCN(S(=O)(=O)CC4CCCC4)CC3)nc2c(N2CC3(CCNC3)C2)n1] | N-(1-((cyclopentylmethyl)sulfonyl)piperidin-4-yl)- 6-methyl-8-(2,6-diazaspiro[3.4]octan-2- yl)pyrido[3,4-d]pyrimidin-2-amine | 500.3 |
| 52 | 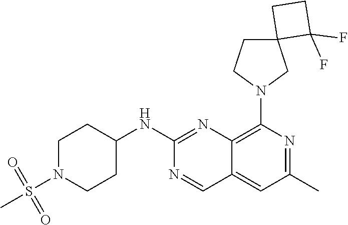 [SMILES: Cc1cc2cnc(NC3CCN(S(C)(=O)=O)CC3)nc2c(N2CCC3(CCC3(F)F)C2)n1] | 8-(1,1-difluoro-6-azaspiro[3.4]octan-6-yl)-6- methyl-N-(1-(methylsulfonyl)piperidin-4- yl)pyrido[3,4-d]pyrimidin-2-amine | 467.3 |
| 53 | 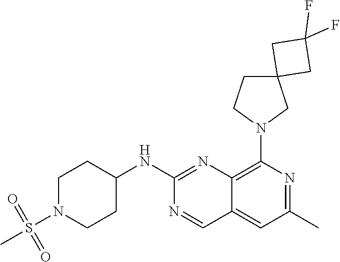 [SMILES: Cc1cc2cnc(NC3CCN(S(C)(=O)=O)CC3)nc2c(N2CCC3(C2)CC(F)(F)C3)n1] | 8-(2,2-difluoro-6-azaspiro[3.4]octan-6-yl)-6- methyl-N-(1-(methylsulfonyl)piperidin-4- yl)pyrido[3,4-d]pyrimidin-2-amine | 467.3 |
| 54 | 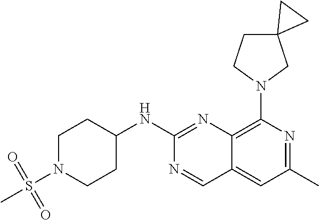 | 6-methyl-N-(1-(methylsulfonyl)piperidin-4-yl)-8- (5-azaspiro[2.4]heptan-5-yl)pyrido[3,4- d]pyrimidin-2-amine | 417.3 |
| 55 | 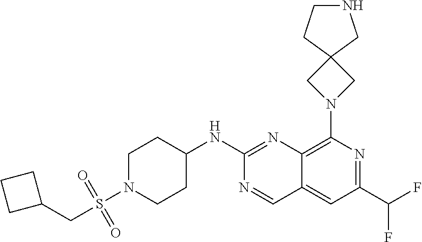 [SMILES: O=S(=O)(CC1CCC1)N1CCC(Nc2ncc3cc(C(F)F)nc(N4CC5(CCNC5)C4)c3n2)CC1] | N-(1-((cyclobutylmethyl)sulfonyl)piperidin-4-yl)- 6-(difluoromethyl)-8-(2,6-diazaspiro[3.4]octan-2- yl)pyrido[3,4-d]pyrimidin-2-amine | 522.3 |
| 56 | 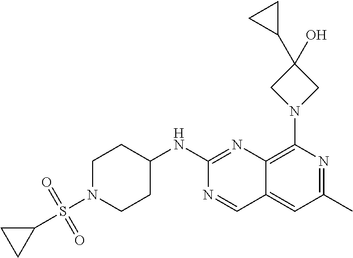 [SMILES: Cc1cc2cnc(NC3CCN(S(=O)(=O)C4CC4)CC3)nc2c(N2CC(O)(C3CC3)C2)n1] | 3-cyclopropyl-1-(2-((1- (cyclopropylsulfonyl)piperidin-4-yl)amino)-6- methylpyrido[3,4-d]pyrimidin-8-yl)azetidin-3-ol | 459.2 |
| 57 | 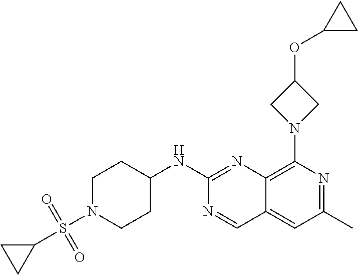 [SMILES: Cc1cc2cnc(NC3CCN(S(=O)(=O)C4CC4)CC3)nc2c(N2CC(OC3CC3)C2)n1] | 8-(3-cyclopropoxyazetidin-1-yl)-N-(1- (cyclopropylsulfonyl)piperidin-4-yl)-6- methylpyrido[3,4-d]pyrimidin-2-amine | 459.3 |
| 58 | 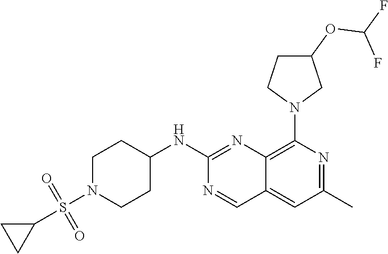 [SMILES: Cc1cc2cnc(NC3CCN(S(=O)(=O)C4CC4)CC3)nc2c(N2CCC(OC(F)F)C2)n1] | N-(1-(cyclopropylsulfonyl)piperidin-4-yl)-8-(3- (difluoromethoxy)pyrrolidin-1-yl)-6- methylpyrido[3,4-d]pyrimidin-2-amine | 483.3 |
| 59 | 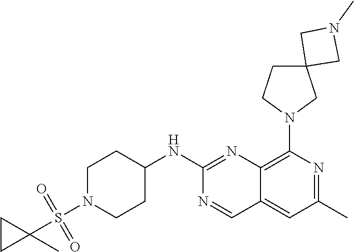 [SMILES: Cc1cc2cnc(NC3CCN(S(=O)(=O)C4(C)CC4)CC3)nc2c(N2CCC3(CN(C)C3)C2)n1] | 6-methyl-8-(2-methyl-2,6-diazaspiro[3.4]octan-6- yl)-N-(1-((1-methylcyclopropyl)sulfonyl)piperidin- 4-yl)pyrido[3,4-d]pyrimidin-2-amine | 486.3 |
| 60 | 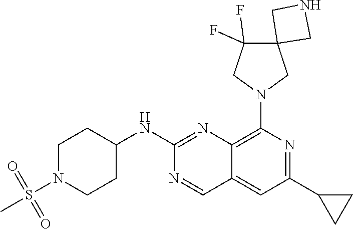 [SMILES: CS(=O)(=O)N1CCC(Nc2ncc3cc(C4CC4)nc(N4CC(F)(F)C5(CNC5)C4)c3n2)CC1] | 6-cyclopropyl-8-(8,8-difluoro-2,6- diazaspiro[3.4]octan-6-yl)-N-(1- (methylsulfonyl)piperidin-4-yl)pyrido[3,4- d]pyrimidin-2-amine | 494.3 |
| 61 | 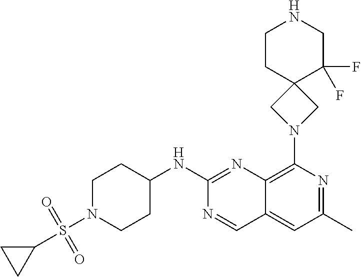 [SMILES: Cc1cc2cnc(NC3CCN(S(=O)(=O)C4CC4)CC3)nc2c(N2CC3(CCNCC3(F)F)C2)n1] | N-(1-(cyclopropylsulfonyl)piperidin-4-yl)-8-(5,5- difluoro-2,7-diazaspiro[3.5]nonan-2-yl)-6- methylpyrido[3,4-d]pyrimidin-2-amine | 508.3 |
| 62 | 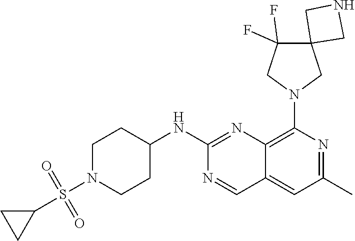 [SMILES: Cc1cc2cnc(NC3CCN(S(=O)(=O)C4CC4)CC3)nc2c(N2CC(F)(F)C3(CNC3)C2)n1] | N-(1-(cyclopropylsulfonyl)piperidin-4-yl)-8-(8,8- difluoro-2,6-diazaspiro[3.4]octan-6-yl)-6- methylpyrido[3,4-d]pyrimidin-2-amine | 494.2 |
| 63 | 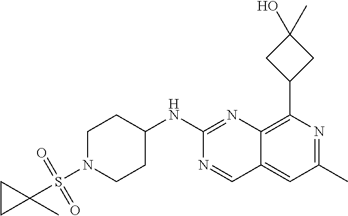 [SMILES: Cc1cc2cnc(NC3CCN(S(=O)(=O)C4(C)CC4)CC3)nc2c(C2CC(C)(O)C2)n1] | 1-methyl-3-(6-methyl-2-((1-((1- methylcyclopropyl)sulfonyl)piperidin-4- yl)amino)pyrido[3,4-d]pyrimidin-8-yl)cyclobutan- 1-ol | 446.2 |
| 64 | 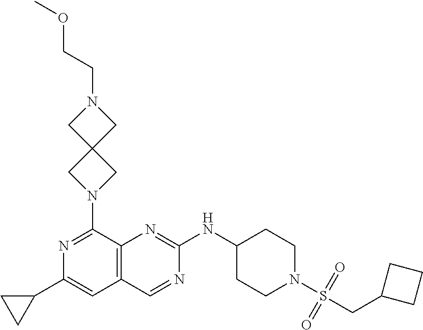 [SMILES: COCCN1CC2(C1)CN(c1nc(C3CC3)cc3cnc(NC4CCN(S(=O)(=O)CC5CCC5)CC4)nc13)C2] | N-(1-((cyclobutylmethyl)sulfonyl)piperidin-4-yl)- 6-cyclopropyl-8-(6-(2-methoxyethyl)-2,6- diazaspiro[3.3]heptan-2-yl)pyrido[3,4-d]pyrimidin- 2-amine | 556.3 |
| 65 | 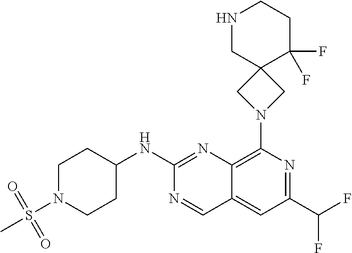 [SMILES: CS(=O)(=O)N1CCC(Nc2ncc3cc(C(F)F)nc(N4CC5(CNCCC5(F)F)C4)c3n2)CC1] | 8-(9,9-difluoro-2,6-diazaspiro[3.5]nonan-2-yl)-6- (difluoromethyl)-N-(1-(methylsulfonyl)piperidin- 4-yl)pyrido[3,4-d]pyrimidin-2-amine | 518.2 |
| 66 | 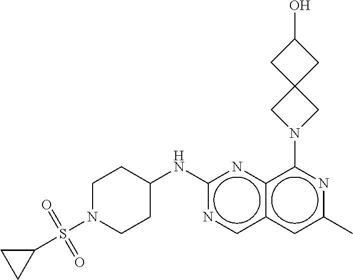 | 2-(2-((1-(cyclopropylsulfonyl)piperidin-4- yl)amino)-6-methylpyrido[3,4-d]pyrimidin-8-yl)- 2-azaspiro[3.3]heptan-6-ol | 459.2 |
| 67 | 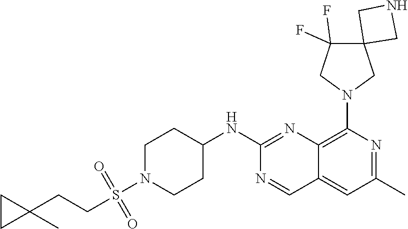 [SMILES: Cc1cc2cnc(NC3CCN(S(=O)(=O)CCC4(C)CC4)CC3)nc2c(N2CC(F)(F)C3(CNC3)C2)n1] | 8-(8,8-difluoro-2,6-diazaspiro[3.4]octan-6-yl)-6- methyl-N-(1-((2-(1- methylcyclopropyl)ethyl)sulfonyl)piperidin-4- yl)pyrido[3,4-d]pyrimidin-2-amine | 536.3 |
| 68 | 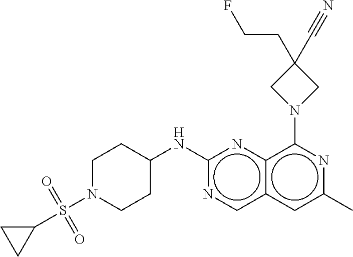 | 1-(2-((1-(cyclopropylsulfonyl)piperidin-4- yl)amino)-6-methylpyrido[3,4-d]pyrimidin-8-yl)- 3-(2-fluoroethyl)azetidine-3-carbonitrile | 474.3 |
| 69 | 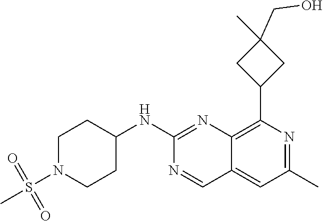 | (1-methyl-3-(6-methyl-2-((1- (methylsulfonyl)piperidin-4-yl)amino)pyrido[3,4- d]pyrimidin-8-yl)cyclobutyl)methanol | 421.2 |
| 70 | 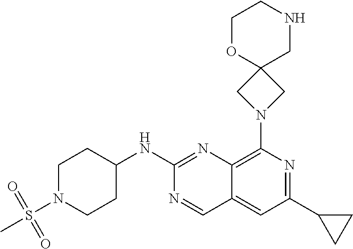 [SMILES: CS(=O)(=O)N1CCC(Nc2ncc3cc(C4CC4)nc(N4CC5(CNCCO5)C4)c3n2)CC1] | 6-cyclopropyl-N-(1-(methylsulfonyl)piperidin-4- yl)-8-(5-oxa-8-azaspiro[3.5]nonan-2-yl)pyrido[3,4- d]pyrimidin-2-amine | 473.2 |
| 71 | 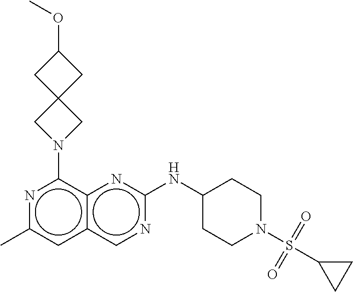 | N-(1-(cyclopropylsulfonyl)piperidin-4-yl)-8-(6- methoxy-2-azaspiro[3.3]heptan-2-yl)-6- methylpyrido[3,4-d]pyrimidin-2-amine | 473.3 |
| 72 | 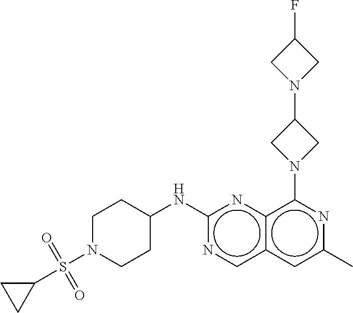 | N-(1-(cyclopropylsulfonyl)piperidin-4-yl)-8-(3- fluoro-[1,3′-biazetidin]-l′-yl)-6-methylpyrido[3,4- d]pyrimidin-2-amine | 476.2 |
| 73 | 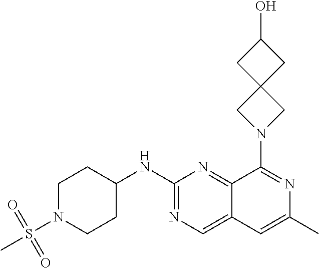 | 2-(6-methyl-2-((1-(methylsulfonyl)piperidin-4- yl)amino)pyrido[3,4-d]pyrimidin-8-yl)-2- azaspiro[3.3]heptan-6-ol | 433.2 |
| 74 | 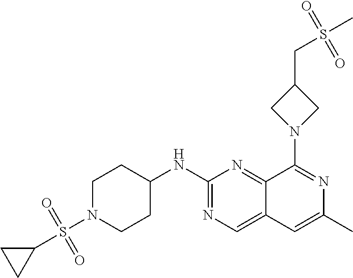 [SMILES: Cc1cc2cnc(NC3CCN(S(=O)(=O)C4CC4)CC3)nc2c(N2CC(CS(C)(=O)=O)C2)n1] | N-(1-(cyclopropylsulfonyl)piperidin-4-yl)-6- methyl-8-(3-((methylsulfonyl)methyl)azetidin-1- yl)pyrido[3,4-d]pyrimidin-2-amine | 495.2 |
| 75 | 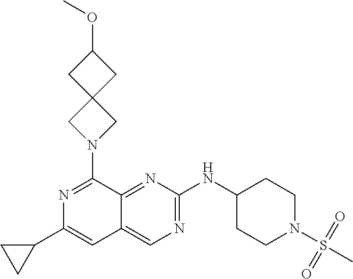 [SMILES: COC1CC2(C1)CN(c1nc(C3CC3)cc3cnc(NC4CCN(S(C)(=O)=O)CC4)nc13)C2] | 6-cyclopropyl-8-(6-methoxy-2- azaspiro[3.3]heptan-2-yl)-N-(1- (methylsulfonyl)piperidin-4-yl)pyrido[3,4- d]pyrimidin-2-amine | 473.3 |
| 76 | 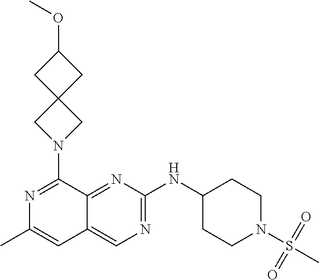 | 8-(6-methoxy-2-azaspiro[3.3]heptan-2-yl)-6- methyl-N-(1-(methylsulfonyl)piperidin-4- yl)pyrido[3,4-d]pyrimidin-2-amine | 447.2 |
| 77 | 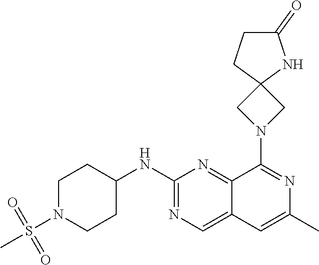 | 2-(6-methyl-2-((1-(methylsulfonyl)piperidin-4- yl)amino)pyrido[3,4-d]pyrimidin-8-yl)-2,5- diazaspiro[3.4]octan-6-one | 446.2 |
| 78 | 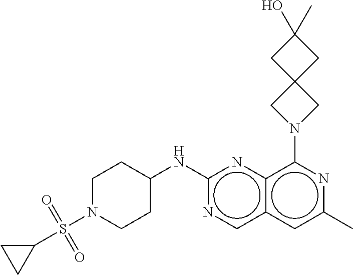 | 2-(2-((1-(cyclopropylsulfonyl)piperidin-4- yl)amino)-6-methylpyrido[3,4-d]pyrimidin-8-yl)- 6-methyl-2-azaspiro[3.3]heptan-6-ol | 473.2 |
| 79 | 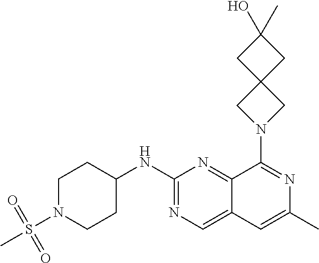 | 6-methyl-2-(6-methyl-2-((1- (methylsulfonyl)piperidin-4-yl)amino)pyrido[3,4- d]pyrimidin-8-yl)-2-azaspiro[3.3]heptan-6-ol | 447.2 |
| 80 | 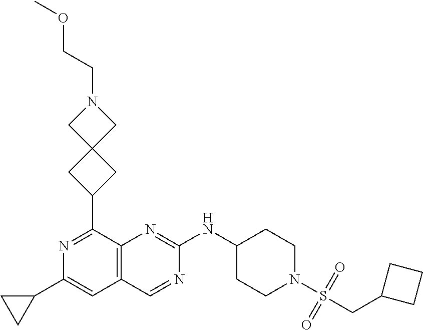 [SMILES: COCCN1CC2(CC(c3nc(C4CC4)cc4cnc(NC5CCN(S(=O)(=O)CC6CCC6)CC5)nc34)C2)C1] | N-(1-((cyclobutylmethyl)sulfonyl)piperidin-4-yl)- 6-cyclopropyl-8-(2-(2-methoxyethyl)-2- azaspiro[3.3]heptan-6-yl)pyrido[3,4-d]pyrimidin- 2-amine | 555.4 |
| 81 | 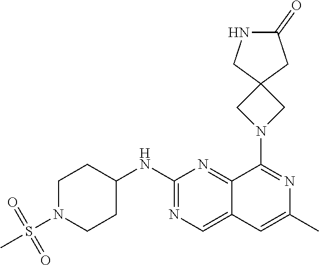 | 2-(6-methyl-2-((1-(methylsulfonyl)piperidin-4- yl)amino)pyrido[3,4-d]pyrimidin-8-yl)-2,6- diazaspiro[3.4]octan-7-one | 446.2 |
| 82 | 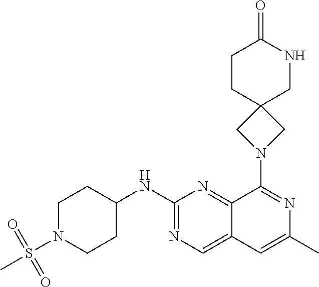 | 2-(6-methyl-2-((1-(methylsulfonyl)piperidin-4- yl)amino)pyrido[3,4-d]pyrimidin-8-yl)-2,6- diazaspiro[3.5]nonan-7-one | 460.2 |
| 83 | 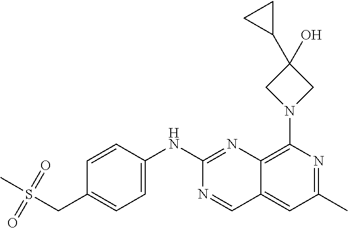 | 3-cyclopropyl-1-(6-methyl-2-((4- ((methylsulfonyl)methyl)phenyl)amino)pyrido[3,4- d]pyrimidin-8-yl)azetidin-3-ol | 440.1 |
| 84 | 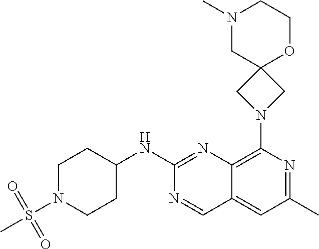 | 6-methyl-8-(8-methyl-5-oxa-8-azaspiro[3.5]nonan- 2-yl)-N-(1-(methylsulfonyl)piperidin-4- yl)pyrido[3,4-d]pyrimidin-2-amine | 461.3 |
| 85 | 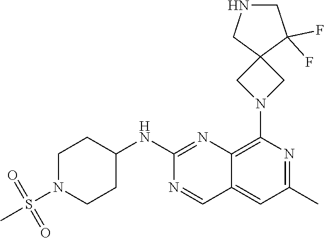 [SMILES: Cc1cc2cnc(NC3CCN(S(C)(=O)=O)CC3)nc2c(N2CC3(CNCC3(F)F)C2)n1] | 8-(8,8-difluoro-2,6-diazaspiro[3.4]octan-2-yl)-6- methyl-N-(1-(methylsulfonyl)piperidin-4- yl)pyrido[3,4-d]pyrimidin-2-amine | 468.3 |
| 86 | 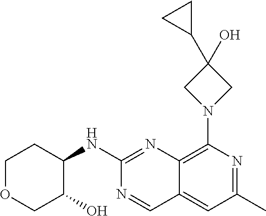 [SMILES: Cc1cc2cnc(N[C@@H]3CCOC[C@H]3O)nc2c(N2CC(O)(C3CC3)C2)n1] | 3-cyclopropyl-1-(2-(((3S,4R)-3- hydroxytetrahydro-2H-pyran-4-yl)amino)-6- methylpyrido[3,4-d]pyrimidin-8-yl)azetidin-3-ol | 372.2 |
| 87 | 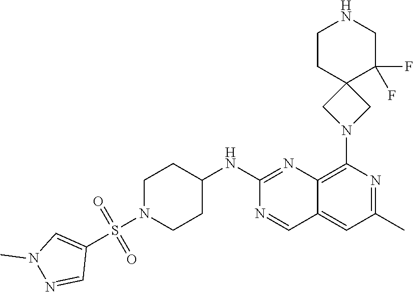 [SMILES: Cc1cc2cnc(NC3CCN(S(=O)(=O)c4cnn(C)c4)CC3)nc2c(N2CC3(CCNCC3(F)F)C2)n1] | 8-(5,5-difluoro-2,7-diazaspiro[3.5]nonan-2-yl)-6- methyl-N-(1-((1-methyl-1H-pyrazol-4- yl)sulfonyl)piperidin-4-yl)pyrido[3,4-d]pyrimidin- 2-amine | 548.3 |
| 88 | 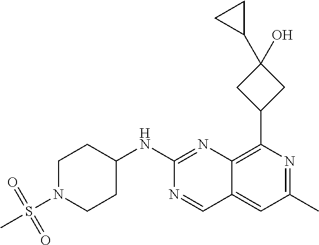 | 1-cyclopropyl-3-(6-methyl-2-((1- (methylsulfonyl)piperidin-4-yl)amino)pyrido[3,4- d]pyrimidin-8-yl)cyclobutan-1-ol | 432.2 |
| 89 | 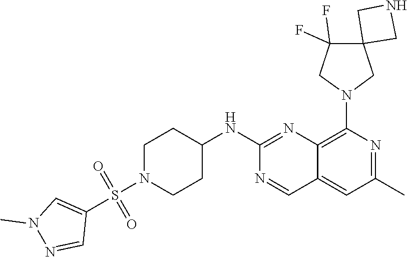 | 8-(8,8-difluoro-2,6-diazaspiro[3.4]octan-6-yl)-6- methyl-N-(1-((1-methyl-1H-pyrazol-4- yl)sulfonyl)piperidin-4-yl)pyrido[3,4-d]pyrimidin- 2-amine | 534.2 |
| 90 | 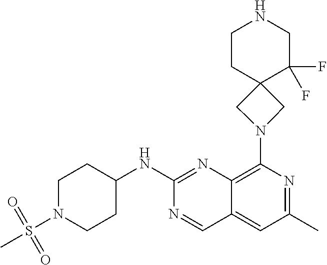 [SMILES: Cc1cc2cnc(NC3CCN(S(C)(=O)=O)CC3)nc2c(N2CC3(CCNCC3(F)F)C2)n1] | 8-(5,5-difluoro-2,7-diazaspiro[3.5]nonan-2-yl)-6- (difluoromethyl)-N-(1-(methylsulfonyl)piperidin- 4-yl)pyrido[3,4-d]pyrimidin-2-amine | 518.2 |
| 91 | 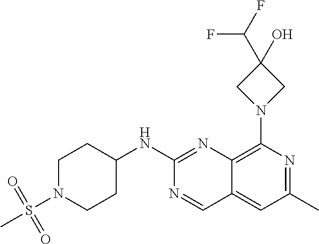 | 3-(difluoromethyl)-1-(6-methyl-2-((1- (methylsulfonyl)piperidin-4-yl)amino)pyrido[3,4- d]pyrimidin-8-yl)azetidin-3-ol | 443.1 |
| 92 | 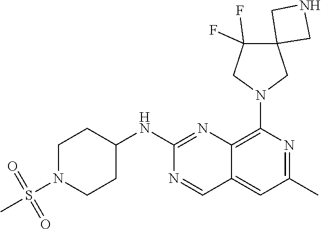 | 8-(8,8-difluoro-2,6-diazaspiro[3.4]octan-6-yl)-6- methyl-N-(1-(methylsulfonyl)piperidin-4- yl)pyrido[3,4-d]pyrimidin-2-amine | 468.1 |
| 93 | 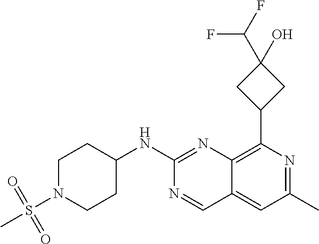 | 1-(difluoromethyl)-3-(6-methyl-2-((1- (methylsulfonyl)piperidin-4-yl)amino)pyrido[3,4- d]pyrimidin-8-yl)cyclobutan-1-ol | 442.1 |
| 94 | 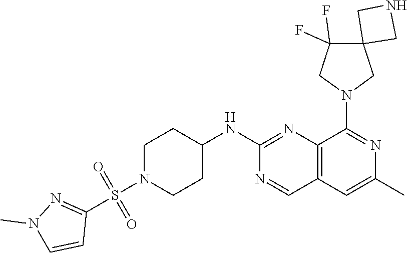 | 8-(8,8-difluoro-2,6-diazaspiro[3.4]octan-6-yl)-6- methyl-N-(1-((1-methyl-1H-pyrazol-3- yl)sulfonyl)piperidin-4-yl)pyrido[3,4-d]pyrimidin- 2-amine | 534 |
| 95 | 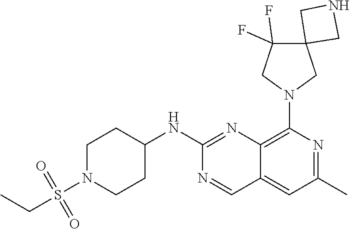 [SMILES: CCS(=O)(=O)N1CCC(Nc2ncc3cc(C)nc(N4CC(F)(F)C5(CNC5)C4)c3n2)CC1] | 8-(8,8-difluoro-2,6-diazaspiro[3.4]octan-6-yl)-N- (1-(ethylsulfonyl)piperidin-4-yl)-6- methylpyrido[3,4-d]pyrimidin-2-amine | 482 |
| 96 | 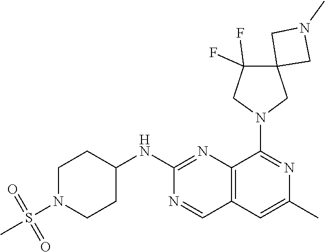 | 8-(8,8-difluoro-2-methyl-2,6-diazaspiro[3.4]octan- 6-yl)-6-methyl-N-(1-(methylsulfonyl)piperidin-4- yl)pyrido[3,4-d]pyrimidin-2-amine | 482.2 |
| 97 | 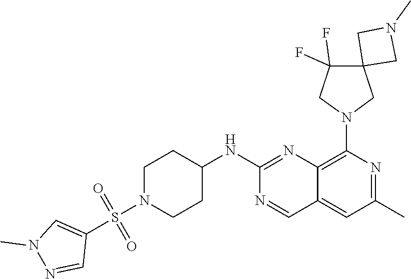 | 8-(8,8-difluoro-2-methyl-2,6-diazaspiro[3.4]octan- 6-yl)-6-methyl-N-(1-((1-methyl-1H-pyrazol-4- yl)sulfonyl)piperidin-4-yl)pyrido[3,4-d]pyrimidin- 2-amine | 548.4 |
| 98 | 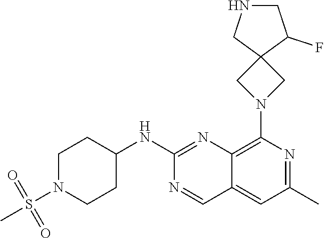 [SMILES: Cc1cc2cnc(NC3CCN(S(C)(=O)=O)CC3)nc2c(N2CC3(CNCC3F)C2)n1] | 8-(8-fluoro-2,6-diazaspiro[3.4]octan-2-yl)-6- methyl-N-(1-(methylsulfonyl)piperidin-4- yl)pyrido[3,4-d]pyrimidin-2-amine | 450.1 |
| 99 | 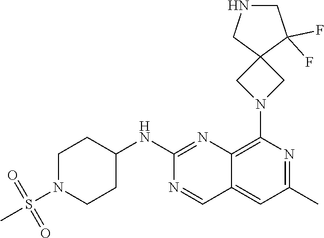 [SMILES: Cc1cc2cnc(NC3CCN(S(C)(=O)=O)CC3)nc2c(N2CC3(CNCC3(F)F)C2)n1] | 8-(8,8-difluoro-6-azaspiro[3.4]octan-2-yl)-6- methyl-N-(1-(methylsulfonyl)piperidin-4- yl)pyrido[3,4-d]pyrimidin-2-amine | 467.3 |
| 100 | 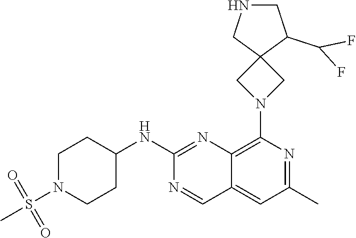 [SMILES: Cc1cc2cnc(NC3CCN(S(C)(=O)=O)CC3)nc2c(N2CC3(CNCC3C(F)F)C2)n1] | 8-(8-(difluoromethyl)-2,6-diazaspiro[3.4]octan-2- yl)-6-methyl-N-(1-(methylsulfonyl)piperidin-4- yl)pyrido[3,4-d]pyrimidin-2-amine | 482.4 |
| 101 | 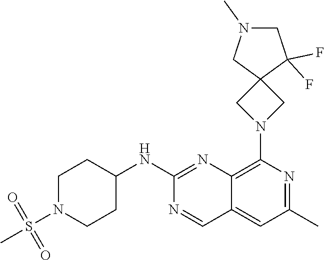 [SMILES: Cc1cc2cnc(NC3CCN(S(C)(=O)=O)CC3)nc2c(N2CC3(CN(C)CC3(F)F)C2)n1] | 8-(8,8-difluoro-6-methyl-2,6-diazaspiro[3.4]octan- 2-yl)-6-methyl-N-(1-(methylsulfonyl)piperidin-4- yl)pyrido[3,4-d]pyrimidin-2-amine | 482.3 |
| 102 | 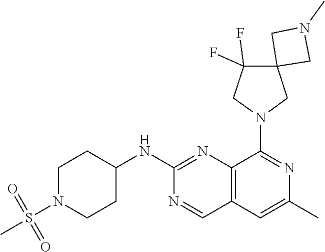 | N-(1-(cyclopropylsulfonyl)piperidin-4-yl)-8-(8,8- difluoro-2-methyl-2,6-diazaspiro[3.4]octan-6-yl)- 6-methylpyrido[3,4-d]pyrimidin-2-amine | 508.2 |
| 103 | 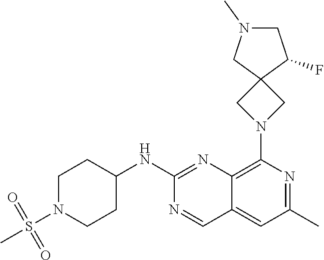 [SMILES: Cc1cc2cnc(NC3CCN(S(C)(=O)=O)CC3)nc2c(N2CC3(CN(C)C[C@@H]3F)C2)n1] | (R)-8-(8-fluoro-6-methyl-2,6-diazaspiro[3.4]octan- 2-yl)-6-methyl-N-(1-(methylsulfonyl)piperidin-4- yl)pyrido[3,4-d]pyrimidin-2-amine | 464.2 |
| 104 | 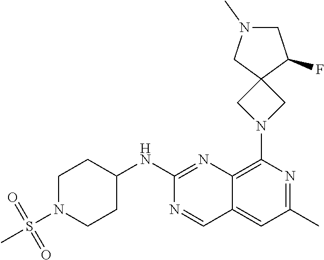 [SMILES: Cc1cc2cnc(NC3CCN(S(C)(=O)=O)CC3)nc2c(N2CC3(CN(C)C[C@H]3F)C2)n1] | (S)-8-(8-fluoro-6-methyl-2,6-diazaspiro[3.4]octan- 2-yl)-6-methyl-N-(1-(methylsulfonyl)piperidin-4- yl)pyrido[3,4-d]pyrimidin-2-amine | 464.2 |

The NMR of the compounds of Table 1 are provided in Table 1B below.

TABLE 1B

| Compound | |
|---|---|
| No. | ¹H NMR (ppm) |
| 1 | ¹H NMR (400 MHz, DMSO-d₆) δ (ppm) 9.09 (s, 1H), 7.95-7.80 (m,1H), 7.79- |
| | 7.32 (m, 1H), 7.12-6.96 (m, 1H), 4.00-3.71 (m, 5H), 3.70-3.50 (m, 2H), 3.02- |
| | 2.81 (m, 5H), 2.80-2.61 (m, 4H), 2.15-1.92 (m, 2H), 1.78-1.55 (m, 3H), 0.55- |
| | 0.41 (m, 2H), 0.40-0.25 (m, 2H). |
| 2 | 1H NMR (400 MHz, DMSO-d6) δ 8.99 (s, 1H), 7.73 (d, J = 5.6 Hz, 1H), 7.52 |
| | (s, 1H), 6.81 (d, J = 5.2 Hz, 1H), 4.10 (S, 2H), 3.89 (S, 3H), 3.59 (d, J = 12.4 |
| | Hz, 2H), 2.93-2.86 (m, 5H), 2.75-2.56 (m, 4H), 2.13-1.96 (m, 4H), 1.69-1.56 |
| | (m, 2H). |
| 3 | ¹H NMR (300 MHz, DMSO-d₆) δ(ppm) 9.03 (s, 1H), 7.76 (d, J = 6.0 Hz, 1H), |
| | 7.57-7.51 (m, 1H), 6.87 (d, J = 6.0 Hz, 1H), 4.45-4.27 (m, 1H), 4.11-3.98 (m, |
| | 3H), 3.91-3.70 (m, 1H), 3.59-3.55 (m, 2H), 2.98-2.80 (m, 5H), 2.25-2.07 (m, |
| | 1H), 2.16-1.99 (m, 3H), 1.73-1.60 (m, 4H). |
| 4 | ¹H NMR (400 MHz, DMSO-d₆) δ (ppm) 9.48 (s, 1H), 9.10 (s, 1H), 7.90 (d, |
| | j = 5.6 Hz, 1H), 7.51 (d, j = 9.2 Hz, 2H), 7.01-6.82 (m, 3H), 4.12-3.95 (m, 2H), |
| | 3.92-3.72 (m, 2H), 3.20-3.01 (m, 4H), 2.70-2.55 (m, 4H), 2.50-2.38 (m, 4H), |
| | 2.30-2.15 (m, 3H), 2.10-1.92 (m, 2H). |
| 5 | ¹H NMR (400 MHz, DMSO-d₆) δ (ppm) 9.04 (s, 1H), 7.77 (d, j = 5.2 Hz, 1H), |
| | 7.62 (s, 1H), 6.90 (d, j = 5.6 Hz, 1H), 4.75-4.41 (m, 1H), 4.20-3.96 (m, 3H), |
| | 3.92-3.72 (m, 1H), 3.71-3.55 (m, 2H), 2.98-2.80 (m, 5H), 2.45-2.38 (m, 1H), |
| | 2.25-2.16 (m, 1H), 2.08-1.99 (m, 2H), 1.70-1.51 (m, 2H), 1.36-1.25 (m, 1H), |
| | 0.70-0.55 (m, 2H), 0.54-0.41 (m, 2H). |
| 6 | 1H NMR (400 MHz, DMSO-d6) δ (ppm) 9.13 (s, 1H), 7.94-7.62 (m, 3H), |
| | 7.14-6.97 (m, 2H), 4.39 (s, 4H), 4.12-4.18 (m, 6H), 3.98-3.92 (m, 2H), 3.27- |
| | 3.25 (m, 2H), 2.67-2.61 (m, 4H), 2.16-2.13 (m, 2H), 1.42-1.34 (m, 1H), 1.31- |
| | 1.23 (m, 3H). |
| 7 | ¹H NMR (400 MHz, DMSO-d₆) δ(ppm) 8.98 (s, 1H), 7.72 (d, J = 7.2 Hz, 1H), |
| | 7.46 (s, 1H), 6.78 (d, J = 7.2 Hz, 1H), 4.00-3.80 (m, 5H), 3.75-3.52 (m, 6H), |
| | 2.95-2.86 (m, 5H), 2.12-2.00 (m, 2H), 1.98-1.80 (m, 2H), 1.70-1.50 (m, 6H). |
| 8 | ¹H NMR (300 MHz, CDCl₃) δ(ppm) 8.97-8.69 (m, 1H), 7.97-7.75 (m, 1H), |
| | 6.84-6.66 (m, 1H), 5.35-5.13 (m, 1H), 4.71-4.31 (m, 4H), 4.15-3.88 (m, 1H), |
| | 3.82-3.68(m, 2H), 3.65-3.44 (m, 4H), 3.19-2.99 (m, 2H), 2.96-2.75 (m, 3H), |
| | 2.33-2.15 (m, 2H), 2.14-1.85 (m, 3H), 0.56-0.30 (m, 4H). |
| 9 | ¹H NMR (300 MHz, CDCl₃) δ(ppm) 8.94 (d, J = 3.8 Hz, 1H), 7.91 (d, J = 5.5 Hz, |
| | 1H), 7.45-7.36 (m, 2H), 7.12 (s, 1H), 6.97 (dd, J = 9.5, 2.8 Hz, 2H), 6.78 (d, |
| | J = 5.5 Hz, 1H), 4.11-3.95 (m, 3H), 3.89 (d, J = 11.7 Hz, 1H), 3.52 (dt, J = 9.9, 6.8 |
| | Hz, 1H), 3.45-3.30 (m, 3H), 3.23 (d, J = 5.7 Hz, 4H), 2.81 (d, J = 2.7 Hz, 3H), |
| | 2.69-2.57 (m, 4H), 2.39 (d, J = 5.6 Hz, 3H), 2.00 (td, J = 9.2, 7.5, 3.8 Hz, 4H). |
| 10 | ¹H-NMR (400 MHz, Chloroform-d) δ (ppm) 9.18-8.91 (m, 1H), 8.08-7.80 (m, |
| | 2H), 6.90-6.69 (m, 1H), 6.44-6.24 (m, 1H), 4.48-4.32 (m, 1H), 4.28-3.99 (m, |
| | 5H), 3.75-3.55 (m, 2H), 2.94 (s, 2H), 2.63-2.45 (m, 3H), 2.30 (s, 1H), 2.05 (s, |
| | 1H), 1.52-1.36 (m, 2H). |
| 11 | ¹H NMR (400 MHz, DMSO-d₆) δ (ppm) 8.96 (s, 1H), 8.34 (s, 1H), 7.77 (s, |
| | 1H), 7.53 (s, 1H), 6.76 (s, 1H), 4.33-4.24 (m, 2H), 4.23-4.15 (m, 1H), 3.92 (s, |
| | 3H), 3.70 (m, 1H), 3.60-3.50 (m, 2H), 3.31-3.25 (m, 2H), 3.18-2.99 (m, 2H), |
| | 2.49-2.40 (m, 3H), 2.33 (s, 3H), 2.08-1.97 (m, 2H), 1.70-1.54 (m, 2H). |
| 12 | ¹H NMR (400 MHz, DMSO-d₆) δ (ppm) 8.98 (s, 1H), 7.54 (s, 1H), 6.78 (s, |
| | 1H), 4.40-4.22 (m, 4H), 3.90-3.79 (m, 1H), 3.61 (d, J = 11.6 Hz, 3H), 3.26- |
| | 3.15 (m, 3H), 2.97-2.84 (m, 5H), 2.34 (s, 3H), 2.13-1.98 (m, 2H), 1.69-1.55 |
| | (m, 2H) |
| 13 | ¹H NMR (400 MHz, DMSO-d₆) δ (ppm) 8.98 (s, 1H), 7.54 (s, 1H), 6.78 (s, |
| | 1H), 4.40-4.19 (m, 4H), 3.90-3.79 (m, 1H), 3.72-3.63 (m, 2H), 3.38-3.35 (m, |
| | 1H), 3.20 (d, J = 7.6 Hz, 2H), 3.00 (t, J = 11.6 Hz, 2H), 2.70-2.56 (m, 2H), |
| | 2.34 (s, 3H), 2.09-2.00 (m, 2H), 1.67-1.51 (m, 2H), 1.03-0.90 (m, 4H) |
| 14 | ¹H NMR (400 MHz, CDCL₃) δ (ppm) 8.92 (s, 1H), 7.92-7.90 (m, 1H), 6.81- |
| | 6.79 (m, 1H), 5.70 (s, 1H), 4.82-4.75 (m, 1H), 4.32-4.08 (m, 6H), 3.96-3.91 |
| | (m, 2H), 2.93 (s, 3H), 2.36-2.27 (m, 1H), 2.17-2.01 (m, 1H), 1.65-1.60 (m, |
| | 2H). |
| 15 | ¹H-NMR (400 MHz, Chloroform-d) δ (ppm) 8.83 (s, 1H), 7.87 (d, J = 5.5 Hz, |
| | 1H), 6.74 (d, J = 5.5 Hz, 1H), 5.35 (s, 1H), 4.31 (s, 2H), 4.16-3.96 (m, 2H), |
| | 3.62-3.44 (m, 2H), 3.41 (s, 2H), 3.22-3.06 (m, 3H), 2.90 (s, 3H), 2.53-2.46 (m, |
| | 2H), 2.37 (s, 3H), 2.13-2.01 (m, 4H), 1.64 (d, J = 2.6 Hz, 2H). |
| 16 | ¹H NMR (400 MHz, DMSO-d6) δ 9.04 (s, 1H), 7.85-7.71 (m, 4H), 7.69-7.53 |
| | (m, 2H), 6.96 (d, J = 5.6 Hz, 1H), 5.22-5.02 (m, 1H), 4.53-4.36 (m, 3H), 4.15- |
| | 3.96 (m, 3H), 3.65-3.50 (m, 2H), 3.03-2.86 (m, 5H), 2.07-1.93 (m, 2H), 1.70- |
| | 1.51 (m, 2H). |
| 17 | ¹H-NMR (400 MHz, Chloroform-d) δ (ppm) 8.93-8.77 (m, 1H), 7.97-7.80 (m, |
| | 1H), 6.86-6.67 (m, 1H), 5.49-5.30 (m, 1H), 4.51-4.11 (m, 4H), 3.25-3.01 (m, |
| | 3H), 2.55-2.05 (m, 8H), 1.54-1.23 (m, 3H). |
| 18 | ¹H NMR (400 MHz, DMSO-d6) δ 9.14 (s, 1H), 7.83 (d, J = 5.2 Hz, 1H), 7.73- |
| | 7.66 (m, 1H), 7.60 (d, J = 2.8 Hz, 1H), 6.97-6.91 (m, 1H), 6.87 (d, J = 5.2 Hz, |
| | 1H), 3.92 (s, 3H), 3.90-3.79 (m, 4H), 3.68-3.57 (m, 3H), 3.56-3.42 (m, 5H), |
| | 2.68-2.53 (m, 4H), 1.89-1.78 (m, 2H), 1.61-1.45 (m, 4H), 1.00-0.83 (m, 3H). |
| 19 | ¹H-NMR (400 MHz, Chloroform-d) δ (ppm) 9.05-8.94 (m, 1H), 8.16-8.01 |
| | (m, 1H), 7.27-7.19 (m, 1H), 7.15-7.04 (m, 2H), 6.60 (t, J = 9.1 Hz, 1H), 5.44- |
| | 5.25 (m, 1H), 4.45-4.30 (m, 2H), 3.96-3.71 (m, 3H), 2.86-2.72 (m, 5H), |
| | 2.30-2.16 (m, 4H), 1.96-1.83 (m, 4H), 1.73-1.60 (m, 4H). |
| 20 | ¹H NMR (300 MHz, Chloroform-d) δ (ppm) 8.96 (s, 1H), 7.94 (d, J = 5.5 Hz, |
| | 1H), 7.75 (s, 1H), 7.59 (s, 1H), 6.85-6.75 (m, 2H), 4.29-3.93 (m, 5H), 3.12 (s, |
| | 2H), 2.45 (s, 3H), 2.35-1.98 (m, 8H), 1.48-1.41 (m, 2H). |
| 21 | ¹H NMR (300 MHz, Chloroform-d) δ (ppm) 9.04-8.98 (m, 1H), 8.09-8.01 (m, |
| | 1H), 7.99-7.88 (m, 2H), 7.69-7.60 (m, 1H), 6.91-6.76 (m, 2H), 4.40-4.29 (m, |
| | 1H), 4.26-4.07 (m, 3H), 4.04-3.95 (m, 4H), 3.43 (s, 4H), 2.59-2.41 (m, 2H), |
| | 2.36-2.27 (m, 1H), 2.11-2.00 (m, 1H), 1.48-1.38 (m, 2H), 1.02 (t, J = 7.2 Hz, |
| | 3H). |
| 22 | ¹H-NMR (400 MHz, Chloroform-d) δ (ppm) 9.04-8.87 (m, 1H), 8.04-7.89 (m, |
| | 1H), 7.50-7.29 (m, 3H), 7.22-7.12 (m, 1H), 6.90-6.74 (m, 1H), 4.32-4.05 (m, |
| | 3H), 4.03-3.82 (m, 5H), 2.77-2.50 (m, 3H), 2.31-2.01 (m, 2H), 1.50-1.35 (m, |
| | 2H). |
| 23 | ¹H-NMR (400 MHz, Chloroform-d) δ (ppm) 8.99-8.92 (m, 1H), 7.93 (dd, J = |
| | 5.5, 1.5 Hz, 1H), 7.46-7.36 (m, 2H), 7.35-7.29 (m, 1H), 7.24-7.15 (m, 1H), |
| | 6.80 (dd, J = 5.6, 1.5 Hz, 1H), 4.12-4.04 (m, 2H), 4.04-3.97 (m, 1H), 3.93 (s, |
| | 4H), 3.88-3.83 (m, 1H), 3.57-3.48 (m, 1H), 3.43-3.36 (m, 1H), 3.36-3.29 (m, |
| | 2H), 2.86-2.76 (m, 3H), 2.66-2.60 (m, 3H), 2.09-1.92 (m, 4H). |
| 24 | ¹H NMR (300 MHz, DMSO-d6) δ (ppm) 9.89 (s, 1H), 9.22 (s, 1H), 7.87 (d, J = |
| | 5.6 Hz, 1H), 7.72 (s, 1H), 7.45 (d, J = 8.1 Hz, 1H), 7.20 (d, J = 8.1 Hz, 1H), |
| | 7.02 (d, J = 5.5 Hz, 1H), 4.57 (s, 4H), 4.52 (s, 4H), 3.86 (s, 2H), 3.78 (s, 2H), |
| | 3.33-3.30 (s, 3H). |
| 25 | ¹H NMR (400 MHz, DMSO-d6) δ 8.93 (d, J = 2.7 Hz, 1H), 7.45 (s, 1H), 6.67 |
| | (d, J = 6.0 Hz, 1H), 4.25 (s, 4H), 3.81 (s, 1H), 3.63 (d, J = 12.1 Hz, 2H), 3.47(s, |
| | 1H), 3.07-2.78 (m, 5H), 2.68-2.54 (m, 2H), 2.30 (s, 3H), 2.17-2.07 (m, 1H), |
| | 2.06-1.92 (m, 3H), 1.66-1.48 (m, 2H), 1.07-0.86 (m, 4H). |
| 26 | ¹H NMR (300 MHz, DMSO-d6) δ (ppm) 9.47 (s, 1H), 9.04 (s, 1H), 7.94 (s, |
| | 1H), 7.53 (s, 1H), 6.77 (s, 1H), 4.82-4.67 (m, 1H), 4.48 (s, 4H), 4.10 (s, 4H), |
| | 3.21 (s, 2H), 3.12 (s, 2H), 3.03 (s, 3H), 2.65-2.56 (m, 2H), 2.56-2.54 (m, 2H), |
| | 2.34 (s, 3H), 2.17 (s, 3H). |
| 27 | ¹H NMR (300 MHz, DMSO-d6) δ (ppm) 9.88 (s, 1H), 9.11 (s, 1H), 7.87 (d, J = |
| | 1.9 Hz, 1H), 7.55-7.46 (m, 1H), 7.29 (d, J = 8.3 Hz, 1H), 6.78 (d, J = 0.9 Hz, |
| | 1H), 4.65 (d, J = 17.0 Hz, 4H), 4.38 (s, 4H), 3.30-3.25 (s, 4H), 3.00 (s, 3H), |
| | 2.38-2.32 (m, 3H), 2.19 (s, 3H). |
| 28 | ¹H NMR (300 MHz, DMSO-d₆) δ: 9.37(s, 1H), 9.03(s, 1H), 7.38(d, J = 8.7 Hz, |
| | 2H), 6.77 (s, 1H), 6.42 (d, J = 8.7 Hz, 2H), 4.49-4.45 (m, 1H), 3.95-3.91(m, |
| | 3H), 3.87(s, 4H), 3.24(s, 4H), 2.35-2.27(m, 4H), 2.18-2.06 (m, 4H), 1.27- |
| | 1.21(m, 1H), 0.60-0.58 (m, 2H), 0.46-0.41 (m, 2H). |
| 29 | ¹H NMR (400 MHz, Chloroform-d) δ (ppm) 8.89 (s, 1H), 7.69-7.61 (m, 1H), |
| | 7.39-7.31 (m, 1H), 7.28-7.22 (m, 2H), 6.65 (s, 1H), 4.51 (s, 4H), 4.12-4.00 (m, |
| | 8H), 3.87-3.75 (m, 1H), 3.46 (s, 4H), 2.94 (s, 3H), 2.47 (s, 3H), 2.37 (s, 3H). |
| 30 | ¹H-NMR (400 MHz, Chloroform-d) δ (ppm) 9.44 (s, 1H), 9.04 (s, 1H), 7.46 (d, |
| | J = 8.7 Hz, 2H), 6.79 (d, J = 0.9 Hz, 1H), 6.50-6.39 (m, 2H), 4.56-4.40 (m, |
| | 8H), 3.82 (s, 4H), 3.25 (s, 4H), 2.35 (s, 3H), 2.19 (s, 3H). |
| 31 | ¹H NMR (400 MHz, DMSO-d₆) δ: 9.36(s, 1H), 9.17-9.09(m, 1H), 7.85-7.50(m, |
| | 1H), 7.30-7.21(m, 1H), 6.78(s, 1H), 4.65-4.55(m, 1H), 4.15(s, 4H), 3.11- |
| | 2.98(m, 6H), 2.91-2.85(m, 4H), 2.35(s, 3H), 2.11-1.95(m, 3H) |
| 32 | ¹H NMR (400 MHz, DMSO-d₆) δ: 8.95(s, 1H), 7.45(s, 1H), 6.68(s, 1H), 4.41(s, |
| | 4H), 3.99(s, 1H), 3.88(s, 1H), 3.75-3.61(m, 4H), 3.60-3.50(m, 2H), 3.15-3.0(m, |
| | 2H), 2.70-2.55(m, 1H), 2.35-2.25(m, 3H), 2.10-1.95(m, 2H), 1.65-1.55(m, 2H), |
| | 1.08-0.91(m, 4H). |
| 33 | ¹H-NMR (400 MHz, Chloroform-d) δ (ppm) 8.94-8.77 (m, 1H), 7.76-7.64 (m, |
| | 1H), 7.62-7.53 (m, 1H), 6.81-6.70 (m, 1H), 6.67-6.60 (m, 1H), 4.69-4.56 (m, |
| | 1H), 4.27-4.11 (m, 2H), 4.13-4.05 (m, 1H), 4.03-3.89 (m, 1H), 3.46-3.34 (m, |
| | 3H), 2.75-2.70 (m, 3H), 2.48-2.43 (m, 3H), 2.42-2.34 (m, 3H), 2.26-2.19 (m, |
| | 1H), 2.09-1.96 (m, 2H), 1.46-1.37 (m, 2H), 1.31-1.21 (m, 1H). |
| 34 | ¹H-NMR (400 MHz, DMSO-d₆) δ (ppm) 9.31 (s, 1H), 9.01 (s, 1H), 7.40-7.30 |
| | (m, 2H), 6.74 (d, J = 0.9 Hz, 1H), 6.45-6.27 (m, 2H), 4.14-3.99 (m, 2H), 3.99- |
| | 3.87 (m, 2H), 3.81 (s, 4H), 3.27 (s, 4H), 2.34 (s, 3H), 2.20 (s, 3H), 2.16-2.08 |
| | (m, 1H), 2.06-1.95 (m, 1H), 1.68-1.51 (m, 2H). |
| 35 | ¹H NMR (300 MHz, DMSO-d6) δ (ppm) 9.24 (s, 1H), 8.96 (s, 1H), 7.36 (d, J = |
| | 8.6 Hz, 2H), 6.64 (s, 1H), 6.38 (d, J = 8.7 Hz, 2H), 3.78 (d, J = 3.8 Hz, 8H), |
| | 3.63-3.43 (m, 4H), 3.23(s, 4H), 2.30 (s, 3H), 2.17 (s, 3H), 1.82-1.72 (m, 2H), |
| | 1.53-1.44 (m, 4H). |
| 36 | ¹H NMR (400 MHz, DMSO-d₆) δ (ppm) 9.42 (s, 1H), 9.06 (s, 1H), 7.85 (s, |
| | 1H), 7.52 (s, 1H), 6.79 (s, 1H), 4.18-3.87 (m, 6H), 2.36 (s, 3H), 2.18 (s, 7H), |
| | 2.12-2.04 (m, 1H), 1.69-1.60 (m, 2H), 0.69-0.64 (m, 2H), 0.59-0.54 (m, 2H). |
| 37 | ¹H NMR (400 MHz, DMSO-d₆) δ 9.35(s, 1H), 9.11(s, 1H), 7.85-7.71(m, 1H), |
| | 7.35-7.25(m, 1H), 6.78(s, 1H), 4.65(s, 4H), 4.38-4.21(m, 4H), 3.61(s, 4H), |
| | 3.05-2.95(m, 3H), 2.65(s. 3H), 2.10 (s, 1H). |
| 38 | ¹H NMR (400 MHz, Chloroform-d) δ (ppm) 8.91 (s, 1H), 7.79 (s, 1H), 7.59 (s, |
| | 1H), 6.77 (s, 1H), 6.70 (s, 1H), 4.45 (d, J = 12.0 Hz, 1H), 4.31-4.16 (m, 4H), |
| | 4.01 (d, J = 12.0 Hz, 1H), 2.48-2.34 (m, 10H), 2.21-2.11 (m, 1H), 1.16-1.07 |
| | (m, 1H), 0.77 (s, 4H), 0.71-0.59 (m, 4H). |
| 39 | ¹H NMR (400 MHz, DMSO-d₆) δ 9.38 (s, 1H), 9.18-9.09 (m, 1H), 7.82-7.55 |
| | (m, 1H), 7.32-7.21 (m, 1H), 6.81-6.75 (m, 1H), 4.70-4.60 (m, 5H), 4.15 (s, |
| | 4H), 3.10-2.98 (m, 5H), 2.95-2.88 (m, 2H), 2.35 (s, 3H), 2.01-1.95 (m, 2H). |
| 40 | ¹H NMR (300 MHz, DMSO-d6) δ (ppm) 8.88 (s, 1H), 7.20 (s, 1H), 6.66 (s, |
| | 1H), 6.60 (s, 1H), 4.27 (s, 1H), 4.00 (s, 3H), 2.31 (s, 3H), 2.20-2.13 (m, 1H), |
| | 2.09 (s, 6H), 1.99 (d, J = 7.0 Hz, 4H), 1.80 (d, J = 13.2 Hz, 2H), 1.66-1.52 (m, |
| | 2H), 1.39 (s, 1H), 1.22 (s, 3H), 0.96-0.84 (m, 2H). |
| 43 | ¹H NMR (400 MHz, Chloroform-d) δ (ppm) 8.85-8.74 (m, 1H), 8.60 (s, 1H), |
| | 6.64-6.55 (m, 1H), 4.59-4.32 (m, 2H), 4.27-3.92 (m, 4H), 3.84-3.67 (m, 1H), |
| | 3.12-2.94 (m, 1H), 2.84-2.68 (m, 4H), 2.55 (d, J = 11.6 Hz, 1H), 2.43 (d, J = |
| | 3.2 Hz, 3H), 2.35-2.21 (m, 2H), 2.13-1.96 (m, 2H), 1.95-1.83 (m, 1H), 1.54- |
| | 1.37 (m, 2H). |
| 45 | ¹H-NMR (300 MHz, DMSO-d₆) δ (ppm) 8.76 (s, 1H), 6.66-6.48 (m, 1H), 5.33 |
| | (d, J = 7.3 Hz, 1H), 4.39-4.30 (m, 1H), 4.29-4.19 (m, 1H), 4.18-4.00 (m, 3H), |
| | 2.55-2.36 (m, 5H), 2.19 (d, J = 6.8 Hz, 2H), 2.09-2.01 (m, 1H), 1.88-1.80 (m, |
| | 2H), 1.77-1.63 (m, 6H), 1.48-1.39 (m, 3H), 1.39-1.29 (m, 3H), 1.28-1.21 (m, |
| | 2H). |
| 46 | ¹H NMR (400 MHz, DMSO-d6) δ 9.12 (s, 1H), 8.80 (s, 1H), 7.88 (s, 1H), 7.19 |
| | (s, 1H), 6.75 (t, J = 55.5 Hz, 1H), 4.37 (s, 4H), 3.87 (s, 1H), 3.63 (dd, J = 12.5, |
| | 4.5 Hz, 2H), 3.36 (s, 2H), 3.22-3.14 (m, 2H), 3.11-3.01 (m, 2H), 2.60 (tt, J = |
| | 7.8, 4.9 Hz, 1H), 2.21 (t, J = 7.2 Hz, 2H), 2.02 (d, J = 12.8 Hz, 2H), 1.63 (q, |
| | J = 11.3 Hz, 2H), 1.01 (dt, J = 7.9, 2.8 Hz, 2H), 0.98-0.92 (m, 2H). |
| 47 | ¹H NMR (400 MHz, DMSO-d6) δ (ppm) 9.71 (s, 1H), 9.08 (s, 1H), 7.76 (d, J = |
| | 8.5 Hz, 1H), 7.35 (d, J = 8.1 Hz, 1H), 7.13 (d, J = 8.1 Hz, 1H), 6.76 (d, J = 5.1 |
| | Hz, 1H), 4.24 (s, 4H), 3.95 (d, J = 8.0 Hz, 4H), 3.47 (s, 2H), 3.32 (d, J = 7.1 |
| | Hz, 1H), 2.96 (s, 1H), 2.86-2.77 (m, 1H), 2.35 (s, 3H), 2.14-2.01 (m, 2H), |
| | 1.98-1.91 (m, 1H), 0.49-0.43 (m, 2H), 0.42-0.37 (m, 2H). |
| 48 | ¹H-NMR (400 MHz, DMSO-d₆) δ (ppm) 8.93 (s, 1H), 7.45 (s, 1H), 6.67 (s, |
| | 1H), 4.37 (s, 4H), 3.86 (s, 1H), 3.63 (d, J = 11.9 Hz, 2H), 3.45-3.37(m, 4H), |
| | 3.05 (t, J = 11.2 Hz, 2H), 2.68-2.56 (m, 1H), 2.31 (s, 3H), 2.06-1.98 (m, 2H), |
| | 1.87-1.80 (m, 1H), 1.70-1.55 (m, 2H), 1.06-0.91 (m, 4H), 0.39-0.29 (m, 2H), |
| | 0.24-0.17 (m, 2H). |
| 49 | ¹H-NMR (400 MHz, DMSO-d₆) δ (ppm) 9.11-8.55 (m, 1H), 7.80-7.21 (m, |
| | 1H), 6.76-6.51 (m, 1H), 4.68-3.95 (m, 4H), 3.83 (s, 1H), 3.73-3.53 (m, 2H), |
| | 3.08-2.94 (m, 2H), 2.69 (s, 2H), 2.65-2.57 (m, 1H), 2.49-2.44 (m, 2H), 2.30 (s, |
| | 3H), 2.27-2.22 (m, 3H), 2.11-1.93 (m, 4H), 1.66-1.53 (m, 2H), 1.04-0.91 (m, |
| | 4H). |
| 50 | ¹H NMR (300 MHz, DMSO-d₆) δ 10.01 (s, 1H), 9.28 (s, 1H), 8.31 (s, 2H), |
| | 7.67 (d, J = 1.9 Hz, 1H), 7.44 (dd, J = 8.1, 2.0 Hz, 1H), 7.27 (s, 1H), 7.21 (d, |
| | J = 8.1 Hz, 1H), 6.80 (t, J = 55.5 Hz, 1H), 4.34 (s, 4H), 3.98 (s, 2H), 3.94 (s, |
| | 2H), 3.25 (s, 2H), 3.10 (t, J = 7.1 Hz, 2H), 2.16 (t, J = 7.1 Hz, 2H), 2.11-2.01 |
| | (m, 1H), 0.53-0.38 (m, 4H). |
| 51 | ¹H NMR (400 MHz, DMSO-d₆) δ 8.92 (s, 1H), 7.40 (s, 1H), 6.66 (d, J = 7.9 |
| | Hz, 1H), 4.24 (s, 4H), 3.82 (s, 1H), 3.63-3.55 (m, 2H), 3.06 (d, J = 6.9 Hz, 2H), |
| | 2.94 (d, J = 14.2 Hz, 4H), 2.84-2.77 (m, 2H), 2.30 (s, 3H), 2.26-2.15 (m, 1H), |
| | 2.03-1.91 (m, 4H), 1.90-1.81 (m, 2H), 1.67-1.48 (m, 6H), 1.35-1.21 (m, 3H). |
| 52 | ¹H-NMR (400 MHz, Chloroform-d) δ (ppm) 8.93-8.67 (m, 1H), 6.77-6.43 (m, |
| | 1H), 5.96 (s, H), 5.25-5.03 (m, 1H), 4.70-4.42 (m, 1H), 4.16-3.72 (m, 6H), |
| | 3.01-2.89 (m, 2H), 2.83 (s, 3H), 2.68-2.36 (m, 6H), 2.28-2.18 (m, 2H), 2.06- |
| | 1.82 (m, 4H). |
| 53 | ¹H NMR (400 MHz, DMSO-d₆) δ (ppm) 8.91 (s, 1H), 7.37 (s, 1H), 6.63 (s, |
| | 1H), 4.08 (s, 2H), 3.90 (s, 2H), 3.82 (d, J = 9.5 Hz, 1H), 3.59 (d, J = 12.0 Hz, |
| | 2H), 2.93-2.83 (m, 5H), 2.70-2.58 (m, 4H), 2.30 (s, 3H), 2.07-1.99 (m, 4H), |
| | 1.59 (q, J = 11.3 Hz, 2H). |
| 54 | ¹H NMR (400 MHz, DMSO-d₆) δ (ppm) 8.90 (s, 1H), 7.32 (s, 1H), 6.61 (s, |
| | 1H), 4.04 (s, 2H), 3.89-3.70 (m, 3H), 3.57 (d, J = 12.0 Hz, 2H), 2.91-2.78 (m, |
| | 5H), 2.30 (s, 3H), 2.04-1.93 (m, 2H), 1.89-1.80 (m, 2H), 1.64-1.57 (m, 2H), |
| | 0.62 (d, J = 6.4 Hz, 4H). |
| 55 | 1H NMR (400 MHz, DMSO-d6) δ 9.09 (s, 1H), 7.46 (d, J = 7.6 Hz, 1H), 7.16 |
| | (s, 1H), 6.68 (t, J = 55.7 Hz, 1H), 4.39 (q, J = 9.5 Hz, 4H), 3.94 (s, 1H), 3.64 |
| | (d, J = 12.6 Hz, 2H), 3.27 (s, 2H), 3.17 (d, J = 7.2 Hz, 2H), 3.11 (t, J = 7.1 Hz, |
| | 2H), 3.01 (ddd, J = 13.0, 10.6, 2.9 Hz, 2H), 2.75 (p, J = 7.5 Hz, 1H), 2.17 (dd, |
| | J = 8.3, 6.4 Hz, 4H), 2.02 (dd, J = 13.2, 3.7 Hz, 2H), 1.97-1.81 (m, 4H), 1.72- |
| | 1.59 (m, 2H). |
| 56 | ¹H NMR (400 MHz, DMSO-d₆) δ (ppm) 8.94 (s, 1H), 7.43 (s, 1H), 6.69 (s, |
| | 1H), 5.47 (s, 1H), 4.39-4.08 (m, 3H), 3.82-3.71 (m, 1H), 3.69-3.58 (m, 2H), |
| | 3.04-2.90 (m, 2H), 2.68-2.61 (m, 1H), 2.35-2.21 (m, 3H), 2.15-1.97 (m, 2H), |
| | 1.70-1.52 (m, 2H), 1.30-1.17 (m, 2H), 1.08-0.92 (m, 4H), 0.51-0.39 (m, 4H). |
| 57 | ¹H NMR (400 MHz, DMSO-d6) δ (ppm) 8.93 (s, 1H), 7.47 (s, 1H), 6.68 (d, J = |
| | 0.9 Hz, 1H), 4.67-4.44 (m, 3H), 4.16 (s, 2H), 3.84 (s, 1H), 3.63 (d, J = 11.9 Hz, |
| | 2H), 3.39-3.35 (m, 1H), 3.06-2.95 (m, 2H), 2.64-2.53 (m, 1H), 2.31 (s, 3H), |
| | 2.02 (d, J = 12.8 Hz, 2H), 1.65-1.50 (m, 2H), 1.03-0.89 (m, 4H), 0.60-0.56 (m, |
| | 2H), 0.55-0.42 (m, 2H). |
| 58 | ¹H NMR (400 MHz, DMSO-d₆) δ (ppm) 8.93 (s, 1H), 7.41 (s, 1H), 7.09-6.67 |
| | (m, 2H), 4.93-4.89 (m, 1H), 4.28-4.12 (m, 1H), 4.04-3.79 (m, 3H), 3.67-3.58 |
| | (m, 2H), 3.02-2.91 (m, 2H), 2.59-2.56 (m, 1H), 2.32 (s, 3H), 2.23-2.11 (m, |
| | 2H), 2.09-1.90 (m, 2H), 1.70-1.53 (m, 2H), 1.23 (s, 1H), 1.02-0.98 (m, 2H), |
| | 0.97-0.90(m, 2H). |
| 59 | ¹H NMR (400 MHz, DMSO-d6) δ (ppm) 8.89 (s, 1H), 7.38 (s, 1H), 6.61 (s, |
| | 1H), 4.14 (s, 2H), 3.91-3.68 (m, 5H), 3.15 (d, J = 6.7 Hz, 2H), 3.13-3.04 (m, |
| | 4H), 2.29 (s, 3H), 2.23 (s, 3H), 2.07-1.98 (m, 4H), 1.59-1.47 (m, 2H), 1.43 (s, |
| | 3H), 1.21-1.13 (m, 2H), 0.86-0.80 (m, 2H). |
| 60 | ¹H NMR (DMSO-d₆, 499 MHz) δ 8.98 (s, 1H), 7.4-7.6 (m, 1H), 6.8-6.9 (m, |
| | 1H), 4.9-5.0 (m, 1H), 4.2-4.4 (m, 4H), 3.80 (d, 2H, J = 8.5 Hz), 3.60 (d, 2H, |
| | J = 12.3 Hz), 3.46 (d, 1H, J = 8.5 Hz), 2.8-3.0 (m, 5H), 2.04 (d, 2H, J = 12.3 |
| | Hz), 1.9-2.0 (m, 2H), 1.5-1.7 (m, 3H), 0.92 (m, 2H), 0.7-0.8 (m, 2H) |
| 61 | ¹H NMR (400 MHz, DMSO-d6) δ (ppm) 8.95 (s, 1H), 7.48 (s, 1H), 6.69 (s, |
| | 1H), 4.67-4.13 (s, 3H), 3.84 (s, 1H), 3.65 (d, J = 12.1 Hz, 2H), 2.98 (t, J = 11.3 |
| | Hz, 2H), 2.88 (t, J = 11.7 Hz, 2H), 2.71-2.63 (s, 2H), 2.61-2.54 (m, 2H), 2.31 (s, |
| | 3H), 2.07 (s, 1H), 2.04 (d, J = 12.4 Hz, 2H), 1.97 (s, 2H), 1.64-1.51 (m, 2H), |
| | 1.04-0.96 (m, 2H), 0.96-0.90 (m, 2H). |
| 62 | ¹H NMR (400 MHz, DMSO-d6) δ 9.20 (s, 1H), 9.00 (s, 1H), 7.60 (s, 1H), 6.82 |
| | (s, 1H), 4.58-4.41 (m, 2H), 4.38-4.32 (m, 2H), 4.31-4.21 (m, 2H), 4.19-4.09 |
| | (m, 2H), 3.87 (s, 1H), 3.72-3.57 (m, 2H), 3.09-2.97 (m, 2H), 2.63-2.54 (m, |
| | 1H), 2.36 (s, 3H), 2.10-1.97 (m, 2H), 1.69-1.52 (m, 2H), 1.07-0.91 (m, 4H). |
| 63 | ¹H NMR (400 MHz, Chloroform-d) δ (ppm) 9.00 (d, J = 14.1 Hz, 1H), 7.25- |
| | 7.13 (m, 1H), 4.98-4.84 (m, 1H), 4.61-4.46(m, 1H), 4.01 (d, J = 12.0 Hz, 2H), |
| | 3.21-3.16 (m, 3H), 3.09-2.98 (m, 2H), 2.76-2.56 (m, 5H), 2.48-2.39 (m, 1H), |
| | 2.38-2.29 (m, 1H), 2.07-1.94(m, 2H), 1.92-1.82 (m, 2H), 1.51 (s, 2H), 1.42 (s, |
| | 1H), 1.35-1.26 (m, 2H), 1.25-1.20(m, 2H), 1.08-1.00 (m, 2H). |
| 64 | 1H NMR (400 MHz, Chloroform-d) δ 8.79 (s, 1H), 6.66 (s, 1H), 5.18 (d, J = |
| | 7.6 Hz, 1H), 4.52 (s, 4H), 3.95 (s, 5H), 3.78 (d, J = 12.6 Hz, 2H), 3.57 (t, J = |
| | 5.1 Hz, 2H), 3.38 (s, 3H), 3.12 (dd, J = 18.5, 9.5 Hz, 4H), 2.99 (s, 2H), 2.89 (p, |
| | J = 7.6 Hz, 1H), 2.32-2.20 (m, 2H), 2.23-2.13 (m, 2H), 2.04-1.85 (m, 5H), |
| | 1.75 (t, J = 11.5 Hz, 2H), 0.99 (dt, J = 4.8, 2.9 Hz, 2H), 0.92-0.81 (m, 2H). |
| 65 | 1H NMR (300 MHz, Chloroform-d) δ 8.92 (s, 1H), 7.09 (s, 1H), 6.52 (t, J = |
| | 56.1 Hz, 1H), 5.36-5.20 (m, 1H), 4.78-4.13 (m, 4H), 4.09-3.95 (m, 1H), |
| | 3.88-3.64 (m, 2H), 3.22 (s, 2H), 3.14-2.95 (m, 3H), 2.86 (s, 3H), 2.32- |
| | 2.18 (m, 2H), 1.98 (m, 2H), 1.83-1.54 (m, 4H). |
| 67 | ¹H NMR (DMSO-d₆, 499 MHz) δ 8.99 (d, 1H, J = 8.2 Hz), 7.5-7.8 (m, 1H), |
| | 6.78 (s, 1H), 4.50 (br d, 1H, J = 7.4 Hz), 4.2-4.4 (m, 2H), 4.0-4.1 (m, 1H), 3.8- |
| | 3.9 (m, 1H), 3.80 (br d, 1H, J = 8.8 Hz), 3.72 (br d, 1H, J = 8.2 Hz), 3.65 (br d, |
| | 2H, J = 11.0 Hz), 3.49 (br d, 1H, J = 8.2 Hz), 3.09 (td, 2H, J = 4.2, 8.1 Hz), |
| | 3.0-3.0 (m, 2H), 2.35 (d, 4H, J = 2.7 Hz), 2.0-2.1 (m, 2H), 1.5-1.6 (m, 5H), |
| | 1.23 (s, 3H), 1.04 (d, 4H, J = 2.5 Hz) |
| 68 | ¹H NMR (METHANOL-d4, 499 MHz) δ 8.9-9.0 (m, 1H), 6.8-6.9 (m, 1H), 5.1- |
| | 5.3 (m, 2H), 4.9-4.7 (m, 2H), 3.8-3.8 (m, 2H), 3.0-3.1 (m, 2H), 2.60 (br d, 2H, |
| | J = 3.6 Hz), 2.5-2.5 (m, 1H), 2.46 (s, 2H), 2.1-2.2 (m, 2H), 1.6-1.7 (m, 2H), 1.51 |
| | (d, 1H, J = 4.7 Hz), 1.0-1.1 (m, 4H) |
| 69 | ¹H NMR (400 MHz, CDCl3) δ 8.95 (s, 1H), 7.22-7.11 (m, 1H), 5.31 (s, 1H), |
| | 4.65-4.51 (m, 1H), 4.41-4.26 (m, 1H), 4.18-3.99 (m, 1H), 3.87-3.68 (m, 3H), |
| | 3.52 (s, 1H), 3.11-2.93 (m, 2H), 2.93-2.78 (m, 4H), 2.76-2.68 (m, 2H), 2.44- |
| | 2.16 (m, 5H), 1.87-1.60 (m, 5H), 1.19 (s, 1H). |
| 70 | ¹H NMR (400 MHz, CDCl₃) δ 8.93 (s, 1H), 7.16 (s, 1H), 5.30-5.20 (m, 1H), |
| | 4.12-3.91 (m, 2H), 3.87-3.67 (m, 4H), 3.19-2.98 (m, 4H), 2.96-2.80 (m, 5H), |
| | 2.61-2.49 (m, 4H), 2.34-2.20 (m, 3H), 2.17-2.00 (m, 1H), 1.83-1.65 (m, 2H), |
| | 1.15-1.05 (m, 2H), 1.02-0.93 (m, 2H). |
| 80 | 1H NMR (400 MHz, DMSO-d6) δ 9.80 (s, 1H), 9.15 (s, 1H), 7.42 (s, 1H), 4.17 |
| | (dd, J = 10.4, 6.4 Hz, 2H), 4.07 (d, J = 6.3 Hz, 2H), 3.92 (s, 1H), 3.60 (d, J = |
| | 12.3 Hz, 2H), 3.51 (t, J = 4.8 Hz, 2H), 3.35 (t, J = 5.6 Hz, 2H), 3.29 (s, 3H), |
| | 3.18 (d, J = 7.3 Hz, 2H), 3.00 (s, 2H), 2.73 (q, J = 8.0, 7.0 Hz, 2H), 2.60 (d, J = |
| | 8.6 Hz, 2H), 2.24-2.07 (m, 4H), 2.01 (s, 2H), 1.95-1.77 (m, 5H), 1.62 (m, |
| | 2H), 1.00-0.91 (m, 4H). |
| 83 | ¹H NMR (400 MHz, DMSO-d6) δ (ppm) 9.88 (s, 1H), 9.13 (s, 1H), 7.77 (d, J = |
| | 8.3 Hz, 2H), 7.35 (d, J = 8.2 Hz, 2H), 6.79 (s, 1H), 5.52 (s, 1H), 4.44 (s, 2H), |
| | 4.21 (s, 2H), 4.10 (d, J = 9.5 Hz, 2H), 2.90 (s, 3H), 2.36 (s, 3H), 1.29-1.18 (m, |
| | 1H), 0.50-0.42 (m, 2H), 0.41-0.35 (m, 2H). |
| 84 | ¹H NMR (400 MHz, DMSO-d₆) δ (ppm) 8.93 (s, 1H), 7.14 (s, 1H), 5.25 (s, |
| | 1H), 4.20-3.90 (m, 2H), 3.87-3.64 (m, 5H), 3.14-2.97 (m, 2H), 2.85 (s, 3H), |
| | 2.76-2.54 (m, 9H), 2.53-2.38 (m, 4H), 2.33-2.22 (m, 2H), 1.81-1.68 (m, 2H). |
| 85 | ¹H-NMR (400 MHz, DMSO-d₆) δ (ppm) 8.96 (s, 1H), 7.48 (s, 1H), 6.73 (s, |
| | 1H), 4.57-4.36 (m, 2H), 4.28-4.07 (m, 2H), 3.92-3.76 (m, 1H), 3.66-3.52 (m, |
| | 2H), 3.25 (s, 2H), 3.16 (t, J = 14.3 Hz, 2H), 3.02-2.81 (m, 6H), 2.33 (s, 3H), |
| | 2.08-1.95 (m, 2H), 1.70-1.53 (m, 2H). |
| 86 | ¹H NMR (400 MHz, CDCL₃) δ (ppm) 8.69 (s, 1H), 6.48 (s, 1H), 5.68-5.61 (m, |
| | 1H), 4.62-4.50 (m, 2H), 4.42-4.38 (m, 2H), 4.31-4.20 (m, 2H), 4.14-4.08 (m, |
| | 1H), 4.04-3.96 (m, 2H), 3.72-3.65 (m, 1H), 3.52-3.46 (m, 1H), 3.30-3.22 (m, |
| | 1H), 2.42 (s, 3H), 2.13-2.08 (m, 1H), 1.75-1.61 (m, 1H), 1.33-1.27 (m, 1H), |
| | 0.64-0.52 (m, 2H), 0.50-0.44 (m, 2H). |
| 87 | ¹H NMR (400 MHz, DMSO-d₆) δ (ppm) 8.92 (s, 1H), 8.30 (s, 1H), 7.77 (s, |
| | 1H), 7.46 (s, 1H), 6.67 (s, 1H), 4.37-4.24 (m, 2H), 4.19-4.15 (m, 1H), 3.93 (s, |
| | 3H), 3.67-3.50 (m, 3H), 2.83-2.78(m, 2H), 2.67-2.61 (m, 2H), 2.50-2.41 (m, |
| | 4H), 2.33-2.30 (m, 3H), 2.04-2.01 (m, 2H), 1.92 (s, 2H), 1.65-1.57 (m, 2H). |
| 88 | ¹H NMR (400 MHz, Chloroform-d) δ 8.97-8.95(m, 2H), 7.29-7.26 (m, 1H), |
| | 4.63-4.60 (m, 1H), 4.10-3.83(m, 3H), 2.99-2.93 (m, 4H), 2.89-2.87 (m, |
| | 4H), 2.72-2.70 (m, 3H), 2.54-2.20 (m, 4H), 1.84-1.78 (m, 3H), 0.47-0.41(m, 4H). |
| 89 | ¹H NMR (400 MHz, DMSO-d6) δ 9.09(s, 2H), 8.98 (s, 1H), 8.34 (s, 1H), 7.78 |
| | (s, 1H), 7.56 (s, 1H), 6.81 (s, 1H), 4.42 (s, 2H), 4.29 (s, 2H), 4.22 (d, J = 11.6 |
| | Hz, 2H), 4.09 (d, J = 11.5 Hz, 2H), 3.92 (s, 3H), 3.72 (s, 1H), 3.49 (d, J = 11.3 |
| | Hz, 2H), 2.53 (s, 2H), 2.35 (s, 3H), 2.02 (d, J = 12.2 Hz, 2H), 1.69-1.57 (m, |
| | 2H). |
| 90 | ¹H-NMR (400 MHz, Chloroform-d) δ (ppm) 8.92 (s, 1H), 7.09 (s, 1H), 6.52 (t, |
| | J = 56.1 Hz, 1H), 5.46-5.20 (m, 1H), 5.01-4.45 (m, 2H), 4.40-4.13 (m, 2H), |
| | 4.06-3.94 (m, 1H), 3.90-3.75 (m, 2H), 3.10-2.79 (m, 10H), 2.32-2.21 (m, 2H), |
| | 2.18-2.04 (m, 2H), 1.85-1.74 (m, 2H). |
| 91 | ¹H NMR (400 MHz, DMSO-d₆) δ (ppm) 8.97 (s, 1H), 7.49 (s, 1H), 6.73 (s, |
| | 1H), 6.58 (s, 1H), 6.16 (s, 1H), 4.49 (s, 2H), 4.19 (s, 2H), 3.82 (s, 1H), 3.58 (d, |
| | J = 12.2 Hz, 2H), 2.95-2.84 (m, 5H), 2.33 (s, 3H), 2.02 (d, J = 12.0 Hz, 2H), |
| | 1.66-1.54 (m, 2H). |
| 92 | ¹H NMR (400 MHz, CDCL₃) δ (ppm) 8.83 (s, 1H), 6.68 (s, 1H), 5.25-5.24 (m, |
| | 1H), 4.47-4.41 (m, 2H), 4.37-4.34 (m, 2H), 4.12-4.09 (m, 2H), 3.84-3.81 (m, |
| | 2H), 3.62-3.51 (m, 2H), 3.03-2.97 (m, 2H), 2.86 (s, 3H), 2.45 (s, 4H), 2.26- |
| | 2.24 (m, 2H), 1.78-1.70 (m, 2H), 1.28 (s, 1H). |
| 93 | ¹H NMR (400 MHz, DMSO-d₆) δ 9.16 (s, 1H), 7.70 (s, 1H), 7.37 (s, 1H), 6.06- |
| | 5.85 (m, 2H), 4.08-3.86 (m, 2H), 3.60-3.50 (m, 2H), 2.96-2.83 (m, 5H), 2.72- |
| | 2.62 (m, 2H), 2.59-2.52 (m, 4H), 2.14-2.01 (m, 3H), 1.67-1.55 (m, 2H). |
| 94 | 1H NMR (400 MHz, DMSO-d6) δ 8.96 (s, 1H), 7.96 (s, 1H), 7.52 (s, 1H), 8.76 |
| | (s, 1H), 8.67 (s, 1H), 4.40-4.11 (m, 4H), 3.96 (s, 3H), 3.82-3.59 (m, 5H), 3.38- |
| | 3.32 (m, 2H), 2.73-2.57 (m, 3H), 2.33 (s, 3H), 2.09-1.95 (m, 2H), 1.70-1.46 |
| | (m, 2H). |
| 95 | ¹H NMR (400 MHz, DMSO-d₆) δ (ppm) 8.99 (s, 1H), 7.56 (s, 1H), 6.81 (s, |
| | 1H), 4.40-4.37 (m, 4H), 4.32-4.06 (m, 2H), 4.03-3.85 (m, 3H), 3.66-3.63 (m, |
| | 2H), 3.10-2.97 (m, 4H), 2.35 (s, 3H), 2.03-2.01 (m, 2H), 1.61-1.53 (m, 2H), |
| | 1.25-1.21 (m, 4H). |
| 96 | ¹H NMR (400 MHz, DMSO-d₆) δ (ppm) 8.98 (s, 1H), 7.54 (s, 1H), 6.78 (s, |
| | 1H), 4.40-4.20 (m, 4H), 4.10 (q, J = 5.2 Hz, 1H), 3.84 (s, 1H), 3.66-3.55 (m, |
| | 2H), 3.21 (d, J = 7.7 Hz, 2H), 3.17 (d, J = 5.2 Hz, 3H), 2.88 (s, 3H), 2.34 (s, |
| | 3H), 2.25 (s, 3H), 2.10-1.98 (m, 2H), 1.67-1.52 (m, 2H). |
| 97 | ¹H NMR (400 MHz, DMSO-d₆) δ 8.96 (s, 1H), 8.34 (s, 1H), 7.77 (d, J = 0.6 |
| | Hz, 1H), 7.53 (s, 1H), 6.76 (d, J = 0.9 Hz, 1H), 4.28 (s, 2H), 4.23 (s, 1H), 3.92 |
| | (s, 3H), 3.70 (s, 1H), 3.55 (d, J = 11.6 Hz, 2H), 3.39 (d, J = 7.8 Hz, 2H), 3.18 |
| | (s, 2H), 2.45 (d, J = 11.4 Hz, 1H), 2.33 (s, 3H), 2.26 (s, 3H), 2.08 (s, 2H), 2.06- |
| | 1.99 (m, 2H), 1.69-1.59 (m, 2H). |
| 98 | ¹H NMR (400 MHz, DMSO-d₆) δ (ppm) 8.97-8.95 (m, 1H), 7.54-7.44 (m, 1H), |
| | 6.74-6.72 (m, 1H), 5.75-5.33 (m, 4H), 4.38-4.20 (m, 4H), 3.84-3.80 (m, 1H), |
| | 3.58-3.49 (m, 4H), 2.88-2.86 (m, 5H), 2.33 (s, 3H), 2.03-2.00 (m, 2H), 1.60- |
| | 1.57 (m, 2H). |
| 99 | ¹H NMR (400 MHz, CD₃OD) δ 8.04 (s, 1H), 7.32 (s, 1H), 4.10-3.99 (m, 1H), |
| | 3.80-3.70 (m, 2H), 3.24-3.13 (m, 2H), 3.09-2.96 (m, 2H), 2.91-2.88 (m, 4H), |
| | 2.84-2.77 (m, 2H), 2.61 (s, 3H), 2.52-2.44 (m, 1H), 2.36-2.15 (m, 4H), 1.84- |
| | 1.66 (m, 2H), 0.94-0.83 (m, 2H). |
| 100 | ¹H NMR (400 MHz, Chloroform-d) δ 9.04-8.68 (m, 1H), 7.45-7.11 (m, 1H), |
| | 6.64 (d, J = 4.1 Hz, 1H), 6.03 (t, J = 55.8 Hz, 1H), 5.32-5.06 (m, 1H), 4.92- |
| | 4.68 (m, 1H), 4.34 (s, 3H), 4.10-3.91 (m, 1H), 3.89-3.73 (m, 2H), 3.51-3.07 |
| | (m, 4H), 3.05-2.92 (m, 2H), 2.91-2.78 (m, 3H), 2.73-2.56 (m, 1H), 2.56-2.35 |
| | (m, 3H), 2.22 (s, 2H), 1.83-1.65 (m, 2H). |
| 101 | ¹H NMR (400 MHz, DMSO-d₆) δ (ppm) 8.97 (s, 1H), 7.49 (s, 1H), 6.74 (s, |
| | 1H), 4.48 (s, 2H), 4.19 (s, 2H), 3.83 (s, 1H), 3.61-3.52 (m, 2H), 3.03-2.92 (m, |
| | 4H), 2.91-2.83 (m, 5H), 2.33 (s, 3H), 2.29 (s, 3H), 2.06-1.97 (m, 2H), 1.67- |
| | 1.54 (m, 2H). |
| 102 | ¹H NMR (400 MHz, DMSO-d₆) δ (ppm) 9.03-8.92 (m, 1H), 7.55 (s, 1H), 6.81- |
| | 6.72 (m, 1H), 4.44-4.15 (m, 4H), 3.84 (s, 1H), 3.67 (d, J = 12.0 Hz, 2H), 3.34- |
| | 3.30 (m, 2H), 3.20 (d, J = 7.6 Hz, 2H), 3.05-2.93 (m, 2H), 2.63-2.54 (m, 1H), |
| | 2.34 (s, 3H), 2.25 (s, 3H), 2.12-1.98 (m, 2H), 1.67-1.50 (m, 2H), 1.04-0.91 (m, |
| | 4H). |
| 103 | ¹H NMR (400 MHz, DMSO-d₆) δ 8.94 (s, 1H), 7.45-7.41 (m, 1H), 6.70 (s, |
| | 1H), 5.30-5.15 (m, 1H), 4.54-4.17 (m, 3H), 3.82 (s, 1H), 3.57-3.54 (m, 2H), |
| | 2.96-2.88 (m, 5H), 2.84-2.82 (m, 2H), 2.67-2.50 (m, 3H), 2.49-2.28 (m, 6H), |
| | 2.12-1.95 (m, 2H), 1.71-1.52 (m, 2H). |
| 104 | ¹H NMR (400 MHz, DMSO-d₆) δ 8.94 (s, 1H), 7.49-7.43 (m, 1H), 6.70 (s, |
| | 1H), 5.30-5.15 (m, 1H), 4.53-4.13 (m, 3H), 3.82 (s, 1H), 3.58-3.56 (m, 2H), |
| | 3.01-2.82 (m, 7H), 2.67-2.63 (m, 3H), 2.50-2.28 (m, 6H), 2.02-1.99 (m, 2H), |
| | 1.71-1.59 (m, 2H). |

In some embodiments, the compounds of this disclosure are in Table 2.

TABLE 2

| Cmpd | | |
|---|---|---|
| No. | Structure | Name |
| 8A | 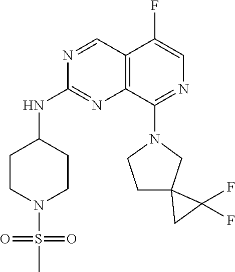 [SMILES: CS(=O)(=O)N1CCC(Nc2ncc3c(F)cnc(N4CCC5(C4)CC5(F)F)c3n2)CC1] | 8-(1,1-difluoro-5- azaspiro[2.4]heptan-5-yl)- 5-fluoro-N-(1- (methylsulfonyl)piperidin- 4-yl)pyrido[3,4- d]pyrimidin-2-amine |
| 9A | 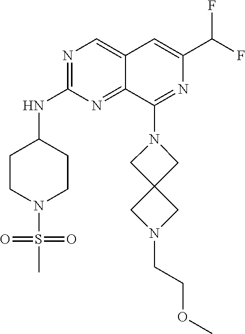 [SMILES: COCCN1CC2(C1)CN(c1nc(C(F)F)cc3cnc(NC4CCN(S(C)(=O)=O)CC4)nc13)C2] | 6-(difluoromethyl)-8-(6- (2-methoxyethyl)-2,6- diazaspiro[3.3]heptan-2- yl)-N-(1- (methylsulfonyl)piperidin- 4-yl)pyrido[3,4- d]pyrimidin-2-amine |
| 10A | 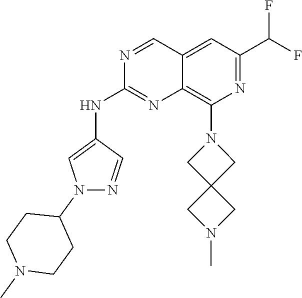 [SMILES: CN1CCC(n2cc(Nc3ncc4cc(C(F)F)nc(N5CC6(CN(C)C6)C5)c4n3)cn2)CC1] | 6-(difluoromethyl)-8-(6- methyl-2,6- diazaspiro[3.3]heptan-2- yl)-N-(1-(1- methylpiperidin-4-yl)- 1H-pyrazol-4- yl)pyrido[3,4- d]pyrimidin-2-amine |
| 11A | 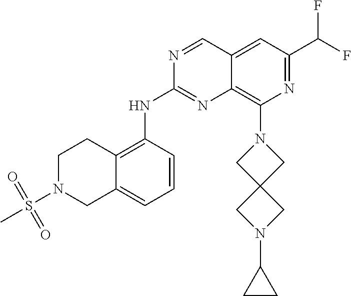 [SMILES: CS(=O)(=O)N1CCc2c(cccc2Nc2ncc3cc(C(F)F)nc(N4CC5(C4)CN(C4CC4)C5)c3n2)C1] | 8-(6-cyclopropyl-2,6- diazaspiro[3.3]heptan-2- yl)-6-(difluoromethyl)-N- (2-(methylsulfonyl)- 1,2,3,4- tetrahydroisoquinolin-5- yl)pyrido[3,4- d]pyrimidin-2-amine |
| 12A | 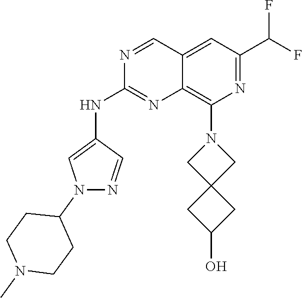 [SMILES: CN1CCC(n2cc(Nc3ncc4cc(C(F)F)nc(N5CC6(CC(O)C6)C5)c4n3)cn2)CC1] | 2-(6-(difluoromethyl)-2- ((1-(1-methylpiperidin-4- yl)-1H-pyrazol-4- yl)amino)pyrido[3,4- d]pyrimidin-8-yl)-2- azaspiro[3.3]heptan-6-ol |
| 13A | 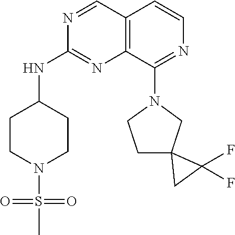 [SMILES: CS(=O)(=O)N1CCC(Nc2ncc3ccnc(N4CCC5(C4)CC5(F)F)c3n2)CC1] | 8-(1,1-difluoro-5- azaspiro[2.4]heptan-5-yl)- N-(1- (methylsulfonyl)piperidin- 4-yl)pyrido[3,4- d]pyrimidin-2-amine |
| 14A | 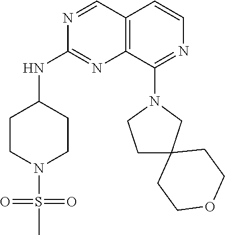 [SMILES: CS(=O)(=O)N1CCC(Nc2ncc3ccnc(N4CCC5(CCOCC5)C4)c3n2)CC1] | N-(1- (methylsulfonyl)piperidin- 4-yl)-8-(8-oxa-2- azaspiro[4.5]decan-2- yl)pyrido[3,4- d]pyrimidin-2-amine |
| 15A | 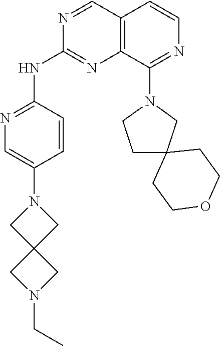 [SMILES: CCN1CC2(C1)CN(c1ccc(Nc3ncc4ccnc(N5CCC6(CCOCC6)C5)c4n3)nc1)C2] | N-(5-(6-ethyl-2,6- diazaspiro[3.3]heptan-2- yl)pyridin-2-yl)-8-(8-oxa- 2-azaspiro[4.5]decan-2- yl)pyrido[3,4- d]pyrimidin-2-amine |
| 16A | 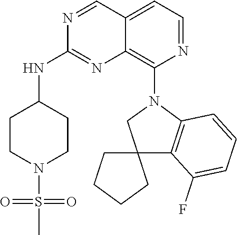 [SMILES: CS(=O)(=O)N1CCC(Nc2ncc3ccnc(N4CC5(CCCC5)c5c(F)cccc54)c3n2)CC1] | 8-(4′- fluorospiro[cyclopentane- 1,3′-indolin]-1′-yl)-N-(1- (methylsulfonyl)piperidin- 4-yl)pyrido[3,4- d]pyrimidin-2-amine |
| 17A | 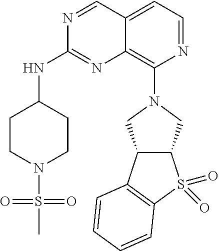 [SMILES: CS(=O)(=O)N1CCC(Nc2ncc3ccnc(N4C[C@H]5c6ccccc6S(=O)(=O)[C@H]5C4)c3n2)CC1] | (3aR,8bR)-2-(2-((1- (methylsulfonyl)piperidin- 4-yl)amino)pyrido[3,4- d]pyrimidin-8-yl)- 2,3,3a,8b-tetrahydro-1H- benzo[4,5]thieno[2,3- c]pyrrole 4,4-dioxide |
| 18A | 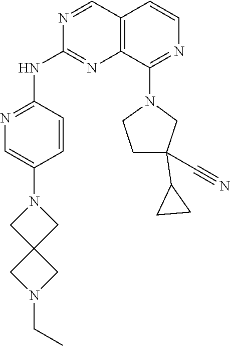 [SMILES: CCN1CC2(C1)CN(c1ccc(Nc3ncc4ccnc(N5CCC(C#N)(C6CC6)C5)c4n3)nc1)C2] | 3-cyclopropyl-1-(2-((5- (6-ethyl-2,6- diazaspiro[3.3]heptan-2- yl)pyridin-2- yl)amino)pyrido[3,4- d]pyrimidin-8- yl)pyrrolidine-3- carbonitrile |
| 19A | 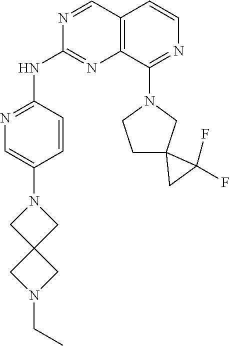 [SMILES: CCN1CC2(C1)CN(c1ccc(Nc3ncc4ccnc(N5CCC6(C5)CC6(F)F)c4n3)nc1)C2] | 8-(1,1-difluoro-5- azaspiro[2.4]heptan-5-yl)- N-(5-(6-ethyl-2,6- diazaspiro[3.3]heptan-2- yl)pyridin-2- yl)pyrido[3,4- d]pyrimidin-2-amine |
| 20A | 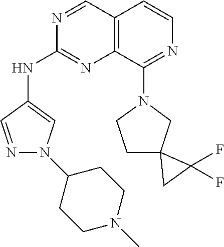 [SMILES: CN1CCC(n2cc(Nc3ncc4ccnc(N5CCC6(C5)CC6(F)F)c4n3)cn2)CC1] | 8-(1,1-difluoro-5- azaspiro[2.4]heptan-5-yl)- N-(1-(1-methylpiperidin- 4-yl)-1H-pyrazol-4- yl)pyrido[3,4- d]pyrimidin-2-amine |
| 21A | 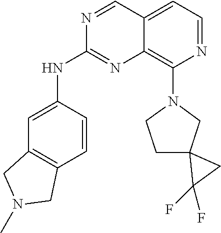 [SMILES: CN1Cc2ccc(Nc3ncc4ccnc(N5CCC6(C5)CC6(F)F)c4n3)cc2C1] | 8-(1,1-difluoro-5- azaspiro[2.4]heptan-5-yl)- N-(2-methylisoindolin-5- yl)pyrido[3,4- d]pyrimidin-2-amine |
| 22A | 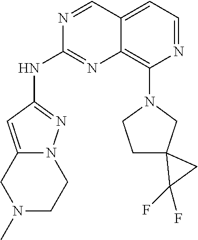 [SMILES: CN1CCn2nc(Nc3ncc4ccnc(N5CCC6(C5)CC6(F)F)c4n3)cc2C1] | 8-(1,1-difluoro-5- azaspiro[2.4]heptan-5-yl)- N-(5-methyl-4,5,6,7- tetrahydropyrazolo[1,5- alpyrazin-2- yl)pyrido[3,4- d]pyrimidin-2-amine |
| 23A | 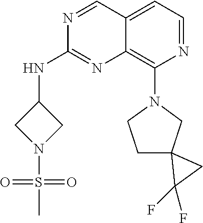 [SMILES: CS(=O)(=O)N1CC(Nc2ncc3ccnc(N4CCC5(C4)CC5(F)F)c3n2)C1] | 8-(1,1-difluoro-5- azaspiro[2.4]heptan-5-yl)- N-(1- (methylsulfonyl)azetidin- 3-yl)pyrido[3,4- d]pyrimidin-2-amine |
| 24A | 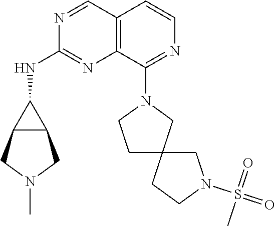 [SMILES: CN1C[C@@H]2[C@H](C1)[C@H]2Nc1ncc2ccnc(N3CCC4(CCN(S(C)(=O)=O)C4)C3)c2n1] | N-((1R,5S,6s)-3-methyl- 3-azabicyclo[3.1.0]hexan- 6-yl)-8-(7- (methylsulfonyl)-2,7- diazaspiro[4.4]nonan-2- yl)pyrido[3,4- d]pyrimidin-2-amine |
| 25A | 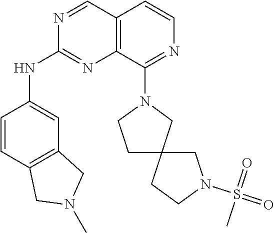 [SMILES: CN1Cc2ccc(Nc3ncc4ccnc(N5CCC6(CCN(S(C)(=O)=O)C6)C5)c4n3)cc2C1] | N-(2-methylisoindolin-5- yl)-8-(7-(methylsulfonyl)- 2,7-diazaspiro[4.4]nonan- 2-yl)pyrido[3,4- d]pyrimidin-2-amine |
| 26A | 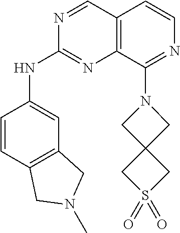 [SMILES: CN1Cc2ccc(Nc3ncc4ccnc(N5CC6(C5)CS(=O)(=O)C6)c4n3)cc2C1] | 6-(2-((2- methylisoindolin-5- yl)amino)pyrido[3,4- d]pyrimidin-8-yl)-2-thia- 6-azaspiro[3.3]heptane 2,2-dioxide |
| 27A | 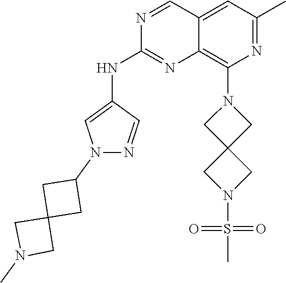 | 6-methyl-N-(1-(2-methyl- 2-azaspiro[3.3]heptan-6- yl)-1H-pyrazol-4-yl)-8- (6-(methylsulfonyl)-2,6- diazaspiro[3.3]heptan-2- yl)pyrido[3,4- d]pyrimidin-2-amine |
| 28A | 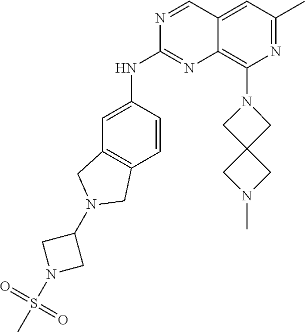 [SMILES: Cc1cc2cnc(Nc3ccc4c(c3)CN(C3CN(S(C)(=O)=O)C3)C4)nc2c(N2CC3(CN(C)C3)C2)n1] | 6-methyl-8-(6-methyl- 2,6- diazaspiro[3.3]heptan-2- yl)-N-(2-(1- (methylsulfonyl)azetidin- 3-yl)isoindolin-5- yl)pyrido[3,4- d]pyrimidin-2-amine |
| 29A | 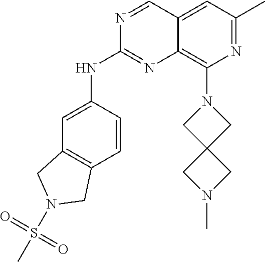 [SMILES: Cc1cc2cnc(Nc3ccc4c(c3)CN(S(C)(=O)=O)C4)nc2c(N2CC3(CN(C)C3)C2)n1] | 6-methyl-8-(6-methyl- 2,6- diazaspiro[3.3]heptan-2- yl)-N-(2- (methylsulfonyl)isoindolin- 5-yl)pyrido[3,4- d]pyrimidin-2-amine |
| 30A | 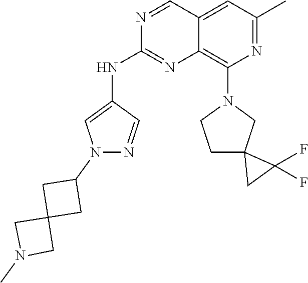 [SMILES: Cc1cc2cnc(Nc3cnn(C4CC5(C4)CN(C)C5)c3)nc2c(N2CCC3(C2)CC3(F)F)n1] | 8-(1,1-difluoro-5- azaspiro[2.4]heptan-5-yl)- 6-methyl-N-(1-(2-methyl- 2-azaspiro[3.3]heptan-6- yl)-1H-pyrazol-4- yl)pyrido[3,4- d]pyrimidin-2-amine |
| 31A | 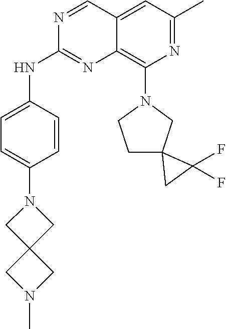 [SMILES: Cc1cc2cnc(Nc3ccc(N4CC5(CN(C)C5)C4)cc3)nc2c(N2CCC3(C2)CC3(F)F)n1] | 8-(1,1-difluoro-5- azaspiro[2.4]heptan-5-yl)- 6-methyl-N-(4-(6-methyl- 2,6- diazaspiro[3.3]heptan-2- yl)phenyl)pyrido[3,4- d]pyrimidin-2-amine |
| 32A | 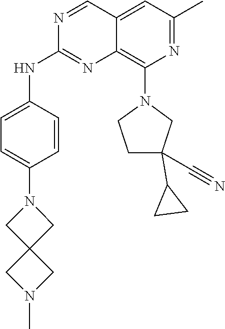 [SMILES: Cc1cc2cnc(Nc3ccc(N4CC5(CN(C)C5)C4)cc3)nc2c(N2CCC(C#N)(C3CC3)C2)n1] | 3-cyclopropyl-1-(6- methyl-2-((4-(6-methyl- 2,6- diazaspiro[3.3]heptan-2- yl)phenyl)amino)pyrido[3, 4-d]pyrimidin-8- yl)pyrrolidine-3- carbonitrile |
| 33A | 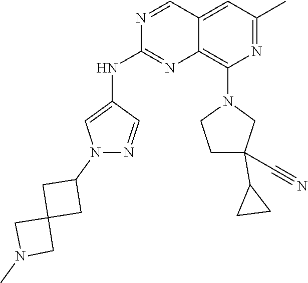 [SMILES: Cc1cc2cnc(Nc3cnn(C4CC5(C4)CN(C)C5)c3)nc2c(N2CCC(C#N)(C3CC3)C2)n1] | 3-cyclopropyl-1-(6- methyl-2-((1-(2-methyl- 2-azaspiro[3.3]heptan-6- yl)-1H-pyrazol-4- yl)amino)pyrido[3,4- d]pyrimidin-8- yl)pyrrolidine-3- carbonitrile |
| 34A | 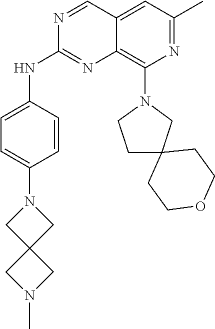 [SMILES: Cc1cc2cnc(Nc3ccc(N4CC5(CN(C)C5)C4)cc3)nc2c(N2CCC3(CCOCC3)C2)n1] | 6-methyl-N-(4-(6-methyl- 2,6- diazaspiro[3.3]heptan-2- yl)phenyl)-8-(8-oxa-2- azaspiro[4.5]decan-2- yl)pyrido[3,4- d]pyrimidin-2-amine |
| 35A | 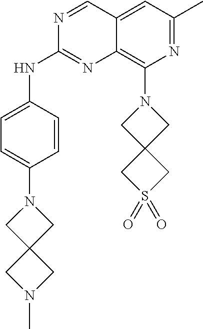 [SMILES: Cc1cc2cnc(Nc3ccc(N4CC5(CN(C)C5)C4)cc3)nc2c(N2CC3(C2)CS(=O)(=O)C3)n1] | 6-(6-methyl-2-((4-(6- methyl-2,6- diazaspiro[3.3]heptan-2- yl)phenyl)amino)pyrido[3, 4-d]pyrimidin-8-yl)-2- thia-6- azaspiro[3.3]heptane 2,2- dioxide |

### Example 5: Biochemical Assays

### CDK1/Cyclin B1 ADP-Glo Kinase Assay

The purpose of CDK1/Cyclin B1 assay is to evaluate the inhibition (% inhibition and IC₅₀ values) of small molecule inhibitors by using a Luminescent based ADP-Glo assay. CDK1/Cyclin B1 catalyzes the production of ADP from ATP. ADP-Glo assay monitors ADP producing biochemical reactions. ADP-Glo is performed in 2 steps upon completion of kinase reaction: a combined termination of kinase reaction and depletion of remaining ATP in the first step, and conversion of generated ADP to ATP and the newly produced ATP to light output using luciferase/luciferin reaction in the second step. The luminescent signal generated is proportional to the ADP concentration produced and is correlated with the kinase activity. CDK1/Cyclin B1 was purchased from Carna (Cat 04-102). Typical reaction solutions (10 uL final reaction volume) contained 2% DMSO (±inhibitor), 10 mM MgCI2, 1 mM EGTA, 0.05% BSA, 2 mM DTT, 80 uM ATP (ATP Km=78.6 uM), 0.01% Brig-35, 0.75 uM substrate, and 4.917 nM CDK1/Cyclin B1 enzyme complex in 50 mM HEPES buffer at pH 7.5. The assay was initiated with the addition of ATP-containing substrate solution, following a 30-minute pre-incubation of enzyme and inhibitor at room temperature in the reaction mixture. The reaction was stopped after 90 minutes at room temperature by the addition of 10 uL of ADP-GLO Reagent. After a 90 minute incubation, 20 uL of Kinase Detection Reagent was added. Samples were incubated for 40 minutes, after which plate well luminescence was measured on a Envision microplate reader. The IC₅₀ determinations were made from a plot of the fractional velocity as a function of inhibitor concentration fit to the 4 parameters IC₅₀ equation.

CDK2/Cyclin E1 Full length ADP-Glo Kinase Assay

The purpose of CDK2/Cyclin E1 assay is to evaluate the inhibition (% inhibition and IC₅₀ values) of small molecule inhibitors by using a Luminescent based ADP-Glo assay. CDK2/Cyclin E1 full length catalyzes the production of ADP from ATP. ADP-Glo assay monitors ADP producing biochemical reactions. ADP-Glo is performed in 2 steps upon completion of kinase reaction: a combined termination of kinase reaction and depletion of remaining ATP in the first step, and conversion of generated ADP to ATP and the newly produced ATP to light output using luciferase/luciferin reaction in the second step. The luminescent signal generated is proportional to the ADP concentration produced and is correlated with the kinase activity. CDK2/Cyclin E1 was purchased from Eurofins (Cat 14-475M). Typical reaction solutions (10 uL final reaction volume) contained 2% DMSO (±inhibitor), 10 mM MgCI2, 1 mM EGTA, 0.05% BSA, 2 mM DTT, 20 uM ATP (ATP Km=64.78 uM), 0.01% Brig-35, 0.75 uM substrate, and 0.328 nM wild-type full length CDK2/Cyclin E1 enzyme complex in 50 mM HEPES buffer at pH 7.5. The assay was initiated with the addition of ATP-containing substrate solution, following a 30-minute pre-incubation of enzyme and inhibitor at room temperature in the reaction mixture. The reaction was stopped after 90 minutes at room temperature by the addition of 10 uL of ADP-GLO Reagent. After a 90 minute incubation, 20 uL of Kinase Detection Reagent was added. Samples were incubated for 40 minutes, after which plate well luminescence was measured on a Envision microplate reader. The IC₅₀ determinations were made from a plot of the fractional velocity as a function of inhibitor concentration fit to the 4 parameters IC₅₀ equation.

### CDK4/Cyclin D1 CHEF Assay

The purpose of CDK4/Cyclin D1 assay is to evaluate the inhibition (% inhibition and IC₅₀ values) of small molecule inhibitors by using a Chelation-Enhance Fluorescence (CHEF) assay. In a CHEF assay, phosphorylation of a peptide substrate results in proportional increase in fluorescence. CHEF kinase assay use peptide substrates containing a synthetic alpha-amino acid with a side chain bearing an 8-hydroxyquinoline derivative (sulfonamido-oxide, Sox). Upon phosphorylation of a nearby serine, threonine or tyrosine and in the presence of Mg(II), the spectral properties of the Sox residue are altered, emitting 485 nm wavelength light when excited with a 360 nm wavelength light source. CDK4/Cyclin D1 catalyzes the phosphoryl transfer to the SOX-labeled substrate peptide AQT0258 from Assayquant Technologies. Typical reaction solutions contained 2% DMSO (+/− inhibitor), 10 mM MgCl2, 1 mM DTT, 200 uM ATP (ATP Km=195.2 uM), 0.012% Brig-35, 10 uM AQT0258 peptide, 0.02% BSA, 1% Glycerol, 0.55 mM EGTA, 2.5 nM CDK4/Cyclin D1 in 54 mM HEPES buffer at pH 7.5. The reaction was initiated with the addition of substrate solution, following a 30-minute pre-incubation of enzyme and inhibitor at 22° C. in the reaction mix. Reactions were allowed to proceed for 3 hrs at 22° C., followed by fluorescence read of the reaction. The IC₅₀ determinations were made from a plot of the fractional velocity as a function of inhibitor concentration fit to the 4 parameters IC₅₀ equation.

### CDK4/Cyclin D1 Mobility Shift Assay (MSA)

The purpose CDK4/Cyclin D1 assay is to evaluate the inhibition (% inhibition and IC₅₀ values) in the presence of small molecule inhibitors by using a fluorescence based microfluidic mobility shift assay. CDK4/Cyclin D1 catalyzes the production of ADP from ATP that accompanies the phosphoryl transfer to the substrate peptide 5-FAM-Dyrktide (5-FAM-RRRFRPASPLRGPPK) (Perkin Elmer Peptide 34). The mobility shift assay (MSA) electrophoretically separates the fluorescently labelled peptides (substrate and phosphorylated product) following the kinase reaction. Both substrate and product are measured, and the ratio of these values is used to generate % conversion of substrate to product by the LabChip EZ Reader. Typical reaction solutions contained 2% DMSO (+/− inhibitor), 10 mM MgCl2, 1 mM EGTA, 0.05% BSA, 2 mM DTT, 0.2 mM ATP, 0.01% Brig-35, 1.5 uM 5-FAM-Dyrktide, 2.5 nM CDK4/Cyclin D1 in 50 mM HEPES buffer at pH 7.5. The reaction was initiated with the addition of substrate solution, following a 30-minute pre-incubation of enzyme and inhibitor at 22° C. in the reaction mix. The reaction was stopped after 180 minutes by the addition of 75 uL of 500 mM EDTA and measured on a Perkin Elmer EZ reader instrument. IC₅₀ determinations were made from a plot of the fractional velocity as a function of inhibitor concentration fit to the 4 parameters IC₅₀ equation.

### CDK6/Cyclin D3 ADP-Glo Kinase Assay

The purpose of the CDK6/Cyclin D3 assay is to evaluate the inhibition (% inhibition and IC₅₀ values) in the presence of small molecule inhibitors by using a Luminescent based ADP-Glo assay. CDK6/Cyclin D3 catalyzes the production of ADP from ATP. ADP-Glo assay monitors ADP producing biochemical reactions. ADP-Glo is performed in 2 steps upon completion of kinase reaction: a combined termination of kinase reaction and depletion of remaining ATP in the first step, and conversion of generated ADP to IP and the newly produced ATP to light output using luciferase/luciferin reaction in the second step. The luminescent signal generated is proportional to the ADP concentration produced and is correlated with the kinase activity CDK6/Cyclin D3 was purchased from Carna. Typical reaction solutions (10 uL final reaction volume) contained 2% DMSO (±inhibitor), 10 mM MgCI2, 1 mM EGTA, 0.05% BSA, 2 mM DTT, 100 uM ATP (ATP Km=291.7 uM), 0.01% Brig-35, 0.75 uM substrate, and 5 nM wild-type CDK6/Cyclin D3 enzyme complex in 50 mM HEPES buffer at pH 7.5. The assay was initiated with the addition of ATP-containing substrate solution, following a 30-minute pre-incubation of enzyme and inhibitor at room temperature in the reaction mixture. The reaction was stopped after 90 minutes at room temperature by the addition of 10 uL of ADP-GLO Reagent. After a 90-minute incubation, 20 uL of Kinase Detection Reagent was added. Samples were incubated for 40 minutes, after which plate well luminescence was measured on a Envision microplate reader. The IC₅₀ determinations were made from a plot of the fractional velocity as a function of inhibitor concentration fit to the 4 parameters IC₅₀ equation.

### Cell Growth Inhibition

MCF-7 and OVCAR-3 cells were used to evaluate the anti-proliferation activity of the CDK inhibitors. MCF-7 (ATCC, HTB-22) cells are epithelial cells from a female patient with ER+ metastatic adenocarcinoma. OVCAR-3 (ATCC, HTB-161) cells were derived from malignant ascites of a patient with ovarian cancer and are known to have CCNE1 amplification. Both cell lines were maintained in RPMI media supplemented with 10% fetal bovine serum. For cell growth inhibition assay, CDK inhibitors in DMSO solution were dispensed with either Echo 655 (Beckman Coulter) or Tecan D300e (HP) into 384-well plates (Corning #3765) and the 384-well plates were UV-sterilized prior to the assay. The inhibitors were typically tested in the 10-10,000 nM concentration range with half-log serial dilutions. MCF-7 or OVCAR-3 (500 cells/30 μL/well) were added to each well using Multidrop Combi (ThermoFisher) using standard cassettes. The assay plates with cells were cultured at 37° C., 5% CO₂ for 6 days. At the end of the 6-day treatment, 30 μL of CellTiterGlo 2.0 (Promega) was added to each well and the luminescent signal was read using CLARIOstar plus (BMG). The percentage of cell growth inhibition (% CGI) was calculated using the following formula 00 CGI=100-100×luminescencesamₚie/luminescencecontroi. The half maximal inhibitory concentration (IC50) was determined by nonlinear curve fitting (four parameters, variable slope).

Certain compounds of the disclosure have IC₅₀ values as in Table 3.

TABLE 3
All IC₅₀ values in Table 3 are reported
as the following: ++++ = IC₅₀ < 200 nM; +++ = 200 nM < IC50 <
500 nM; ++ = 500 nM < IC50 < 2000 nM; + = IC50 > 2000 nM

| | | | CDK4/ | CDK4/ | | | |
|---|---|---|---|---|---|---|---|
| | CDK1 | CDK2/ | CyclinD1 | CyclinD1 | CDK6/ | MCF-7 | OVCAR-3 |
| Cmpd | Cyclin B1 | CyclinE1 | MSA | AQT | CyclinD3 | Cell | Cell |
| # | IC₅₀ nM | IC₅₀ nM | IC₅₀ nM | IC₅₀ nM | IC₅₀ nM | IC₅₀ nM | IC₅₀ nM |
| 1 | ++ | ++++ | ++++ | | +++ | ++ | + |
| 2 | ++++ | ++++ | ++++ | | ++++ | ++ | ++ |
| 3 | ++++ | ++++ | ++++ | | ++++ | ++ | ++++ |
| 4 | +++ | ++++ | ++++ | | ++++ | +++ | ++ |
| 5 | ++++ | ++++ | ++++ | | ++++ | ++ | ++ |
| 6 | + | ++ | ++++ | | ++++ | ++ | + |
| 7 | +++ | ++++ | ++++ | | ++++ | ++ | ++ |
| 8 | +++ | | ++++ | | | ++ | ++++ |
| 9 | +++ | | ++++ | | | ++ | ++ |
| 10 | + | ++++ | ++++ | | ++++ | ++ | ++ |
| 11 | ++++ | ++++ | | ++++ | ++++ | ++ | +++ |
| 12 | ++++ | ++++ | | ++++ | ++++ | | |
| 13 | ++++ | ++++ | | ++++ | ++++ | | |
| 14 | +++ | ++++ | ++++ | | ++++ | + | + |
| 15 | + | ++ | ++ | | + | + | + |
| 16 | ++++ | ++++ | ++++ | | ++++ | ++ | ++ |
| 17 | + | +++ | ++++ | | +++ | ++ | ++ |
| 18 | + | ++ | ++++ | | ++++ | +++ | + |
| 19 | + | ++++ | ++ | | + | + | + |
| 20 | ++ | ++++ | ++++ | | ++++ | ++ | +++ |
| 21 | + | +++ | ++++ | | ++++ | | |
| 22 | ++++ | ++++ | ++++ | | ++++ | +++ | +++ |
| 23 | ++ | ++++ | | ++++ | ++++ | ++++ | ++++ |
| 24 | ++ | ++++ | | ++++ | ++++ | ++ | ++ |
| 25 | +++ | ++++ | | ++++ | ++++ | +++ | +++ |
| 26 | + | +++ | | +++ | ++ | + | + |
| 27 | ++ | ++++ | | ++ | ++ | + | ++ |
| 28 | ++ | ++++ | | ++++ | ++++ | ++ | ++ |
| 29 | + | +++ | | ++++ | ++ | + | + |
| 30 | + | +++ | | +++ | +++ | + | + |
| 31 | + | ++++ | | + | + | + | + |
| 32 | ++ | ++++ | | ++++ | ++++ | ++ | ++ |
| 33 | ++ | +++ | | ++++ | ++++ | ++ | ++ |
| 34 | ++ | ++++ | | ++++ | ++++ | ++ | ++ |
| 35 | + | +++ | | ++++ | ++++ | ++ | ++ |
| 36 | + | +++ | | +++ | ++ | + | + |
| 37 | + | +++ | | + | + | + | + |
| 38 | + | ++++ | | ++ | + | + | ++ |
| 39 | + | ++++ | | +++ | + | ++ | + |
| 40 | + | ++++ | | ++++ | ++++ | ++ | ++ |
| 41 | ++++ | ++++ | | ++++ | ++++ | + | + |
| 42 | + | ++++ | | ++++ | ++ | ++ | ++ |
| 43 | + | ++ | | ++++ | +++ | ++ | ++ |
| 44 | + | + | | +++ | ++ | + | + |
| 45 | 1 | + | | ++++ | ++ | + | + |
| 46 | ++++ | ++++ | | ++++ | ++++ | ++ | ++ |
| 47 | ++ | ++++ | | ++++ | ++++ | ++ | ++ |
| 48 | ++ | ++++ | | ++++ | +++ | + | + |
| 49 | ++ | ++++ | | ++++ | +++ | ++ | ++ |
| 50 | ++ | ++++ | | ++++ | ++++ | ++ | ++ |
| 51 | ++ | ++++ | | ++++ | +++ | ++ | ++ |
| 52 | ++++ | ++++ | | ++++ | ++++ | + | ++ |
| 53 | + | ++++ | | ++++ | ++++ | + | + |
| 54 | ++++ | ++++ | | ++++ | ++++ | + | ++ |
| 55 | ++++ | ++++ | | ++++ | ++++ | +++ | ++ |
| 56 | ++++ | ++++ | | ++++ | ++++ | ++ | ++ |
| 57 | +++ | ++++ | | ++++ | ++++ | + | + |
| 58 | ++++ | ++++ | | ++++ | ++++ | ++ | ++ |
| 59 | ++ | ++++ | | ++++ | ++++ | ++ | ++ |
| 60 | ++++ | ++++ | | ++++ | ++++ | ++ | + |
| 61 | ++++ | ++++ | | ++++ | ++++ | ++ | ++ |
| 62 | ++++ | ++++ | | ++++ | ++++ | ++ | ++ |
| 63 | + | +++ | | ++ | + | + | + |
| 64 | ++ | ++++ | | ++++ | +++ | + | + |
| 65 | ++++ | ++++ | | ++++ | ++++ | +++ | +++ |
| 66 | ++ | ++++ | | ++++ | ++++ | ++ | ++ |
| 67 | +++ | ++++ | | ++++ | +++ | + | + |
| 68 | + | ++++ | | +++ | ++ | | |
| 69 | ++++ | ++++ | | ++++ | ++++ | | |
| 70 | ++++ | ++++ | | ++++ | ++++ | ++ | ++ |
| 71 | +++ | ++++ | | ++++ | ++++ | + | + |
| 72 | ++ | ++++ | | +++ | +++ | + | + |
| 73 | ++ | ++++ | | ++++ | +++ | + | + |
| 74 | ++ | ++++ | | +++ | +++ | + | + |
| 75 | + | ++++ | | ++++ | ++++ | + | + |
| 76 | +++ | ++++ | | ++++ | ++++ | ++ | + |
| 77 | +++ | ++++ | | ++++ | ++++ | + | + |
| 78 | +++ | ++++ | | ++++ | +++ | ++ | ++ |
| 79 | +++ | ++++ | | ++++ | +++ | + | ++ |
| 80 | ++ | ++++ | | ++++ | ++++ | + | + |
| 81 | +++ | ++++ | | ++++ | +++ | | |
| 82 | +++ | ++++ | | +++ | +++ | | |
| 83 | +++ | ++++ | | ++++ | ++++ | + | + |
| 84 | +++ | ++++ | | ++++ | ++++ | ++ | ++ |
| 85 | ++++ | ++++ | | ++++ | ++++ | ++ | ++ |
| 86 | + | +++ | | ++++ | +++ | + | + |
| 87 | ++++ | ++++ | | ++++ | ++++ | ++ | +++ |
| 88 | +++ | ++++ | | ++++ | ++++ | | |
| 89 | ++++ | ++++ | | ++++ | ++++ | ++ | +++ |
| 90 | ++++ | ++++ | | ++++ | ++++ | ++ | ++ |
| 91 | ++++ | ++++ | | ++++ | ++++ | ++ | +++ |
| 92 | ++++ | ++++ | | ++++ | ++++ | ++ | ++ |
| 93 | ++++ | ++++ | | ++++ | ++++ | ++ | ++ |
| 94 | ++++ | ++++ | | ++++ | ++++ | ++ | +++ |
| 95 | ++++ | ++++ | | ++++ | ++++ | ++ | ++ |
| 96 | ++++ | ++++ | | ++++ | ++++ | +++ | ++ |
| 97 | ++++ | ++++ | | ++++ | ++++ | ++ | +++ |
| 98 | +++ | ++++ | | ++++ | +++ | | |
| 99 | ++++ | ++++ | | ++++ | ++++ | | |
| 100 | ++++ | ++++ | | ++++ | ++++ | | |
| 101 | +++ | ++++ | | ++++ | ++++ | | |
| 102 | ++++ | ++++ | | ++++ | ++++ | | |
| 103 | ++ | ++++ | | ++++ | ++++ | | |
| 104 | ++ | ++++ | | ++++ | +++ | | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A compound, or a pharmaceutically acceptable salt or solvate thereof, having the structure of Formula (I0): 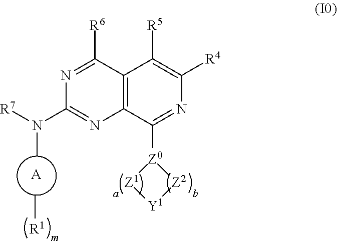
wherein,
A is a ring selected from optionally substituted carbocycle, optionally substituted 4- to 8-membered heterocycle, optionally substituted tetrahydro-triazolopyrazine, and optionally substituted isoindoline;
Z⁰ is —C(H)— or nitrogen;
each of Z¹, Z², and Y¹ are independently selected from —C(R²)₂—, —C(O)—, —NR³—, —N(C(O)R²)—, —NS(O₂)R², —O—, —S—, —S(O)—, and —S(O)₂—;
each of a and b are independently selected from 1, 2, 3, and 4;
each R¹ is independently selected from halogen, —CN, —NO₂, optionally substituted alkyl, optionally substituted alkenyl, optionally substituted alkynyl, optionally substituted carbocycle, and optionally substituted heterocycle;
m is selected from 0 to 5;
each R² is independently selected from hydrogen, halogen, —CN, —OH, optionally substituted alkoxy, optionally substituted alkyl, optionally substituted cycloalkyl, optionally substituted —O-cycloalkyl, and optionally substituted heterocycloalkyl, or two R² substituents come together to form an optionally substituted heterocycle or an optionally substituted carbocycle, or R² and R³ substituents come together to form an optionally substituted heterocycle;
each R³ is independently selected from hydrogen, optionally substituted alkyl, optionally substituted C₃₋₄ carbocycle, and optionally substituted 3- to 4-membered heterocycloalkyl;
R⁴ is selected from hydrogen, halogen, —CN, optionally substituted C₁₋₄ alkyl, optionally substituted C₃₋₄ carbocycle, and optionally substituted 3- to 4-membered heterocycloalkyl;
each of R⁵, R⁶, is independently selected from hydrogen, halogen, —CN, optionally substituted C₁₋₄ alkyl, optionally substituted C₃₋₄ carbocycle, and optionally substituted 3- to 4-membered heterocyclcoalkyl; and
R⁷ is selected from hydrogen and optionally substituted C₁₋₄ alkyl, and
wherein if A is optionally substituted phenyl, m is from 1 to 5 and at least one R¹ is a heterocycloalkyl,
wherein if A is optionally substituted pyridine, optionally substituted pyridazine, or optionally substituted pyrimidine, R⁴ is selected from hydrogen, halogen, and —CN,
wherein if A is an optionally substituted piperidine sulfonamide, then either (i) Y¹ is —C(R²)₂— and the two R² substituents come together to form a ring selected from an optionally substituted heterocycle and an optionally substituted carbocycle, or (ii) R⁴ is selected from hydrogen, halogen, methyl, and —CN, and
wherein if A-R¹ is 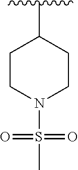 [SMILES: *C1CCN(S(C)(=O)=O)CC1]
and Z⁰ is CH, then R⁴ is methyl or cyclopropyl.

2. The compound, or a pharmaceutically acceptable salt or solvate thereof, of claim 1 having the structure of Formula (I): 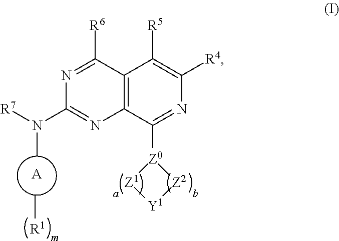
wherein, A is a ring selected from optionally substituted carbocycle, optionally substituted 4- to 6-membered heterocycle, and optionally substituted isoindoline.

3. The compound, or a pharmaceutically acceptable salt or solvate thereof, of claim 1 having the structure of one or more of Formulae (IA), (IB), (IC), (ID), or (IE): 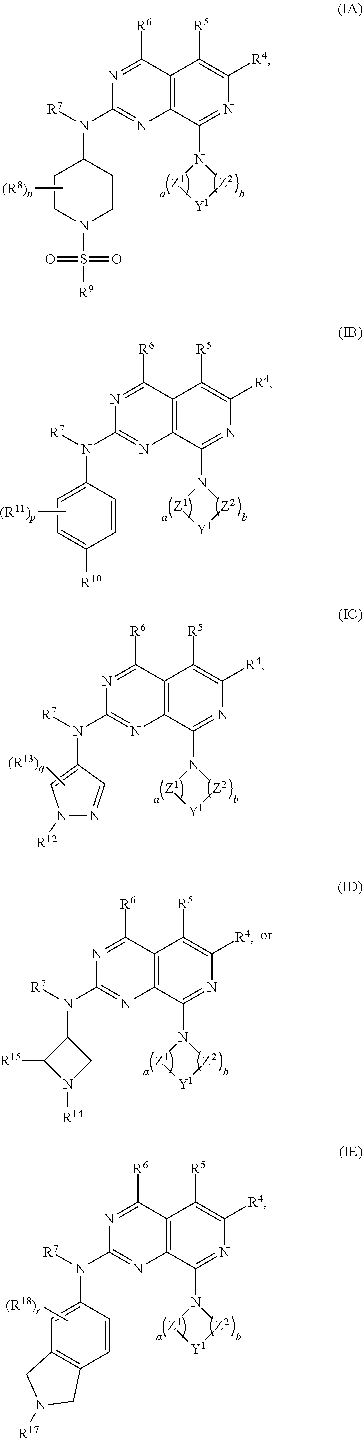
wherein,
R⁸ is selected from halogen, —CN, and optionally substituted C₁₋₄ alkyl;
R⁹ is selected from optionally substituted C₃₋₆ carbocycle, optionally substituted C₅₋₆ heteroaryl, and 3- to 6-membered heterocycloalkyl;
R¹⁰ is optionally substituted alkyl or optionally substituted heterocycloalkyl;
R¹¹ is selected from halogen, —CN, and optionally substituted C₁₋₄ alkyl;
R¹² is selected from an optionally substituted heterocycloalkyl, optionally substituted heterocycloalkylalkyl, optionally substituted cycloalkyl, and optionally substituted cycloalkylalkyl; or R¹² and R¹³ come together to form an optionally substituted heterocycle;
R¹³ is selected from halogen, —CN, and optionally substituted C₁₋₄ alkyl;
n is selected from 0 to 9;
R¹⁴ is selected from —SOR¹⁶—, and optionally substituted heterocycloalkyl;
R¹⁵ is selected from hydrogen, halogen, —CN, and optionally substituted C₁₋₄ alkyl;
R¹⁶ is selected from optionally substituted C₁₋₄ alkyl, optionally substituted C₃₋₆ carbocycle, and optionally substituted 3- to 6-membered heterocycloalkyl;
R¹⁷ is selected from —SOR¹⁹—, optionally substituted alkyl, optionally substituted carbocycle, and optionally substituted heterocycloalkyl;
R¹⁸ is selected from halogen, —CN, and optionally substituted C₁₋₄ alkyl;
R¹⁹ is selected from optionally substituted C₁₋₄ alkyl, optionally substituted C₃₋₆ carbocycle, and optionally substituted 3- to 6-membered heterocycloalkyl;
r is selected from 0 to 5.
p is selected from 0 to 4;
q is selected from 0 to 2; and
wherein when the compound is of formula (IA), either (i) Y¹ is —C(R²)₂— and the two R² substituents come together to form a ring selected from an optionally substituted heterocycle and an optionally substituted carbocycle, or (ii) R⁴ is selected from hydrogen, halogen, and —CN.

4. The compound or pharmaceutically acceptable salt of claim 1, wherein Z¹ and Z² are independently selected from —C(R²)₂—, —NR³—, —O—, and —S—.

5. The compound or pharmaceutically acceptable salt of claim 4, wherein Z¹ and Z² are independently —C(R²)₂—.

6. The compound or pharmaceutically acceptable salt of claim 1, wherein each of a and b are independently selected from 1 and 2.

7. The compound or pharmaceutically acceptable salt of claim 1, wherein Y¹ is selected from —C(R²)₂—, —NR³—, —NS(O₂)R², —O—, —S(O)₂—, and —S—.

8. The compound or pharmaceutically acceptable salt of claim 7, wherein Y¹ is selected from —C(R²)₂— and —NR³.

9. The compound or pharmaceutically acceptable salt of claim 1, wherein each R² is independently selected from hydrogen, OH, halogen, —CN, optionally substituted alkyl, optionally substituted —O-alkyl, optionally substituted cycloalkyl, and optionally substituted heterocycloalkyl.

10. The compound or pharmaceutically acceptable salt of claim 9, wherein each R² is independently selected from hydrogen, halogen, —CN, cyclopropyl, cyclobutyl, optionally substituted cycloalkyl, and optionally substituted heterocycloalkyl.

11. The compound or pharmaceutically acceptable salt of claim 1, wherein two R² substituents come together to form an optionally substituted heterocycle or an optionally substituted carbocycle.

12. The compound or pharmaceutically acceptable salt of claim 1, wherein each R³ is selected from hydrogen, optionally substituted alkyl, cyclopropyl, cyclobutyl, optionally substituted oxetane, and optionally substituted azetidine.

13. The compound, or a pharmaceutically acceptable salt or solvate thereof of claim 1 having the structure of one or more of Formulae (IAA), (IBB), (ICC), (IDD), or (IEE): 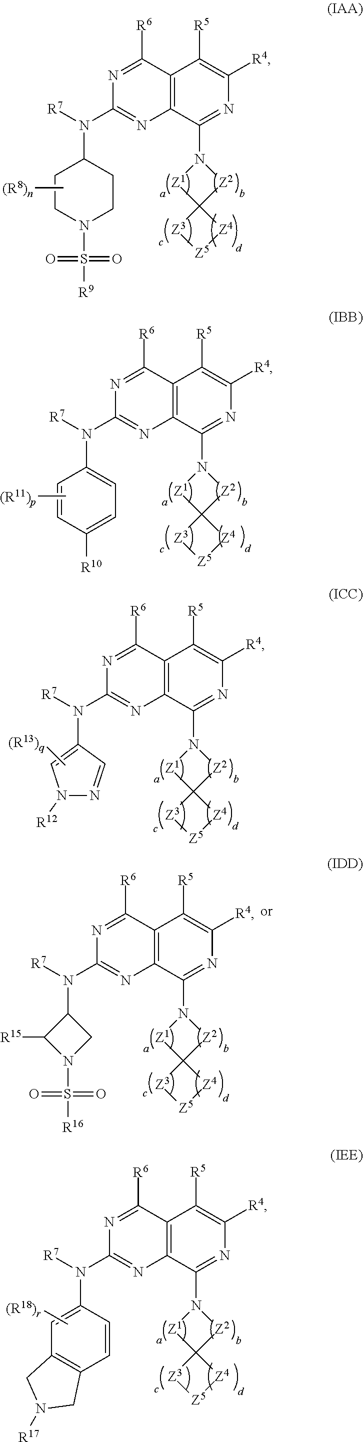
wherein,
R⁸ is selected from halogen, —CN, and optionally substituted C₁₋₄ alkyl;
R⁹ is selected from optionally substituted C₃₋₆ carbocycle, optionally substituted C₅₋₆ heteroaryl, and 3- to 6-membered heterocycloalkyl;
R¹⁰ is optionally substituted heterocycloalkyl;
R¹¹ is selected from halogen, —CN, and optionally substituted C₁₋₄ alkyl;
R¹² is optionally substituted heterocycloalkyl; or R¹² and R¹³ come together to form an optionally substituted heterocycle;
R¹³ is selected from halogen, —CN, and optionally substituted C₁₋₄ alkyl;
R¹⁵ is selected from hydrogen, halogen, —CN, and optionally substituted C₁₋₄ alkyl;
R¹⁶ is selected from optionally substituted C₁₋₄ alkyl, optionally substituted C₃₋₆ carbocycle, and optionally substituted 3- to 6-membered heterocycloalkyl;
R¹⁹ is selected from optionally substituted C₁₋₄ alkyl, optionally substituted C₃₋₆ carbocycle, and optionally substituted 3- to 6-membered heterocycloalkyl;
n is selected from 0 to 9;
p is selected from 0 to 4;
q is selected from 0 to 2;
each of Z¹, Z², Z³, Z⁴ and Z⁵ are independently selected from —C(R²)₂—, —C(O)—, —NR³—, —N(C(O)R²)—, —NS(O₂)R³, —O—, —S—, —S(O)—, and —S(O)₂—, wherein Z⁵ is additionally selected from a bond; and
each of a, b, c, and d are independently selected from 1, 2, 3, and 4.

14. The compound or pharmaceutically acceptable salt of claim 13, wherein each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond.

15. The compound or pharmaceutically acceptable salt of claim 14, wherein each of Z¹, Z², Z³, Z⁴ and Z⁵ are independently selected from —C(R²)₂—, —NR³—, —O—, and —S(O)₂—, wherein Z⁵ is additionally selected from a bond.

16. The compound or pharmaceutically acceptable salt of 13, wherein each R² is independently selected from hydrogen, halogen, —CN, OH, optionally substituted alkyl, optionally substituted cycloalkyl, and optionally substituted heterocycloalkyl, or two R² substituents come together to form an optionally substituted heterocycle or an optionally substituted carbocycle.

17. The compound or pharmaceutically acceptable salt of claim 16, wherein each R² is selected from hydrogen, fluoro, CN, cyclopropyl, cyclobutyl, —C₁₋₄ alkyl, —C₁₋₄ haloalkyl, —O—C₁₋₄ alkyl, —C₁₋₄ alkylene-O—C₁₋₃ alkyl, —C₁₋₄ alkylene-OH, optionally substituted oxetane, and optionally substituted azetidine.

18. The compound or pharmaceutically acceptable salt of claim 17, wherein R³ is selected from hydrogen, methyl, ethyl, propyl, CD3, and cyclopropyl.

19. The compound or pharmaceutically acceptable salt of claim 13, wherein each a, b, c, and d are each independently selected from 1 and 2.

20. The compound or pharmaceutically acceptable salt of claim 1, wherein A is selected from optionally substituted cyclohexane, optionally substituted pyridine, optionally substituted piperidine, optionally substituted tetrahydropyran, optionally substituted azabicyclo[3.1.0]hexane, optionally substituted azetidine, and optionally substituted tetrahydroisoquinoline.

21. The compound, or pharmaceutically acceptable salt thereof, of claim 20, wherein A is optionally substituted piperidine.

22. The compound or pharmaceutically acceptable salt of claim 21, wherein A is substituted with SO₂R⁹, wherein R⁹ is selected from optionally substituted C₃₋₆ carbocycle and optionally substituted C₅₋₆ heteroaryl.

23. The compound or pharmaceutically acceptable salt of claim 1, wherein m is selected from 0 to 1.

24. The compound or pharmaceutically acceptable salt of claim 1, wherein each R¹ is independently selected from optionally substituted alkyl, optionally substituted carbocycle, and optionally substituted heterocycle.

25. The compound or pharmaceutically acceptable salt of claim 1, wherein the N containing heterocyclic ring depicted as 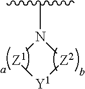
in Formula (IA) is selected from optionally substituted azetidine, optionally substituted pyrrolidine, optionally substituted piperidine, optionally substituted piperazine, optionally substituted morpholine, optionally substituted tetrahydrothienopyrroledioxide, and optionally substituted dihydroindole.

26. The compound or pharmaceutically acceptable salt of claim 25, wherein the N containing heterocyclic ring depicted as 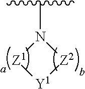
in Formula (IA) is selected from

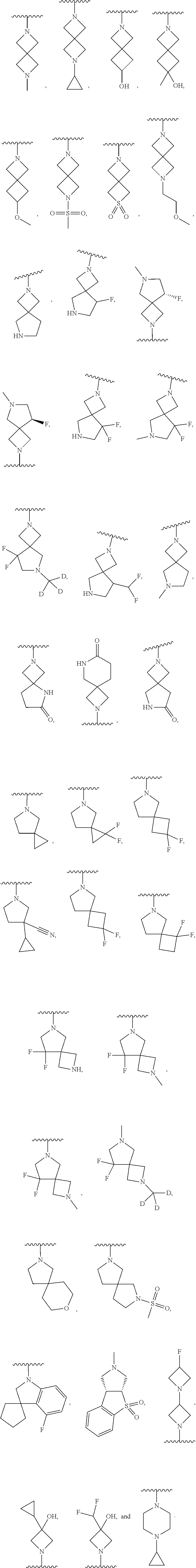

27. The compound or pharmaceutically acceptable salt of claim 1, wherein R⁴ is selected from hydrogen, halogen, optionally substituted C₁₋₂ alkyl, optionally substituted C₃₋₄ carbocycle, and optionally substituted 3- to 4 membered heterocycloalkyl.

28. The compound or pharmaceutically acceptable salt of claim 1, wherein R⁴ is selected from hydrogen, methyl, halogen, and —CN.

29. The compound or pharmaceutically acceptable salt of claim 1, wherein R⁵ is selected from hydrogen, halogen, optionally substituted C₁₋₂ alkyl, optionally substituted C₃₋₄ carbocycle, and optionally substituted 3- to 4 membered heterocycloalkyl.

30. The compound or pharmaceutically acceptable salt of claim 1, wherein R⁶ is selected from hydrogen, halogen, optionally substituted C₁₋₂ alkyl, optionally substituted C₃₋₄ carbocycle, and optionally substituted 3- to 4 membered heterocycloalkyl.

31. The compound or pharmaceutically acceptable salt of claim 1, wherein R⁷ is hydrogen.

32. The compound or pharmaceutically acceptable salt of claim 3, wherein R⁹ is selected from cyclopentyl, methylcyclopentyl, cyclobutylmethylene, cyclopentylmethylene, and n-methyl pyrazolyl.

33. The compound, or pharmaceutically acceptable salt thereof, of claim 1, wherein the compound is selected from Table 1.

34. A pharmaceutical composition comprising a compound or pharmaceutically acceptable salt of claim 1 and a pharmaceutically acceptable excipient.

35. A method of treating cancer, comprising administering to a subject in need thereof a compound, or a pharmaceutically acceptable salt thereof, according to claim 1.

36. The method of claim 35, wherein the cancer is a solid tumor.

37. The method of claim 36, wherein the cancer is selected from ovarian cancer, breast cancer, colon cancer, and brain cancer.

38. A method of inhibiting a cyclin dependent kinase (CDK) in a cell with a compound, or pharmaceutically acceptable salt thereof, of claim 1.

39. The method of claim 38, wherein the CDK is selected from CDK 2, CDK 4, CD6, or any combination thereof.

40. The method of claim 38, wherein the CDK is selected from CDK 2/4, CDK 2/6, CDK 4/6, and CDK 2/4/6.
